SEQUENCE LISTING (234 sequences)

SEQ ID NO: 1 - protein, 133 aa, Rattus norvegicus
MESQTHVLISLLLSVSGTYGDIAITQSPSSVAVSVGETVTLSCKSSQSLLYSENQKDYLGWYQQKPGQTPKPLIYWATNRHTGVPDRFTGSGSGTDFTLIISSVQAEDLADYYCGQYLVYPFTFGPGTKLELK

SEQ ID NO: 2 - protein, 137 aa, Rattus norvegicus
MGWSQIILFLVAAATCVHSQVQLQQSGAELVKPGSSVKISCKASGYTFTSNFMHWVKQQPGNGLEWIGWIYPEYGNTKYNQKFDGKATLTADKSSSTAYMQLSSLTSEDSAVYFCASEEAVISLVYWGQGTLVTVSS

SEQ ID NO: 3 - protein, 105 aa, Canis lupus
QPKASPSVTLFPPSSEELGANKATLVCLISDFYPSGVTVAWKASGSPVTQGVETTKPSKQSNNKYAASSYLSLTPDKWKSHSSFSCLVTHEGSTVEKKVAPAECS

SEQ ID NO: 4 - protein, 331 aa, Canis lupus
ASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 5 - DNA, 399 bp, Rattus norvegicus
  source 1..399: mol_type = other DNA
atggaatcacagacgcatgtcctcatttcccttctgctctcggtatctggtacctatggggacattgcgataacccagtctccatcctctgtggctgtgtcagtaggagagacggtcactctgagctgcaagtccagtcagagtcttttatacagtgaaaaccaaaaggactatttgggctggtaccagcagaaaccagggcagactcctaaaccccttatctactgggcaaccaaccggcacactggggtccctgatcgcttcacaggtagtggatccgggacagacttcactctgatcatcagcagtgtgcaggctgaagacctggctgattattactgtgggcagtaccttgtctatccgttcacgtttggacctgggaccaagctggaactgaaa

SEQ ID NO: 6 - DNA, 411 bp, Rattus norvegicus
  source 1..411: mol_type = other DNA
atgggatggagccagatcatcctctttctggtggcagcagctacatgtgttcactcccaggtacagctgcagcaatctggggctgaattagtgaagcctgggtcctcagtgaaaatttcctgcaaggcttctggctacaccttcaccagtaactttatgcactgggtaaagcagcagcctggaaatggccttgagtggattgggtggatttatcctgaatatggtaatactaagtacaatcaaaagttcgatgggaaggcaacactcactgcagacaaatcctccagcacagcctatatgcagctcagcagcctgacatctgaggactctgcagtctatttctgtgcaagtgaggaggcagttatatcccttgtttactggggccaaggcactctggtcactgtctcttca

SEQ ID NO: 7 - DNA, 318 bp, Canis lupus
  source 1..318: mol_type = other DNA
cagcccaaggcctccccctcggtcacactcttcccgccctcctctgaggagctcggcgccaacaaggccaccctggtgtgcctcatcagcgacttctaccccagcggcgtgacggtggcctggaaggcaagcggcagccccgtcacccagggcgtggagaccaccaagccctccaagcagagcaacaacaagtacgcggccagcagctacctgagcctgacgcctgacaagtggaaatctcacagcagcttcagctgcctggtcacgcacgaggggagcaccgtggagaagaaggtggcccccgcagagtgctcttag

SEQ ID NO: 8 - DNA, 996 bp, Canis lupus
  source 1..996: mol_type = other DNA
gcctccaccacggccccctcggttttcccactggcccccagctgcgggtccacttccggctccacggtggccctggcctgcctggtgtcaggctacttccccgagcctgtaactgtgtcctggaattccggctccttgaccagcggtgtgcacaccttcccgtccgtcctgcagtcctcagggctctactccctcagcagcacggtgacagtgccctccagcaggtggcccagcgagaccttcacctgcaacgtggtccacccggccagcaacactaaagtagacaagccagtgcccaaagagtccacctgcaagtgtatatccccatgcccagtccctgaatcactgggagggccttcggtcttcatctttcccccgaaacccaaggacatcctcaggattacccgaacacccgagatcacctgtgtggtgttagatctgggccgtgaggaccctgaggtgcagatcagctggttcgtggatggtaaggaggtgcacacagccaagacgcagcctcgtgagcagcagttcaacagcacctaccgtgtggtcagcgtcctccccattgagcaccaggactggctcaccggaaaggagttcaagtgcagagtcaaccacataggcctcccgtcccccatcgagaggactatctccaaagccagagggcaagcccatcagcccagtgtgtatgtcctgccaccatccccaaaggagttgtcatccagtgacacggtcaccctgacctgcctgatcaaagacttcttcccacctgagattgatgtggagtggcagagcaatggacagccggagcccgagagcaagtaccacacgactgcgccccagctggacgaggacgggtcctacttcctgtacagcaagctctctgtggacaagagccgctggcagcagggagacaccttcacatgtgcggtgatgcatgaagctctacagaaccactacacagatctatccctctcccattctccgggtaaatga

SEQ ID NO: 9 - protein, 238 aa, synthetic construct
  REGION 1..238: note = chimeric L chain
MESQTHVLISLLLSVSGTYGDIAITQSPSSVAVSVGETVTLSCKSSQSLLYSENQKDYLGWYQQKPGQTPKPLIYWATNRHTGVPDRFTGSGSGTDFTLIISSVQAEDLADYYCGQYLVYPFTFGPGTKLELKQPKASPSVTLFPPSSEELGANKATLVCLISDFYPSGVTVAWKASGSPVTQGVETTKPSKQSNNKYAASSYLSLTPDKWKSHSSFSCLVTHEGSTVEKKVAPAECS

SEQ ID NO: 10 - protein, 468 aa, synthetic construct
  REGION 1..468: note = chimeric H chain
MGWSQIILFLVAAATCVHSQVQLQQSGAELVKPGSSVKISCKASGYTFTSNFMHWVKQQPGNGLEWIGWIYPEYGNTKYNQKFDGKATLTADKSSSTAYMQLSSLTSEDSAVYFCASEEAVISLVYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 11 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 326 aa, Homo sapiens
STKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - DNA, 321 bp, Homo sapiens
  source 1..321: mol_type = other DNA
actgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 14 - DNA, 981 bp, Homo sapiens
  source 1..981: mol_type = other DNA
tccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 15 - DNA, 399 bp, synthetic construct
  misc_feature 1..399: note = codon-optimized sequence
  source 1..399: mol_type = other DNA
atggaatctcaaactcatgttttgatttcattacttctgagtgtttccggaacctacggtgatatcgctatcactcaatctccctcctctgttgctgtgtctgtgggcgaaaccgttaccctgtcctgcaagtccagtcagtctcttctctactccgagaatcaaaaggactacctgggctggtaccaacagaagcccggccagaccccaaagccactgatatactgggcaaccaacaggcacaccggagtgcccgacaggttcacaggcagtggatctggcaccgactttaccttgatcatttcaagcgtgcaggctgaagatctggccgactactactgtggtcagtatctggtgtatcctttcactttcgggccagggacaaaattggaattgaag

SEQ ID NO: 16 - DNA, 411 bp, synthetic construct
  misc_feature 1..411: note = codon-optimized sequence
  source 1..411: mol_type = other DNA
atgggttggtctcaaattatcttgtttttggttgctgcagccacttgtgttcattctcaggtgcagctgcaacaaagcggcgcagaactggtgaaacctggcagcagcgtgaaaatatcttgtaaggccagcggatatactttcacctccaatttcatgcattgggtcaaacagcagcccggcaacggactcgagtggatcggctggatctaccccgagtatggcaacacaaaatataaccaaaaatttgatggaaaggctaccctgactgccgataagtcctccagcaccgcatacatgcaactctcctccctgacctccgaggatagcgctgtctacttctgtgcttccgaagaggctgtcatatccttggtctattggggccaaggaactctggtgaccgtctcatct

SEQ ID NO: 17 - DNA, 318 bp, synthetic construct
  misc_feature 1..318: note = codon-optimized sequence
  source 1..318: mol_type = other DNA
cagcccaaagcctctcccagcgtcaccctcttcccaccttccagtgaggagctgggggcaaacaaagccactttggtgtgtctcatctccgatttttacccctccggggtcacagtcgcatggaaggcctccggatcccctgtgacacagggagtggagacaacaaaacctagcaagcagagtaacaataagtatgccgcctcaagctatctcagccttactcctgataagtggaagtcacatagcagttttagttgcctcgtaacacatgagggttcaactgtggagaaaaaagtagctccagctgagtgctcatga

SEQ ID NO: 18 - DNA, 996 bp, synthetic construct
  misc_feature 1..996: note = codon-optimized sequence
  source 1..996: mol_type = other DNA
gctagcacaaccgctccctccgtttttcccctcgccccatcctgcgggtcaaccagcggatccaccgtcgctctggcttgtctggtgtcaggatacttccccgagcctgtcaccgtttcttggaatagcggcagccttacttccggcgtgcataccttccctagcgtgcttcagtcctccggtctgtattccctcagctccaccgtaactgtcccaagctcaaggtggccctctgagacatttacctgcaatgtggtccatcctgcttcaaataccaaagtggacaagcccgtcccaaaagagtctacctgcaaatgtatcagtccttgtcccgtgcccgagtctctgggcggaccctcagtctttatcttcccacccaagccaaaggacatattgcgcattacacggacacccgaaatcacctgtgttgtgttggatctcggccgggaagatcctgaggtgcagattagttggtttgttgatggcaaggaggtgcacacagcaaaaacacagcccagagaacagcagttcaacagtacttatagagtagtgagtgtgttgcctatagagcatcaggactggctgacaggcaaagaattcaaatgtagggttaaccacattggcctccctagtccaatcgagaggacaatctctaaagcccgaggccaggctcatcagccttctgtgtacgttctgcctcctagtcctaaggaactgtcttcttcagacacagtaacactcacttgcctgattaaggacttttttcctccagagattgatgtggaatggcagtctaacgggcagccagagccagaatctaagtaccacactactgcaccacagctggatgaggatgggtcttacttcctgtacagtaagctgagtgtggacaagtctcgatggcagcagggggatacttttacttgcgcagtaatgcacgaagcattgcagaaccactacactgacctgtcacttagtcactcaccagggaagtaa

SEQ ID NO: 19 - DNA, 717 bp, synthetic construct
  misc_feature 1..717: note = codon-optimized sequence
  source 1..717: mol_type = other DNA
atggaatctcaaactcatgttttgatttcattacttctgagtgtttccggaacctacggtgatatcgctatcactcaatctccctcctctgttgctgtgtctgtgggcgaaaccgttaccctgtcctgcaagtccagtcagtctcttctctactccgagaatcaaaaggactacctgggctggtaccaacagaagcccggccagaccccaaagccactgatatactgggcaaccaacaggcacaccggagtgcccgacaggttcacaggcagtggatctggcaccgactttaccttgatcatttcaagcgtgcaggctgaagatctggccgactactactgtggtcagtatctggtgtatcctttcactttcgggccagggacaaaattggaattgaagcagcccaaagcctctcccagcgtcaccctcttcccaccttccagtgaggagctgggggcaaacaaagccactttggtgtgtctcatctccgatttttacccctccggggtcacagtcgcatggaaggcctccggatcccctgtgacacagggagtggagacaacaaaacctagcaagcagagtaacaataagtatgccgcctcaagctatctcagccttactcctgataagtggaagtcacatagcagttttagttgcctcgtaacacatgagggttcaactgtggagaaaaaagtagctccagctgagtgctcatga

SEQ ID NO: 20 - DNA, 1407 bp, synthetic construct
  misc_feature 1..1407: note = codon-optimized sequence
  source 1..1407: mol_type = other DNA
atgggttggtctcaaattatcttgtttttggttgctgcagccacttgtgttcattctcaggtgcagctgcaacaaagcggcgcagaactggtgaaacctggcagcagcgtgaaaatatcttgtaaggccagcggatatactttcacctccaatttcatgcattgggtcaaacagcagcccggcaacggactcgagtggatcggctggatctaccccgagtatggcaacacaaaatataaccaaaaatttgatggaaaggctaccctgactgccgataagtcctccagcaccgcatacatgcaactctcctccctgacctccgaggatagcgctgtctacttctgtgcttccgaagaggctgtcatatccttggtctattggggccaaggaactctggtgaccgtctcatctgctagcacaaccgctccctccgtttttcccctcgccccatcctgcgggtcaaccagcggatccaccgtcgctctggcttgtctggtgtcaggatacttccccgagcctgtcaccgtttcttggaatagcggcagccttacttccggcgtgcataccttccctagcgtgcttcagtcctccggtctgtattccctcagctccaccgtaactgtcccaagctcaaggtggccctctgagacatttacctgcaatgtggtccatcctgcttcaaataccaaagtggacaagcccgtcccaaaagagtctacctgcaaatgtatcagtccttgtcccgtgcccgagtctctgggcggaccctcagtctttatcttcccacccaagccaaaggacatattgcgcattacacggacacccgaaatcacctgtgttgtgttggatctcggccgggaagatcctgaggtgcagattagttggtttgttgatggcaaggaggtgcacacagcaaaaacacagcccagagaacagcagttcaacagtacttatagagtagtgagtgtgttgcctatagagcatcaggactggctgacaggcaaagaattcaaatgtagggttaaccacattggcctccctagtccaatcgagaggacaatctctaaagcccgaggccaggctcatcagccttctgtgtacgttctgcctcctagtcctaaggaactgtcttcttcagacacagtaacactcacttgcctgattaaggacttttttcctccagagattgatgtggaatggcagtctaacgggcagccagagccagaatctaagtaccacactactgcaccacagctggatgaggatgggtcttacttcctgtacagtaagctgagtgtggacaagtctcgatggcagcagggggatacttttacttgcgcagtaatgcacgaagcattgcagaaccactacactgacctgtcacttagtcactcaccagggaagtaa

SEQ ID NO: 21 - DNA, 20 bp, synthetic construct
  misc_feature 1..20: note = primer
  source 1..20: mol_type = other DNA
aggatggctcctagactccc

SEQ ID NO: 22 - DNA, 20 bp, synthetic construct
  misc_feature 1..20: note = primer
  source 1..20: mol_type = other DNA
agacgatggtggcatactcg

SEQ ID NO: 23 - DNA, 20 bp, synthetic construct
  misc_feature 1..20: note = primer
  source 1..20: mol_type = other DNA
atgagaatgtttagtgtctt

SEQ ID NO: 24 - DNA, 24 bp, synthetic construct
  misc_feature 1..24: note = primer
  source 1..24: mol_type = other DNA
ttatgtctcttcaaattgtatatc

SEQ ID NO: 25 - DNA, 16 bp, synthetic construct
  misc_feature 1..16: note = primer
  source 1..16: mol_type = other DNA
gttgatctgtgtgttg

SEQ ID NO: 26 - DNA, 20 bp, synthetic construct
  misc_feature 1..20: note = primer
  source 1..20: mol_type = other DNA
cgggacttccacatgagcat

SEQ ID NO: 27 - DNA, 17 bp, synthetic construct
  misc_feature 1..17: note = primer
  source 1..17: mol_type = other DNA
ttttagacagaaagtga

SEQ ID NO: 28 - DNA, 20 bp, synthetic construct
  misc_feature 1..20: note = primer
  source 1..20: mol_type = other DNA
gaccagctcttcttggggaa

SEQ ID NO: 29 - DNA, 29 bp, synthetic construct
  misc_feature 1..29: note = primer
  source 1..29: mol_type = other DNA
ccgctcgagatggggagccggcgggggcc

SEQ ID NO: 30 - DNA, 30 bp, synthetic construct
  misc_feature 1..30: note = primer
  source 1..30: mol_type = other DNA
cgcggatcctgaggggccacaggccgggtc

SEQ ID NO: 31 - DNA, 26 bp, synthetic construct
  misc_feature 1..26: note = primer
  source 1..26: mol_type = other DNA
gaagatctatgagaatgtttagtgtc

SEQ ID NO: 32 - DNA, 28 bp, synthetic construct
  misc_feature 1..28: note = primer
  source 1..28: mol_type = other DNA
ggaattctgtctcttcaaattgtatatc

SEQ ID NO: 33 - DNA, 30 bp, synthetic construct
  misc_feature 1..30: note = primer
  source 1..30: mol_type = other DNA
cgcggctagcatggggagccggcgggggcc

SEQ ID NO: 34 - DNA, 30 bp, synthetic construct
  misc_feature 1..30: note = primer
  source 1..30: mol_type = other DNA
cgcggatatccagcccctgcaactggccgc

SEQ ID NO: 35 - DNA, 30 bp, synthetic construct
  misc_feature 1..30: note = primer
  source 1..30: mol_type = other DNA
cgcggctagcatgagaatgtttagtgtctt

SEQ ID NO: 36 - DNA, 30 bp, synthetic construct
  misc_feature 1..30: note = primer
  source 1..30: mol_type = other DNA
cgcggatatcagtcctctcacttgctggaa

SEQ ID NO: 37 - protein, 12 aa, Rattus norvegicus
QSLLYSENQKDY

SEQ ID NO: 38 - protein, 9 aa, Rattus norvegicus
GQYLVYPFT

SEQ ID NO: 39 - protein, 8 aa, Rattus norvegicus
GYTFTSNF

SEQ ID NO: 40 - protein, 8 aa, Rattus norvegicus
IYPEYGNT

SEQ ID NO: 41 - protein, 11 aa, Rattus norvegicus
ASEEAVISLVY

SEQ ID NO: 42 - protein, 331 aa, Ovis aries
ASTTPPKVYPLTSCCGDTSSSIVTLGCLVSSYMPEPVTVTWNSGALTSGVHTFPAILQSSGLYSLSSVVTVPASTSGAQTFICNVAHPASSTKVDKRVEPGCPDPCKHCRCPPPELPGGPSVFIFPPKPKDTLTISGTPEVTCVVVDVGQDDPEVQFSWFVDNVEVRTARTKPREEQFNSTFRVVSALPIQHQDWTGGKEFKCKVHNEALPAPIVRTISRTKGQAREPQVYVLAPPQEELSKSTLSVTCLVTGFYPDYIAVEWQKNGQPESEDKYGTTTSQLDADGSYFLYSRLRVDKNSWQEGDTYACVVMHEALHNHYTQKSISKPPGK

SEQ ID NO: 43 - DNA, 996 bp, Ovis aries
  source 1..996: mol_type = other DNA
gcctcaacaacacccccgaaagtctaccctctgacttcttgctgcggggacacgtccagctccatcgtgaccctgggctgcctggtctccagctatatgcccgagccggtgaccgtgacctggaactctggtgccctgaccagcggcgtgcacaccttcccggccatcctgcagtcctccgggctctactctctcagcagcgtggtgaccgtgccggccagcacctcaggagcccagaccttcatctgcaacgtagcccacccggccagcagcaccaaggtggacaagcgtgttgagcccggatgcccggacccatgcaaacattgccgatgcccaccccctgagctccccggaggaccgtctgtcttcatcttcccaccgaaacccaaggacacccttacaatctctggaacgcccgaggtcacgtgtgtggtggtggacgtgggccaggatgaccccgaggtgcagttctcctggttcgtggacaacgtggaggtgcgcacggccaggacaaagccgagagaggagcagttcaacagcaccttccgcgtggtcagcgccctgcccatccagcaccaagactggactggaggaaaggagttcaagtgcaaggtccacaacgaagccctcccggcccccatcgtgaggaccatctccaggaccaaagggcaggcccgggagccgcaggtgtacgtcctggccccaccccaggaagagctcagcaaaagcacgctcagcgtcacctgcctggtcaccggcttctacccagactacatcgccgtggagtggcagaaaaatgggcagcctgagtcggaggacaagtacggcacgaccacatcccagctggacgccgacggctcctacttcctgtacagcaggctcagggtggacaagaacagctggcaagaaggagacacctacgcgtgtgtggtgatgcacgaggctctgcacaaccactacacacagaagtcgatctctaagcctccgggtaaatga

SEQ ID NO: 44 - protein, 329 aa, Ovis aries
ASTTAPKVYPLTSCCGDTSSSSSIVTLGCLVSSYMPEPVTVTWNSGALTSGVHTFPAILQSSGLYSLSSVVTVPASTSGAQTFICNVAHPASSAKVDKRVGISSDYSKCSKPPCVSRPSVFIFPPKPKDSLMITGTPEVTCVVVDVGQGDPEVQFSWFVDNVEVRTARTKPREEQFNSTFRVVSALPIQHDHWTGGKEFKCKVHSKGLPAPIVRTISRAKGQAREPQVYVLAPPQEELSKSTLSVTCLVTGFYPDYIAVEWQRARQPESEDKYGTTTSQLDADGSYFLYSRLRVDKSSWQRGDTYACVVMHEALHNHYTQKSISKPPGK

SEQ ID NO: 45 - DNA, 990 bp, Ovis aries
  source 1..990: mol_type = other DNA
gcctccaccacagccccgaaagtctaccctctgacttcttgctgcggggacacgtccagctccagctccatcgtgaccctgggctgcctggtctccagctatatgcccgagccggtgaccgtgacctggaactctggtgccctgaccagcggcgtgcacaccttcccggccatcctgcagtcctccgggctctactctctcagcagcgtggtgaccgtgccggccagcacctcaggagcccagaccttcatctgcaacgtagcccacccggccagcagcgccaaggtggacaagcgtgttgggatctccagtgactactccaagtgttctaaaccgccttgcgtgagccgaccgtctgtcttcatcttccccccgaaacccaaggacagcctcatgatcacaggaacgcccgaggtcacgtgtgtggtggtggacgtgggccagggtgaccccgaggtgcagttctcctggttcgtggacaacgtggaggtgcgcacggccaggacaaagccgagagaggagcagttcaacagcaccttccgcgtggtcagcgccctgcccatccagcacgaccactggactggaggaaaggagttcaagtgcaaggtccacagcaaaggcctcccggcccccatcgtgaggaccatctccagggccaaagggcaggcccgggagccgcaggtgtacgtcctggccccaccccaggaagagctcagcaaaagcacgctcagcgtcacctgcctggtcaccggcttctacccagactacatcgccgtggagtggcagagagcgcggcagcctgagtcggaggacaagtacggcacgaccacatcccagctggacgccgacggctcctacttcctgtacagcaggctcagggtggacaagagcagctggcaaagaggagacacctacgcgtgtgtggtgatgcacgaggctctgcacaaccactacacacagaagtcgatctctaagcctccgggtaaatga

SEQ ID NO: 46 - protein, 102 aa, Ovis aries
PSVFLFKPSEEQLRTGTVSVVCLVNDFYPKDINVKVKVDGVTQNSNFQNSFTDQDSKKSTYSLSSTLTLSSSEYQSHNAYACEVSHKSLPTALVKSFNKNEC

SEQ ID NO: 47 - DNA, 309 bp, Ovis aries
  source 1..309: mol_type = other DNA
ccatccgtcttcctcttcaaaccatctgaggaacagctgaggaccggaactgtctctgtcgtgtgcttggtgaatgatttctaccccaaagatatcaatgtcaaggtgaaagtggatggggttacccagaacagcaacttccagaacagcttcacagaccaggacagcaagaaaagcacctacagcctcagcagcaccctgacactgtccagctcagagtaccagagccataacgcctatgcgtgtgaggtcagccacaagagcctgcccaccgccctcgtcaagagcttcaataagaatgaatgttag

SEQ ID NO: 48 - protein, 106 aa, Ovis aries
GQPKSAPSVTLFPPSTEELSTNKATVVCLINDFYPGSVNVVWKADGSTINQNVKTTQASKQSNSKYAASSYLTLTGSEWKSKSSYTCEVTHEGSTVTKTVKPSECS

SEQ ID NO: 49 - DNA, 321 bp, Ovis aries
  source 1..321: mol_type = other DNA
ggtcagcccaagtccgcaccctcggtcaccctgttcccgccttccacggaggagctcagtaccaacaaggccaccgtggtgtgtctcatcaacgacttctacccgggtagcgtgaacgtggtctggaaggcagatggcagcaccatcaatcagaacgtgaagaccacccaggcctccaaacagagcaacagcaagtacgcggccagcagctacctgaccctgacgggcagcgagtggaagtctaagagcagttacacctgcgaggtcacgcacgaggggagcaccgtgacgaagacagtgaagccctcagagtgttcttag

SEQ ID NO: 50 - protein, 328 aa, Sus scrofa
APKTAPSVYPLAPCGRDTSGPNVALGCLASSYFPEPVTMTWNSGALTSGVHTFPSVLQPSGLYSLSSMVTVPASSLSSKSYTCNVNHPATTTKVDKRVGTKTKPPCPICPGCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTVTCLVIGFYPPDIHVEWKSNGQPEPEGNYRTTPPQQDVDGTFFLYSKLAVDKARWDHGETFECAVMHEALHNHYTQKSISKTQGK

SEQ ID NO: 51 - DNA, 987 bp, Sus scrofa
  source 1..987: mol_type = other DNA
gcccccaagacggccccatcggtctaccctctggccccctgcggcagggacacgtctggccctaacgtggccttgggctgcctggcctcaagctacttccccgagccagtgaccatgacctggaactcgggcgccctgaccagtggcgtgcataccttcccatccgtcctgcagccgtcagggctctactccctcagcagcatggtgaccgtgccggccagcagcctgtccagcaagagctacacctgcaatgtcaaccacccggccaccaccaccaaggtggacaagcgtgttggaacaaagaccaaaccaccatgtcccatatgcccaggctgtgaagtggccgggccctcggtcttcatcttccctccaaaacccaaggacaccctcatgatctcccagacccccgaggtcacgtgcgtggtggtggacgtcagcaaggagcacgccgaggtccagttctcctggtacgtggacggcgtagaggtgcacacggccgagacgagaccaaaggaggagcagttcaacagcacctaccgtgtggtcagcgtcctgcccatccagcaccaggactggctgaaggggaaggagttcaagtgcaaggtcaacaacgtagacctcccagcccccatcacgaggaccatctccaaggctatagggcagagccgggagccgcaggtgtacaccctgcccccacccgccgaggagctgtccaggagcaaagtcaccgtaacctgcctggtcattggcttctacccacctgacatccatgttgagtggaagagcaacggacagccggagccagagggcaattaccgcaccaccccgccccagcaggacgtggacgggaccttcttcctgtacagcaagctcgcggtggacaaggcaagatgggaccatggagaaacatttgagtgtgcggtgatgcacgaggctctgcacaaccactacacccagaagtccatctccaagactcagggtaaatga

SEQ ID NO: 52 - protein, 328 aa, Sus scrofa
APKTAPSVYPLAPCGRDVSGPNVALGCLASSYFPEPVTVTWNSGALTSGVHTFPSVLQPSGLYSLSSMVTVPASSLSSKSYTCNVNHPATTTKVDKRVGIHQPQTCPICPGCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTLTCLVIGFYPPDIHVEWKSNGQPEPENTYRTTPPQQDVDGTFFLYSKLAVDKARWDHGDKFECAVMHEALHNHYTQKSISKTQGK

SEQ ID NO: 53 - DNA, 987 bp, Sus scrofa
  source 1..987: mol_type = other DNA
gcccccaagacggccccatcggtctaccctctggccccctgcggcagggacgtgtctggccctaacgtggccttgggctgcctggcctcaagctacttccccgagccagtgaccgtgacctggaactcgggcgccctgaccagtggcgtgcacaccttcccatccgtcctgcagccgtcagggctctactccctcagcagcatggtgaccgtgccggccagcagcctgtccagcaagagctacacctgcaatgtcaaccacccggccaccaccaccaaggtggacaagcgtgttggaatacaccagccgcaaacatgtcccatatgcccaggctgtgaagtggccgggccctcggtcttcatcttccctccaaaacccaaggacaccctcatgatctcccagacccccgaggtcacgtgcgtggtggtggacgtcagcaaggagcacgccgaggtccagttctcctggtacgtggacggcgtagaggtgcacacggccgagacgagaccaaaggaggagcagttcaacagcacctaccgtgtggtcagcgtcctgcccatccagcaccaggactggctgaaggggaaggagttcaagtgcaaggtcaacaacgtagacctcccagcccccatcacgaggaccatctccaaggctatagggcagagccgggagccgcaggtgtacaccctgcccccacccgccgaggagctgtccaggagcaaagtcacgctaacctgcctggtcattggcttctacccacctgacatccatgttgagtggaagagcaacggacagccggagccagagaacacataccgcaccaccccgccccagcaggacgtggacgggaccttcttcctgtacagcaaactcgcggtggacaaggcaagatgggaccatggagacaaatttgagtgtgcggtgatgcacgaggctctgcacaaccactacacccagaagtccatctccaagactcagggtaaatga

SEQ ID NO: 54 - protein, 328 aa, Sus scrofa
APKTAPSVYPLAPCSRDTSGPNVALGCLASSYFPEPVTVTWNSGALSSGVHTFPSVLQPSGLYSLSSMVTVPASSLSSKSYTCNVNHPATTTKVDKRVGTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 55 - DNA, 987 bp, Sus scrofa
  source 1..987: mol_type = other DNA
gcccccaagacggccccatcggtctaccctctggccccctgcagcagggacacgtctggccctaacgtggccttgggctgcctggcctcaagctacttccccgagccagtgaccgtgacctggaactcgggcgccctgtccagtggcgtgcataccttcccatccgtcctgcagccgtcagggctctactccctcagcagcatggtgaccgtgccggccagcagcctgtccagcaagagctacacctgcaatgtcaaccacccggccaccaccaccaaggtggacaagcgtgttggaacaaagaccaaaccaccatgtcccatatgcccagcctgtgaatcaccagggccctcggtcttcatcttccctccaaaacccaaggacaccctcatgatctcccggacaccccaggtcacgtgcgtggtggttgatgtgagccaggagaacccggaggtccagttctcctggtacgtggacggcgtagaggtgcacacggcccagacgaggccaaaggaggagcagttcaacagcacctaccgcgtggtcagcgtcctacccatccagcaccaggactggctgaacgggaaggagttcaagtgcaaggtcaacaacaaagacctcccagcccccatcacaaggatcatctccaaggccaaagggcagacccgggagccgcaggtgtacaccctgcccccacacgccgaggagctgtccaggagcaaagtcagcataacctgcctggtcattggcttctacccacctgacatcgatgtcgagtggcaaagaaacggacagccggagccagagggcaattaccgcaccaccccgccccagcaggacgtggacgggacctacttcctgtacagcaagttctcggtggacaaggccagctggcagggtggaggcatattccagtgtgcggtgatgcacgaggctctgcacaaccactacacccagaagtctatctccaagactccgggtaaatga

SEQ ID NO: 56 - protein, 328 aa, Sus scrofa
APKTAPLVYPLAPCGRDTSGPNVALGCLASSYFPEPVTVTWNSGALTSGVHTFPSVLQPSGLYSLSSMVTVPASSLSSKSYTCNVNHPATTTKVDKRVGTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 57 - DNA, 987 bp, Sus scrofa
  source 1..987: mol_type = other DNA
gcccccaagacggccccattggtctaccctctggccccctgcggcagggacacgtctggccctaacgtggccttgggctgcctggcctcaagctacttccccgagccagtgaccgtgacctggaactcgggcgccctgaccagtggcgtgcataccttcccatccgtcctgcagccgtcagggctctactccctcagcagcatggtgaccgtgccggccagcagcctgtccagcaagagctacacctgcaatgtcaaccacccggccaccaccaccaaggtggacaagcgtgttggaacaaagaccaaaccaccatgtcccatatgcccagcctgtgaatcgccagggccctcggtcttcatcttccctccaaaacccaaggacaccctcatgatctcccggacaccccaggtcacgtgcgtggtagttgatgtgagccaggagaacccggaggtccagttctcctggtacgtggacggcgtagaggtgcacacggcccagacgaggccaaaggaggagcagttcaacagcacctaccgcgtggtcagcgtcctgcccatccagcaccaggactggctgaacgggaaggagttcaagtgcaaggtcaacaacaaagacctcccagcccccatcacaaggatcatctccaaggccaaagggcagacccgggagccgcaggtgtacaccctgcccccacacgccgaggagctgtccaggagcaaagtcagcataacctgcctggtcattggcttctacccacctgacatcgatgtcgagtggcaaagaaacggacagccggagccagagggcaattaccgcaccaccccgccccagcaggacgtggacgggacctacttcctgtacagcaagttctcggtggacaaggccagctggcagggtggaggcatattccagtgtgcggtgatgcacgaggctctgcacaaccactacacccagaagtctatctccaagactccgggtaaatga

SEQ ID NO: 58 - protein, 333 aa, Sus scrofa
AYNTAPSVYPLAPCGRDVSDHNVALGCLVSSYFPEPVTVTWNSGALSRVVHTFPSVLQPSGLYSLSSMVIVAASSLSTLSYTCNVYHPATNTKVDKRVDIEPPTPICPEICSCPAAEVLGAPSVFLFPPKPKDILMISRTPKVTCVVVDVSQEEAEVQFSWYVDGVQLYTAQTRPMEEQFNSTYRVVSVLPIQHQDWLKGKEFKCKVNNKDLLSPITRTISKATGPSRVPQVYTLPPAWEELSKSKVSITCLVTGFYPPDIDVEWQSNGQQEPEGNYRTTPPQQDVDGTYFLYSKLAVDKVRWQRGDLFQCAVMHEALHNHYTQKSISKTQGK

SEQ ID NO: 59 - DNA, 1002 bp, Sus scrofa
  source 1..1002: mol_type = other DNA
gcctacaacacagctccatcggtctaccctctggccccctgtggcagggacgtgtctgatcataacgtggccttgggctgccttgtctcaagctacttccccgagccagtgaccgtgacctggaactcgggtgccctgtccagagtcgtgcataccttcccatccgtcctgcagccgtcagggctctactccctcagcagcatggtgatcgtggcggccagcagcctgtccaccctgagctacacgtgcaacgtctaccacccggccaccaacaccaaggtggacaagcgtgttgacatcgaaccccccacacccatctgtcccgaaatttgctcatgcccagctgcagaggtcctgggagcaccgtcggtcttcctcttccctccaaaacccaaggacatcctcatgatctcccggacacccaaggtcacgtgcgtggtggtggacgtgagccaggaggaggctgaagtccagttctcctggtacgtggacggcgtacagttgtacacggcccagacgaggccaatggaggagcagttcaacagcacctaccgcgtggtcagcgtcctgcccatccagcaccaggactggctgaaggggaaggagttcaagtgcaaggtcaacaacaaagacctcctttcccccatcacgaggaccatctccaaggctacagggccgagccgggtgccgcaggtgtacaccctgcccccagcctgggaagagctgtccaagagcaaagtcagcataacctgcctggtcactggcttctacccacctgacatcgatgtcgagtggcagagcaacggacaacaagagccagagggcaattaccgcaccaccccgccccagcaggacgtggatgggacctacttcctgtacagcaagctcgcggtggacaaggtcaggtggcagcgtggagacctattccagtgtgcggtgatgcacgaggctctgcacaaccactacacccagaagtccatctccaagactcagggtaaatga

SEQ ID NO: 60 - protein, 277 aa, Sus scrofa
TFPSVLQPSGLYSLSSMVTVPASSLSSKSYTCNVNHPATTTKVDKRVGTKTKPPCPICPACEGPGPSAFIFPPKPKDTLMISRTPKVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPPTEELSRSKVTLTCLVTGFYPPDIDVEWQRNGQPEPEGNYRTTPPQQDVDGTYFLYSKLAVDKASWQRGDTFQCAVMHEALHNHYTQKSIFKTPGK

SEQ ID NO: 61 - DNA, 834 bp, Sus scrofa
  source 1..834: mol_type = other DNA
accttcccatccgtcctgcagccgtcagggctctactccctcagcagcatggtgaccgtgccggccagcagcctgtccagcaagagctacacctgcaatgtcaaccacccggccaccaccaccaaggtggacaagcgtgttggaacaaagaccaaaccaccatgtcccatatgcccagcctgtgaagggcccgggccctcggccttcatcttccctccaaaacccaaggacaccctcatgatctcccggacccccaaggtcacgtgcgtggtggtagatgtgagccaggagaacccggaggtccagttctcctggtacgtggacggcgtagaggtgcacacggcccagacgaggccaaaggaggagcagttcaacagcacctaccgcgtggtcagcgtcctgcccatccagcaccaggactggctgaacgggaaggagttcaagtgcaaggtcaacaacaaagacctcccagcccccatcacaaggatcatctccaaggccaaagggcagacccgggagccgcaggtgtacaccctgcccccacccaccgaggagctgtccaggagcaaagtcacgctaacctgcctggtcactggcttctacccacctgacatcgatgtcgagtggcaaagaaacggacagccggagccagagggcaattaccgcaccaccccgccccagcaggacgtggacgggacctacttcctgtacagcaagctcgcggtggacaaggccagctggcagcgtggagacacattccagtgtgcggtgatgcacgaggctctgcacaaccactacacccagaagtccatcttcaagactccgggtaaatga

SEQ ID NO: 62 - protein, 318 aa, Sus scrofa
APKTAPSVYPLAPCGRDVSGPNVALGCLASSYFPEPVTVTWNSGALTSGVHTFPSVLQPSGLYSLSSMVTVPASSLSSKSYTCNVNHPATTTKVDKRVGIHQPQTCPICPACEGPGPSAFIFPPKPKDTLMISRTPKVTCVVVDVSQENPEVQFSWYVDGVEVHTAQTRPKEEQFNSTYRVVSVLLIQHQDWLNGKEFKCKVNNKDLPAPITRIISKAKGQTREPQVYTLPPPTEELSRSKVTLTCLVTGFYPPDIDVEWQRNGQPEPEGNYRTTPPQQDVDGTYFLYSKLAVDKASWQRGDTFQCAVMHEALHNHYT

SEQ ID NO: 63 - DNA, 955 bp, Sus scrofa
  source 1..955: mol_type = other DNA
gcccccaagacggccccatcggtctaccctctggccccctgcggcagggacgtgtctggccctaacgtggccttgggctgcctggcctcaagctacttccccgagccagtgaccgtgacctggaactcgggcgccctgaccagtggcgtgcacaccttcccatccgtcctgcagccgtcagggctctactccctcagcagcatggtgaccgtgccggccagcagcctgtccagcaagagctacacctgcaatgtcaaccacccggccaccaccaccaaggtggacaagcgtgttggaatacaccagccgcaaacatgtcccatatgcccagcctgtgaagggcccgggccctcggccttcatcttccctccaaaacccaaggacaccctcatgatctcccggacccccaaggtcacgtgcgtggtggttgatgtgagccaggagaacccggaggtccagttctcctggtacgtggacggcgtagaggtgcacacggcccagacgaggccaaaggaggagcagttcaacagcacctaccgcgtggtcagcgtcctgctcatccagcaccaggactggctgaacgggaaggagttcaagtgcaaggtcaacaacaaagacctcccagcccccatcacaaggatcatctccaaggccaaagggcagacccgggagccgcaggtgtacaccctgcccccacccaccgaggagctgtccaggagcaaagtcacgctaacctgcctggtcactggcttctacccacctgacatcgatgtcgagtggcaaagaaacggacagccggagccagagggcaattaccgcaccaccccgccccagcaggacgtggacgggacctacttcctgtacagcaagctcgcggtggacaaggccagctggcagcgtggagacacattccagtgtgcggtgatgcacgaggctctgcacaaccactacaccc

SEQ ID NO: 64 - protein, 323 aa, Sus scrofa
APKTAPSVYPLAPCSRDTSGPNVALGCLVSSYFPEPVTVTWNSGALTSGVHTFPSVLQPSGLYSLSSMVTVPAHSLSSKRYTCNVNHPATKTKVDLCVGRPCPICPGCEVAGPSVFIFPPKPKDILMISRTPEVTCVVVDVSKEHAEVQFSWYVDGEEVHTAETRPKEEQFNSTYRVVSVLPIQHEDWLKGKEFECKVNNEDLPGPITRTISKAKGVVRSPEVYTLPPPAEELSKSIVTLTCLVKSIFPFIHVEWKINGKPEPENAYRTTPPQEDEDRTYFLYSKLAVDKARWDHGETFECAVMHEALHNHYTQKSISKTQGK

SEQ ID NO: 65 - DNA, 975 bp, Sus scrofa
  misc_feature 748: note = n is a, c, g, or t
  source 1..975: mol_type = other DNA
gcccccaagacggccccatcggtctaccctctggccccctgcagcagggacacgtctggccctaacgtggccttgggctgcctggtctcaagctacttccccgagccagtgaccgtgacctggaactcgggcgccctgaccagtggcgtgcacaccttcccatccgtcctgcagccgtcagggctctactccctcagcagcatggtgaccgtgccggcccacagcttgtccagcaagcgctatacgtgcaatgtcaaccacccagccaccaaaaccaaggtggacctgtgtgttggacgaccatgtcccatatgcccaggctgtgaagtggccgggccctcggtcttcatcttccctccaaaacccaaggacatcctcatgatctcccggacccccgaggtcacgtgcgtggtggtggacgtcagcaaggagcacgccgaggtccagttctcctggtacgtggacggcgaagaggtgcacacggccgagacgaggccaaaggaggagcagttcaacagcacctaccgcgtggtcagcgtcctgcccatccagcacgaggactggctgaaggggaaggagttcgagtgcaaggtcaacaacgaagacctcccaggccccatcacgaggaccatctccaaggccaaaggggtggtacggagcccggaggtgtacaccctgcccccacccgccgaggagctgtccaagagcatagtcacgctaacctgcctggtcaaaagcatcttcccgnctttcatccatgttgagtggaaaatcaacggaaaaccagagccagagaacgcatatcgcaccaccccgcctcaggaggacgaggacaggacctacttcctgtacagcaagctcgcggtggacaaggcaagatgggaccatggagaaacatttgagtgtgcggtgatgcacgaggctctgcacaaccactacacccagaagtccatctccaagactcagggtaaatga

SEQ ID NO: 66 - protein, 317 aa, Sus scrofa
AYNTAPSVYPLAPCGRDVSDHNVALGCLVSSYFPEPVTVTWNWGAQTSGVHTFPSVLQPSGLYSLSSTVTVPAHSLSSKCFTCNVNHPATTTKVDLCVGKKTKPRCPICPGCEVAGPSVFIFPPKPKDILMISRTPEVTCVVVDVSKEHAEVQFSWYVDGEEVHTAETRPKEEQFNSTYRVVSVLPIQHEDWLKGKEFECKVNNEDLPGPITRTISKAKGVVRSPEVYTLPPPAEELSKSIVTLTCLVKSFFPPFIHVEWKINGKPEPENAYRTTPPQEDEDGTYFLYSKFSVEKFRWHSGGIHCAVMHEALHNHYT

SEQ ID NO: 67 - DNA, 952 bp, Sus scrofa
  source 1..952: mol_type = other DNA
gcctacaacacagctccatcggtctaccctctggccccctgtggcagggacgtgtctgatcataacgtggccttgggctgcctggtctcaagctacttccccgagccagtgaccgtgacctggaactggggcgcccagaccagtggcgtgcacaccttcccatccgtcctgcagccgtcagggctctactccctcagcagcacggtgaccgtgccggcccacagcttgtccagcaagtgcttcacgtgcaatgtcaaccacccggccaccaccaccaaggtggacctgtgtgttggaaaaaagaccaagcctcgatgtcccatatgcccaggctgtgaagtggccgggccctcggtcttcatcttccctccaaaacccaaggacatcctcatgatctcccggacccccgaggtcacgtgcgtggtggtggacgtcagcaaggagcacgccgaggtccagttctcctggtacgtggacggcgaagaggtgcacacggccgagacgagaccaaaggaggagcagttcaacagcacttaccgcgtggtcagcgtcctgcccatccagcacgaggactggctgaaggggaaggagttcgagtgcaaggtcaacaacgaagacctcccaggccccatcacgaggaccatctccaaggccaaaggggtggtacggagcccggaggtgtacaccctgcccccacccgccgaggagctgtccaagagcatagtcacgctaacctgcctggtcaaaagcttcttcccgcctttcatccatgttgagtggaaaatcaacggaaaaccagagccagagaacgcataccgcaccaccccgccccaggaggacgaggacgggacctacttcctgtacagcaagttctcggtggaaaagttcaggtggcacagtggaggcatccactgtgcggtgatgcacgaggctctgcacaaccactacaccc

SEQ ID NO: 68 - protein, 314 aa, Sus scrofa
APKTAPSVYPLAPCGRDTSGPNVALGCLASSYFPEPVTLTWNSGALTSGVHTFPSVLQPSGLYSLSSMVTVPASSLSSKSYTCNVNHPATTTKVDLCVGRPCPICPACEGPGPSVFIFPPKPKDTLMISRTPQVTCVVVDVSQENPEVQFSWYVDGVEVHTAQTRPKEAQFNSTYRVVSVLPIQHEDWLKGKEFECKVNNKDLPAPITRIISKAKGPSREPQVYTLSPSAEELSRSKVSITCLVTGFYPPDIDVEWKSNGQPEPEGNYRTTPPQQDVDGTYFLYSKLAVDKASWQRGDPFQCAVMHEALHNHYT

SEQ ID NO: 69 - DNA, 943 bp, Sus scrofa
  source 1..943: mol_type = other DNA
gcccccaagacggccccatcggtctaccctctggccccctgcggcagggacacgtctggccctaacgtggccttgggctgcctggcctcaagctacttccccgagccagtgaccctgacctggaactcgggcgccctgaccagtggcgtgcataccttcccatccgtcctgcagccgtcagggctctactccctcagcagcatggtgaccgtgccggccagcagcctgtccagcaagagctacacctgcaatgtcaaccacccggccaccaccaccaaggtggacctgtgtgttggacgaccatgtcccatatgcccagcctgtgaagggcccgggccctcggtcttcatcttccctccaaaacccaaggacaccctcatgatctcccggacaccccaggtcacgtgcgtggtggtagatgtgagccaggaaaacccggaggtccagttctcctggtatgtggacggtgtagaggtgcacacggcccagacgaggccaaaggaggcgcagttcaacagcacctaccgtgtggtcagcgtcctgcccatccagcacgaggactggctgaaggggaaggagttcgagtgcaaggtcaacaacaaagacctcccagcccccatcacaaggatcatctccaaggccaaagggccgagccgggagccgcaggtgtacaccctgtccccatccgccgaggagctgtccaggagcaaagtcagcataacctgcctggtcactggcttctacccacctgacatcgatgtcgagtggaagagcaacggacagccggagccagagggcaattaccgcaccaccccgccccagcaggacgtggacgggacctacttcctgtacagcaagctcgcggtggacaaggccagctggcagcgtggagacccattccagtgtgcggtgatgcacgaggctctgcacaaccactacaccc

SEQ ID NO: 70 - protein, 320 aa, Sus scrofa
APKTAPSVYPLAPCGRDTSGPNVALGCLASSYFPEPVTVTWNSGALTSGVHTFPSVLQPSGLYSLSSTVTVPARSSSRKCFTCNVNHPATTTKVDLCVGRPCPICPACEGNGPSVFIFPPKPKDTLMISRTPEVTCVVVDVSQENPEVQFSWYVDGEEVHTAETRPKEEQFNSTYRVVSVLPIQHQDWLKGKEFECKVNNKDLPAPITRIISKAKGPSREPQVYTLSPSAEELSRSKVSITCLVTGFYPPDIDVEWKSNGQPEPEGNYRSTPPQEDEDGTYFLYSKLAVDKARLQSGGIHCAVMHEALHNHYTQKSISKT

SEQ ID NO: 71 - DNA, 960 bp, Sus scrofa
  source 1..960: mol_type = other DNA
gcccccaagacggccccatcggtctaccctctggccccctgcggcagggacacgtctggccctaacgtggccttgggctgcctggcctcaagctacttccccgagccagtgaccgtgacctggaactcgggcgccctgaccagtggcgtgcacaccttcccatccgtcctgcagccgtcagggctctactccctcagcagcacggtgaccgtgccggccaggagctcgtccagaaagtgcttcacgtgcaatgtcaaccacccggccaccaccaccaaggtggacctgtgtgttggacgaccatgtcccatatgcccagcctgtgaagggaacgggccctcggtcttcatcttccctccaaaacccaaggacaccctcatgatctcccggacccccgaggtcacgtgcgtggtggtagatgtgagccaggaaaacccggaggtccagttctcctggtacgtggacggcgaagaggtgcacacggccgagacgaggccaaaggaggagcagttcaacagcacctaccgtgtggtcagcgtcctgcccatccagcaccaggactggctgaagggaaaggagttcgagtgcaaggtcaacaacaaagacctcccagcccccatcacaaggatcatctccaaggccaaagggccgagccgggagccgcaggtgtacaccctgtccccatccgccgaggagctgtccaggagcaaagtcagcataacctgcctggtcactggcttctacccacctgacatcgatgtcgagtggaagagcaacggacagccggagccagagggcaattaccgctccaccccgccccaggaggacgaggacgggacctacttcctgtacagcaaactcgcggtggacaaggcgaggttgcagagtggaggcatccactgtgcggtgatgcacgaggctctgcacaaccactacacccagaagtccatctccaagact

SEQ ID NO: 72 - protein, 266 aa, Bubalus bubalis
SGVHTFPAVLQSSGLYSLSSTVTAPASATKSQTFTCNVAHPASSTKVDKAVVPPCRPKPCDCCPPPELPGGPSVFIFPPKPKDTLTISGTPEVTCVVVDVGHDDPEVKFSWFVDDVEVNTARTKPREEQFNSTYRVVSALPIQHNDWTGGKEFKCKVYNEGLPAPIVRTISRTKGQAREPQVYVLAPPQDELSKSTVSITCMVTGFYPDYIAVEWQKDGQPESEDKYGTTPPQLDSDGSYFLYSRLRVNKNSWQEGGAYTCVVMHE

SEQ ID NO: 73 - DNA, 801 bp, Bubalus bubalis
  source 1..801: mol_type = other DNA
gagcggcgtgcacaccttcccggccgtccttcagtcctccgggctctactctctcagcagcacggtgaccgcgcccgccagcgccacaaaaagccagaccttcacctgcaacgtagcccacccggccagcagcaccaaggtggacaaggctgttgttcccccatgcagaccgaaaccctgtgattgctgcccaccccctgagctccccggaggaccctctgtcttcatcttcccaccaaaacccaaggacaccctcacaatctctggaactcctgaggtcacgtgtgtggtggtggacgtgggccacgatgaccccgaggtgaagttctcctggttcgtggacgatgtggaggtaaacacagccaggacgaagccaagagaggagcagttcaacagcacctaccgcgtggtcagcgccctgcccatccagcacaacgactggactggaggaaaggagttcaagtgcaaggtctacaatgaaggcctcccagcccccatcgtgaggaccatctccaggaccaaagggcaggcccgggagccgcaggtgtacgtcctggccccaccccaggacgagctcagcaaaagcacggtcagcatcacttgcatggtcactggcttctacccagactacatcgccgtagagtggcagaaagatgggcagcctgagtcagaggacaaatatggcacgaccccgccccagctggacagcgatggctcctacttcctgtacagcaggctcagggtgaacaagaacagctggcaagaaggaggcgcctacacgtgtgtagtgatgcatgaggc

SEQ ID NO: 74 - protein, 309 aa, Bubalus bubalis
ASITAPKVYPLTSCRGETSSSTVTLGCLVSSYMPEPVTVTWNSGALKSGVHTFPAVLQSSGLYSLSSTVTAPASATKSQTFTCNVAHPASSTKVDTAVGFSSDCCKFPKPCVRGPSVFIFPPKPKDTLMITGNPEVTCVVVDVGRDNPEVQFSWFVGDVEVHTGRSKPREEQFNSTYRVVSTLPIQHNDWTGGKEFKCKVNNKGLPAPIVRTISRTKGQAREPQVYVLAPPQEELSKSTVSVTCMVTGFYPDYIAVEWHRDRQAESEDKYRTTPPQLDSDGSYFLYSRLKVNKNSWQEGGAYTCVVMHE

SEQ ID NO: 75 - DNA, 929 bp, Bubalus bubalis
  source 1..929: mol_type = other DNA
gcctccatcacagccccgaaagtctaccctctgacttcttgccgcggggaaacgtccagctccaccgtgaccctgggctgcctggtctccagctacatgcccgagccggtgaccgtgacctggaactcgggtgccctgaagagcggcgtgcacaccttcccggccgtccttcagtcctctgggctctactctctcagcagcacggtgaccgcgcccgccagcgccacaaaaagccagaccttcacctgcaacgtagcccacccggccagcagcaccaaggtggacacggctgttgggttctccagtgactgctgcaagtttcctaagccttgtgtgaggggaccatctgtcttcatcttcccgccgaaacccaaagacaccctgatgatcacaggaaatcccgaggtcacatgtgtggtggtggacgtgggccgggataaccccgaggtgcagttctcctggttcgtgggtgatgtggaggtgcacacgggcaggtcgaagccgagagaggagcagttcaacagcacctaccgcgtggtcagcaccctgcccatccagcacaatgactggactggaggaaaggagttcaagtgcaaggtcaacaacaaaggcctcccagcccccatcgtgaggaccatctccaggaccaaagggcaggcccgggagccgcaggtgtacgtcctggccccaccccaggaagagctcagcaaaagcacggtcagcgtcacttgcatggtcactggcttctacccagactacatcgccgtagagtggcatagagaccggcaggctgagtcggaggacaagtaccgcacgaccccgccccagctggacagcgatggctcctacttcctgtacagcaggctcaaggtgaacaagaacagctggcaagaaggaggcgcctacacgtgtgtagtgatgcatgaggc

SEQ ID NO: 76 - protein, 352 aa, Bubalus bubalis
ASTTAPKVYPLASSCGDTSSSTVTLGCLVSSYMPEPVTVTWNSGALKNGVHTFPAVRQSSGLYSLSSMVTMPTSTAGTQTFTCNVAHPASSTKVDTAVTARHPVPKTPETPIHPVKPPTQEPRDEKTPCQCPKCPEPLGGLSVFIFPPKPKDTLTISGTPEVTCVVVDVGQDDPEVQFSWFVDDVEVHTARMKPREEQFNSTYRVVSALPIQHQDWLREKEFKCKVNNKGLPAPIVRTISRTKGQAREPQVYVLAPPREELSKSTLSLTCLITGFYPEEVDVEWQRNGQPESEDKYHTTPPQLDADGSYFLYSRLRVNRSSWQEGDHYTCAVMHEALRNHYKEKPISRSPGK

SEQ ID NO: 77 - DNA, 1059 bp, Bubalus bubalis
  source 1..1059: mol_type = other DNA
gcctccaccacagccccgaaagtctaccctctggcatccagctgcggggacacgtccagctccaccgtgaccctgggctgcctggtctccagctacatgcccgagccggtgaccgtgacctggaactcgggtgccctgaagaacggcgtgcacaccttcccggccgtccggcagtcctccgggctctactctctcagcagcatggtgaccatgcccaccagcaccgcaggaacccagaccttcacctgcaacgtagcccacccggccagcagcaccaaggtggacacggctgtcactgcaaggcatccggtcccgaagacaccagagacacctatccatcctgtaaaacccccaacccaggagcccagagatgaaaagacaccctgccagtgtcccaaatgcccagaacctctgggaggactgtctgtcttcatcttcccaccgaaacccaaggacaccctcacaatctctggaacgcccgaggtcacgtgtgtggtggtggacgtgggccaggatgaccccgaagtgcagttctcctggttcgtggatgacgtggaggtgcacacagccaggatgaagccaagagaggagcagttcaacagcacctaccgcgtggtcagcgccctgcccatccagcaccaggactggctgcgggaaaaggagttcaagtgcaaggtcaacaacaaaggcctcccggcccccatcgtgaggaccatctccaggaccaaagggcaggcccgggagccacaggtgtatgtcctggccccaccccgggaagagctcagcaaaagcacgctcagcctcacctgcctaatcaccggcttctacccagaagaggtagacgtggagtggcagagaaatgggcagcctgagtcagaggacaagtaccacacgaccccaccccagctggacgctgacggctcctacttcctgtacagcaggctcagggtgaacaggagcagctggcaggaaggagaccactacacgtgtgcagtgatgcatgaagctttacggaatcactacaaagagaagcccatctcgaggtctccgggtaaatga

SEQ ID NO: 78 - protein, 105 aa, Bubalus bubalis
QPKSAPSVTLFPPSTEELSANKATLVCLISDFYPGSMTVARKADGSTITRNVETTRASKQSNSKYAASSYLSLTGSEWKSKGSYSCEVTHEGSTVTKTVKPSECS

SEQ ID NO: 79 - DNA, 318 bp, Bubalus bubalis
  source 1..318: mol_type = other DNA
cagcccaagtccgcaccctcagtcaccctgttcccaccctccacggaggagctcagcgccaacaaggccaccctggtgtgtctcatcagcgacttctacccgggtagcatgaccgtggccaggaaggcagacggcagcaccatcacccggaacgtggagaccacccgggcctccaaacagagcaacagcaagtacgcggccagcagctacctgagcctgacgggcagcgagtggaaatcgaaaggcagttacagctgcgaggtcacgcacgaggggagcaccgtgacaaagacagtgaagccctcagagtgttcttag

SEQ ID NO: 80 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - DNA, 690 bp, Homo sapiens
  source 1..690: mol_type = other DNA
gagtccaaatatggtcccccgtgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcgtgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaatga

SEQ ID NO: 82 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 83 - DNA, 654 bp, Homo sapiens
  source 1..654: mol_type = other DNA
gcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctcaccgtggacaagagcaggtggcaggaggggaacgtcttctcatgctccgtgatgcatgaggctctgcacaaccactacacgcagaagagcctctccctgtctctgggtaaatga

SEQ ID NO: 84 - protein, 329 aa, Bos taurus
ASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 85 - protein, 329 aa, Bos taurus; identical to SEQ ID NO: 84

SEQ ID NO: 86 - protein, 329 aa, Bos taurus
ASTTAPKVYPLSSCCGDKSSSTVTLGCLVSSYMPEPVTVTWNSGALKSGVHTFPAVLQSSGLYSLSSMVTVPGSTSGTQTFTCNVAHPASSTKVDKAVDPRCKTTCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADGSYFLYSRLRVDRNSWQEGDTYTCVVMHEALHNHYTQKSTSKSAGK

SEQ ID NO: 87 - protein, 326 aa, Bos taurus
ASTTAPKVYPLASSCGDTSSSTVTLGCLVSSYMPEPVTVTWNSGALKSGVHTFPAVLQSSGLYSLSSMVTVPASSSGQTFTCNVAHPASSTKVDKAVGVSIDCSKCHNQPCVREPSVFIFPPKPKDTLMITGTPEVTCVVVNVGHDNPEVQFSWFVDDVEVHTARSKPREEQFNSTYRVVSALPIQHQDWTGGKEFKCKVNNKGLSAPIVRIISRSKGPAREPQVYVLDPPKEELSKSTLSVTCMVTGFYPEDVAVEWQRNRQTESEDKYRTTPPQLDTDRSYFLYSKLRVDRNSWQEGDAYTCVVMHEALHNHYMQKSTSKSAGK

SEQ ID NO: 88 - protein, 326 aa, Bos taurus
ASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVGVSSDCSKPNNQHCVREPSVFIFPPKPKDTLMITGTPEVTCVVVNVGHDNPEVQFSWFVDDVEVHTARTKPREEQFNSTYRVVSALPIQHQDWTGGKEFKCKVNIKGLSASIVRIISRSKGPAREPQVYVLDPPKEELSKSTVSVTCMVIGFYPEDVDVEWQRDRQTESEDKYRTTPPQLDADRSYFLYSKLRVDRNSWQRGDTYTCVVMHEALHNHYMQKSTSKSAGK

SEQ ID NO: 89 - protein, 327 aa, Bos taurus
ASTTAPKVYPLSSCCGDKSSSGVTLGCLVSSYMPEPVTVTWNSGALKSGVHTFPAVLQSSGLYSLSSMVTVPASSSGTQTFTCNVAHPASSTKVDKAVGVSSDCSKPNNQHCVREPSVFIFPPKPKDTLMITGTPEVTCVVVNVGHDNPEVQFSWFVDDVEVHTARTKPREEQFNSTYRVVSALPIQHQDWTGGKEFKCKVNIKGLSASIVRIISRSKGPAREPQVYVLDPPKEELSKSTVSLTCMVIGFYPEDVDVEWQRDRQTESEDKYRTTPPQLDADRSYFLYSKLRVDRNSWQRGDTYTCVVMHEALHNHYMQKSTSKSAGK

SEQ ID NO: 90 - protein, 352 aa, Bos taurus
ASTTAPKVYPLASSCGDTSSSTVTLGCLVSSYMPEPVTVTWNSGALKSGVHTFPAVRQSSGLYSLSSMVTVPASSSETQTFTCNVAHPASSTKVDKAVTARRPVPTTPKTTIPPGKPTTPKSEVEKTPCQCSKCPEPLGGLSVFIFPPKPKDTLTISGTPEVTCVVVDVGQDDPEVQFSWFVDDVEVHTARTKPREEQFNSTYRVVSALRIQHQDWLQGKEFKCKVNNKGLPAPIVRTISRTKGQAREPQVYVLAPPREELSKSTLSLTCLITGFYPEEIDVEWQRNGQPESEDKYHTTAPQLDADGSYFLYSKLRVNKSSWQEGDHYTCAVMHEALRNHYKEKSISRSPGK

SEQ ID NO: 91 - protein, 352 aa, Bos taurus
ASTTAPKVYPLASRCGDTSSSTVTLGCLVSSYMPEPVTVTWNSGALKSGVHTFPAVLQSSGLYSLSSMVTVPASTSETQTFTCNVAHPASSTKVDKAVTARRPVPTTPKTTIPPGKPTTQESEVEKTPCQCSKCPEPLGGLSVFIFPPKPKDTLTISGTPEVTCVVVDVGQDDPEVQFSWFVDDVEVHTARTKPREEQFNSTYRVVSALRIQHQDWLQGKEFKCKVNNKGLPAPIVRTISRTKGQAREPQVYVLAPPREELSKSTLSLTCLITGFYPEEIDVEWQRNGQPESEDKYHTTAPQLDADGSYFLYSRLRVNKSSWQEGDHYTCAVMHEALRNHYKEKSISRSPGK

SEQ ID NO: 92 - DNA, 990 bp, Bos taurus
  source 1..990: mol_type = other DNA
gcctccaccacagccccgaaagtctaccctctgagttcttgctgcggggacaagtccagctccaccgtgaccctgggctgcctggtctccagctacatgcccgagccggtgaccgtgacctggaactcgggtgccctgaagagcggcgtgcacaccttcccggctgtccttcagtcctccgggctgtactctctcagcagcatggtgaccgtgcccggcagcacctcaggacagaccttcacctgcaacgtagcccacccggccagcagcaccaaggtggacaaggctgttgatcccacatgcaaaccatcaccctgtgactgttgcccaccccctgagctccccggaggaccctctgtcttcatcttcccaccgaaacccaaggacaccctcacaatctcgggaacgcccgaggtcacgtgtgtggtggtggacgtgggccacgatgaccccgaggtgaagttctcctggttcgtggacgacgtggaggtaaacacagccacgacgaagccgagagaggagcagttcaacagcacctaccgcgtggtcagcgccctgcgcatccagcaccaggactggactggaggaaaggagttcaagtgcaaggtccacaacgaaggcctcccggcccccatcgtgaggaccatctccaggaccaaagggccggcccgggagccgcaggtgtatgtcctggccccaccccaggaagagctcagcaaaagcacggtcagcctcacctgcatggtcaccagcttctacccagactacatcgccgtggagtggcagagaaacgggcagcctgagtcggaggacaagtacggcacgaccccgccccagctggacgccgacagctcctacttcctgtacagcaagctcagggtggacaggaacagctggcaggaaggagacacctacacgtgtgtggtgatgcacgaggccctgcacaatcactacacgcagaagtccacctctaagtctgcgggtaaatga

SEQ ID NO: 93 - DNA, 990 bp, Bos taurus
  source 1..990: mol_type = other DNA
gcctccaccacagccccgaaagtctaccctctgagttcttgctgcggggacaagtccagctccaccgtgaccctgggctgcctggtctccagctacatgcccgagccggtgaccgtgacctggaactcgggtgccctgaagagcggcgtgcacaccttcccggccgtccttcagtcctccgggctgtactctctcagcagcatggtgaccgtgcccggcagcacctcaggacagaccttcacctgcaacgtagcccacccggccagcagcaccaaggtggacaaggctgttgatcccacatgcaaaccatcaccctgtgactgttgcccaccccctgagctccccggaggaccctctgtcttcatcttcccaccgaaacccaaggacaccctcacaatctcgggaacgcccgaggtcacgtgtgtggtggtggacgtgggccacgatgaccccgaggtgaagttctcctggttcgtggacgacgtggaggtaaacacagccacgacgaagccgagagaggagcagttcaacagcacctaccgcgtggtcagcgccctgcgcatccagcaccaggactggactggaggaaaggagttcaagtgcaaggtccacaacgaaggcctcccggcccccatcgtgaggaccatctccaggaccaaagggccggcccgggagccgcaggtgtatgtcctggccccaccccaggaagagctcagcaaaagcacggtcagcctcacctgcatggtcaccagcttctacccagactacatcgccgtggagtggcagagaaacgggcagcctgagtcggaggacaagtacggcacgaccccgccccagctggacgccgacagctcctacttcctgtacagcaagctcagggtggacaggaacagctggcaggaaggagacacctacacgtgtgtggtgatgcacgaggccctgcacaatcactacacgcagaagtccacctctaagtctgcgggtaaatga

SEQ ID NO: 94 - DNA, 990 bp, Bos taurus
  source 1..990: mol_type = other DNA
gcctccaccacagccccgaaagtctaccctctgagttcttgctgcggggacaagtccagctccaccgtgaccctgggctgcctggtctccagctacatgcccgagccggtgaccgtgacctggaactcgggtgccctgaagagcggcgtgcacaccttcccggccgtccttcagtcctccgggctctactctctcagcagcatggtgaccgtgcccggcagcacctcaggaacccagaccttcacctgcaacgtagcccacccggccagcagcaccaaggtggacaaggctgttgatcccagatgcaaaacaacctgtgactgttgcccaccgcctgagctccctggaggaccctctgtcttcatcttcccaccgaaacccaaggacaccctcacaatctcgggaacgcccgaggtcacgtgtgtggtggtggacgtgggccacgatgaccccgaggtgaagttctcctggttcgtggacgacgtggaggtaaacacagccacgacgaagccgagagaggagcagttcaacagcacctaccgcgtggtcagcgccctgcgcatccagcaccaggactggactggaggaaaggagttcaagtgcaaggtccacaacgaaggcctcccagcccccatcgtgaggaccatctccaggaccaaagggccggcccgggagccgcaggtgtatgtcctggccccaccccaggaagagctcagcaaaagcacggtcagcctcacctgcatggtcaccagcttctacccagactacatcgccgtggagtggcagagaaatgggcagcctgagtcagaggacaagtacggcacgacccctccccagctggacgccgacggctcctacttcctgtacagcaggctcagggtggacaggaacagctggcaggaaggagacacctacacgtgtgtggtgatgcacgaggccctgcacaatcactacacgcagaagtccacctctaagtctgcgggtaaatga

SEQ ID NO: 95 - DNA, 981 bp, Bos taurus
  source 1..981: mol_type = other DNA
gcctccaccacagccccgaaagtctaccctctggcatccagctgcggagacacatccagctccaccgtgaccctgggctgcctggtgtccagctacatgcccgagccggtgaccgtgacctggaactcgggtgccctgaagagcggcgtgcacaccttcccggctgtccttcagtcctccgggctctactctctcagcagcatggtgaccgtgcccgccagcagctcaggacagaccttcacctgcaacgtagcccacccggccagcagcaccaaggtggacaaggctgttggggtctccattgactgctccaagtgtcataaccagccttgcgtgagggaaccatctgtcttcatcttcccaccgaaacccaaagacaccctgatgatcacaggaacgcccgaggtcacgtgtgtggtggtgaacgtgggccacgataaccccgaggtgcagttctcctggttcgtggatgacgtggaggtgcacacggccaggtcgaagccaagagaggagcagttcaacagcacgtaccgcgtggtcagcgccctgcccatccagcaccaggactggactggaggaaaggagttcaagtgcaaggtcaacaacaaaggcctctcggcccccatcgtgaggatcatctccaggagcaaagggccggcccgggagccgcaggtgtatgtcctggacccacccaaggaagagctcagcaaaagcacgctcagcgtcacctgcatggtcaccggcttctacccagaagatgtagccgtggagtggcagagaaaccggcagactgagtcggaggacaagtaccgcacgaccccgccccagctggacaccgaccgctcctacttcctgtacagcaagctcagggtggacaggaacagctggcaggaaggagacgcctacacgtgtgtggtgatgcacgaggccctgcacaatcactacatgcagaagtccacctctaagtctgcgggtaaatga

SEQ ID NO: 96 - DNA, 981 bp, Bos taurus
  source 1..981: mol_type = other DNA
gcctccaccacagccccgaaagtctaccctctgagttcttgctgcggggacaagtccagctccaccgtgaccctgggctgcctggtgtccagctacatgcccgagccggtgaccgtgacctggaactcgggtgccctgaagagcggcgtgcacaccttcccggccgtccttcagtcctccgggctctactctctcagcagcatggtgaccgtgcccggcagcacctcaggacagaccttcacctgcaacgtagcccacccggccagcagcaccaaggtggacaaggctgttggggtctccagtgactgctccaagcctaataaccagcattgcgtgagggaaccatctgtcttcatcttcccaccgaaacccaaagacaccctgatgatcacaggaacgcccgaggtcacgtgtgtggtggtgaacgtgggccacgataaccccgaggtgcagttctcctggttcgtggacgacgtggaggtgcacacggccaggacgaagccgagagaggagcagttcaacagcacgtaccgcgtggtcagcgccctgcccatccagcaccaggactggactggaggaaaggagttcaagtgcaaggtcaacatcaaaggcctctcggcctccatcgtgaggatcatctccaggagcaaagggccggcccgggagccgcaggtgtatgtcctggacccacccaaggaagagctcagcaaaagcacggtcagcgtcacctgcatggtcatcggcttctacccagaagatgtagacgtggagtggcagagagaccggcagactgagtcggaggacaagtaccgcacgaccccgccccagctggacgccgaccgctcctacttcctgtacagcaagctcagggtggacaggaacagctggcagagaggagacacctacacgtgtgtggtgatgcacgaggccctgcacaatcactacatgcagaagtccacctctaagtctgcgggtaaatga

SEQ ID NO: 97 - DNA, 984 bp, Bos taurus
  source 1..984: mol_type = other DNA
gcctccaccacagccccgaaagtctaccctctgagttcttgctgcggggacaagtccagctcgggggtgaccctgggctgcctggtctccagctacatgcccgagccggtgaccgtgacctggaactcgggtgccctgaagagcggcgtgcacaccttcccggccgtccttcagtcctccgggctctactctctcagcagcatggtgaccgtgcccgccagcagctcaggaacccagaccttcacctgcaacgtagcccacccggccagcagcaccaaggtggacaaggctgttggggtctccagtgactgctccaagcctaataaccagcattgcgtgagggaaccatctgtcttcatcttcccaccgaaacccaaagacaccctgatgatcacaggaacgcccgaggtcacgtgtgtggtggtgaacgtgggccacgataaccccgaggtgcagttctcctggttcgtggacgacgtggaggtgcacacggccaggacgaagccgagagaggagcagttcaacagcacgtaccgcgtggtcagcgccctgcccatccagcaccaggactggactggaggaaaggagttcaagtgcaaggtcaacatcaaaggcctctcggcctccatcgtgaggatcatctccaggagcaaagggccggcccgggagccgcaggtgtatgtcctggacccacccaaggaagagctcagcaaaagcacggtcagcctcacctgcatggtcatcggcttctacccagaagatgtagacgtggagtggcagagagaccggcagactgagtcggaggacaagtaccgcacgaccccgccccagctggacgccgaccgctcctacttcctgtacagcaagctcagggtggacaggaacagctggcagagaggagacacctacacgtgtgtggtgatgcacgaggccctgcacaatcactacatgcagaagtccacctctaagtctgcgggtaaatga

SEQ ID NO: 98 - DNA, 1059 bp, Bos taurus
  source 1..1059: mol_type = other DNA
gcctccaccacagccccgaaagtctaccctctggcatccagctgcggagacacatccagctccaccgtgaccctgggctgcctggtctccagctacatgcccgagccggtgaccgtgacctggaactcgggtgccctgaagagcggcgtgcacaccttcccggccgtccggcagtcctctgggctgtactctctcagcagcatggtgactgtgcccgccagcagctcagaaacccagaccttcacctgcaacgtagcccacccggccagcagcaccaaggtggacaaggctgtcactgcaaggcgtccagtcccgacgacgccaaagacaactatccctcctggaaaacccacaaccccaaagtctgaagttgaaaagacaccctgccagtgttccaaatgcccagaacctctgggaggactgtctgtcttcatcttcccaccgaaacccaaggacaccctcacaatctcgggaacgcccgaggtcacgtgtgtggtggtggacgtgggccaggatgaccccgaggtgcagttctcctggttcgtggacgacgtggaggtgcacacggccaggacgaagccgagagaggagcagttcaacagcacctaccgcgtggtcagcgccctgcgcatccagcaccaggactggctgcagggaaaggagttcaagtgcaaggtcaacaacaaaggcctcccggcccccattgtgaggaccatctccaggaccaaagggcaggcccgggagccgcaggtgtatgtcctggccccaccccgggaagagctcagcaaaagcacgctcagcctcacctgcctgatcaccggtttctacccagaagagatagacgtggagtggcagagaaatgggcagcctgagtcggaggacaagtaccacacgaccgcaccccagctggatgctgacggctcctacttcctgtacagcaagctcagggtgaacaagagcagctggcaggaaggagaccactacacgtgtgcagtgatgcacgaagctttacggaatcactacaaagagaagtccatctcgaggtctccgggtaaatga

SEQ ID NO: 99 - DNA, 1059 bp, Bos taurus
  source 1..1059: mol_type = other DNA
gcctccaccacagccccgaaagtctaccctctggcatcccgctgcggagacacatccagctccaccgtgaccctgggctgcctggtctccagctacatgcccgagccggtgaccgtgacctggaactcgggtgccctgaagagtggcgtgcacaccttcccggccgtccttcagtcctccgggctgtactctctcagcagcatggtgaccgtgcccgccagcacctcagaaacccagaccttcacctgcaacgtagcccacccggccagcagcaccaaggtggacaaggctgtcactgcaaggcgtccagtcccgacgacgccaaagacaaccatccctcctggaaaacccacaacccaggagtctgaagttgaaaagacaccctgccagtgttccaaatgcccagaacctctgggaggactgtctgtcttcatcttcccaccgaaacccaaggacaccctcacaatctcgggaacgcccgaggtcacgtgtgtggtggtggacgtgggccaggatgaccccgaggtgcagttctcctggttcgtggacgacgtggaggtgcacacggccaggacgaagccgagagaggagcagttcaacagcacctaccgcgtggtcagcgccctgcgcatccagcaccaggactggctgcagggaaaggagttcaagtgcaaggtcaacaacaaaggcctcccggcccccattgtgaggaccatctccaggaccaaagggcaggcccgggagccgcaggtgtatgtcctggccccaccccgggaagagctcagcaaaagcacgctcagcctcacctgcctgatcaccggtttctacccagaagagatagacgtggagtggcagagaaatgggcagcctgagtcggaggacaagtaccacacgaccgcaccccagctggatgctgacggctcctacttcctgtacagcaggctcagggtgaacaagagcagctggcaggaaggagaccactacacgtgtgcagtgatgcatgaagctttacggaatcactacaaagagaagtccatctcgaggtctccgggtaaatga

SEQ ID NO: 100 - protein, 105 aa, Bos taurus
QPKSPPSVTLFPPSTEELNGNKATLVCLISDFYPGSVTVVWKADGSTITRNVETTRASKQSNSKYAASSYLSLTSSDWKSKGSYSCEVTHEGSTVTKTVKPSECS

SEQ ID NO: 101 - DNA, 318 bp, Bos taurus
  source 1..318: mol_type = other DNA
cagcccaagtccccaccctcggtcaccctgttcccgccctccacggaggagctcaacggcaacaaggccaccctggtgtgtctcatcagcgacttctacccgggtagcgtgaccgtggtctggaaggcagacggcagcaccatcacccgcaacgtggagaccacccgggcctccaaacagagcaacagcaagtacgcggccagcagctacctgagcctgacgagcagcgactggaaatcgaaaggcagttacagctgcgaggtcacgcacgaggggagcaccgtgacgaagacagtgaagccctcagagtgttcttag

SEQ ID NO: 102 - protein, 328 aa, Bos taurus
ASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPPVAGPSVFIFPPKPKDTLTISGTPEVTCVVVDVGHDDPEVKFSWFVDDVEVNTATTKPREEQFNSTYRVVSALRIQHQDWTGGKEFKCKVHNEGLPSSIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 103 - DNA, 987 bp, Bos taurus
  source 1..987: mol_type = other DNA
gctagcacaactgctcctaaggtgtaccccctgagctcttgctgcggcgacaagtctagcagcaccgtgaccctcggatgcctcgtcagcagctatatgcctgagccagttacagtgacatggaattctggtgcccttaagtccggcgtccataccttccctgctgtgctgcagtcctctggcctgtacagtttgtcctctatggtgacagtacccggttccacctccggacagacctttacctgtaatgtggctcatcccgcctcctccacaaaggtggataaggctgttgaccctacctgtaaacccagtccatgcgactgctgtcccccccctccagttgccggaccctcagtctttattttcccacccaaacccaaagacaccctgacaatctctggaacaccagaagtcacctgcgtcgtcgtggatgtgggccacgacgatcctgaggtaaaattctcatggttcgtcgacgatgtggaagtgaatacagctactacaaaacctcgcgaagagcagtttaactctacctatcgagtggtttctgctttgcggattcagcatcaggattggacaggcggcaaagagtttaaatgtaaagtccataacgagggacttccttctagtatcgtgcgcactatcagtagaactaaagggcctgctcgggaacctcaggtgtacgtcctggcacctccacaggaagagctgagtaagtctacagtttctctgacttgtatggtaacatctttttatccagattacatcgcagttgaatggcagaggaacgggcagccagagagtgaggataagtacgggactactccaccacagctggacgcagactcaagttacttcctgtactcaaagctgagggttgacagaaactcatggcaggagggggacacttacacttgcgtagttatgcacgaggcacttcacaaccactacactcagaagagtacttcaaagagtgcagggaagtaa

SEQ ID NO: 104 - DNA, 30 bp, synthetic construct
  misc_feature 1..30: note = primer
  source 1..30: mol_type = other DNA
cgcggatatcatggattacacagcgaagtg

SEQ ID NO: 105 - DNA, 29 bp, synthetic construct
  misc_feature 1..29: note = primer
  source 1..29: mol_type = other DNA
cggggtaccccagagctgttgctggttat

SEQ ID NO: 106 - DNA, 30 bp, synthetic construct; identical to SEQ ID NO: 35
  misc_feature 1..30: note = primer
  source 1..30: mol_type = other DNA

SEQ ID NO: 107 - DNA, 49 bp, synthetic construct
  misc_feature 1..49: note = primer
  source 1..49: mol_type = other DNA
cgcggatatcttaatggtgatggtgatggtgagtcctctcacttgctgg

SEQ ID NO: 108 - DNA, 22 bp, synthetic construct
  misc_feature 1..22: note = primer
  source 1..22: mol_type = other DNA
aagcttcgattgaccagagcag

SEQ ID NO: 109 - DNA, 21 bp, synthetic construct
  misc_feature 1..21: note = primer
  source 1..21: mol_type = other DNA
tcaccataaagggcctccaac

SEQ ID NO: 110 - DNA, 21 bp, synthetic construct
  misc_feature 1..21: note = primer
  source 1..21: mol_type = other DNA
tggcgtcgtgattagtgatga

SEQ ID NO: 111 - DNA, 21 bp, synthetic construct
  misc_feature 1..21: note = primer
  source 1..21: mol_type = other DNA
cagagggctacgatgtgatgg

SEQ ID NO: 112 - DNA, 399 bp, synthetic construct
  misc_feature 1..399: note = codon-optimized sequence
  source 1..399: mol_type = other DNA
atggaatctcaaactcatgttttgatttcattacttctgagtgtttccggaacctacggtgatatcgctatcactcaatctccctcctctgttgctgtgtctgtgggcgaaaccgttaccctgtcctgcaagtccagtcagtctcttctctactccgagaatcaaaaggactacctgggctggtaccaacagaagcccggccagaccccaaagccactgatatactgggcaaccaacaggcacaccggagtgcccgacaggttcacaggcagtggatctggcaccgactttaccttgatcatttcaagcgtgcaggctgaagatctggccgactactactgtggtcagtatctggtgtatcctttcactttcgggccagggacaaaactcgagctcaaa

SEQ ID NO: 113 - DNA, 411 bp, synthetic construct
  misc_feature 1..411: note = codon-optimized sequence
  source 1..411: mol_type = other DNA
atggggtggtcccagattatattgttcctcgtcgccgccgccacttgcgtacacagccaagtgcaacttcaacaaagcggtgcagaactggtaaagcccggtagctctgtgaaaatatcctgtaaagccagtggctacacatttaccagcaactttatgcactgggtgaagcaacagcccggaaatggcttggagtggattggctggatctatcccgaatatggtaacaccaagtataatcagaagttcgacggtaaggccaccctcaccgccgataagtcatcctccaccgcctatatgcagctcagcagcctgaccagcgaggattccgctgtgtacttctgtgccagcgaagaggctgtgatctcattggtgtattggggacagggcaccctcgtcaccgtgtccagc

SEQ ID NO: 114 - DNA, 318 bp, synthetic construct
  misc_feature 1..318: note = codon-optimized sequence
  source 1..318: mol_type = other DNA
cagcctaagagtcctccttctgtaacactctttcccccctctaccgaggaactcaacggcaataaagctaccttggtttgccttatttctgatttctaccccgggtctgtgaccgtggtgtggaaagctgatgggtccaccattactcggaatgtggaaaccacccgggcttctaagcagtccaactctaaatacgcagcatcctcctatttgagtcttactagtagtgactggaagtcaaagggtagttacagttgcgaagtcacacatgaaggttcaacagtgacaaagacagtcaagccctcagagtgctcatag

SEQ ID NO: 115 - protein, 238 aa, synthetic construct
  REGION 1..238: note = chimeric L chain
MESQTHVLISLLLSVSGTYGDIAITQSPSSVAVSVGETVTLSCKSSQSLLYSENQKDYLGWYQQKPGQTPKPLIYWATNRHTGVPDRFTGSGSGTDFTLIISSVQAEDLADYYCGQYLVYPFTFGPGTKLELKQPKSPPSVTLFPPSTEELNGNKATLVCLISDFYPGSVTVVWKADGSTITRNVETTRASKQSNSKYAASSYLSLTSSDWKSKGSYSCEVTHEGSTVTKTVKPSECS

SEQ ID NO: 116 - protein, 465 aa, synthetic construct
  REGION 1..465: note = chimeric H chain
MGWSQIILFLVAAATCVHSQVQLQQSGAELVKPGSSVKISCKASGYTFTSNFMHWVKQQPGNGLEWIGWIYPEYGNTKYNQKFDGKATLTADKSSSTAYMQLSSLTSEDSAVYFCASEEAVISLVYWGQGTLVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPPVAGPSVFIFPPKPKDTLTISGTPEVTCVVVDVGHDDPEVKFSWFVDDVEVNTATTKPREEQFNSTYRVVSALRIQHQDWTGGKEFKCKVHNEGLPSSIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 117 - DNA, 717 bp, synthetic construct
  misc_feature 1..717: note = codon-optimized sequence
  source 1..717: mol_type = other DNA
atggaatctcaaactcatgttttgatttcattacttctgagtgtttccggaacctacggtgatatcgctatcactcaatctccctcctctgttgctgtgtctgtgggcgaaaccgttaccctgtcctgcaagtccagtcagtctcttctctactccgagaatcaaaaggactacctgggctggtaccaacagaagcccggccagaccccaaagccactgatatactgggcaaccaacaggcacaccggagtgcccgacaggttcacaggcagtggatctggcaccgactttaccttgatcatttcaagcgtgcaggctgaagatctggccgactactactgtggtcagtatctggtgtatcctttcactttcgggccagggacaaaactcgagctcaaacagcctaagagtcctccttctgtaacactctttcccccctctaccgaggaactcaacggcaataaagctaccttggtttgccttatttctgatttctaccccgggtctgtgaccgtggtgtggaaagctgatgggtccaccattactcggaatgtggaaaccacccgggcttctaagcagtccaactctaaatacgcagcatcctcctatttgagtcttactagtagtgactggaagtcaaagggtagttacagttgcgaagtcacacatgaaggttcaacagtgacaaagacagtcaagccctcagagtgctcatag

SEQ ID NO: 118 - DNA, 1398 bp, synthetic construct
  misc_feature 1..1398: note = codon-optimized sequence
  source 1..1398: mol_type = other DNA
atggggtggtcccagattatattgttcctcgtcgccgccgccacttgcgtacacagccaagtgcaacttcaacaaagcggtgcagaactggtaaagcccggtagctctgtgaaaatatcctgtaaagccagtggctacacatttaccagcaactttatgcactgggtgaagcaacagcccggaaatggcttggagtggattggctggatctatcccgaatatggtaacaccaagtataatcagaagttcgacggtaaggccaccctcaccgccgataagtcatcctccaccgcctatatgcagctcagcagcctgaccagcgaggattccgctgtgtacttctgtgccagcgaagaggctgtgatctcattggtgtattggggacagggcaccctcgtcaccgtgtccagcgctagcacaactgctcctaaggtgtaccccctgagctcttgctgcggcgacaagtctagcagcaccgtgaccctcggatgcctcgtcagcagctatatgcctgagccagttacagtgacatggaattctggtgcccttaagtccggcgtccataccttccctgctgtgctgcagtcctctggcctgtacagtttgtcctctatggtgacagtacccggttccacctccggacagacctttacctgtaatgtggctcatcccgcctcctccacaaaggtggataaggctgttgaccctacctgtaaacccagtccatgcgactgctgtcccccccctccagttgccggaccctcagtctttattttcccacccaaacccaaagacaccctgacaatctctggaacaccagaagtcacctgcgtcgtcgtggatgtgggccacgacgatcctgaggtaaaattctcatggttcgtcgacgatgtggaagtgaatacagctactacaaaacctcgcgaagagcagtttaactctacctatcgagtggtttctgctttgcggattcagcatcaggattggacaggcggcaaagagtttaaatgtaaagtccataacgagggacttccttctagtatcgtgcgcactatcagtagaactaaagggcctgctcgggaacctcaggtgtacgtcctggcacctccacaggaagagctgagtaagtctacagtttctctgacttgtatggtaacatctttttatccagattacatcgcagttgaatggcagaggaacgggcagccagagagtgaggataagtacgggactactccaccacagctggacgcagactcaagttacttcctgtactcaaagctgagggttgacagaaactcatggcaggagggggacacttacacttgcgtagttatgcacgaggcacttcacaaccactacactcagaagagtacttcaaagagtgcagggaagtaa

SEQ ID NO: 119 - DNA, 20 bp, synthetic construct
  misc_feature 1..20: note = primer
  source 1..20: mol_type = other DNA
ttttacgtgcccaaggttaa

SEQ ID NO: 120 - DNA, 20 bp, synthetic construct
  misc_feature 1..20: note = primer
  source 1..20: mol_type = other DNA
cgtttactgttgcatcatca

SEQ ID NO: 121 - DNA, 19 bp, synthetic construct
  misc_feature 1..19: note = primer
  source 1..19: mol_type = other DNA
tgctggatgactttaaggg

SEQ ID NO: 122 - DNA, 19 bp, synthetic construct
  misc_feature 1..19: note = primer
  source 1..19: mol_type = other DNA
agggcagaaagcgatgaca

SEQ ID NO: 123 - DNA, 20 bp, synthetic construct
  misc_feature 1..20: note = primer
  source 1..20: mol_type = other DNA
ataaccaggtcattcaaagg

SEQ ID NO: 124 - DNA, 20 bp, synthetic construct
  misc_feature 1..20: note = primer
  source 1..20: mol_type = other DNA
attctgacttctcttccgct

SEQ ID NO: 125 - DNA, 20 bp, synthetic construct
  misc_feature 1..20: note = primer
  source 1..20: mol_type = other DNA
taacaagccagtagcccacg

SEQ ID NO: 126 - DNA, 21 bp, synthetic construct
  misc_feature 1..21: note = primer
  source 1..21: mol_type = other DNA
gcaagggctcttgatggcaga

SEQ ID NO: 127 - DNA, 24 bp, synthetic construct
  misc_feature 1..24: note = primer
  source 1..24: mol_type = other DNA
ctgctgaggctcaagttaaaagtg

SEQ ID NO: 128 - DNA, 19 bp, synthetic construct
  misc_feature 1..19: note = primer
  source 1..19: mol_type = other DNA
cagccggttgctgaggtag

SEQ ID NO: 129 - DNA, 20 bp, synthetic construct
  misc_feature 1..20: note = primer
  source 1..20: mol_type = other DNA
cacaacctgagcctgcacaa

SEQ ID NO: 130 - DNA, 22 bp, synthetic construct
  misc_feature 1..22: note = primer
  source 1..22: mol_type = other DNA
tcttgcggaactcaaactcatc

SEQ ID NO: 131 - DNA, 21 bp, synthetic construct
  misc_feature 1..21: note = primer
  source 1..21: mol_type = other DNA
atggaaacaaccagtcggtga

SEQ ID NO: 132 - DNA, 22 bp, synthetic construct
  misc_feature 1..22: note = primer
  source 1..22: mol_type = other DNA
tttctgcacatactccatcgct

SEQ ID NO: 133 - DNA, 20 bp, synthetic construct
  misc_feature 1..20: note = primer
  source 1..20: mol_type = other DNA
tcttccagccttccttcctg

SEQ ID NO: 134 - DNA, 20 bp, synthetic construct
  misc_feature 1..20: note = primer
  source 1..20: mol_type = other DNA
accgtgttggcgtagaggtc

SEQ ID NO: 135 - DNA, 23 bp, synthetic construct
  misc_feature 1..23: note = primer
  source 1..23: mol_type = other DNA
ggcgtgaaccacgagaagtataa

SEQ ID NO: 136 - DNA, 19 bp, synthetic construct
  misc_feature 1..19: note = primer
  source 1..19: mol_type = other DNA
ccctccacgatgccaaagt

SEQ ID NO: 137 - DNA, 20 bp, synthetic construct
  misc_feature 1..20: note = primer
  source 1..20: mol_type = other DNA
gggggtttactgttgcttga

SEQ ID NO: 138 - DNA, 20 bp, synthetic construct
  misc_feature 1..20: note = primer
  source 1..20: mol_type = other DNA
gccactcaggacttggtgat

SEQ ID NO: 139 - DNA, 20 bp, synthetic construct
  misc_feature 1..20: note = primer
  source 1..20: mol_type = other DNA
acgttttctcgtgaagccct

SEQ ID NO: 140 - DNA, 20 bp, synthetic construct
  misc_feature 1..20: note = primer
  source 1..20: mol_type = other DNA
tctaccagaagggcgggata

SEQ ID NO: 141 - DNA, 20 bp, synthetic construct
  misc_feature 1..20: note = primer
  source 1..20: mol_type = other DNA
gtgaccatcgccacctactt

SEQ ID NO: 142 - DNA, 20 bp, synthetic construct
  misc_feature 1..20: note = primer
  source 1..20: mol_type = other DNA
ctcatcgcacsgatgatgct

SEQ ID NO: 143 - DNA, 32 bp, synthetic construct
  misc_feature 1..32: note = primer
  source 1..32: mol_type = other DNA
atatgcggccgcatggggaccccgcgggcgct

SEQ ID NO: 144 - DNA, 30 bp, synthetic construct
  misc_feature 1..30: note = primer
  source 1..30: mol_type = other DNA
gcgcaagctttcagaggggccaggagcagt

SEQ ID NO: 145 - DNA, 35 bp, synthetic construct
  misc_feature 1..35: note = primer
  source 1..35: mol_type = other DNA
ctagctagcaccatgaggatatatagtgtcttaac

SEQ ID NO: 146 - DNA, 31 bp, synthetic construct
  misc_feature 1..31: note = primer
  source 1..31: mol_type = other DNA
caatctcgagttacagacagaagatgactgc

SEQ ID NO: 147 - DNA, 29 bp, synthetic construct
  misc_feature 1..29: note = primer
  source 1..29: mol_type = other DNA
gctagcatgaggatatatagtgtcttaac

SEQ ID NO: 148 - DNA, 26 bp, synthetic construct
  misc_feature 1..26: note = primer
  source 1..26: mol_type = other DNA
gatatcattcctcttttttgctggat

SEQ ID NO: 149 - protein, 131 aa, Rattus norvegicus
MKVPGRLLVLLFWIPASRSDVVLTQTPVSLSVTLGDQASISCRSSQSLEYSDGYTYLEWYLQKPGQSPQLLIYGVSNRFSGVPDRFIGSGSGTDFTLKISRVEPEDLGVYYCFQATHDPDTFGAGTKLELK

SEQ ID NO: 150 - protein, 138 aa, Rattus norvegicus
MAILVLLLCLVTIPHSVLSQVQLKETGPGLVQPTQTLSITCTVSGFSLTSYYIQWVRQTPGKGLEWMGFIRSGGSTEYNSEFKSRLSINRDTSKNQVFLKMNSLKTEDTGVYYCARTSSGYEGGFDYWGQGVMVTVSS

SEQ ID NO: 151 - protein, 105 aa, Bos taurus; identical to SEQ ID NO: 100

SEQ ID NO: 152 - protein, 328 aa, Bos taurus; identical to SEQ ID NO: 102

SEQ ID NO: 153 - DNA, 393 bp, Rattus norvegicus
  source 1..393: mol_type = other DNA
atgaaagtgcctggtaggctgctggtgctgttgttttggattccagcttccaggagtgatgttgtgttgacacaaactccagtttccctgtctgtcacacttggagatcaagcttctatatcttgcaggtctagtcagagcctggaatatagtgatggatacacttatttggaatggtacctacagaagccaggccagtctccacagctcctcatctatggagtttccaaccgattttctggggtcccagacaggttcattggcagtgggtcagggacagatttcaccctcaagatcagcagagtagagcctgaggacttgggagtttattactgcttccaagctacacatgatccggacacgtttggagctgggaccaagctggaactgaaa

SEQ ID NO: 154 - DNA, 414 bp, Rattus norvegicus
  source 1..414: mol_type = other DNA
atggctatcctggtgctgcttctctgcctggtgaccattccacactctgtcttgtcccaggtgcagctgaaggagacaggacctggcctggtgcaaccaacacagaccctgtccatcacatgtactgtttctgggttctcattaaccagctattatatacagtgggttcgccagactccaggaaagggactagaatggatgggatttatacggagtggtggaagcacagagtataattcagagttcaaatcccgacttagcatcaacagggacacctccaagaaccaagttttcttaaaaatgaacagtctgaaaacagaggacacaggcgtgtactactgtgccagaacctcttcggggtacgaagggggttttgattactggggccaaggagtcatggtcacagtctcctca

SEQ ID NO: 155 - DNA, 318 bp, Bos taurus; identical to SEQ ID NO: 101
  source 1..318: mol_type = other DNA

SEQ ID NO: 156 - DNA, 987 bp, synthetic construct
  misc_feature 1..987: note = codon-optimized sequence
  source 1..987: mol_type = other DNA
gctagcaccacagcacctaaagtttaccctctgtcttcctgctgcggcgacaagtcttcatcaactgttactcttggatgcctggtctcaagttacatgcccgagcccgtgacagtgacctggaactcaggcgctctgaagtctggagtgcacacatttccagctgtgcttcagtctagcggcctgtattccctcagctctatggttactgtacctggtagcaccagcggacagactttcacctgtaatgttgcccatcccgcatcttctaccaaggtcgataaagccgttgaccccacttgcaaaccatccccttgtgattgttgtccaccccctccagtggctggcccttccgtcttcattttccctcctaaacctaaggatactctgaccatctcagggacacccgaggtcacctgtgtcgtcgtggacgtgggacatgacgacccagaagtcaagttctcatggttcgtggacgatgtggaggtgaacacagcaacaacaaagcccagagaagaacagtttaacagcacatatcgggtggtcagcgccttgcgtattcagcaccaggactggactggtggcaaggagtttaagtgcaaggtgcataacgaaggtctgccctcttctatagtgagaactatctcccgaactaagggccccgctcgggagccccaggtttacgtccttgctccccctcaggaggaactgagtaaatcaaccgtgagtctcacctgtatggttacctcattttacccagactacatcgccgtagagtggcagaggaatggacagccagagtctgaggacaaatacggcactactcctccccaactggatgccgactcttcctacttcctctactccaaattgcgagttgaccggaactcatggcaggagggggacacatacacatgcgtcgttatgcacgaggccctgcacaaccattacacccagaagtccacatctaaaagtgcaggtaagtaa

SEQ ID NO: 157 - protein, 236 aa, synthetic construct
  REGION 1..236: note = chimeric L chain
MKVPGRLLVLLFWIPASRSDVVLTQTPVSLSVTLGDQASISCRSSQSLEYSDGYTYLEWYLQKPGQSPQLLIYGVSNRFSGVPDRFIGSGSGTDFTLKISRVEPEDLGVYYCFQATHDPDTFGAGTKLELKQPKSPPSVTLFPPSTEELNGNKATLVCLISDFYPGSVTVVWKADGSTITRNVETTRASKQSNSKYAASSYLSLTSSDWKSKGSYSCEVTHEGSTVTKTVKPSECS

SEQ ID NO: 158 - protein, 466 aa, synthetic construct
  REGION 1..466: note = chimeric H chain
MAILVLLLCLVTIPHSVLSQVQLKETGPGLVQPTQTLSITCTVSGFSLTSYYIQWVRQTPGKGLEWMGFIRSGGSTEYNSEFKSRLSINRDTSKNQVFLKMNSLKTEDTGVYYCARTSSGYEGGFDYWGQGVMVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPPVAGPSVFIFPPKPKDTLTISGTPEVTCVVVDVGHDDPEVKFSWFVDDVEVNTATTKPREEQFNSTYRVVSALRIQHQDWTGGKEFKCKVHNEGLPSSIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 159 - DNA, 393 bp, synthetic construct
  misc_feature 1..393: note = codon-optimzed sequence
  source 1..393: mol_type = other DNA
atgaaggtccctggtaggctgctggttctcttgttctggatccctgcttccagaagtgacgtggtgctgactcaaacaccagtgagtctcagtgtgaccctcggcgaccaggcctccatttcttgccgtagcagccagtccttggaatactctgatggttatacttatctggagtggtacctccagaagcccgggcagtcaccccagcttcttatctacggtgtgagcaacagattttctggggttcctgatcggtttattggatctggatccggtaccgacttcacattgaaaatttcacgcgtcgaacccgaggatctgggggtctactattgcttccaagccacccacgatcccgacaccttcggcgctggcactaagctggagctgaaa

SEQ ID NO: 160 - DNA, 414 bp, synthetic construct
  misc_feature 1..414: note = codon-optimized sequence
  source 1..414: mol_type = other DNA
atggcaatcctcgtgttgcttctgtgcttggtgaccattccacactctgtgctttcccaggtgcagctcaaggaaacagggccaggactcgtccaacctacacaaaccctgtcaatcacctgtaccgtatccggttttagcctcaccagctattatatacaatgggtgaggcagacccccgggaaaggactggaatggatgggcttcattcgcagcggtgggagtaccgagtacaatagcgagtttaaaagtcgcttgagtatcaatagagatacttccaagaatcaggtgttcttgaagatgaactccctcaagaccgaagatacaggggtctattactgcgccaggacctccagtggatatgaaggaggctttgattattgggggcagggcgtcatggtaactgtgagctca

SEQ ID NO: 161 - DNA, 318 bp, synthetic construct
  misc_feature 1..318: note = codon-optimized sequence
  source 1..318: mol_type = other DNA
caacccaagtcaccaccatccgtgactctgtttcctccatctacagaggagctgaacggaaacaaagctaccttggtgtgtctcatctctgacttttaccccggatctgtcactgtggtatggaaagcagatggcagcacaataaccaggaatgttgaaaccacacgagcctccaagcagtccaatagtaagtatgccgcatcttcatatctgtcccttacaagctcagactggaaatccaaaggcagctacagttgcgaggtcacacatgaaggcagcaccgtgacaaagaccgtaaagccatctgagtgtagctag

SEQ ID NO: 162 - DNA, 711 bp, synthetic construct
  misc_feature 1..711: note = codon-optimized sequence
  source 1..711: mol_type = other DNA
atgaaggtccctggtaggctgctggttctcttgttctggatccctgcttccagaagtgacgtggtgctgactcaaacaccagtgagtctcagtgtgaccctcggcgaccaggcctccatttcttgccgtagcagccagtccttggaatactctgatggttatacttatctggagtggtacctccagaagcccgggcagtcaccccagcttcttatctacggtgtgagcaacagattttctggggttcctgatcggtttattggatctggatccggtaccgacttcacattgaaaatttcacgcgtcgaacccgaggatctgggggtctactattgcttccaagccacccacgatcccgacaccttcggcgctggcactaagctggagctgaaacaacccaagtcaccaccatccgtgactctgtttcctccatctacagaggagctgaacggaaacaaagctaccttggtgtgtctcatctctgacttttaccccggatctgtcactgtggtatggaaagcagatggcagcacaataaccaggaatgttgaaaccacacgagcctccaagcagtccaatagtaagtatgccgcatcttcatatctgtcccttacaagctcagactggaaatccaaaggcagctacagttgcgaggtcacacatgaaggcagcaccgtgacaaagaccgtaaagccatctgagtgtagctag

SEQ ID NO: 163 - DNA, 1401 bp, synthetic construct
  misc_feature 1..1401: note = codon-optimized sequence
  source 1..1401: mol_type = other DNA
atggcaatcctcgtgttgcttctgtgcttggtgaccattccacactctgtgctttcccaggtgcagctcaaggaaacagggccaggactcgtccaacctacacaaaccctgtcaatcacctgtaccgtatccggttttagcctcaccagctattatatacaatgggtgaggcagacccccgggaaaggactggaatggatgggcttcattcgcagcggtgggagtaccgagtacaatagcgagtttaaaagtcgcttgagtatcaatagagatacttccaagaatcaggtgttcttgaagatgaactccctcaagaccgaagatacaggggtctattactgcgccaggacctccagtggatatgaaggaggctttgattattgggggcagggcgtcatggtaactgtgagctcagctagcaccacagcacctaaagtttaccctctgtcttcctgctgcggcgacaagtcttcatcaactgttactcttggatgcctggtctcaagttacatgcccgagcccgtgacagtgacctggaactcaggcgctctgaagtctggagtgcacacatttccagctgtgcttcagtctagcggcctgtattccctcagctctatggttactgtacctggtagcaccagcggacagactttcacctgtaatgttgcccatcccgcatcttctaccaaggtcgataaagccgttgaccccacttgcaaaccatccccttgtgattgttgtccaccccctccagtggctggcccttccgtcttcattttccctcctaaacctaaggatactctgaccatctcagggacacccgaggtcacctgtgtcgtcgtggacgtgggacatgacgacccagaagtcaagttctcatggttcgtggacgatgtggaggtgaacacagcaacaacaaagcccagagaagaacagtttaacagcacatatcgggtggtcagcgccttgcgtattcagcaccaggactggactggtggcaaggagtttaagtgcaaggtgcataacgaaggtctgccctcttctatagtgagaactatctcccgaactaagggccccgctcgggagccccaggtttacgtccttgctccccctcaggaggaactgagtaaatcaaccgtgagtctcacctgtatggttacctcattttacccagactacatcgccgtagagtggcagaggaatggacagccagagtctgaggacaaatacggcactactcctccccaactggatgccgactcttcctacttcctctactccaaattgcgagttgaccggaactcatggcaggagggggacacatacacatgcgtcgttatgcacgaggccctgcacaaccattacacccagaagtccacatctaaaagtgcaggtaagtaa

SEQ ID NO: 164 - protein, 11 aa, Rattus norvegicus
QSLEYSDGYTY

SEQ ID NO: 165 - protein, 9 aa, Rattus norvegicus
FQATHDPDT

SEQ ID NO: 166 - protein, 8 aa, Rattus norvegicus
GFSLTSYY

SEQ ID NO: 167 - protein, 7 aa, Rattus norvegicus
IRSGGST

SEQ ID NO: 168 - protein, 13 aa, Rattus norvegicus
ARTSSGYEGGFDY

SEQ ID NO: 169 - protein, 329 aa, Bos taurus; identical to SEQ ID NO: 84

SEQ ID NO: 170 - protein, 329 aa, Bos taurus; identical to SEQ ID NO: 84

SEQ ID NO: 171 - protein, 329 aa, Bos taurus; identical to SEQ ID NO: 86

SEQ ID NO: 172 - protein, 326 aa, Bos taurus; identical to SEQ ID NO: 87

SEQ ID NO: 173 - protein, 326 aa, Bos taurus; identical to SEQ ID NO: 88

SEQ ID NO: 174 - protein, 327 aa, Bos taurus; identical to SEQ ID NO: 89

SEQ ID NO: 175 - protein, 352 aa, Bos taurus; identical to SEQ ID NO: 90

SEQ ID NO: 176 - protein, 352 aa, Bos taurus; identical to SEQ ID NO: 91

SEQ ID NO: 177 - DNA, 990 bp, Bos taurus; identical to SEQ ID NO: 92
  source 1..990: mol_type = other DNA

SEQ ID NO: 178 - DNA, 990 bp, Bos taurus; identical to SEQ ID NO: 93
  source 1..990: mol_type = other DNA

SEQ ID NO: 179 - DNA, 990 bp, Bos taurus; identical to SEQ ID NO: 94
  source 1..990: mol_type = other DNA

SEQ ID NO: 180 - DNA, 981 bp, Bos taurus; identical to SEQ ID NO: 95
  source 1..981: mol_type = other DNA

SEQ ID NO: 181 - DNA, 981 bp, Bos taurus; identical to SEQ ID NO: 96
  source 1..981: mol_type = other DNA

SEQ ID NO: 182 - DNA, 984 bp, Bos taurus; identical to SEQ ID NO: 97
  source 1..984: mol_type = other DNA

SEQ ID NO: 183 - DNA, 1059 bp, Bos taurus; identical to SEQ ID NO: 98
  source 1..1059: mol_type = other DNA

SEQ ID NO: 184 - DNA, 1059 bp, Bos taurus; identical to SEQ ID NO: 99
  source 1..1059: mol_type = other DNA

SEQ ID NO: 185 - protein, 331 aa, Ovis aries; identical to SEQ ID NO: 42

SEQ ID NO: 186 - DNA, 996 bp, Ovis aries; identical to SEQ ID NO: 43
  source 1..996: mol_type = other DNA

SEQ ID NO: 187 - protein, 329 aa, Ovis aries; identical to SEQ ID NO: 44

SEQ ID NO: 188 - DNA, 990 bp, Ovis aries; identical to SEQ ID NO: 45
  source 1..990: mol_type = other DNA

SEQ ID NO: 189 - protein, 102 aa, Ovis aries; identical to SEQ ID NO: 46

SEQ ID NO: 190 - DNA, 309 bp, Ovis aries; identical to SEQ ID NO: 47
  source 1..309: mol_type = other DNA

SEQ ID NO: 191 - protein, 106 aa, Ovis aries; identical to SEQ ID NO: 48

SEQ ID NO: 192 - DNA, 321 bp, Ovis aries; identical to SEQ ID NO: 49
  source 1..321: mol_type = other DNA

SEQ ID NO: 193 - protein, 266 aa, Bubalus bubalis; identical to SEQ ID NO: 72

SEQ ID NO: 194 - DNA, 801 bp, Bubalus bubalis; identical to SEQ ID NO: 73
  source 1..801: mol_type = other DNA

SEQ ID NO: 195 - protein, 309 aa, Bubalus bubalis; identical to SEQ ID NO: 74

SEQ ID NO: 196 - DNA, 929 bp, Bubalus bubalis; identical to SEQ ID NO: 75
  source 1..929: mol_type = other DNA

SEQ ID NO: 197 - protein, 352 aa, Bubalus bubalis; identical to SEQ ID NO: 76

SEQ ID NO: 198 - DNA, 1059 bp, Bubalus bubalis; identical to SEQ ID NO: 77
  source 1..1059: mol_type = other DNA

SEQ ID NO: 199 - protein, 105 aa, Bubalus bubalis; identical to SEQ ID NO: 78

SEQ ID NO: 200 - DNA, 318 bp, Bubalus bubalis; identical to SEQ ID NO: 79
  source 1..318: mol_type = other DNA

SEQ ID NO: 201 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 202 - DNA, 690 bp, Homo sapiens
  source 1..690: mol_type = other DNA
gagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 203 - protein, 229 aa, Homo sapiens; identical to SEQ ID NO: 80

SEQ ID NO: 204 - DNA, 690 bp, Homo sapiens; identical to SEQ ID NO: 81
  source 1..690: mol_type = other DNA

SEQ ID NO: 205 - protein, 217 aa, Homo sapiens; identical to SEQ ID NO: 82

SEQ ID NO: 206 - DNA, 654 bp, Homo sapiens; identical to SEQ ID NO: 83
  source 1..654: mol_type = other DNA

SEQ ID NO: 207 - protein, 106 aa, Homo sapiens; identical to SEQ ID NO: 11

SEQ ID NO: 208 - DNA, 321 bp, Homo sapiens; identical to SEQ ID NO: 13
  source 1..321: mol_type = other DNA

SEQ ID NO: 209 - DNA, 32 bp, synthetic construct; identical to SEQ ID NO: 143
  misc_feature 1..32: note = primer
  source 1..32: mol_type = other DNA

SEQ ID NO: 210 - DNA, 30 bp, synthetic construct; identical to SEQ ID NO: 144
  misc_feature 1..30: note = primer
  source 1..30: mol_type = other DNA

SEQ ID NO: 211 - DNA, 35 bp, synthetic construct; identical to SEQ ID NO: 145
  misc_feature 1..35: note = primer
  source 1..35: mol_type = other DNA

SEQ ID NO: 212 - DNA, 31 bp, synthetic construct; identical to SEQ ID NO: 146
  misc_feature 1..31: note = primer
  source 1..31: mol_type = other DNA

SEQ ID NO: 213 - DNA, 40 bp, synthetic construct
  misc_feature 1..40: note = primer
  source 1..40: mol_type = other DNA
ataagaatgcggccgccaccatggggaccccgcgggcgct

SEQ ID NO: 214 - DNA, 50 bp, synthetic construct
  misc_feature 1..50: note = primer
  source 1..50: mol_type = other DNA
gccctcgagttaatggtgatggtgatggtggatgaccaggctctgcatct

SEQ ID NO: 215 - DNA, 20 bp, synthetic construct
  misc_feature 1..20: note = primer
  source 1..20: mol_type = other DNA
atggggaccccgcgggcgcc

SEQ ID NO: 216 - DNA, 25 bp, synthetic construct
  misc_feature 1..25: note = primer
  source 1..25: mol_type = other DNA
tcagaggggccaggagcagtgtcca

SEQ ID NO: 217 - DNA, 20 bp, synthetic construct
  misc_feature 1..20: note = primer
  source 1..20: mol_type = other DNA
atggggaccccgcgggcgct

SEQ ID NO: 218 - DNA, 20 bp, synthetic construct
  misc_feature 1..20: note = primer
  source 1..20: mol_type = other DNA
gatgaccaggctctgcatct

SEQ ID NO: 219 - DNA, 20 bp, synthetic construct
  misc_feature 1..20: note = primer
  source 1..20: mol_type = other DNA
aatgacagcggcgtctactt

SEQ ID NO: 220 - DNA, 20 bp, synthetic construct
  misc_feature 1..20: note = primer
  source 1..20: mol_type = other DNA
tcagaggggccaggagcagt

SEQ ID NO: 221 - DNA, 29 bp, synthetic construct
  misc_feature 1..29: note = primer
  source 1..29: mol_type = other DNA
gaagatctatggggaccccgcgggcgccg

SEQ ID NO: 222 - DNA, 29 bp, synthetic construct
  misc_feature 1..29: note = primer
  source 1..29: mol_type = other DNA
gacccggggaggggccaggagcagtgtcc

SEQ ID NO: 223 - DNA, 1404 bp, synthetic construct
  misc_feature 1..1404: note = DNA encoding chimeric H chain
  source 1..1404: mol_type = other DNA
atggcaatcctcgtgttgcttctgtgcttggtgaccattccacactctgtgctttcccaggtgcagctcaaggaaacagggccaggactcgtccaacctacacaaaccctgtcaatcacctgtaccgtatccggttttagcctcaccagctattatatacaatgggtgaggcagacccccgggaaaggactggaatggatgggcttcattcgcagcggtgggagtaccgagtacaatagcgagtttaaaagtcgcttgagtatcaatagagatacttccaagaatcaggtgttcttgaagatgaactccctcaagaccgaagatacaggggtctattactgcgccaggacctccagtggatatgaaggaggctttgattattgggggcagggcgtcatggtaactgtgagctcagcctccaccacagccccgaaagtctaccctctgagttcttgctgcggggacaagtccagctccaccgtgaccctgggctgcctggtctccagctacatgcccgagccggtgaccgtgacctggaactcgggtgccctgaagagcggcgtgcacaccttcccggctgtccttcagtcctccgggctgtactctctcagcagcatggtgaccgtgcccggcagcacctcaggacagaccttcacctgcaacgtagcccacccggccagcagcaccaaggtggacaaggctgttgatcccacatgcaaaccatcaccctgtgactgttgcccaccccctgagctccccggaggaccctctgtcttcatcttcccaccgaaacccaaggacaccctcacaatctcgggaacgcccgaggtcacgtgtgtggtggtggacgtgggccacgatgaccccgaggtgaagttctcctggttcgtggacgacgtggaggtaaacacagccacgacgaagccgagagaggagcagttcaacagcacctaccgcgtggtcagcgccctgcgcatccagcaccaggactggactggaggaaaggagttcaagtgcaaggtccacaacgaaggcctcccggcccccatcgtgaggaccatctccaggaccaaagggccggcccgggagccgcaggtgtatgtcctggccccaccccaggaagagctcagcaaaagcacggtcagcctcacctgcatggtcaccagcttctacccagactacatcgccgtggagtggcagagaaacgggcagcctgagtcggaggacaagtacggcacgaccccgccccagctggacgccgacagctcctacttcctgtacagcaagctcagggtggacaggaacagctggcaggaaggagacacctacacgtgtgtggtgatgcacgaggccctgcacaatcactacacgcagaagtccacctctaagtctgcgggtaaataa

SEQ ID NO: 224 - protein, 467 aa, synthetic construct
  REGION 1..467: note = chimeric H chain
MAILVLLLCLVTIPHSVLSQVQLKETGPGLVQPTQTLSITCTVSGFSLTSYYIQWVRQTPGKGLEWMGFIRSGGSTEYNSEFKSRLSINRDTSKNQVFLKMNSLKTEDTGVYYCARTSSGYEGGFDYWGQGVMVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVDPTCKPSPCDCCPPPELPGGPSVFIFPPKPKDTLTISGTPEVTCVVVDVGHDDPEVKFSWFVDDVEVNTATTKPREEQFNSTYRVVSALRIQHQDWTGGKEFKCKVHNEGLPAPIVRTISRTKGPAREPQVYVLAPPQEELSKSTVSLTCMVTSFYPDYIAVEWQRNGQPESEDKYGTTPPQLDADSSYFLYSKLRVDRNSWQEGDTYTCVVMHEALHNHYTQKSTSKSAGK

SEQ ID NO: 225 - DNA, 28 bp, synthetic construct
  misc_feature 1..28: note = primer
  source 1..28: mol_type = other DNA
ctagctagcaccacagccccgaaagtct

SEQ ID NO: 226 - DNA, 30 bp, synthetic construct
  misc_feature 1..30: note = primer
  source 1..30: mol_type = other DNA
tgctctagattatttacccgcagacttaga

SEQ ID NO: 227 - DNA, 40 bp, synthetic construct
  misc_feature 1..40: note = primer
  source 1..40: mol_type = other DNA
ataagaatgcggccgccaccatgtggctcataatagctct

SEQ ID NO: 228 - DNA, 50 bp, synthetic construct
  misc_feature 1..50: note = primer
  source 1..50: mol_type = other DNA
gccctcgagttaatggtgatggtgatggtgaggagttgttgactggaggc

SEQ ID NO: 229 - DNA, 38 bp, synthetic construct
  misc_feature 1..38: note = primer
  source 1..38: mol_type = other DNA
ataagaatgctagccaccatggggatcccctcattcct

SEQ ID NO: 230 - DNA, 50 bp, synthetic construct
  misc_feature 1..50: note = primer
  source 1..50: mol_type = other DNA
gccgatatcttaatggtgatggtgatggtgcgatgaggggccgctcgagc

SEQ ID NO: 231 - DNA, 40 bp, synthetic construct
  misc_feature 1..40: note = primer
  source 1..40: mol_type = other DNA
ataagaatgcggccgccaccatgtggcaactgctaccacc

SEQ ID NO: 232 - DNA, 50 bp, synthetic construct
  misc_feature 1..50: note = primer
  source 1..50: mol_type = other DNA
gccctcgagttaatggtgatggtgatggtggtgccaaggtagaaagaatg

SEQ ID NO: 233 - DNA, 48 bp, synthetic construct
  misc_feature 1..48: note = primer
  source 1..48: mol_type = other DNA
ataagaatgcggccgccaccatggcccccaccctccctgccttgctct

SEQ ID NO: 234 - DNA, 50 bp, synthetic construct
  misc_feature 1..50: note = primer
  source 1..50: mol_type = other DNA
gccctcgagttaatggtgatggtgatggtgattctgcatcgtgtagtctg

(19) United States
(12) Patent Application Publication
(10) Pub. No.: US 2025/0066483 A1
(43) Pub. Date: Feb. 27, 2025

(54) ANTI-PD-L1 ANTIBODY, ANTI-PD-1 ANTIBODY, AND THE USE OF INHIBITOR TARGETING PD-1/PD-L1 AND COX-2 INHIBITOR

(71) Applicants: Fuso Pharmaceutical Industries, Ltd., Osaka (JP); National University Corporation Hokkaido University, Sapporoshi, Hokkaido (JP)

(72) Inventors: Satoru KONNAI, Sapporoshi (JP); Kazuhiko OHASHI, Sapporoshi (JP); Shiro MURATA, Sapporoshi (JP); Tomohiro OKAGAWA, Sapporoshi (JP); Asami NISHIMORI, Sapporoshi (JP); Naoya MAEKAWA, Sapporoshi (JP); Yasuhiko SUZUKI, Sapporoshi (JP); Chie NAKAJIMA, Sapporoshi (JP)

(73) Assignees: Fuso Pharmaceutical Industries, Ltd., Osaka (JP); National University Corporation Hokkaido University, Sapporoshi, Hokkaido (JP)

(21) Appl. No.: 18/914,727

(22) Filed: Oct. 14, 2024

Related U.S. Application Data
(63) Continuation of application No. 17/579,517, filed on Jan. 19, 2022.
Application No. 17/579,517 is a continuation of application No. 16/325,040, filed on Feb. 12, 2019, now Pat. No. 11,312,773.

(30) Foreign Application Priority Data
Aug. 15, 2016 (JP) 2016-159088
Aug. 15, 2016 (JP) 2016-159089
Mar. 27, 2017 (JP) 2017-061454
Jun. 5, 2017 (JP) 2017-110723

(51) Int. Cl. C07K 16/28

(52) U.S. Cl. CPC C07K 16/2827; C07K 2317/24; C07K 2317/565; C07K 2317/76; C07K 2317/92

(57) ABSTRACT

The present disclosure provides an anti-PD-L1 antibody capable of repeated administration even to animals other than rat. The present disclosure further provides a novel therapeutic strategy using an inhibitor targeting PD-1/PD-L1. A pharmaceutical composition which comprises a COX-2 inhibitor and is administered before, after or simultaneously with the administration of an inhibitor targeting PD-1/PD-L1. A potentiator for the immunostimulatory effect of an inhibitor targeting PD-1/PD-L1, which comprises a COX-2 inhibitor. The present disclosure also provides an anti-PD-1 antibody capable of repeated administration even to animals other than rat. An anti-PD-1 antibody comprising (a) a light chain comprising a light chain variable region containing CDR1 having the amino acid sequence of QSLEYSDGYTY (SEQ ID NO: 164), CDR2 having the amino acid sequence of GVS and CDR3 having the amino acid sequence of FQATHDPDT (SEQ ID NO: 165) and the light chain constant region of an antibody of an animal other than rat; and (b) a heavy chain comprising a heavy chain variable region containing CDR1 having the amino acid sequence of GFSLTSYY (SEQ ID NO: 166), CDR2 having the amino acid sequence of IRSGGST (SEQ ID NO: 167) and CDR3 having the amino acid sequence of ARTSSGYEGGFDY (SEQ ID NO: 168) and the heavy chain constant region of an antibody of an animal other than rat. A pharmaceutical composition comprising the above-described anti-PD-1 antibody as an active ingredient. A method for preparing the anti-PD-1 antibody is also provided.

### TECHNICAL FIELD

The present disclosure relates to an anti-PD-L1 antibody and an anti-PD-1 antibody. More specifically, one aspect of the present disclosure relates to an anti-PD-L1 antibody comprising a variable region containing complementarity-determining regions (CDR) of a rat anti-bovine PD-L1 antibody and a constant region of an antibody of an animal other than rat. The present disclosure also relates to an anti-PD-1 antibody. Moreover, one aspect of the present disclosure relates to an anti-PD-1 antibody comprising a variable region containing complementarity-determining regions (CDRs) of a rat anti-bovine PD-1 antibody and a constant region of an antibody of an animal other than rat. The present disclosure further relates to combined use of an inhibitor targeting PD-1/PD-L1 and a COX-2 inhibitor.

### BACKGROUND ART

Programmed cell death 1 (PD-1), an immunoinhibitory receptor, and its ligand programmed cell death ligand 1 (PD-L1) are molecules identified by Prof Tasuku Honjo et al., Kyoto University, as factors which inhibit excessive immune response and are deeply involved in immunotolerance (Non-Patent Document No. 1: Ishida Y, Agata Y, Shibahara K, Honjo T The EMBO Journal. 1992 November; 11(11):3887-3895). Recently, it has been elucidated that these molecules are also involved in immunosuppression in tumors. In the field of human medical care, an antibody drug that inhibits the effect of PD-1 has been developed and put into practical use (Opdivo™, Ono Pharmaceutical Co., Ltd.).

To date, the present inventors have been developing an immunotherapy for animal refractory diseases targeting PD-1 or PD-L1, and have revealed that this novel immunotherapy is applicable to multiple-diseases and multiple-animals. (Non-Patent Document No. 2: Ikebuchi R, Konnai S, Okagawa T, Yokoyama K, Nakajima C, Suzuki Y, Murata S, Ohashi K. Immunology. 2014 August; 142(4):551-61; Non-Patent Document No. 3: Maekawa N, Konnai S, Ikebuchi R, Okagawa T, Adachi M, Takagi S, Kagawa Y, Nakajima C, Suzuki Y, Murata S, Ohashi K. PLoS One. 2014 Jun. 10; 9(6):e98415; Non-Patent Document No. 4: Mingala C N, Konnai S, Ikebuchi R, Ohashi K. Comp. Immunol. Microbiol. Infect. Dis. 2011 January; 34(1):55-63.). Further, the interaction between PD-1 and PD-L1 is one of the major molecular mechanisms through which tumors and infections evade immune responses. It has been reported that inhibition of the above interaction by using an antibody which specifically binds to either of those molecules can produce antitumor effects and anti-pathogenic effects.

However, the antibodies which the present inventors have prepared to date are rat antibodies, and therefore it is impossible to administer those antibodies repeatedly to animals other than rat.

### DISCLOSURE OF THE INVENTION

### Problem for Solution by the Invention

It is one object of the present disclosure to provide an anti-PD-L1 antibody capable of repeated administration even to animals other than rat. It is also an object of the present disclosure to provide an anti-PD-1 antibody capable of repeated administration even to animals other than rat. It is another object of the present disclosure to provide a novel therapeutic strategy using inhibitors targeting PD-1/PD-L1.

### Means to Solve the Problem

It has been determined that the variable regions of a rat anti-bovine PD-L1 monoclonal antibody (4G12) capable of inhibiting the binding of canine PD-1 to PD-L1, and then combined genes encoding the resultant variable regions with genes encoding the constant regions of a canine immunoglobulin (IgG-D equivalent to human IgG4) to thereby obtain a chimeric antibody gene, which was introduced into Chinese hamster ovary cells (CHO cells). By culturing/proliferating the resultant CHO cells, the present inventors have succeeded in preparing a rat-canine chimeric anti-PD-L1 antibody. Further, it has been determined that the CDRs of the variable region of the rat anti-bovine PD-L1 monoclonal antibody 4G12.

Furthermore, it has been determined the variable regions of the rat anti-bovine PD-L1 monoclonal antibody 4G12 capable of inhibiting the binding of bovine PD-1 to PD-L1, and then combined genes encoding the resultant variable regions with genes encoding the constant regions of a bovine immunoglobulin (bovine IgG1, with mutations having been introduced into the putative binding sites of Fcγ receptors in CH2 domain in order to inhibit ADCC activity; see FIG. 19 for amino acid numbers and mutations: 250 E->P, 251 L->V, 252 P->A, 253 G->deletion, 347 A->S, 348 P->S; Ikebuchi R, Konnai S, Okagawa T, Yokoyama K, Nakajima C, Suzuki Y, Murata S, Ohashi K. Immunology 2014 August; 142(4):551-561) to thereby obtain a chimeric antibody gene. This gene was introduced into Chinese hamster ovary cells (CHO cells). By culturing/proliferating the resultant cells, a rat-bovine chimeric anti-PD-L1 antibody have been prepared successfully. The present disclosure has been achieved based on these findings.

Furthermore, toward establishment of a novel control method for canine tumors and bovine infections, it has been confirmed in in vitro tests an immunostimulatory effect induced by COX-2 inhibitors and enhancement of that effect when such inhibitors are used in combination with anti-PD-L1 antibody. The present disclosure provides what has been achieved based on these findings.

Furthermore, it has been determined the variable regions of a rat anti-bovine PD-1 monoclonal antibody (5D2) capable of inhibiting the binding of bovine PD-1 to PD-L1, and then combined genes encoding the resultant variable regions with genes encoding the constant regions of a bovine immunoglobulin (bovine IgG1, with mutations having been introduced into the putative binding sites of Fcγ receptors in CH2 domain in order to inhibit ADCC activity; see FIGS. 12 and 22 for amino acid numbers and mutations: 251 E->P, 252 L->V, 253 P->A, 254 G->deletion, 348 A->S, 349 P->S; Ikebuchi R, Konnai S, Okagawa T, Yokoyama K, Nakajima C, Suzuki Y, Murata S, Ohashi K. Immunology 2014 August; 142(4):551-561) to thereby obtain a chimeric antibody gene. This gene was introduced into Chinese hamster ovary cells (CHO cells). By culturing/proliferating the resultant cells, the present inventors have succeeded in preparing a rat-bovine chimeric anti-bovine PD-1 antibody. Further, the present inventors have determined the CDRs of the variable regions of rat anti-bovine PD-1 monoclonal antibody (5D2). The present disclosure has been achieved based on these findings.

A summary of the present disclosure according to some embodiments is as described below.
(1) A pharmaceutical composition which comprises a COX-2 inhibitor and is administered before, after or simultaneously with the administration of an inhibitor targeting PD-1/PD-L1.
(2) The pharmaceutical composition of (1) above, wherein the inhibitor targeting PD-1/PD-L1 is an antibody.
(3) The pharmaceutical composition of (1) or (2) above, wherein the antibody is at least one antibody selected from the group consisting of anti-PD-1 antibody and anti-PD-L1 antibody.
(4) The pharmaceutical composition of any one of (1) to (3) above, wherein the COX-2 inhibitor is at least one compound selected from the group consisting of meloxicam, piroxicam, celecoxib, firocoxib, robenacoxib, carprofen and etodolac.
(5) The pharmaceutical composition of any one of (1) to (4) above for use in prevention and/or treatment of cancer and/or infection.
(6) The pharmaceutical composition of any one of (1) to (5) above, wherein the inhibitor targeting PD-1/PD-L1 and the COX-2 inhibitor are administered separately.
(7) The pharmaceutical composition of any one of (1) to (5) above, which is a combination drug comprising the inhibitor targeting PD-1/PD-L1 and the COX-2 inhibitor.
(8) A potentiator for the immunostimulatory effect of an inhibitor targeting PD-1/PD-L1, which comprises a COX-2 inhibitor.
(9) A method of preventing and/or treating cancer and/or infection, comprising administering to a human or animal subject a pharmaceutically effective amount of a COX-2 inhibitor before, after or simultaneously with the administration of an inhibitor targeting PD-1/PD-L1.
(10) Use of a COX-2 inhibitor for preventing and/or treating cancer and/or infection, wherein the COX-2 inhibitor is administered before, after or simultaneously with the administration of an inhibitor targeting PD-1/PD-L1.
(11) Use of a COX-2 inhibitor for use in a method of preventing and/or treating cancer and/or infection, wherein the COX-2 inhibitor is administered before, after or simultaneously with the administration of an inhibitor targeting PD-1/PD-L1.
(12) An anti-PD-1 antibody comprising (a) a light chain comprising a light chain variable region containing CDR1 having the amino acid sequence of QSLEYSDGYTY (SEQ ID NO: 164), CDR2 having the amino acid sequence of GVS and CDR3 having the amino acid sequence of FQATHDPDT (SEQ ID NO: 165) and the light chain constant region of an antibody of an animal other than rat; and (b) a heavy chain comprising a heavy chain variable region containing CDR1 having the amino acid sequence of GFSLTSYY (SEQ ID NO: 166), CDR2 having the amino acid sequence of IRSGGST (SEQ ID NO: 167) and CDR3 having the amino acid sequence of ARTSSGYEGGFDY (SEQ ID NO: 168) and the heavy chain constant region of an antibody of an animal other than rat.
(13) The antibody of (12) above, wherein the light chain variable region and the heavy chain variable region are derived from rat.
(14) The antibody of (13) above, wherein the light chain variable region is the light chain variable region of a rat anti-bovine PD-1 antibody and the heavy chain variable region is the heavy chain variable region of a rat anti-bovine PD-1 antibody.
(15) The antibody of (14) above, wherein the light chain variable region has the amino acid sequence as shown in SEQ ID NO. 149 and the heavy chain variable region has the amino acid sequence as shown in SEQ ID NO: 150.
(16) The antibody of any one of (12) to (15) above, wherein the light chain constant region of an antibody of an animal other than rat has the amino acid sequence of the constant region of lambda chain or kappa chain.
(17) The antibody of any one of (12) to (16) above, wherein the heavy chain constant region of an antibody of an animal other than rat has the amino acid sequence of the constant region of an immunoglobulin equivalent to human IgG4, or has mutations introduced thereinto that reduce ADCC activity and/or CDC activity.
(18) The antibody of (17) above, wherein the animal other than rat is bovine; the light chain constant region of the bovine antibody has the amino acid sequence of the constant region of lambda chain; and the heavy chain constant region of the bovine antibody has mutations introduced thereinto that reduce ADCC activity and/or CDC activity.
(19) The antibody of (18) above, wherein the light chain constant region of the bovine antibody has the amino acid sequence as shown in SEQ ID NO: 151 and the heavy chain constant region of the bovine antibody has the amino acid sequence as shown in SEQ ID NO: 152.
(20) The antibody of any one of (12) to (19) above which has a four-chain structure comprising two light chains and two heavy chains.
(21) A pharmaceutical composition comprising the antibody of any one of (13) to (20) above as an active ingredient.
(22) The composition of (21) above for prevention and/or treatment of cancers and/or inflammations.
(23) The composition of (22) above, wherein the cancers and/or inflammations are selected from the group consisting of neoplastic diseases, leukemia, Johne's disease, anaplasmosis, bacterial mastitis, mycotic mastitis, mycoplasma infections (such as mycoplasma mastitis, mycoplasma pneumonia or the like), tuberculosis, Theileria orientalis infection, cryptosporidiosis, coccidiosis, trypanosomiasis and leishmaniasis.
(24) An artificial genetic DNA comprising (a′) a DNA encoding a light chain comprising a light chain variable region containing CDR1 having the amino acid sequence of QSLEYSDGYTY (SEQ ID NO: 164), CDR2 having the amino acid sequence of GVS and CDR3 having the amino acid sequence of FQATHDPDT (SEQ ID NO: 165) and the light chain constant region of an antibody of an animal other than rat and (b′) a DNA encoding a heavy chain comprising a heavy chain variable region containing CDR1 having the amino acid sequence of GFSLTSYY (SEQ ID NO: 166), CDR2 having the amino acid sequence of IRSGGST (SEQ ID NO: 167) and CDR3 having the amino acid sequence of ARTSSGYEGGFDY (SEQ ID NO: 168) and the heavy chain constant region of an antibody of an animal other than rat.
(25) A vector comprising the artificial genetic DNA of (24) above.
(26) A host cell transformed with the vector of (25) above.
(27) A method of preparing an antibody, comprising culturing the host cell of (26) above and collecting an anti-PD-1 antibody from the resultant culture.
(28) A DNA encoding a light chain comprising a light chain variable region containing CDR1 having the amino acid sequence of QSLEYSDGYTY (SEQ ID NO: 164), CDR2 having the amino acid sequence of GVS and CDR3 having the amino acid sequence of FQATHDPDT (SEQ ID NO: 165) and the light chain constant region of an antibody of an animal other than rat.
(29) A DNA encoding a heavy chain comprising a heavy chain variable region containing CDR1 having the amino acid sequence of GFSLTSYY (SEQ ID NO: 166), CDR2 having the amino acid sequence of IRSGGST (SEQ ID NO: 167) and CDR3 having the amino acid sequence of ARTSSGYEGGFDY (SEQ ID NO: 168) and the heavy chain constant region of an antibody of an animal other than rat.

### Effect of the Invention

Immunostimulatory effect is enhanced by combined use of an inhibitor targeting PD-1/PD-L1 and a COX-2 inhibitor. Moreover, a novel anti-PD-1 antibody has been obtained. This antibody is applicable even to those animals other than rat.

The present specification encompasses the contents disclosed in the specifications and/or drawings of Japanese Patent Application Nos. 2017-140891, No. 2018-016074 2016-159090, and No. 2017-099615, based on which the present patent application claims priority. The present specification further incorporates by reference U.S. Patent Application Publication Nos. 2019/0185568, 2022/0227871 and 2020/0131270 in their entirety.

### BRIEF DESCRIPTION OF THE DRAWINGS

FIG. 1 PGE₂ production from canine tumor cell lines CMeC, LMeC, CMM-1, CMM-2 (these four are derived from melanoma) and HM-POS (derived from osteosarcoma). PGE₂ production tended to be high in CMM-1 and HM-POS.

FIG. 2 COX2 expression levels in canine tumor cell lines CMeC, LMeC, CMM-1, CMM-2 (these four are derived from melanoma) and HM-POS (derived from osteosarcoma). Consistent with PGE₂ production, COX2 expression levels were high in CMM-1 and HM-POS.

FIG. 3 Effect of PGE₂ on canine peripheral blood mononuclear cells (PBMCs). Canine PBMCs were cultured under stimulation for 3 days in the presence of a superantigen SEB and anti-CD28 antibody. Then, IL-2 and IFN-γ concentrations in the resultant culture supernatant were determined by ELISA. PGE₂ inhibited production of IL-2 and IFN-γ from canine PBMCs.

FIG. 4 PGE₂ production inhibitory effect of COX-2 inhibitor upon canine tumor cell lines. Meloxicam showed a tendency to decrease PGE₂ production from canine tumor cell lines CMM-1 (derived from melanoma) and HM-POS (derived from osteosarcoma).

FIG. 5 PGE₂ production inhibitory effect of COX-2 inhibitor upon canine PBMCs. Meloxicam decreased the amount of PGE₂ produced from canine PBMCs cultured for 3 days in the presence of a superantigen SEB and anti-CD28 antibody for stimulation.

FIG. 6 Canine immune cell activating effect of COX-2 inhibitor. Canine PBMCs were cultured for 3 days in the presence of a superantigen SEB and anti-CD28 antibody to stimulate canine lymphocytes. Then, IL-2 concentration in the resultant culture supernatant was determined by ELISA. Meloxicam increased the IL-2 production from canine PBMCs.

FIG. 7 Canine immune cell activating effect by combined use of anti-PD-L1 antibody and COX-2 inhibitor. Canine PBMCs were cultured for 3 days in the presence of a superantigen SEB and anti-CD28 antibody to stimulate canine lymphocytes. Then, IL-2 concentration in the resultant culture supernatant was determined by ELISA. Although anti-PD-L1 antibody taken alone increased the IL-2 production from canine PBMCs, the IL-2 production was further increased when meloxicam was used in combination with the antibody.

FIG. 8 Inhibition of the binding of recombinant canine PD-L1 to recombinant canine PD-1. The binding of canine PD-L1-Ig to canine PD-1-Ig was detected on ELISA plates. The optical density (O.D.) without addition of antibody was taken as 100%. O.D. at each antibody concentration was shown as relative value. Among rat anti-bovine PD-L1 monoclonal antibodies 4G12 (Rat IgG2a (κ)), 5A2 (Rat IgG1 (κ)) and 6G7 (Rat IgM (κ)) which showed cross-reaction with canine PD-L1, clones 4G12 and 6G7 exhibited a high binding inhibition capacity.

FIG. 9 Schematic drawings of pDC6 vector and a rat-canine chimeric anti-PD-L1 antibody.

FIG. 10 Expression and purification of rat-canine chimeric anti-PD-L1 antibodies c4G12 and c6G7. SDS-PAGE was performed under non-reducing conditions, followed by visualization of bands by CBB staining. a: purification with protein A alone. b: a +gel filtration chromatography.

FIG. 11 PD-1/PD-L1 binding inhibition activities of rat-canine chimeric anti-PD-L1 antibodies c4G12 and c6G7.

FIG. 12 Establishment of cell clones capable of high expression of rat-canine chimeric anti-PD-L1 antibody c4G12.

FIG. 13 SDS-PAGE images of rat-canine chimeric anti-PD-L1 antibody c4G12. Rat anti-bovine PD-L1 antibody 4G12 and rat-canine chimeric anti-PD-L1 antibody c4G12 were electrophoresed under reducing conditions and non-reducing conditions, followed by visualization of bands by CBB staining. Under reducing conditions, a band of antibody's heavy chain was detected at around 50 kDa and a band of antibody's light chain at around 25 kDa. No bands other than the bands of interest were detected.

FIG. 14 Inhibitory activities of rat anti-bovine PD-L1 antibody 4G12 and rat-canine chimeric anti-PD-L1 antibody c4G12 against canine PD-1/PD-L1 binding and CD80/PD-L1 binding. Rat anti-bovine PD-L1 monoclonal antibody 4G12 and rat-canine chimeric anti-PD-L1 antibody c4G12 reduced the amounts of binding of PD-L1-Ig to canine PD-1-Ig and CD80-Ig. No change due to chimerization of the antibody was observed in binding inhibition activity

FIG. 15 Canine immune cell activation effect by rat-canine chimeric anti-PD-L1 antibody c4G12. Canine PBMCs were cultured under stimulation for 3 days, followed by determination of IL-2 and IFN-γ concentrations in the supernatant by ELISA. Further, nucleic acid analogue EdU was added to the culture medium at day 2 of the culture under stimulation, followed by determination of the EdU uptake by flow cytometry. Rat-canine chimeric anti-PD-L1 antibody c4G12 increased the production of IL-2 and IFN-γ from canine PBMCs and enhanced proliferation of CD4+ and CD8+ lymphocytes.

FIG. 16 Expression of PD-L1 in oral melanoma (A) and undifferentiated sarcoma (B)

FIG. 17 CT images and appearances of tumor in a test of treatment by administering rat-canine chimeric anti-PD-L1 antibody c4G12 to a dog with oral melanoma. (a,d) Before the start of the treatment, (b,e) at week 10 of the treatment, and (c,f) at week 34 of the treatment. A remarkable antitumor effect was recognized upon five administrations of the antibody (at week 10 from the start of the treatment). At week 34, a further reduction of tumor was confirmed.

FIG. 18 Time-dependent changes in the longest diameter of the tumor in the dog with oral melanoma shown in FIG. 17. Reduction by 30% or more compared to the baseline longest diameter was regarded as partial response (PR).

FIG. 19 CT images in a test of treatment by administering rat-canine chimeric anti-PD-L1 antibody c4G12 to a dog with undifferentiated sarcoma. (a,c) Before the start of the treatment, (b,d) at week 3 of the treatment. A remarkable reduction of tumor was recognized upon two administrations of the antibody.

FIG. 20 CT images in a test of treatment by administering rat-canine chimeric anti-PD-L1 antibody c4G12 to dogs with oral melanoma (pulmonary metastatic cases). (a,d,g) Before the start of the treatment, (b,e,h) at week 6 of the treatment, and (c,f,i) at week 18 of the treatment. A plurality of pulmonary metastatic lesions disappeared upon nine administrations of the antibody.

FIG. 21 Time-dependent changes in the proportion survival of dogs with oral melanoma after the occurrence of pulmonary metastasis. In the antibody administration group, the survival duration may have been prolonged compared to the control group.

FIG. 22 CDR1, CDR2 and CDR3 regions in the light chain variable region and the heavy chain variable region of rat anti-bovine PD-L1 antibody 4G12 are illustrated.

FIG. 23 Effect of PGE₂ on bovine T cell responses.

FIG. 24 Effect of PGE₂ on expression of immune-related genes in bovine PBMCs.

FIG. 25 Effect of PGE₂ on expression of PD-L1 in bovine PBMCs.

FIG. 26 Immunostimulatory effect of COX-2 inhibitor in bovine PBMCs.

FIG. 27 Kinetic analyses of PGE₂ in cattle infected with M. avium subsp. paratuberculosis.

FIG. 28 Changes in PD-L1 expression level by antigen stimulation in cattle infected with M. avium subsp. paratuberculosis.

FIG. 29 Analyses of expression of PGE₂, EP2 and PD-L1 in M. avium subsp. paratuberculosis-infected lesions.

FIG. 30 Activation of M. avium subsp. paratuberculosis-specific immune responses by COX-2 inhibitor.

FIG. 31 Immunostimulatory effect of rat anti-bovine PD-L1 antibody in cattle infected with M. avium subsp. paratuberculosis.

FIG. 32 Combined effect of COX-2 inhibitor and rat anti-bovine PD-L1 antibody on activation of M. avium subsp. paratuberculosis-specific immune responses.

FIG. 33 Combined effect of COX-2 inhibitor and rat-bovine chimeric anti-bovine PD-L1 antibody on activation of M. avium subsp. paratuberculosis-specific immune responses.

FIG. 34 Kinetic analyses of PGE₂ in BLV-infected cattle.

FIG. 35 Expression analyses of COX-2 and EP4 in BLV-infected cattle.

FIG. 36 Changes in PGE₂ production by antigen stimulation in BLV-infected cattle.

FIG. 37 Effect of PGE₂ on BLV provirus in PBMCs derived from BLV-infected cattle.

FIG. 38 Changes in PD-L1 expression by antigen stimulation in BLV-infected cattle.

FIG. 39 Activation of BLV-specific immune responses by COX-2 inhibitor.

FIG. 40 Antiviral effect of COX-2 inhibitor in BLV-infected cattle.

FIG. 41 Combined effect of COX-2 inhibitor and rat anti-bovine PD-L1 antibody on activation of BLV-specific immune responses.

FIG. 42 Combined effect of COX-2 inhibitor and rat-bovine chimeric anti-bovine PD-L1 antibody on activation of BLV-specific immune responses.

FIG. 43 Antiviral effect from combined use of COX-2 inhibitor and rat-bovine chimeric anti-bovine PD-L1 antibody in BLV-infected cattle.

FIG. 44 Kinetic analyses of PGE₂ in Mycoplasma bovis-infected cattle.

FIG. 45 Correlation between plasma PGE₂ and indicators of immunosuppression in Mycoplasma bovis-infected cattle.

FIG. 46 Expression analyses of COX-2 and EP4 in Mycoplasma bovis-infected cattle.

FIG. 47 Combined immunostimulatory effect of COX-2 inhibitor and rat anti-bovine PD-L1 antibody in Mycoplasma bovis-infected cattle.

FIG. 48 The amino acid sequence of rat-bovine chimeric anti-bovine PD-L1 antibody ch4G12. CDR1, CDR2 and CDR3 regions in the light chain variable region and the heavy chain variable region of rat anti-bovine PD-L1 antibody 4G12 are shown. Further, amino acids introduced as mutations to bovine IgG1 (CH2 domain) are also shown (amino acid numbers and mutations: 250 E->P, 251 L->V, 252 P->A, 253 G->deletion, 347 A->S, 348 P->S).

FIG. 49 Schematic drawings of pDC6 vector and rat-bovine chimeric anti-bovine PD-L1 antibody ch4G12.

FIG. 50 Confirmation of the purity of purified rat-bovine chimeric anti-bovine PD-L1 antibody ch4G12.

FIG. 51 Binding specificity of rat-bovine chimeric anti-bovine PD-L1 antibody ch4G12.

FIG. 52 Inhibitory activity of rat-bovine chimeric anti-bovine PD-L1 antibody ch4G12 against bovine PD-1/PD-L1 binding (the test results of inhibition against binding of bovine PD-L1 expressing cells and soluble bovine PD-1).

FIG. 53 Inhibitory activity of rat-bovine chimeric anti-bovine PD-L1 antibody ch4G12 against bovine PD-1/PD-L1 binding (the test results of inhibition against binding of bovine PD-1 expressing cells and soluble bovine PD-L1).

FIG. 54 Responsivity of rat-bovine chimeric anti-bovine PD-L1 antibody ch4G12 to BLV antigen (in terms of cell proliferation).

FIG. 55 Responsivity of rat-bovine chimeric anti-bovine PD-L1 antibody ch4G12 to BLV antigen (in terms of IFN-γ production).

FIG. 56 The proliferation response of T cells against BLV antigen in a calf experimentally infected with BLV through administration of rat-bovine chimeric anti-bovine PD-L1 antibody ch4G12.

FIG. 57 Changes in BLV provirus loads in the calf experimentally infected with BLV through administration of rat-bovine chimeric anti-bovine PD-L1 antibody ch4G12.

FIG. 58 The amino acid sequence of rat-bovine chimeric anti-bovine PD-1 antibody ch5D2. CDR1, CDR2 and CDR3 in the light chain and the heavy chain variable regions of rat anti-bovine PD-1 antibody 5D2 are shown. Further, amino acids introduced as mutations to bovine IgG1 (CH2 domain) are also shown (amino acid numbers and mutations: 251 E->P, 252 L->V, 253 P->A, 254 G->deletion, 348 A->S, 349 P->S).

FIG. 59 Schematic drawings of pDN112 vector and rat-bovine chimeric anti-bovine PD-1 antibody ch5D2.

FIG. 60 The amount of production and the purity after purification of rat-bovine chimeric anti-bovine PD-1 antibody ch5D2.

FIG. 61 Binding property of rat-bovine chimeric anti-bovine PD-1 antibody ch5D2.

FIG. 62 Inhibitory activity of rat-bovine chimeric anti-bovine PD-1 antibody ch5D2 against bovine PD-1/PD-L1 binding.

FIG. 63 Transition in blood concentrations of rat-bovine chimeric anti-bovine PD-1 antibody ch5D2 after administration to the cattle experimentally infected with BLV.

FIG. 64 Proliferative response of T cells against BLV antigen in the cattle experimentally infected with BLV through administration of rat-bovine chimeric anti-bovine PD-1 antibody ch5D2.

FIG. 65 Changes in the BLV provirus load in the cattle experimentally infected with BLV through administration of rat-bovine chimeric anti-bovine PD-1 antibody ch5D2.

FIG. 66 Cross-reactivity of rat anti-bovine PD-1 antibody 5D2 with ovine PD-1

FIG. 67 Cross-reactivity of rat anti-bovine PD-1 antibody 5D2 with water buffalo T cells

FIG. 68]3D structure of bovine IgG1 constant region and putative binding site for Fcγ receptors

FIG. 69 pDC6 vector

FIG. 70 Purities after purification of rat-bovine chimeric anti-bovine PD-1 antibodies ch5D2 IgG1 WT and IgG1 ADCC—.

FIG. 71 Binding of rat-bovine chimeric anti-bovine PD-1 antibodies ch5D2 IgG1 WT and IgG1 ADCC—to individual bovine Fcγ receptors.

FIG. 72 Sequences disclosed in the present disclosure.

### BEST MODES FOR CARRYING OUT THE INVENTION

Hereinbelow, the present disclosure will be described in detail.

One aspect of the present disclosure provides an anti-PD-L1 antibody comprising a light chain constant region of an antibody of an animal other than rat; and a heavy chain constant region of an antibody of an animal other than rat. The present disclosure provides a pharmaceutical composition which comprises a COX-2 inhibitor and is administered before, after or simultaneously with the administration of an inhibitor targeting PD-1/PD-L1.

Cyclooxygenase 2 (COX-2) is an enzyme involved in a process of biosynthesizing prostanoids including prostaglandin E2 (PGE₂). In contrast to COX-1 that is expressed constitutively, expression of COX-2 is induced by stimulation from cytokines, growth factors, etc. in inflammatory tissues. High expression of COX-2 has been reported in various tumors and infections, and it is believed that COX-2 is involved in the growth and pathogenesis of tumor cells and infected cells. Since PGE₂ inhibits, in particular, the effector function of cytotoxic T-cells via receptors EP2 and EP4, PGE₂ has recently been attracting attention as a humoral factor constituting an immunosuppressive tumor microenvironment. On the other hand, COX-2 inhibitors are expected to decrease PGE₂ production to thereby reduce the suppression upon immune cells. In mouse models, enhancement of antitumor effect and antiviral effect by combined use of a COX-2 inhibitor (such as celecoxib) and an inhibitor targeting PD-1/PD-L1 has been recognized.

COX-2 inhibitor may be an agent that selectively inhibits COX-2. Specific examples of COX-2 inhibitor include, but are not limited to, meloxicam, piroxicam, celecoxib, firocoxib, robenacoxib, carprofen and etodolac.

PD-1 (Programmed cell death-1) is a membrane protein expressed in activated T cells and B cells. Its ligand PD-L1 is expressed in various cells such as antigen-presenting cells (monocytes, dendritic cells, etc.) and cancer cells. PD-1 and PD-L1 work as inhibitory factors which inhibit T cell activation. Certain types of cancer cells and virus-infected cells escape from host immune surveillance by expressing the ligand of PD-1 to thereby inhibit T cell activation.

As inhibitors targeting PD-1/PD-L1, substances which specifically bind to PD-1 or PD-L1 may be given. Such substances include, but are not limited to, proteins, polypeptides, oligopeptides, nucleic acids (including natural-type and artificial nucleic acids), low molecular weight organic compounds, inorganic compounds, cell extracts, and extracts from animals, plants, soils or the like. These substances may be either natural or synthetic products.

Preferable inhibitors targeting PD-1/PD-L1 are antibodies. More preferably, antibodies such as anti-PD-1 antibody and anti-PD-L1 antibody may be given. Any type of antibody may be used as long as it has an inhibitory activity targeting PD-1/PD-L1. The antibody may be any of polyclonal antibody, monoclonal antibody, chimeric antibody, single chain antibody, humanized antibody or human-type antibody. Methods for preparing such antibodies are known. The antibody may be derived from any organisms such as human, mouse, rat, rabbit, goat, guinea pig, dog or cattle. As used herein, the term “antibody” is a concept encompassing antibodies of smaller molecular sizes such as Fab, F(ab)′₂, ScFv, Diabody, V_{H}, V_{L}, Sc(Fv)₂, Bispecific sc(Fv)₂, Minibody, scFv-Fc monomer or scFv-Fc dimer.

As an example of anti-PD-L1 antibody, one comprising (a) a light chain comprising a light chain variable region containing CDR1 having the amino acid sequence of QSLLYSENQKDY (SEQ ID NO: 37), CDR2 having the amino acid sequence of WAT and CDR3 having the amino acid sequence of GQYLVYPFT (SEQ ID NO: 38) and a light chain constant region of an antibody of an animal other than rat; and (b) a heavy chain comprising a heavy chain variable region containing CDR1 having the amino acid sequence of GYTFTSNF (SEQ ID NO: 39), CDR2 having the amino acid sequence of IYPEYGNT (SEQ ID NO: 40) and CDR3 having the amino acid sequence of ASEEAVISLVY (SEQ ID NO: 41) and a heavy chain constant region of an antibody of an animal other than rat may be given.

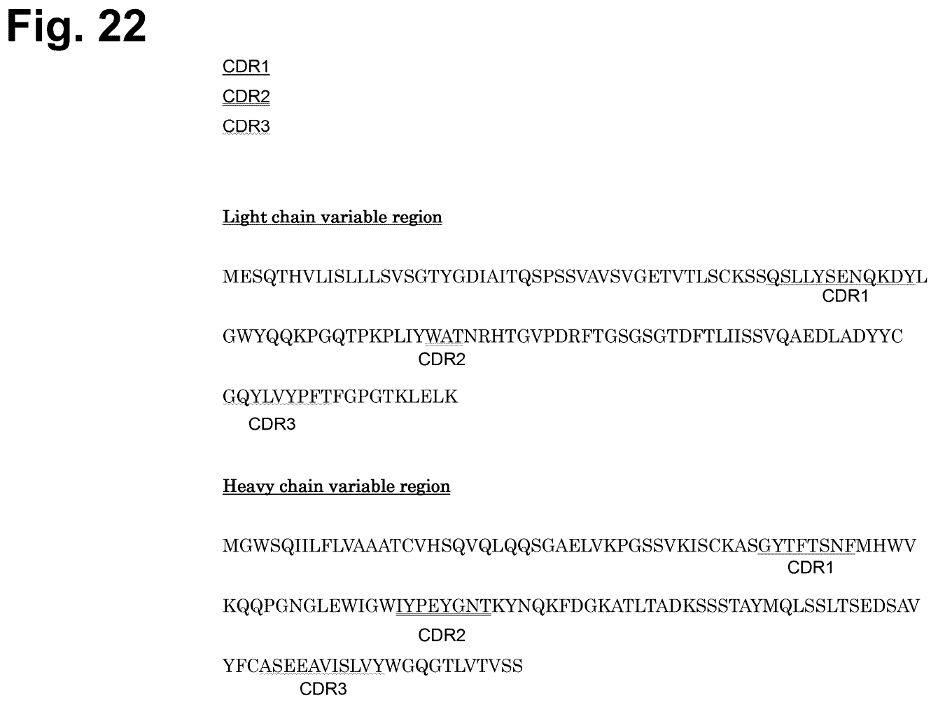

CDR1, CDR2 and CDR3 in the light chain variable region (VL) of rat anti-bovine PD-L1 antibody 4G12 are a region comprising the amino acid sequence of QSLLYSENQKDY (SEQ ID NO: 37), a region comprising the amino acid sequence of WAT and a region comprising the amino acid sequence of GQYLVYPFT (SEQ ID NO: 38), respectively (see FIG. 22).

Further, CDR1, CDR2 and CDR3 in the heavy chain variable region (VH) of rat anti-bovine PD-L1 antibody 4G12 are a region comprising the amino acid sequence of GYTFTSNF (SEQ ID NO: 39), a region comprising the amino acid sequence of IYPEYGNT (SEQ ID NO: 40) and a region comprising the amino acid sequence of ASEEAVISLVY (SEQ ID NO: 41), respectively (see FIG. 22).

In the amino acid sequences of QSLLYSENQKDY (SEQ ID NO: 37), WAT and GQYLVYPFT (SEQ ID NO: 38), as well as the amino acid sequences of GYTFTSNF (SEQ ID NO: 39), IYPEYGNT (SEQ ID NO: 40) and ASEEAVISLVY (SEQ ID NO: 41), one, two, three, four or five amino acids may be deleted, substituted or added.

One aspect of the present disclosure provides an anti-PD-1 antibody comprising (a) a light chain comprising a light chain variable region containing CDR1 having the amino acid sequence of QSLEYSDGYTY (SEQ ID NO: 164), CDR2 having the amino acid sequence of GVS and CDR3 having the amino acid sequence of FQATHDPDT (SEQ ID NO: 165) and the light chain constant region of an antibody of an animal other than rat; and (b) a heavy chain comprising a heavy chain variable region containing CDR1 having the amino acid sequence of GFSLTSYY (SEQ ID NO: 166), CDR2 having the amino acid sequence of IRSGGST (SEQ ID NO: 167) and CDR3 having the amino acid sequence of ARTSSGYEGGFDY (SEQ ID NO: 20) and the heavy chain constant region of an antibody of an animal other than rat.

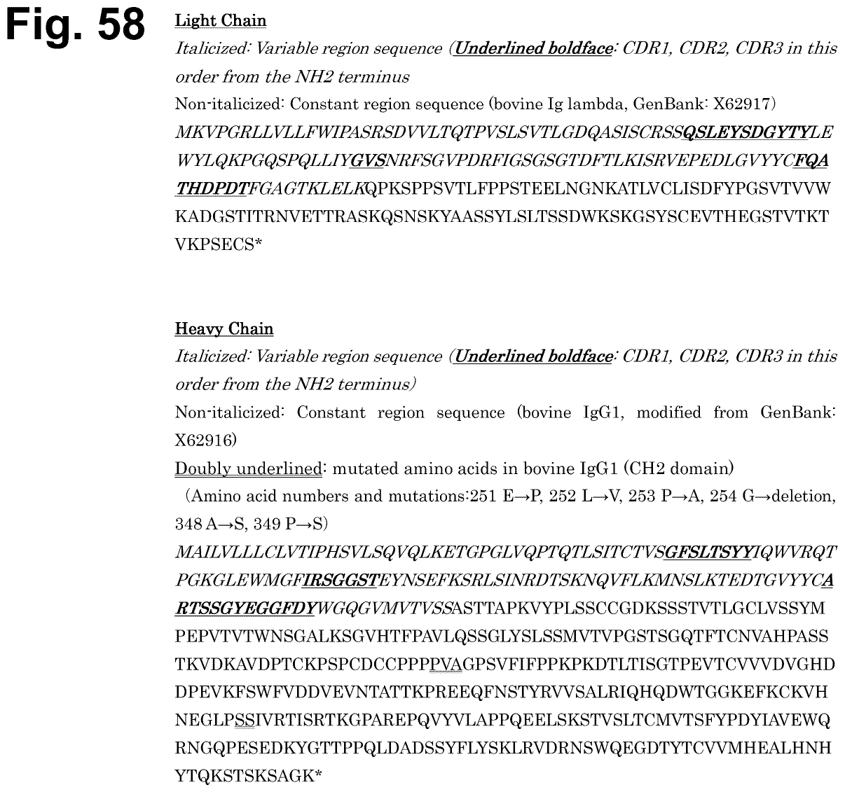

CDR1, CDR2 and CDR3 in the light chain variable region (VL) of rat anti-bovine PD-1 antibody 5D2 (to be described later) are respectively a region consisting of the amino acid sequence of QSLEYSDGYTY (SEQ ID NO: 164), a region consisting of the amino acid sequence of GVS and a region consisting of the amino acid sequence of FQATHDPDT (SEQ ID NO: 165) (see FIG. 58).

Further, CDR1, CDR2 and CDR3 in the heavy chain variable region (VH) of rat anti-bovine PD-1 antibody 5D2 are respectively a region consisting of the amino acid sequence of GFSLTSYY (SEQ ID NO: 166), a region consisting of the amino acid sequence of IRSGGST (SEQ ID NO: 167) and a region consisting of the amino acid sequence of ARTSSGYEGGFDY (SEQ ID NO: 168) (see FIG. 58).

In the amino acid sequences of QSLEYSDGYTY (SEQ ID NO: 164), GVS and FQATHDPDT (SEQ ID NO: 165), as well as the amino acid sequences of GFSLTSYY (SEQ ID NO: 166), IRSGGST (SEQ ID NO: 167) and ARTSSGYEGGFDY (SEQ ID NO: 168), one, two, three, four or five amino acids may be deleted, substituted or added. Even when such mutations are introduced, the resulting amino acid sequences may be capable of having the function of a CDR in the light chain or heavy chain variable region of the PD-1 antibody.

In the above-described anti-PD-L1 antibody, VL and VH thereof may be derived from rat. For example, VL thereof may be the VL of a rat anti-bovine PD-L1 antibody, and VH thereof may be the VH of the rat anti-bovine PD-L1 antibody.

The amino acid sequence of the VL and the amino acid sequence of the VH of the rat anti-bovine PD-L1 antibody are shown in SEQ ID NOS: 1 and 2, respectively. The amino acid sequences as shown in SEQ ID NOS: 1 and 2 may have deletion(s), substitution(s) or addition(s) of one or several (e.g., up to five, about 10 at the most) amino acids. Even when such mutations have been introduced, the resulting amino acid sequences are capable of having the function as VL or VH of the PD-L1 antibody.

The CL and CH of an antibody of an animal other than rat may be derived from an animal which produces a PD-L1 that cross-reacts with rat anti-bovine PD-L1 antibody 4G12.

There are two types of immunoglobulin light chain, which are called Kappa chain (κ) and Lambda chain (λ). In the above-described anti-PD-L1 antibody, the light chain constant region (CL) of an antibody of an animal other than rat may have the amino acid sequence of the constant region of either Kappa chain or Lambda chain. However, the relative abundance of Lambda chain is higher in ovine, feline, canine, equine and bovine, and that of Kappa chain is higher in mouse, rat, human and porcine. Since a chain with a higher relative abundance is considered to be preferable, an ovine, feline, canine, equine or bovine antibody preferably has the amino acid sequence of the constant region of Lambda chain whereas a mouse, rat, human or porcine antibody preferably has the amino acid sequence of the constant region of Kappa chain.

The heavy chain constant region (CH) of an antibody of an animal other than rat may have the amino acid sequence of the constant region of an immunoglobulin equivalent to human IgG4. Immunoglobulin heavy chain is classified into γ chain, μ chain, α chain, δ chain and E chain depending on the difference in constant region. According to the type of heavy chain present, five classes (isotypes) of immunoglobulin are formed; they are IgG, IgM, IgA, IgD and IgE.

Immunoglobulin G (IgG) accounts for 70-75% of human immunoglobulins and is the most abundantly found monomeric antibody in plasma. IgG has a four-chain structure consisting of two light chains and two heavy chains. Human IgG1, IgG2 and IgG4 have molecular weights of about 146,000, whereas human IgG3 has a long hinge region that connects Fab region and Fc region and has a larger molecular weight of 170,000. Human IgG1 accounts for about 65%, human IgG2 about 25%, human IgG3 about 7%, and human IgG4 about 3% of human IgG. They are uniformly distributed inside and outside of blood vessels. Having a strong affinity for Fc receptors and complement factors on effector cell surfaces, human IgG1 induces antibody-dependent cell cytotoxicity (ADCC) and also activates complements to induce complement-dependent cell cytotoxicity (CDC). Human IgG2 and IgG4 are low at ADCC and CDC activities because their affinity for Fc receptors and complement factors is low.

Immunoglobulin M (IgM), which accounts for about 10% of human immunoglobulins, is a pentameric antibody consisting of five basic four-chain structures joined together. It has a molecular weight of 970,000. Usually occurring only in blood, IgM is produced against infectious microorganisms and takes charge of early stage immunity.

Immunoglobulin A (IgA) accounts for 10-15% of human immunoglobulins. It has a molecular weight of 160,000. Secreted IgA is a dimeric antibody consisting of two IgA molecules joined together. IgA1 is found in serum, nasal discharge, saliva and breast milk. In intestinal juice, IgA2 is found abundantly.

Immunoglobulin D (IgD) is a monomeric antibody accounting for no more than 1% of human immunoglobulins. IgD is found on B cell surfaces and involved in induction of antibody production.

Immunoglobulin E (IgE) is a monomeric antibody that occurs in an extremely small amount, accounting for only 0.001% or less of human immunoglobulins. Immunoglobulin E is considered to be involved in immune response to parasites but in advanced countries where parasites are rare, IgE is largely involved in bronchial asthma and allergy among other things.

With respect to canine, sequences of IgG-A (equivalent to human IgG2), IgG-B (equivalent to human IgG1), IgG-C (equivalent to human IgG3) and IgG-D (equivalent to human IgG4) have been identified as the heavy chain of IgG. In the above-described anti-PD-L1 antibody, an IgG's heavy chain constant region with neither ADCC activity nor CDC activity is preferable (IgG4 in human). In the case where the constant region of an immunoglobulin equivalent to human IgG4 has not been identified, one may use a constant region that has lost both ADCC activity and CDC activity as a result of introducing mutations into the relevant region of an immunoglobulin equivalent to human IgG1.

In bovine, the constant region of an immunoglobulin equivalent to human IgG4 has not been identified, so mutations may be added at the relevant region of an immunoglobulin equivalent to human IgG1 and the resultant constant region then used. As one example, the amino acid sequence of the CH of a bovine antibody (IgG1 chain, GenBank: X62916) having mutations introduced into CH2 domain and a nucleotide sequence for such amino acid sequence (after codon optimization) are shown in SEQ ID NOS: 102 and 103, respectively.

When an animal other than rat is canine or bovine, an anti-PD-L1 antibody is more preferable in which (i) the CL of a canine or bovine antibody has the amino acid sequence of the constant region of Lambda chain and (ii) the CH of the canine or bovine antibody has the amino acid sequence of the constant region of an immunoglobulin equivalent to human IgG4.

The above-described anti-PD-L1 antibody encompasses rat-canine chimeric antibodies, caninized antibodies, rat-bovine chimeric antibodies and bovinized antibodies. However, animals are not limited to canine and bovine and may be exemplified by human, porcine, simian, mouse, feline, equine, goat, sheep, water buffalo, rabbit, hamster, guinea pig, bovine and the like.

For example, the anti-PD-L1 antibody described above may be an anti-PD-L1 antibody in which the CL of a canine antibody has the amino acid sequence as shown in SEQ ID NO: 3 and the CH of the canine antibody has the amino acid sequence as shown in SEQ ID NO: 4; or an anti-PD-L1 antibody in which the CL of a bovine antibody has the amino acid sequence as shown in SEQ ID NO: 100 and the CH of the bovine antibody has the amino acid sequence as shown in SEQ ID NO: 102.

The amino acid sequences as shown in SEQ ID NOS: 3 and 4 as well as SEQ ID NOS: 100 and 102 may have deletion(s), substitution(s) or addition(s) of one or several (e.g., up to five, about 10 at the most) amino acids. Even when such mutations have been introduced, the resulting amino acid sequences are capable of having the function as CL or CH of the PD-L1 antibody.

The above-described anti-PD-L1 antibody may have a four-chain structure comprising two light chains and two heavy chains.

The above-described anti-PD-L1 antibody may be prepared as described below. Briefly, an artificial gene is synthesized which comprises (i) the identified variable region sequences of a rat anti-bovine PD-L1 antibody and (ii) the constant region sequences of an antibody of an animal other than rat (e.g., canine or bovine) (preferably, human IgG4 antibody or antibody equivalent to human IgG4 antibody). The resultant gene is inserted into a vector (e.g., plasmid), which is then introduced into a host cell (e.g., mammal cell such as CHO cell). The host cell is cultured, and the antibody of interest is collected from the resultant culture.

The amino acid sequence and the nucleotide sequence of the VL of the rat anti-bovine PD-L1 antibody identified by the present inventors are shown in SEQ ID NOS: 1 and 5, respectively. Further, nucleotide sequences after codon optimization are shown in SEQ ID NOS: 15 and 112.

The amino acid sequence and the nucleotide sequence of the VH of the rat anti-bovine PD-L1 antibody identified by the present inventors are shown in SEQ ID NOS: 2 and 6, respectively. Further, nucleotide sequences after codon optimization are shown in SEQ ID NOS: 16 and 113.

The amino acid sequence and the nucleotide sequence of the CL (Lambda chain, GenBank: E02824.1) of a canine antibody are shown in SEQ ID NOS: 3 and 7, respectively. Further, the nucleotide sequence after codon optimization is shown in SEQ ID NO: 17.

The amino acid sequence and the nucleotide sequence of the CH (IgG-D chain, GenBank: AF354267.1) of the canine antibody are shown in SEQ ID NOS: 4 and 8, respectively. Further, the nucleotide sequence after codon optimization is shown in SEQ ID NO: 18.

Further, SEQ ID NO: 9 shows the amino acid sequence of a chimeric light chain comprising the VL of the rat anti-bovine PD-L1 antibody and the CL (Lambda chain, GenBank: E02824.1) of the canine antibody. The nucleotide sequence (after codon optimization) of the chimeric light chain comprising the VL of the rat anti-bovine PD-L1 antibody and the CL (Lambda chain, GenBank: E02824.1) of the canine antibody is shown in SEQ ID NO: 19.

SEQ ID NO: 10 shows the amino acid sequence of a chimeric heavy chain comprising the VH of the rat anti-bovine PD-L1 antibody and the CH (IgG-D chain, GenBank: AF354267.1) of the canine antibody. The nucleotide sequence (after codon optimization) of the chimeric heavy chain comprising the VH of the rat anti-bovine PD-L1 antibody and the CH (IgG-D chain, GenBank: AF354267.1) of the canine antibody is shown in SEQ ID NO: 20.

The amino acid sequence and the nucleotide sequence of the CL (Lambda chain, GenBank: X62917) of a bovine antibody are shown in SEQ ID NOS: 100 and 101, respectively. Further, the nucleotide sequence after codon optimization is shown in SEQ ID NO: 114.

The amino acid sequence and the nucleotide sequence (after codon optimization) of the CH (IgG1 chain, modified from GenBank: X62916) of the bovine antibody are shown in SEQ ID NOS: 102 and 103, respectively.

Further, SEQ ID NO: 115 shows the amino acid sequence of a chimeric light chain comprising the VL of the rat anti-bovine PD-L1 antibody and the CL (Lambda chain, GenBank: X62917) of the bovine antibody. The nucleotide sequence (after codon optimization) of the chimeric light chain comprising the VL of the rat anti-bovine PD-L1 antibody and the CL (Lambda chain, GenBank: X62917) of the bovine antibody is shown in SEQ ID NO: 117.

SEQ ID NO: 116 shows the amino acid sequence of a chimeric heavy chain comprising the VH of the rat anti-bovine PD-L1 antibody and the CH (IgG1 chain, modified from GenBank: X62916) of the bovine antibody. The nucleotide sequence (after codon optimization) of the chimeric heavy chain comprising the VH of the rat anti-bovine PD-L1 antibody and the CH (IgG1 chain, modified from GenBank: X62916) of the bovine antibody is shown in SEQ ID NO: 118.

Amino acid sequences and nucleotide sequences of CLs and CHs for various animals other than rat, canine and bovine may be obtained from known databases for use in the present disclosure.

Amino acid sequences and nucleotide sequences of CLs and CHs for canine, ovine, porcine, water buffalo, human and bovine are summarized in the table below.

TABLE

| | | | | | GenBank | | |
|---|---|---|---|---|---|---|---|
| | | | | | Accession | IMGT | |
| Species | Ig Domain | | Nucleotide Sequence | Amino Acid Sequence | No. | Database | Reference |
| Canine | Canine | IgG-D | GCCTCCACCACGGCCCCCTCG | ASTTAPSVFPLAPSCGSTS | AF354267 | http://ww | Tang L, et al., |
| (Scienti- | Ig | | GTTTTCCCACTGGCCCCCAGC | GSTVALACLVSGYFPEPVT | | w.imgt.or | Vet. Immunol. |
| fic | heavy | | TGCGGGTCCACTTCCGGCTCC | VSWNSGSLTSGVHTFPSV | | g/IMGTre | Immunopathol, |
| Name: | chain | | ACGGTGGCCCTGGCCTGCCTG | LQSSGLYSLSSTVTVPSSR | | pertoire/i | 80 (3-4), 259-270 |
| Canis | constant | | GTGTCAGGCTACTTCCCCGAG | WPSETFTCNVVHPASNTK | | ndex.php | (2001), PMID: |
| lupus | region | | CCTGTAACTGTGTCCTGGAAT | VDKPVPKESTCKCISPCPV | | ?section = | 11457479 |
| familiaris) | (CH1- | | TCCGGCTCCTTGACCAGCGGT | PESLGGPSVFIFPPKPKDIL | | LocusGe | |
| | CH3) | | GTGCACACCTTCCCGTCCGTC | RITRTPEITCVVLDLGRED | | nes&repe | |
| | | | CTGCAGTCCTCAGGGCTCTAC | PEVQISWFVDGKEVHTAK | | rtoire = ge | |
| | | | TCCCTCAGCAGCACGGTGAC | TQPREQQFNSTYRVVSVL | | netable & s | |
| | | | AGTGCCCTCCAGCAGGTGGC | PIEHQDWLTGKEFKCRVN | | pecies = do | |
| | | | CCAGCGAGACCTTCACCTGCA | HIGLPSPIERTISKARGQAH | | g & group = | |
| | | | ACGTGGTCCACCCGGCCAGC | QPSVYVLPPSPKELSSSDT | | IGHC | |
| | | | AACACTAAAGTAGACAAGCC | VTLTCLIKDFFPPEIDVEW | | | |
| | | | AGTGCCCAAAGAGTCCACCT | QSNGQPEPESKYHTTAPQ | | | |
| | | | GCAAGTGTATATCCCCATGCC | LDEDGSYFLYSKLSVDKS | | | |
| | | | CAGTCCCTGAATCACTGGGAG | RWQQGDTFTCAVMHEAL | | | |
| | | | GGCCTTCGGTCTTCATCTTTCC | QNHYTDLSLSHSPGK* | | | |
| | | | CCCGAAACCCAAGGACATCCT | (SEQ ID NO: 4) | | | |
| | | | CAGGATTACCCGAACACCCGA | | | | |
| | | | GATCACCTGTGTGGTGTTAGA | | | | |
| | | | TCTGGGCCGTGAGGACCCTG | | | | |
| | | | AGGTGCAGATCAGCTGGTTCG | | | | |
| | | | TGGATGGTAAGGAGGTGCAC | | | | |
| | | | ACAGCCAAGACGCAGCCTCG | | | | |
| | | | TGAGCAGCAGTTCAACAGCA | | | | |
| | | | CCTACCGTGTGGTCAGCGTCC | | | | |
| | | | TCCCCATTGAGCACCAGGACT | | | | |
| | | | GGCTCACCGGAAAGGAGTTC | | | | |
| | | | AAGTGCAGAGTCAACCACAT | | | | |
| | | | AGGCCTCCCGTCCCCCATCGA | | | | |
| | | | GAGGACTATCTCCAAAGCCAG | | | | |
| | | | AGGGCAAGCCCATCAGCCCA | | | | |
| | | | GTGTGTATGTCCTGCCACCAT | | | | |
| | | | CCCCAAAGGAGTTGTCATCCA | | | | |
| | | | GTGACACGGTCACCCTGACCT | | | | |
| | | | GCCTGATCAAAGACTTCTTCC | | | | |
| | | | CACCTGAGATTGATGTGGAGT | | | | |
| | | | GGCAGAGCAATGGACAGCCG | | | | |
| | | | GAGCCCGAGAGCAAGTACCA | | | | |
| | | | CACGACTGCGCCCCAGCTGG | | | | |
| | | | ACGAGGACGGGTCCTACTTCC | | | | |
| | | | TGTACAGCAAGCTCTCTGTGG | | | | |
| | | | ACAAGAGCCGCTGGCAGCAG | | | | |
| | | | GGAGACACCTTCACATGTGCG | | | | |
| | | | GTGATGCATGAAGCTCTACAG | | | | |
| | | | AACCACTACACAGATCTATCC | | | | |
| | | | CTCTCCCATTCTCCGGGTAAA | | | | |
| | | | TGA (SEQ ID NO: 8) | | | | |
| | Canine | Ig | CAGCCCAAGGCCTCCCCCT | QPKASPSVTLFPPSSEE | E02824 | Not | None |
| | Ig | lambda | CGGTCACACTCTTCCCGCC | LGANKATLVCLISDFYP | | registered | |
| | light | (CL) | CTCCTCTGAGGAGCTCGGC | SGVTVAWKASGSPVTQ | | | |
| | chain | | GCCAACAAGGCCACCCTGG | GVETTKPSKQSNNKYA | | | |
| | constant | | TGTGCCTCATCAGCGACTTC | ASSYLSLTPDKWKSHSS | | | |
| | region | | TACCCCAGCGGCGTGACGG | FSCLVTHEGSTVEKKVA | | | |
| | | | TGGCCTGGAAGGCAAGCGG | PAECS* (SEQ ID NO: 3) | | | |
| | | | CAGCCCCGTCACCCAGGGC | | | | |
| | | | GTGGAGACCACCAAGCCCT | | | | |
| | | | CCAAGCAGAGCAACAACAA | | | | |
| | | | GTACGCGGCCAGCAGCTAC | | | | |
| | | | CTGAGCCTGACGCCTGACA | | | | |
| | | | AGTGGAAATCTCACAGCAG | | | | |
| | | | CTTCAGCTGCCTGGTCACG | | | | |
| | | | CACGAGGGGAGCACCGTGG | | | | |
| | | | AGAAGAAGGTGGCCCCCGC | | | | |
| | | | AGAGTGCTCTTAG (SEQ ID | | | | |
| | | | NO: 7) | | | | |
| Ovine | Ovine | ‘IgG1 | GCCTCAACAACACCCCCGAA | ASTTPPKVYPLTSCCGDTS | X69797 | http://ww | Dufour V. et al., |
| (Scienti- | heavy | | AGTCTACCCTCTGACTTCTTG | SSIVTLGCLVSSYMPEPVT | | w.imgt.or | J. Immunol., 156. |
| tic | chain | | CTGCGGGGACACGTCCAGCT | VTWNSGALTSGVHTFPAI | | g/IMGTre | 2163-2170 |
| Name: | constant | | CCATCGTGACCCTGGGCTGCC | LQSSGLYSLSSVVTVPAST | | pertoir/li | (1996). PMID: |
| Ovis | region | | TGGTCTCCAGCTATATGCCCG | SGAQTFICNVAHPASSTKV | | ndex.php | 8690905 |
| aries) | (CH1^{~} | | AGCCGGTGACCGTGACCTGG | DKRVEPGCPDPCKHCRCP | | ?section = | |
| | CH3) | | AACTCTGGTGCCCTGACCAGC | PPELPGGPSVFIFPPKPKDT | | LocusGe | |
| | | | GGCGTGCACACCTTCCCGGCC | LTISGTPEVTCVVVDVGQ | | nes & repe | |
| | | | ATCCTGCAGTCCTCCGGGCTC | DDPEVQFSWFVDNVEVRT | | rtoire = ge | |
| | | | TACTCTCTCAGCAGCGTGGTG | ARTKPREEQFNSTFRVVSA | | netable & s | |
| | | | ACCGTGCCGGCCAGCACCTC | LPIQHQDWTGGKEFKCKV | | pecies = sh | |
| | | | AGGAGCCCAGACCTTCATCTG | HNEALPAPIVRTISRTKGQ | | eep & grou | |
| | | | CAACGTAGCCCACCCGGCCA | AREPQVYVLAPPQEELSK | | p = IGHC | |
| | | | GCAGCACCAAGGTGGACAAG | STLSVTCLVTGFYPDYIAV | | | |
| | | | CGTGTTGAGCCCGGATGCCCG | EWQKNGQPESEDKYGTT | | | |
| | | | GACCCATGCAAACATTGCCGA | TSQLDADGSYFLYSRLRV | | | |
| | | | TGCCCACCCCCTGAGCTCCCC | DKNSWQEGDTYACVVMH | | | |
| | | | GGAGGACCGTCTGTCTTCATC | EALHNHYTQKSISKPPGK* | | | |
| | | | TTCCCACCGAAACCCAAGGA | (SEQ ID NO: 42) | | | |
| | | | CACCCTTACAATCTCTGGAAC | | | | |
| | | | GCCCGAGGTCACGTGTGTGGT | | | | |
| | | | GGTGGACGTGGGCCAGGATG | | | | |
| | | | ACCCCGAGGTGCAGTTCTCCT | | | | |
| | | | GGTTCGTGGACAACGTGGAG | | | | |
| | | | GTGCGCACGGCCAGGACAAA | | | | |
| | | | GCCGAGAGAGGAGCAGTTCA | | | | |
| | | | ACAGCACCTTCCGCGTGGTCA | | | | |
| | | | GCGCCCTGCCCATCCAGCACC | | | | |
| | | | AAGACTGGACTGGAGGAAAG | | | | |
| | | | GAGTTCAAGTGCAAGGTCCA | | | | |
| | | | CAACGAAGCCCTCCCGGCCC | | | | |
| | | | CCATCGTGAGGACCATCTCCA | | | | |
| | | | GGACCAAAGGGCAGGCCCGG | | | | |
| | | | GAGCCGCAGGTGTACGTCCTG | | | | |
| | | | GCCCCACCCCAGGAAGAGCT | | | | |
| | | | CAGCAAAAGCACGCTCAGCG | | | | |
| | | | TCACCTGCCTGGTCACCGGCT | | | | |
| | | | TCTACCCAGACTACATCGCCG | | | | |
| | | | TGGAGTGGCAGAAAAATGGG | | | | |
| | | | CAGCCTGAGTCGGAGGACAA | | | | |
| | | | GTACGGCACGACCACATCCCA | | | | |
| | | | GCTGGACGCCGACGGCTCCTA | | | | |
| | | | CTTCCTGTACAGCAGGCTCAG | | | | |
| | | | GGTGGACAAGAACAGCTGGC | | | | |
| | | | AAGAAGGAGACACCTACGCG | | | | |
| | | | TGTGTGGTGATGCACGAGGCT | | | | |
| | | | CTGCACAACCACTACACACA | | | | |
| | | | GAAGTCGATCTCTAAGCCTCC | | | | |
| | | | GGGTAAATGA (SEQ ID NO: 43) | | | | |
| | | IgG2 | GCCTCCACCACAGCCCCGAA | ASTTAPKVYPLTSCCGDTS | X70983 | | Clarkson C.A. et |
| | | | AGTCTACCCTCTGACTTCTTG | SSSSIVTLGCLVSSYMPEP | | | al., Mol. |
| | | | CTGCGGGGACCACGTCCAGCT | VTVTWNSGALTSGVHTFP | | | Immunol., 30, |
| | | | CCAGCTCCATCGTGACCCTGG | AILQSSGLYSLSSVVTVPA | | | 1195-1204 |
| | | | GCTGCCTGGTCTCCAGCTATAT | STSGAQTFICNVAHPASSA | | | (1993). PMID: |
| | | | GCCCGAGCCGGTGACCGTGA | KVDKRVGISSDYSKCSKP | | | 8413324 |
| | | | CCTGGAACTCTGGTGCCCTGA | PCVSRPSVFIFPPKPKDSL | | | |
| | | | CCAGCGGCGTGCACACCTTCC | MITGTPEVTCVVVDVGQG | | | |
| | | | CGGCCATCCTGCAGTCCTCCG | DPEVQFSWFVDNVEVRTA | | | |
| | | | GGCTCTACTCTCTCAGCAGCG | RTKPREEQFNSTFRVVSAL | | | |
| | | | TGGTGACCGTGCCGGCCAGC | PIQHDHWTGGKEFKCKV | | | |
| | | | ACCTCAGGAGCCCAGACCTTC | HSKGLPAPIVRTISRAKGQ | | | |
| | | | ATCTGCAACGTAGCCCACCCG | AREPQVYVLAPPQEELSK | | | |
| | | | GCCAGCAGCGCCAAGGTGGA | STLSVTCLVTGFYPDYIAV | | | |
| | | | CAAGCGTGTTGGGATCTCCAG | EWQRARQPESEDKYGTTT | | | |
| | | | TGACTACTCCAAGTGTTCTAA | SQLDADGSYFLYSRLRVD | | | |
| | | | ACCGCCTTGCGTGAGCCGACC | KSSWQRGDTYACVVMHE | | | |
| | | | GTCTGTCTTCATCTTCCCCCCG | ALHNHYTQKSISKPPGK* | | | |
| | | | AAACCCAAGGACAGCCTCAT | (SEQ ID NO: 44) | | | |
| | | | GATCACAGGAACGCCCGAGG | | | | |
| | | | TCACGTGTGTGGTGGTGGACG | | | | |
| | | | TGGGCCAGGGTGACCCCGAG | | | | |
| | | | GTGCAGTTCTCCTGGTTCGTG | | | | |
| | | | GACAACGTGGAGGTGCGCAC | | | | |
| | | | GGCCAGGACAAAGCCGAGAG | | | | |
| | | | AGGAGCAGTTCAACAGCACC | | | | |
| | | | TTCCGCGTGGTCAGCGCCCTG | | | | |
| | | | CCCATCCAGCACGACCACTGG | | | | |
| | | | ACTGGAGGAAAGGAGTTCAA | | | | |
| | | | GTGCAAGGTCCACAGCAAAG | | | | |
| | | | GCCTCCCGGCCCCCATCGTGA | | | | |
| | | | GGACCATCTCCAGGGCCAAA | | | | |
| | | | GGGCAGGCCCGGGAGCCGCA | | | | |
| | | | GGTGTACGTCCTGGCCCCACC | | | | |
| | | | CCAGGAAGAGCTCAGCAAAA | | | | |
| | | | GCACGCTCAGCGICACCTGCC | | | | |
| | | | TGGTCACCGGCTTCTACCCAG | | | | |
| | | | ACTACATCCICCGTGGAGTGGC | | | | |
| | | | AGAGAGCGCGGCAGCCTGAG | | | | |
| | | | TCGGAGGACAAGTACGGCAC | | | | |
| | | | GACCACATCCCAGCTGGACGC | | | | |
| | | | CGACGGCTCCTACTTCCTGTA | | | | |
| | | | CAGCAGGCTCAGGGTGGACA | | | | |
| | | | AGAGCAGCTGGCAAAGAGGA | | | | |
| | | | GACACCTACGCGTGTGTGGTG | | | | |
| | | | ATGCACGAGGCTCTGCACAAC | | | | |
| | | | CACTACACACAGAACITCGATC | | | | |
| | | | TCTAAGCCTCCGGGTAAATGA | | | | |
| | | | (SEQ ID NO: 45) | | | | |
| | Ovine | Ig | CCATCCGTCTTCCTCTTCAAA | PSVFLFKPSEEQLRTGTVS | X54110 | Not | Jenne C.N. et al., |
| | light | kappa | CCATCTGAGGAACAGCTGAG | VVCLVNDFYPKDINVKVK | | registered | Dev. Comp. |
| | chain | (CK) | GACCGGAACTGTCTCTGTCGT | VDGVTQNSNFQNSFTDQD | | | Immunol. 30 (1- |
| | constant | | GTGCTTGGTGAATGATTTCTA | SKKSTYSLSSTLTLSSSEY | | | 2), 165-174 |
| | region | | CCCCAAAGATATCAATGTCAA | QSHNAYACEVSHKSLPTA | | | (2006). PMID: |
| | | | GGTGAAAGTGGATGGGGTTA | LVKSFNKNEC* (SEQ ID | | | 16083958 |
| | | | CCCAGAACAGCAACTTCCAG | NO: 46) | | | |
| | | | AACAGCTTCACAGACCAGGA | | | | |
| | | | CAGCAAGAAAAGCACCTACA | | | | |
| | | | GCCTCAGCAGCACCCTGACA | | | | |
| | | | CTGTCCAGCTCAGAGTACCAG | | | | |
| | | | AGCCATAACGCCTATGCGTGT | | | | |
| | | | GAGGTCAGCCACAAGAGCCT | | | | |
| | | | GCCCACCGCCCTCGTCAAGA | | | | |
| | | | GCTTCAATAAGAATGAATGTT | | | | |
| | | | AG (SEQ ID NO: 47) | | | | |
| | | Ig | GGTCAGCCCAAGTCCGCACC | GQPKSAPSVTLFPPSTEEL | AY734681 | | |
| | | lambda | CTCGGTCACCCTGTTCCCGCC | STNKATVVCLINDFYPGS | | | |
| | | (CL) | TTCCACGGAGGAGCTCAGTAC | VNVVWKADGSTINQNVK | | | |
| | | | CAACAAGGCCACCGTGGTGT | TTQASKQSNSKYAASSYL | | | |
| | | | GTCTCATCAACGACTTCTACC | TLTGSEWKSKSSYTCEVT | | | |
| | | | CGGGTAGCGTGAACGTGGTCT | HEGSTVTKTVKPSECS* | | | |
| | | | GGAAGGCAGATGGCAGCACC | (SEQ ID NO: 48) | | | |
| | | | ATCAATCAGAACGTGAAGACC | | | | |
| | | | ACCCAGGCCTCCAAACAGAG | | | | |
| | | | CAACAGCAAGTACGCGGCCA | | | | |
| | | | GCAGCTACCTGACCCTGACGG | | | | |
| | | | GCAGCGAGTGGAAGTCTAAG | | | | |
| | | | AGCAGTTACACCTGCGAGGTC | | | | |
| | | | ACGCACGAGGGGAGCACCGT | | | | |
| | | | GACGAAGACAGTGAAGCCCT | | | | |
| | | | CAGAGTGTTCTTAG (SEQ ID | | | | |
| | | | NO: 49) | | | | |
| Porcine | Porcine | IgG1^{a} | GCCCCCAAGACGGCCCCATCG | APKTAPSVYPLAPCGRDT | U03781 | http://ww | Butler J.E. et al., |
| (Scienti- | Ig | | GTCTACCCTCTGGCCCCCTGC | SGPNVALGCASSYFPEPV | | w.imgt.or | Immunogenetics |
| fic | heavy | | GGCAGGGACACGTCTGGCCC | TMTWNSGALTSGVHTFPS | | g/IMGTre | 61(3): 209-230 |
| Name: | chain | | TAACGTGGCCTTGGGCTGCCT | VLQPSGLYSLSSMVTVPAS | | pertoire/i | (2009). PMID: |
| Sus | constant | | GGCCTCAAGCTACTFCCCCGA | SLSSKSYTCNVNHPATTTK | | ndex.php | 19048248 |
| scrofa) | region | | GCCAGTGACCATGACCTGGA | VDKRVGTKTKPPCPICPGC | | ?section = | Kacskovics I. et |
| | (CH1- | | ACTCGGGCGCCCTGACCAGT | EVAGPSVFIFPPKPKDTLM | | LocusGe | al., J. Immunol. |
| | CH3) | | GGCGTGCATACCTTCCCATCC | ISQTPEVTCVVVDVSKEH | | nes & repe | 153(8): 3565- |
| | | | GTCCTGCAGCCGTCAGGGCTC | AEVQFSWYVDGVEVHTA | | rtoire = ge | 3573 (1994). |
| | | | TACTCCCTCAGCAGCATGGTG | ETRPKEEQFNSTYRVVSV | | netable & s | PMID: 7930579 |
| | | | ACCGTGCCGGCCAGCAGCCT | LPIQHQDWLKGKEFKCKV | | pecies = Pi | |
| | | | GTCCAGCAAGAGCTACACCT | NNVDLPAPITRTISKAIGQS | | g & group = | |
| | | | GCAATGTCAACCACCCGGCCA | REPQVYTLPPPAEELSRSK | | IGHC | |
| | | | CCACCACCAAGGTGGACAAG | VTVTCLVIGFYPPDIHVEW | | | |
| | | | CGTGTTGGAACAAAGACCAA | KSNGQPEPEGNYRTTPPQ | | | |
| | | | ACCACCATGTCCCATATGCCC | QDVDGTFFLYSKLAVDKA | | | |
| | | | AGGCTGTGAAGTGGCCGGGC | RWDHGETFECAVMHEAL | | | |
| | | | CCTCGGTCTTCATCTTCCCTCC | HNHYTQKSISKTQGK* | | | |
| | | | AAAACCCAAGGACACCCTCA | (SEQ ID NO: 50) | | | |
| | | | TGATCTCCCAGACCCCCGAGG | | | | |
| | | | TCACGTGCGTGGTGGTGGAC | | | | |
| | | | GTCAGCAAGGAGCACGCCGA | | | | |
| | | | GGTCCAGTTCTCCTGGTACGT | | | | |
| | | | GGACGGCGTAGAGGTGCACA | | | | |
| | | | CGGCCGAGACGAGACCAAAG | | | | |
| | | | GAGGAGCAGTTCAACAGCAC | | | | |
| | | | CTACCGTGTGGTCAGCGICCT | | | | |
| | | | GCCCATCCAGCACCAGGACTG | | | | |
| | | | GCTGAAGGGGAAGGAGTTCA | | | | |
| | | | AGTGCAAGGTCAACAACGTA | | | | |
| | | | GACCTCCCAGCCCCCATCACG | | | | |
| | | | AGGACCATCTCCAAGGCTATA | | | | |
| | | | GGGCAGAGCCGGGAGCCGCA | | | | |
| | | | GGTGTACACCCTGCCCCCACC | | | | |
| | | | CGCCGAGGAGCTGTCCAGGA | | | | |
| | | | GCAAAGTCACCGTAACCTGCC | | | | |
| | | | TGGTCATTGGCTTCTACCCAC | | | | |
| | | | CTGACATCCATGTTGAGTGGA | | | | |
| | | | AGAGCAACGGACAGCCGGAG | | | | |
| | | | CCAGAGGGCAAFTACCGCACC | | | | |
| | | | ACCCCGCCCCAGCAGGACGT | | | | |
| | | | GGACGGGACCTTCTTCCTGTA | | | | |
| | | | CAGCAAGCTCGCGGTGGACA | | | | |
| | | | AGGCAAGATGGGACCATGGA | | | | |
| | | | GAAACATTTGAGTGTGCGGTG | | | | |
| | | | ATGCACGAGGCTCTGCACAAC | | | | |
| | | | CACTACACCCAGAAGTCCATC | | | | |
| | | | TCCAAGACTCAGGGTAAATGA | | | | |
| | | | (SEQ ID NO: 51) | | | | |
| | | IgG1^{b} | GCCCCCAAGACGGCCCCATCG | APKTAPSVYPLAPCGRDV | U03778 | | |
| | | | GTCTACCCTCTGGCCCCCTGC | SGPNVALGCLASSYFPEPV | | | |
| | | | GGCAGGGACGTGTCTGGCCCT | TVTWNSGALTSGVHTFPS | | | |
| | | | AACGTGGCCTTGGGCTGCCTG | VLQPSGLYSLSSMVTVPAS | | | |
| | | | GCCTCAAGCTACTTCCCCGAG | SLSSKSYTCNVNHPATTTK | | | |
| | | | CCAGTGACCGTGACCTGGAA | VDKRVGIHQPQTCPICPGC | | | |
| | | | CTCGGGCGCCCTGACCAGTG | EVAGPSVFIFPPKPKDTLM | | | |
| | | | GCGTGCACACCTTCCCATCCG | ISQTEVTCVVVDVSKEH | | | |
| | | | TCCTGCAGCCGTCAGGGCTCT | AEVQFSWYVDGVEVHTA | | | |
| | | | ACTCCCTCAGCAGCATGGTGA | ETRPKEEQFNSTYRVVSC | | | |
| | | | CCGTGCCGGCCAGCAGCCTGT | LPIQHQDWLKGKEFKCKV | | | |
| | | | CCAGCAAGAGCTACACCTGC | NNVDLPAPITRTISKAIGQS | | | |
| | | | AATGTCAACCACCCGGCCACC | REPQVYTLPPPAEELSRSK | | | |
| | | | ACCACCAAGGTGGACAAGCG | VTLTCLVIGFYPPDIHVEW | | | |
| | | | TGTTGGAATACACCAGCCGCA | KSNGQPEPENTYRTTPPQ | | | |
| | | | AACATGTCCCATATGCCCAGG | QDVDGTFFLYSKLAVDKA | | | |
| | | | CTGTGAAGTGGCCGGGCCCTC | RWDHGDKFECAVMHEAL | | | |
| | | | GGTCTTCATCTTCCCTCCAAA | HNHYTQKSISKTQGK* | | | |
| | | | ACCCAAGGACACCCTCATGAT | (SEQ ID NO: 52) | | | |
| | | | CTCCCAGACCCCCGAGGTCAC | | | | |
| | | | GTGCGTGGTGGTGGACGTCA | | | | |
| | | | GCAAGGAGCACGCCGAGGTC | | | | |
| | | | CAGTTCTCCTGGTACGTGGAC | | | | |
| | | | GGCGTAGAGGTGCACACGGC | | | | |
| | | | CGAGACGAGACCAAAGGAGG | | | | |
| | | | AGCAGTTCAACAGCACCTACC | | | | |
| | | | GTGTGGTCAGCGTCCTGCCCA | | | | |
| | | | TCCAGCACCAGGACTGGCTG | | | | |
| | | | AAGGGGAAGGAGTTCAAGTG | | | | |
| | | | CAAGGTCAACAACGTAGACC | | | | |
| | | | TCCCAGCCCCCATCACGAGGA | | | | |
| | | | CCATCTCCAAGGCTATAGGGC | | | | |
| | | | AGAGCCGGGAGCCGCAGGTG | | | | |
| | | | TACACCCTGCCCCCACCCGCC | | | | |
| | | | GAGGAGCTGTCCAGGAGCAA | | | | |
| | | | AGTCACGCTAACCTGCCTGGT | | | | |
| | | | CATTGGCTTCTACCCACCTGA | | | | |
| | | | CATCCATGTTGAGTGGAAGAG | | | | |
| | | | CAACGGACAGCCGGAGCCAG | | | | |
| | | | AGAACACATACCGCACCACCC | | | | |
| | | | CGCCCCAGCAGGACGTGGAC | | | | |
| | | | GGGACCTTCTTCCTGTACAGC | | | | |
| | | | AAACTCGCGGTGGACAAGGC | | | | |
| | | | AAGATGGGACCATGGAGACA | | | | |
| | | | AATTTGAGTGTGCGGTGATGC | | | | |
| | | | ACGAGGCTCTGCACAACCACT | | | | |
| | | | ACACCCAGAAGTCCATCTCCA | | | | |
| | | | AGACTCAGGGTAAATGA (SEQ | | | | |
| | | | ID NO: 53) | | | | |
| | | IgG2^{a} | GCCCCCAAGACGGCCCCATCG | APKTAPSVYPLAPCSRDTS | U03779 | | |
| | | | GTCTACCCTCTGGCCCCCTGC | GPNVALGCLASSYFPEPVT | | | |
| | | | AGCAGGGACACGTCTGGCCC | VTWNSGALSSGVHTFPSV | | | |
| | | | TAACGTGGCCTTGGGCTGCCT | LQPSGLYSLSSMVTVPASS | | | |
| | | | GGCCTCAAGCTACTTCCCCGA | LSSKSYTCNVNHPATTTK | | | |
| | | | GCCAGTGACCGTGACCTGGA | VDKRVGTKTKPPCPICPAC | | | |
| | | | ACTCGGGCGCCCTGTCCAGTG | ESPGPSVFIFPPKPKDTLMI | | | |
| | | | GCGTGCATACCTTCCCATCCG | SRTPQVTCVVVDVSQENP | | | |
| | | | TCCTGCAGCCGTCAGGGCTCT | EVQFSWYVDGVEVHTAQ | | | |
| | | | ACTCCCTCAGCAGCATGGTGA | TRPKEEQFNSTYRVVSVLP | | | |
| | | | CCGTGCCGGCCAGCAGCCTGT | IQHQDWLNGKEFKCKVN | | | |
| | | | CCAGCAAGAGCTACACCTGC | NKDLPAPITRIISKAKGQT | | | |
| | | | AATGTCAACCACCCGGCCACC | REPQVYTLPPHAEELSRSK | | | |
| | | | ACCACCAAGGTGGACAAGCG | VSITCLVIGFYPPDIDVEW | | | |
| | | | TGTTGGAACAAAGACCAAAC | QRNGQPEPEGNYRTTPPQ | | | |
| | | | CACCATGTCCCATATGCCCAG | QDVDGTYFLYSKFSVDKA | | | |
| | | | CCTGTGAATCCCAGGGCCCT | SWQGGGIFQCAVMHEAL | | | |
| | | | CGGTCTTCATCTTCCCTCCAA | HNHYTQKSISKTPGK* | | | |
| | | | AACCCAAGGACACCCTCATGA | (SEQ ID NO: 54) | | | |
| | | | TCTCCCGGACACCCCAGGTCA | | | | |
| | | | CGTGCGTGGTGGTTGATGTGA | | | | |
| | | | GCCAGGAGAACCCGGAGGTC | | | | |
| | | | CAGTTCTCCTGGTACGTGGAC | | | | |
| | | | GGCGTAGAGGTGCACACGGC | | | | |
| | | | CCAGACGAGGCCAAAGGAGG | | | | |
| | | | AGCAGTTCAACAGCACCTACC | | | | |
| | | | GCGTGGTCAGCGTCCTACCCA | | | | |
| | | | TCCAGCACCAGGACTGGCTG | | | | |
| | | | AACGGGAAGGAGTTCAAGTG | | | | |
| | | | CAAGGTCAACAACAAAGACC | | | | |
| | | | TCCCAGCCCCCATCACAAGGA | | | | |
| | | | TCATCTCCAAGGCCAAAGGGC | | | | |
| | | | AGACCCGGGAGCCGCAGGTG | | | | |
| | | | TACACCCTGCCCCCACACGCC | | | | |
| | | | GAGGAGCTGTCCAGGAGCAA | | | | |
| | | | AGTCAGCATAACCTGCCTGGT | | | | |
| | | | CATTGGCTTCTACCCACCTGA | | | | |
| | | | CATCGATGTCGAGTGGCAAAG | | | | |
| | | | AAACGGACAGCCGGAGCCAG | | | | |
| | | | AGGGCAATTACCGCACCACCC | | | | |
| | | | CGCCCCAGCAGGACGTGGAC | | | | |
| | | | GGGACCTACTTCCTGTACAGC | | | | |
| | | | AAGTTCTCGGTGGACAAGGC | | | | |
| | | | CAGCTGGCAGGGTGGAGGCA | | | | |
| | | | TATTCCAGTGTGCGGTGATGC | | | | |
| | | | ACGAGGCTCTGCACAACCACT | | | | |
| | | | ACACCCAGAAGTCTATCTCCA | | | | |
| | | | AGACTCCGGGTAAATGA (SEQ | | | | |
| | | | ID NO: 55) | | | | |
| | | IgG2^{b} | GCCCCCAAGACGGCCCCATTG | APKTAPLVYPLAPCGRDT | U03780 | | |
| | | | GTCTACCCTCTGGCCCCCTGC | SGPNVALGCLASSYFPEPV | | | |
| | | | GGCAGGGACACGTCTGGCCC | TVTWNSGALTSGVHTFPS | | | |
| | | | TAACGTGGCCTTGGGCTGCCT | VLQPSGLYSLSSMVTVPAS | | | |
| | | | GGCCTCAAGCTACTTCCCCGA | SLSSKSYTCNVNHPATTTK | | | |
| | | | GCCAGTGACCGTGACCTGGA | VDKRVGTKTKPPCPICPAC | | | |
| | | | ACTCGGGCGCCCTGACCAGT | ESPGPSVFIFPPKPKDTLMI | | | |
| | | | GGCGTGCATACCTTCCCATCC | SRTPQVTCVVVDVSQENP | | | |
| | | | GTCCTGCAGCCGTCAGGGCTC | EVQFSWYVDGVEVHTAQ | | | |
| | | | TACTCCCTCAGCAGCATGGTG | TRPKEFQFNSTYRVVSVLP | | | |
| | | | ACCGTGCCGGCCAGCAGCCT | IQHQDWLNGKEFKCKVN | | | |
| | | | GTCCAGCAAGAGCTACACCT | NKDLPAPITRIISKAKGQT | | | |
| | | | GCAATGTCAACCACCCGGCCA | REPQVYTLPPHAEELSRSK | | | |
| | | | CCACCACCAAGGTGGACAAG | VSITCLVIGFYPPDIDVEW | | | |
| | | | CGTGTTGGAACAAAGACCAA | QRNGQPEPEGNYRTTPPQ | | | |
| | | | ACCACCATGTCCCATATGCCC | QDVDGTYFLYSKFSVDKA | | | |
| | | | AGCCTGTGAATCGCCAGGGCC | SWQGGGIFQCAVMHEAL | | | |
| | | | CTCGGTCTTCATCTTCCCTCCA | HNHYTQKSISKTPGK* | | | |
| | | | AAACCCAAGGACACCCTCAT | (SEQ ID NO: 56) | | | |
| | | | GATCTCCCGGACACCCCAGGT | | | | |
| | | | CACGTGCGTGGTAGTTGATGT | | | | |
| | | | GAGCCAGGAGAACCCGGAGG | | | | |
| | | | TCCAGTTCTCCTGGTACGTGG | | | | |
| | | | ACGGCGTAGAGGTGCACACG | | | | |
| | | | GCCCAGACGAGGCCAAAGGA | | | | |
| | | | GGAGCAGTTCAACAGCACCT | | | | |
| | | | ACCGCGTGGTCAGCGTCCTGC | | | | |
| | | | CCATCCAGCACCAGGACTGGC | | | | |
| | | | TGAACGGGAAGGAGTTCAAG | | | | |
| | | | TGCAAGGTCAACAACAAAGA | | | | |
| | | | CCTCCCAGCCCCCATCACAAG | | | | |
| | | | GATCATCTCCAAGGCCAAAGG | | | | |
| | | | GCAGACCCGGGAGCCGCAGG | | | | |
| | | | TGTACACCCTGCCCCCACACG | | | | |
| | | | CCGAGGAGCTGTCCAGGAGC | | | | |
| | | | AAAGTCAGCATAACCTGCCTG | | | | |
| | | | GTCATTGGCTTCTACCCACCT | | | | |
| | | | GACATCGATGTCGAGTGGCAA | | | | |
| | | | AGAAACGGACAGCCGGAGCC | | | | |
| | | | AGAGGGCAATTACCGCACCA | | | | |
| | | | CCCCGCCCCAGCAGGACGTG | | | | |
| | | | GACGGGACCTACTTCCTGTAC | | | | |
| | | | AGCAAGTTCTCGGTGGACAA | | | | |
| | | | GGCCAGCTGGCAGGGTGGAG | | | | |
| | | | GCATATTCCAGTGTGCGGTGA | | | | |
| | | | TGCACGAGGCTCTGCACAAC | | | | |
| | | | CACTACACCCAGAAGTCTATC | | | | |
| | | | TCCAAGACTCCGGGTAAATGA | | | | |
| | | | (SEQ ID NO: 57) | | | | |
| | | IgG3 | GCCTACAACACAGCTCCATCG | AYNTAPSVYPLAPCGRDV | EU372658 | | |
| | | | GTCTACCCTCTGGCCCCCTGT | SDHNVALGCLVSSYFPEPV | | | |
| | | | GGCAGGGACGTGTCTGATCAT | TVTWNSGALSRVVHTFPS | | | |
| | | | AACGTGGCCTTGGGCTGCCTT | VLQPSGLYSLSSMVIVAAS | | | |
| | | | GTCTCAAGCTACTTCCCCGAG | SLSTLSYTCNVYHPATNTK | | | |
| | | | CCAGTGACCGTGACCTGGAA | VDKRVDIEPPTPICPEICSC | | | |
| | | | CTCGGGTGCCCTGTCCAGAGT | PAAEVLGAPSVFLFPPKPK | | | |
| | | | CGTGCATACCTTCCCATCCGT | DILMISRTPKVTCVVVDVS | | | |
| | | | CCTGCAGCCGTCAGGGCTCTA | QEEAEVQFSWYVDGVQL | | | |
| | | | CTCCCTCAGCAGCATGGTGAT | YTAQTRPMEEQFNSTYRV | | | |
| | | | CGTGGCGGCCAGCAGCCTGT | VSVLPIQHQDWLKGKEFK | | | |
| | | | CCACCCTGAGCTACACGTGCA | CKVNNKDLLSPITRTISKA | | | |
| | | | ACGTCTACCACCCGGCCACCA | TGPSRVPQVYTLPPAWEEL | | | |
| | | | ACACCAAGGTGGACAAGCGT | SKSKVSITCLCTGFYPPDI | | | |
| | | | GTTGACATCGAACCCCCCACA | DVEWQSNGQQEPEGNYR | | | |
| | | | CCCATCTGTCCCGAAATTTGC | TTPPQQDVDGTYFLYSKL | | | |
| | | | TCATGCCCAGCTGCAGAGGTC | AVDKVRWQRGDLFQCAV | | | |
| | | | CTGGGAGCACCGTCGGTCTTC | MHEALHNHYTQKSISKTQ | | | |
| | | | CTCTTCCCTCCAAAACCCAAG | GK (SEQ ID NO: 58) | | | |
| | | | GACATCCTCATGATCTCCCGG | | | | |
| | | | ACACCCAAGGTCACGTGCGT | | | | |
| | | | GGTGGTGGACGTGAGCCAGG | | | | |
| | | | AGGAGGCTGAAGTCCAGTTC | | | | |
| | | | TCCTGGTACGTGGACCCCGTA | | | | |
| | | | CAGTTGTACACGGCCCAGAC | | | | |
| | | | GAGGCCAATGGAGGAGCAGT | | | | |
| | | | TCAACAGCACCTACCGCGTGG | | | | |
| | | | TCAGCGTCCTGCCCATCCAGC | | | | |
| | | | ACCAGGACTGGCTGAAGGGG | | | | |
| | | | AAGGAGTTCAAGTGCAAGGT | | | | |
| | | | CAACAACAAAGACCTCCTTTC | | | | |
| | | | CCCCATCACGAGGACCATCTC | | | | |
| | | | CAAGGCTACAGGGCCGAGCC | | | | |
| | | | GGGTGCCGCAGGTGTACACC | | | | |
| | | | CTGCCCCCAGCCTGGGAAGA | | | | |
| | | | GCTGTCCAAGAGCAAAGTCA | | | | |
| | | | GCATAACCTGCCTGGTCACTG | | | | |
| | | | GCTTCTACCCACCTGACATCG | | | | |
| | | | ATGTCGAGTGGCAGAGCAAC | | | | |
| | | | GGACAACAAGAGCCAGAGGG | | | | |
| | | | CAATTACCGCACCACCCCGCC | | | | |
| | | | CCAGCAGGACGTGGATGGGA | | | | |
| | | | CCTACTTCCTGTACAGCAAGC | | | | |
| | | | TCGCGGTGGACAAGGTCAGG | | | | |
| | | | TGGCAGCGTGGAGACCTATTC | | | | |
| | | | CAGTGTGCGGTGATGCACGA | | | | |
| | | | GGCTCTGCACAACCACTACAC | | | | |
| | | | CCAGAAGTCCATCTCCAAGAC | | | | |
| | | | TCAGGGTAAATGA (SEQ ID | | | | |
| | | | NO: 59) | | | | |
| | | IgG4^{a} | ACCTTCCCATCCGTCCTGCAG | TFPSVLQPSGLYSLSSMVT | U03782 | | |
| | | | CCGTCAGGGCTCTACTCCCTC | VPASSLSSKSYTCNVNHPA | | | |
| | | | AGCAGCATGGTGACCGTGCC | TTTKVDKRVGTKTKPPCPI | | | |
| | | | GGCCAGCAGCCTGTCCAGCA | CPACEGPGPSAFIFPPKPK | | | |
| | | | AGAGCTACACCTGCAATGTCA | DTLMISRTPKVTCVVVDV | | | |
| | | | ACCACCCGGCCACCACCACC | SQENPEVQFSWYVDGVE | | | |
| | | | AAGGTGGACAAGCGTGTTGG | VHTAQTRPKEEQFNSTYR | | | |
| | | | AACAAAGACCAAACCACCAT | VVSVLPIQHQDWLNGKEF | | | |
| | | | GTCCCATATGCCCAGCCTGTG | KCKVNNKDLPAPITRIISK | | | |
| | | | AAGGGCCCGGGCCCTCGGCC | AKGQTREPQVYTLPPPTE | | | |
| | | | TTCATCTTCCCTCCAAAACCC | ELSRSKVTLTCLVTGFYPP | | | |
| | | | AAGGACACCCTCATGATCTCC | DIDVEWQRNGQPEPEGNY | | | |
| | | | CGGACCCCCAAGGTCACGTG | RTTPPQQDVDGTYFLYSK | | | |
| | | | CGTGGTGGTAGATGTGAGCCA | LAVDKASWQRGDTFQCA | | | |
| | | | GGAGAACCCGGAGGTCCAGT | VMHEALHNHYTQKSIFKT | | | |
| | | | TCTCCTGGTACGTGGACGGCG | PGK* (SEQ ID NO: 60) | | | |
| | | | TAGAGGTGCACACGGCCCAG | | | | |
| | | | ACGAGGCCAAAGGAGGAGCA | | | | |
| | | | GTTCAACAGCACCTACCGCGT | | | | |
| | | | GGTCAGCGTCCTGCCCATCCA | | | | |
| | | | GCACCAGGACTGGCTGAACG | | | | |
| | | | GGAAGGAGTTCAAGTGCAAG | | | | |
| | | | GTCAACAACAAAGACCTCCC | | | | |
| | | | AGCCCCCATCACAAGGATCAT | | | | |
| | | | CTCCAAGGCCAAAGGGCAGA | | | | |
| | | | CCCGGGAGCCGCAGGTGTAC | | | | |
| | | | ACCCTGCCCCCACCCACCGAG | | | | |
| | | | GAGCTGTCCAGGAGCAAAGT | | | | |
| | | | CACGCTAACCTGCCTGGTCAC | | | | |
| | | | TGGCTTCTACCCACCTGACAT | | | | |
| | | | CGATGTCGAGTGGCAAAGAA | | | | |
| | | | ACGGACAGCCGGAGCCAGAG | | | | |
| | | | GGCAATTACCGCACCACCCCG | | | | |
| | | | CCCCAGCAGGACGTGGACGG | | | | |
| | | | GACCTACTTCCTGTACAGCAA | | | | |
| | | | GCTCGCGGTGGACAAGGCCA | | | | |
| | | | GCTGGCAGCGTGGAGACACA | | | | |
| | | | TTCCAGTGTGCGGTGATGCAC | | | | |
| | | | GAGGCTCTGCACAACCACTAC | | | | |
| | | | ACCCAGAAGTCCATCTTCAAG | | | | |
| | | | ACTCCGGGTAAATGA (SEQ ID | | | | |
| | | | NO: 61) | | | | |
| | | IgG4^{b} | GCCCCCAAGACGGCCCCATCG | APKTAPSVYPLAPCGRDV | EU372654 | | |
| | | | GTCTACCCTCTGGCCCCCTGC | SGPNVALGCLASSYFPEPV | | | |
| | | | GGCAGGGACGTGTCTGGCCCT | TVTWNSGALTSGVHTFPS | | | |
| | | | AACGTGGCCTTGGGCTGCCTG | VLQPSGLYSLSSMVTVPAS | | | |
| | | | GCCTCAAGCTACTTCCCCGAG | SLSSKSYTCNVNHPATTTK | | | |
| | | | CCAGTGACCGTGACCTGGAA | VDKRVGIHQPQTCPICPAC | | | |
| | | | CTCGGGCGCCCTGACCAGTG | EGPGPSAFIFPPKPKDTLM | | | |
| | | | GCGTGCACACCTTCCCATCCG | ISRTPKVTCVVVDVSQEN | | | |
| | | | TCCTGCAGCCGTCAGGGCTCT | PEVQFSWYVDGVEVHTA | | | |
| | | | ACTCCCTCAGCAGCATGGTGA | QTRPKEEQFNSTYRVVSV | | | |
| | | | CCGTGCCGGCCAGCAGCCTGT | LLIQHQDWLNGKEFKCK | | | |
| | | | CCAGCAAGAGCTACACCTGC | VNNKDLPAPITRIISKAKG | | | |
| | | | AATGTCAACCACCCGGCCACC | QTREPQVYTLPPPTEELSR | | | |
| | | | ACCACCAAGGTGGACAAGCG | SKVTLTCLVTGFYPPDIDV | | | |
| | | | TGTTGGAATACACCAGCCGCA | EWQRNGQPEPEGNYRTTP | | | |
| | | | AACATGTCCCATAIGCCCAGC | PQQDVDGTYFLYSKLAVD | | | |
| | | | CTGTGAAGGGCCCGGGCCCT | KASWQRGDTFQCAVMHE | | | |
| | | | CGGCCTTCATCTTCCCTCCAA | ALHNGYT (SEQ ID NO: | | | |
| | | | AACCCAAGGACACCCTCATGA | 62) | | | |
| | | | TCTCCCGGACCCCCAAGGTCA | | | | |
| | | | CGTGCGTGGTGGTTGATGTGA | | | | |
| | | | GCCAGGAGAACCCGGAGGTC | | | | |
| | | | CAGTTCTCCTGGTACGTGGAC | | | | |
| | | | GGCGTAGAGGTGCACACGGC | | | | |
| | | | CCAGACGAGGCCAAAGGAGG | | | | |
| | | | AGCAGTTCAACAGCACCTACC | | | | |
| | | | GCGTGGTCAGCGTCCTGCTCA | | | | |
| | | | TCCAGCACCAGGACTGGCTG | | | | |
| | | | AACGGGAAGGAGTTCAAGTG | | | | |
| | | | CAAGGTCAACAACAAAGACC | | | | |
| | | | TCCCAGCCCCCATCACAAGGA | | | | |
| | | | TCATCTCCAAGGCCAAAGGGC | | | | |
| | | | AGACCCGGGAGCCGCAGGTG | | | | |
| | | | TACACCCTGCCCCCACCCACC | | | | |
| | | | GAGGAGCTGTCCAGGAGCAA | | | | |
| | | | AGTCACGCTAACCTGCCTGGT | | | | |
| | | | CACTGGCTTCTACCCACCTGA | | | | |
| | | | CATCGATGTCGAGTGGCAAAG | | | | |
| | | | AAACGGACAGCCGGAGCCAG | | | | |
| | | | AGGGCAATTACCGCACCACCC | | | | |
| | | | CGCCCCAGCAGGACGTGGAC | | | | |
| | | | GGGACCTACTTCCTGTACAGC | | | | |
| | | | AAGCTCGCGGTGGACAAGGC | | | | |
| | | | CAGCTGGCAGCGTGGAGACA | | | | |
| | | | CATTCCAGTGTGCGGTGATGC | | | | |
| | | | ACGAGGCTCTGCACAACCACT | | | | |
| | | | ACACCC (SEQ ID NO: 63) | | | | |
| | | IgG5^{a} | GCCCCCAAGACGGCCCCATCG | APKTAPSVYPLAPCSRDTS | EU372657 | | |
| | | | GTCTACCCTCTGGCCCCCTGC | GPNVALGCLVSSYFPEPVT | | | |
| | | | AGCAGGGACACGTCTGGCCC | VTWNSGALTSGVHTFPSV | | | |
| | | | TAACGTGGCCTTGGGCTGCCT | LQPSGLYSLSSMVTVPAHS | | | |
| | | | GGTCTCAAGCTACTTCCCCGA | LSSKRYTCNVNHPATKTK | | | |
| | | | GCCAGTGACCGTGACCTGGA | VDLCVGRPCPICPGCEVA | | | |
| | | | ACTCGGGCGCCCTGACCAGT | GPSVFIFPPKPKDILMISRT | | | |
| | | | GGCGTGCACACCTTCCCATCC | PEVTCVVVDVSKEHAEV | | | |
| | | | GTCCTGCAGCCGTCAGGGCTC | QFSWYVDGEEVHTAEFRP | | | |
| | | | TACTCCCTCAGCAGCATGGTG | KEEQFNSTYRVVSVLPIQH | | | |
| | | | ACCGTGCCGGCCCACAGCTTG | EDWLKGKEFECKVNNED | | | |
| | | | TCCAGCAAGCGCTATACGTGC | LPGPITRTISKAKGVVRSP | | | |
| | | | AATGTCAACCACCCAGCCACC | EVYTLPPPAEELSKSIVTLT | | | |
| | | | AAAACCAAGGTGGACCTGTG | CLVKSIFP?FIHVEWKING | | | |
| | | | TGTTGGACGACCATGTCCCAT | KPEPENAYRTTPPQEDEDR | | | |
| | | | ATGCCCAGGCTGTGAAGTGGC | TYFLYSKLAVDKARWDH | | | |
| | | | CGGGCCCTCGGTCTTCATCTT | GETFECAVMHEALHNHY | | | |
| | | | CCCTCCAAAACCCAAGGACAT | TQKSISKTQGK* (SEQ ID | | | |
| | | | CCTCATGATCTCCCGGACCCC | NO: 64) | | | |
| | | | CGAGGTCACGTGCGTGGTGG | | | | |
| | | | TGGACGTCAGCAAGGAGCAC | | | | |
| | | | GCCGAGGTCCAGTTCTCCTGG | | | | |
| | | | TACGTGGACGGCGAAGAGGT | | | | |
| | | | GCACACGGCCGAGACGAGGC | | | | |
| | | | CAAAGGAGGAGCAGTTCAAC | | | | |
| | | | AGCACCTACCGCGTGGTCAGC | | | | |
| | | | GTCCTGCCCATCCAGCACGAG | | | | |
| | | | GACTGGCTGAAGGGGAAGGA | | | | |
| | | | GTTCGAGTGCAAGGTCAACA | | | | |
| | | | ACGAAGACCTCCCAGGCCCC | | | | |
| | | | ATCACGAGGACCATCTCCAAG | | | | |
| | | | GCCAAAGGGGTGGTACGGAG | | | | |
| | | | CCCGGAGGTGTACACCCTGCC | | | | |
| | | | CCCACCCGCCGAGGAGCTGT | | | | |
| | | | CCAAGAGCATAGTCACGCTAA | | | | |
| | | | CCTGCCTGGTCAAAAGCATCT | | | | |
| | | | TCCCGNCTTTCATCCATGTTG | | | | |
| | | | AGTGGAAAATCAACGGAAAA | | | | |
| | | | CCAGAGCCAGAGAACGCATAT | | | | |
| | | | CGCACCACCCCGCCTCAGGA | | | | |
| | | | GGACGAGGACAGGACCTACT | | | | |
| | | | TCCTGTACAGCAAGCTCGCGG | | | | |
| | | | TGGACAAGGCAAGATGGGAC | | | | |
| | | | CATGGAGAAACATTTGAGTGT | | | | |
| | | | GCGGTGATGCACGAGGCTCTG | | | | |
| | | | CACAACCACTACACCCAGAA | | | | |
| | | | GTCCATCTCCAAGACTCAGGG | | | | |
| | | | TAAATGA (SEQ ID NO: 65) | | | | |
| | | IgG5^{b} | GCCTACAACACAGCTCCATCG | AYNTAPSVYPLAPCGRDV | EU372656 | | |
| | | | GTCTACCCTCTGGCCCCCTGT | SDHNVALGCLVSSYFPEPV | | | |
| | | | GGCAGGGACGTGTCTGATCAT | TVTWNWGAQTSGVHTFP | | | |
| | | | AACGTGGCCTTGGGCTGCCTG | SVLQPSGLYSLSSTVTVPA | | | |
| | | | GTCTCAAGCTACTTCCCCGAG | HSLSSKCFTCNVNHPATTT | | | |
| | | | CCAGTGACCGTGACCTGGAA | KVDLCVGKKTKPRCPICP | | | |
| | | | CTGGGGCGCCCAGACCAGTG | GCEVAGPSVFIFPPKPKDIL | | | |
| | | | GCGTGCACACCTTCCCATCCG | MISRTPEVTCVVVDVSKE | | | |
| | | | TCCTGCAGCCGTCAGGGCTCT | HAEVQFSWYVDGEEVHT | | | |
| | | | ACTCCCTCAGCAGCACGGTG | AETRPKEEQFNSTYRVVS | | | |
| | | | ACCGTGCCGGCCCACAGCTTG | VLPIQHEDWLKGKEFECK | | | |
| | | | TCCAGCAAGTGCTTCACGTGC | VNNEDLPGPITRTISKAKG | | | |
| | | | AATGTCAACCACCCGGCCACC | VVRSPEVYTLPPPAEELSK | | | |
| | | | ACCACCAAGGTGGACCTGTG | SIVTLTCLVKSFFPPFIHVE | | | |
| | | | TGTTGGAAAAAAGACCAAGC | WKINGKPEPENAYRTTPP | | | |
| | | | CTCGATGTCCCATATGCCCAG | QEDEDGTYFLYSKFSVEK | | | |
| | | | GCTGTGAAGTGGCCGGGCCC | FRWHSGGIHCAVMHEAL | | | |
| | | | TCGGTCTTCATCTTCCCTCCA | HNHYT (SEQ ID NO: 66) | | | |
| | | | AAACCCAAGGACATCCTCATG | | | | |
| | | | ATCTCCCGGACCCCCGAGGTC | | | | |
| | | | ACGTGCGTGGTGGTGGACGT | | | | |
| | | | CAGCAAGGAGCACGCCGAGG | | | | |
| | | | TCCAGTTCTCCTGGTACGTGG | | | | |
| | | | ACGGCGAAGAGGTGCACACG | | | | |
| | | | GCCGAGACGAGACCAAAGGA | | | | |
| | | | GGAGCAGTTCAACAGCACTT | | | | |
| | | | ACCGCGTGGTCAGCGTCCTGC | | | | |
| | | | CCATCCAGCACGAGGACTGG | | | | |
| | | | CTGAAGGGGAAGGAGTTCGA | | | | |
| | | | GTGCAAGGTCAACAACGAAG | | | | |
| | | | ACCTCCCAGGCCCCATCACGA | | | | |
| | | | GGACCATCTCCAAGGCCAAA | | | | |
| | | | GGGGTGGTACGGAGCCCGGA | | | | |
| | | | GGTGTACACCCTGCCCCCACC | | | | |
| | | | CGCCGAGGAGCTGTCCAAGA | | | | |
| | | | GCATAGTCACGCTAACCTGCC | | | | |
| | | | TGGTCAAAAGCTTCTTCCCGC | | | | |
| | | | CTTTCATCCATGTTGAGTGGA | | | | |
| | | | AAATCAACGGAAAACCAGAG | | | | |
| | | | CCAGAGAACGCATACCGCAC | | | | |
| | | | CACCCCGCCCCAGGAGGACG | | | | |
| | | | AGGACGGGACCTACTTCCTGT | | | | |
| | | | ACAGCAAGTTCTCGGTGGAA | | | | |
| | | | AAGTTCAGGTGGCACAGTGG | | | | |
| | | | AGGCATCCACTGTGCGGTGAT | | | | |
| | | | GCACGAGGCTCTGCACAACC | | | | |
| | | | ACTACACCC (SEQ ID NO: 67) | | | | |
| | | IgG6 | GCCCCCAAGACGGCCCCATCG | APKTAPSVYPLAPCGRDT | EU372655 | | |
| | | | GTCTACCCTCTGGCCCCCTGC | SGPNVALGCLASSYFPEPV | | | |
| | | | GGCAGGGACACGTCTGGCCC | TLTWNSGALTSGVHTFPS | | | |
| | | | TAACGTGGCCTTGGGCTGCCT | VLQPSGLYSLSSMVTVPAS | | | |
| | | | GGCCTCAAGCTACTTCCCCGA | SLSSKSYTCNVNHPATTTK | | | |
| | | | GCCAGTGACCCTGACCTGGA | VDLCVGRPCPICPACEGPG | | | |
| | | | ACTCGGGCGCCCTGACCAGT | PSVFIFPPKPKDTLMISRTP | | | |
| | | | GGCGTGCATACCTTCCCATCC | QVTCVVVDVSQENPEVQF | | | |
| | | | GTCCTGCAGCCGTCAGGGCTC | SWYVDGVEVHTAQTRPK | | | |
| | | | TACTCCCTCAGCAGCATGGTG | EAQFNSTYRVVSVLPIQHE | | | |
| | | | ACCGTGCCGGCCAGCAGCCT | DWLKGKEFECKVNNKDL | | | |
| | | | GTCCAGCAAGAGCTACACCT | PAPITRIISKAKGPSREPQV | | | |
| | | | GCAATGTCAACCACCCGGCCA | YTLSPSAEELSRSKVSITCL | | | |
| | | | CCACCACCAAGGTGGACCTG | VTGFYPPDIDVEWKSNGQ | | | |
| | | | TGTGTTGGACGACCATGTCCC | PEPEGNYRTTPPQQDVDG | | | |
| | | | ATATGCCCAGCCTGTGAAGGG | TYFLYSKLAVDKASWQRG | | | |
| | | | CCCGGGCCCTCGGTCTTCATC | DPFQCAVMHEALHNHYT | | | |
| | | | TTCCCTCCAAAACCCAAGGAC | (SEQ ID NO: 68) | | | |
| | | | ACCCTCATGATCTCCCGGACA | | | | |
| | | | CCCCAGGTCACGTGCGTGGTG | | | | |
| | | | GTAGATGTGAGCCAGGAAAA | | | | |
| | | | CCCGGAGGTCCAGTTCTCCTG | | | | |
| | | | GTATGTGGACGGTGTAGAGGT | | | | |
| | | | GCACACGGCCCAGACGAGGC | | | | |
| | | | CAAAGGAGGCGCAGTTCAAC | | | | |
| | | | AGCACCTACCGTGTGGTCAGC | | | | |
| | | | GTCCTGCCCATCCAGCACGAG | | | | |
| | | | GACTGGCTGAAGGGGAAGGA | | | | |
| | | | GTTCGAGTGCAAGGTCAACA | | | | |
| | | | ACAAAGACCTCCAGCCCCCA | | | | |
| | | | TCACAAGGATCATCTCCAAGG | | | | |
| | | | CCAAAGGGCCGAGCCGGGAG | | | | |
| | | | CCGCAGGTGTACACCCTGTCC | | | | |
| | | | CCATCCGCCGAGGAGCTGTCC | | | | |
| | | | AGGAGCAAAGTCAGCATAAC | | | | |
| | | | CTGCCTGGTCACTGGCTTCTA | | | | |
| | | | CCCACCTGACATCGATGTCGA | | | | |
| | | | GTGGAAGAGCAACGGACAGC | | | | |
| | | | CGGAGCCAGAGGGCAATTAC | | | | |
| | | | CGCACCACCCCGCCCCAGCA | | | | |
| | | | GGACGTGGACGGGACCTACT | | | | |
| | | | TCCTGTACAGCAAGCTCGCGG | | | | |
| | | | TGGACAAGGCCAGCTGGCAG | | | | |
| | | | CGTGGAGACCCATTCCAGTGT | | | | |
| | | | GCGGTGATGCACGAGGCTCTG | | | | |
| | | | CACAACCACTACACCC (SEQ | | | | |
| | | | ID NO: 69) | | | | |
| | | IgG6^{b} | GCCCCCAAGACGGCCCCATCG | APKTAPSVYPLAPCGRDT | EU372653 | | |
| | | | GTCTACCCTCTGGCCCCCTGC | SGPNVALGCLASSYFPEPV | | | |
| | | | GGCAGGGACACGTCTGGCCC | TVTWNSGALTSGVHTFPS | | | |
| | | | TAACGTGGCCTTGGGCTGCCT | VLQPSGLYSLSSTVTVPAR | | | |
| | | | GGCCTCAAGCTACTTCCCCGA | SSSRKCFTCNVNHPATTTK | | | |
| | | | GCCAGTGACCGTGACCTGGA | VDLCVGRPCPICPACEGN | | | |
| | | | ACTCGGGCGCCCTGACCAGT | GPSVFIFPPKPKDTLMISRT | | | |
| | | | GGCGTGCACACCTTCCCATCC | PEVTCVVVDVSQENPEVQ | | | |
| | | | GTCCTGCAGCCGTCAGGGCTC | FSWYVDGEEVHTAETRPK | | | |
| | | | TACTCCCTCAGCAGCACGGTG | EEQFNSTYRVVSVLPIQHQ | | | |
| | | | ACCGTGCCGGCCAGGAGCTC | DWLKGKEFECKVNNKDL | | | |
| | | | GTCCAGAAAGTGCTTCACGTG | PAPITRIISKAKGPSREPQV | | | |
| | | | CAATGTCAACCACCCGGCCAC | YTLSPSAEELSRSKVSITCL | | | |
| | | | CACCACCAAGGTGGACCTGT | VTGFYPPDIDVEWKSNGQ | | | |
| | | | GTGTTGGACGACCATGTCCCA | PEPEGNYRSTPPQEDEDG | | | |
| | | | TATGCCCAGCCTGTGAAGGGA | TYFLYSKLAVDKARLQSG | | | |
| | | | ACGGGCCCTCGGTCTTCATCT | GIHCAVMHEALHNHYTQ | | | |
| | | | TCCCTCCAAAACCCAAGGAC | KSISKT (SEQ ID NO: 70) | | | |
| | | | ACCCTCATGATCTCCCGGACC | | | | |
| | | | CCCGAGGTCACGTGCGTGGT | | | | |
| | | | GGTAGATGTGAGCCAGGAAA | | | | |
| | | | ACCCGGAGGTCCAGTTCTCCT | | | | |
| | | | GGTACGTGGACGGCGAAGAG | | | | |
| | | | GTGCACACGGCCGAGACGAG | | | | |
| | | | GCCAAAGGAGGAGCAGTTCA | | | | |
| | | | ACAGCACCTACCGTGTGGTCA | | | | |
| | | | GCGTCCTGCCCATCCAGCACC | | | | |
| | | | AGGACTGGCTGAAGGGAAAG | | | | |
| | | | GAGTTCGAGTGCAAGGTCAA | | | | |
| | | | CAACAAAGACCTCCCAGCCC | | | | |
| | | | CCATCACAAGGATCATCTCCA | | | | |
| | | | AGGCCAAAGGGCCGAGCCGG | | | | |
| | | | GAGCCGCAGGTGTACACCCT | | | | |
| | | | GTCCCCATCCGCCGAGGAGCT | | | | |
| | | | GTCCAGGAGCAAAGTCAGCA | | | | |
| | | | TAACCTGCCTGGTCACTGGCT | | | | |
| | | | TCTACCCACCTGACATCGATG | | | | |
| | | | TCGAGTGGAAGAGCAACGGA | | | | |
| | | | CAGCCGGAGCCAGAGGGCAA | | | | |
| | | | TTACCGCTCCACCCCGCCCCA | | | | |
| | | | GGAGGACGAGGACGGGGACCT | | | | |
| | | | ACTTCCTGTACAGCAAACTCG | | | | |
| | | | CGGTGGACAAGGCGAGGTTG | | | | |
| | | | CAGAGTGGAGGCATCCACTGT | | | | |
| | | | GCGGTGATGCACGAGGCTCTG | | | | |
| | | | CACAACCACTACACCCAGAA | | | | |
| | | | GTCCATCTCCAAGACT (SEQ | | | | |
| | | | ID NO: 71) | | | | |
| | Porcine | Ig | | | FP312898 | http://ww | Schwartz J.C. et |
| | Ig | kappa | | | | w.imgt.or | al., Immunogeneti |
| | light | (CK) | | | | g/IMGTre | cs, 64, 303-311 |
| | chain | variant | | | | pertoire/i | (2012). PMID: |
| | constant | 1 | | | | ndex.php | 22109540 |
| | region | | | | | ?section = | |
| | | | | | | LocusGe | |
| | | | | | | nes & repe | |
| | | | | | | rtoire = ge | |
| | | | | | | netable & s | |
| | | | | | | pecies = Pi | |
| | | | | | | g & group = | |
| | | | | | | IGLC | |
| | | Ig | | | CU694848 | | |
| | | kappa | | | | | |
| | | (CK) | | | | | |
| | | variant | | | | | |
| | | 2 | | | | | |
| | | Ig | | | CU467669 | http://ww | |
| | | lambda | | | | w.imgt.or | |
| | | (CL) | | | | g/IMGTre | |
| | | variant | | | | pertoire/i | |
| | | 1 | | | | ndex.php | |
| | | | | | | ?section = | |
| | | | | | | LocusGe | |
| | | | | | | nes & repe | |
| | | | | | | rtoire = ge | |
| | | | | | | netable & s | |
| | | | | | | pecies = Pi | |
| | | | | | | g & group = | |
| | | | | | | IGKC | |
| | | Ig | | | CU467599 | | |
| | | lambda | | | | | |
| | | (CK) | | | | | |
| | | variant | | | | | |
| | | 2 | | | | | |
| Water | Water | IgG1? | GAGCGGCGTGCACACCTTCCC | SGVHTFPAVLQSSGLYSLS | NW_005690903 | Not | None |
| buffalo | buffalo | | GGCCGTCCTTCAGTCCTCCGG | STVTAPASATKSQTFTCNV | | registered | |
| (Scienti- | Ig | | GCTCTACTCTCTCAGCAGCAC | AHPASSTKVDKAVVPPCR | | | |
| fic | heavy | | GGTGACCGCGCCCGCCAGCG | PKPCDCCPPPELPGGPSVF | | | |
| Name: | chain | | CCACAAAAAGCCAGACCTTC | IFPPKPKDTLTISGTPEVTC | | | |
| Bubalus | constant | | ACCTGCAACGTAGCCCACCCG | VVVDVGHDDPEVKFSWF | | | |
| bubalis) | region | | GCCAGCAGCACCAAGGTGGA | VDDVEVNTARTKPREEQF | | | |
| | (CH1- | | CAAGGCTGTTGTTCCCCCATG | NSTYRVVSALPIQHNDWT | | | |
| | CH3) | | CAGACCCGAAACCCTGTGATTG | GGKEFKCKVYNEGLPAPI | | | |
| | | | CTGCCCACCCCCTGAGCTCCC | VRTISRTKGQAREPQVYV | | | |
| | | | CGGAGGACCCTCTGTCTTCAT | LAPPQDELSKSTVSITCMV | | | |
| | | | CTTCCCACCAAAACCCAAGG | TGFYPDYIAVEWQKDGQP | | | |
| | | | ACACCCTCACAATCTCTGGAA | ESEDKYGTTPPQLDSDGS | | | |
| | | | CTCCTGAGGTCACGTGTGTGG | YFLYSRLRVNKNSWQEGG | | | |
| | | | TGGTGGACGTGGGCCACGAT | AYTCVVMHE (SEQ ID NO: | | | |
| | | | GACCCCGAGGTGAAGTTCTCC | 72) | | | |
| | | | TGGTTCGTGGACGATGTGGAG | | | | |
| | | | GTAAACACAGCCAGGACGAA | | | | |
| | | | GCCAAGAGAGGAGCAGTTCA | | | | |
| | | | ACAGCACCTACCGCGTGGTCA | | | | |
| | | | GCGCCCTGCCCATCCAGCACA | | | | |
| | | | ACGACTGGACTGGAGGAAAG | | | | |
| | | | GAGTTCAAGTGCAAGGTCTAC | | | | |
| | | | AATGAAGGCCTCCCAGCCCCC | | | | |
| | | | ATCGTGAGGACCATCTCCAGG | | | | |
| | | | ACCAAAGGGCAGGCCCGGGA | | | | |
| | | | GCCGCAGGTGTACGTCCTGGC | | | | |
| | | | CCCACCCCAGGACGAGCTCA | | | | |
| | | | GCAAAAGCACGGTCAGCATC | | | | |
| | | | ACTTGCATGGTCACTGGCTTC | | | | |
| | | | TACCCAGACTACATCGCCGTA | | | | |
| | | | GAGTGGCAGAAAGATGGGCA | | | | |
| | | | GCCTGAGTCAGAGGACAAATA | | | | |
| | | | TGGCACGACCCCGCCCCAGCT | | | | |
| | | | GGACAGCGATGGCTCCTACTT | | | | |
| | | | CCTGTACAGCAGGCTCAGGGT | | | | |
| | | | GAACAAGAACAGCTGGCAAG | | | | |
| | | | AAGGAGGCGCCTACACGTGT | | | | |
| | | | GTAGTGATGCATGAGGC (SEQ | | | | |
| | | | ID NO: 73) | | | | |
| | | IgG2? | GCCTCCATCACAGCCCCGAAA | ASITAPKVYPLTSCRGETS | NW_005766143 | | |
| | | | GTCTACCCTCTGACTTCTTGC | SSTVTLGCLVSSYMPEPVT | | | |
| | | | CGCGGGGAAACGTCCAGCTC | VTWNSGALKSGVHTFPAV | | | |
| | | | CACCGTGACCCTGGGCTGCCT | LQSSGLYSLSSTVTAPASA | | | |
| | | | GGTCTCCAGCTACATGCCCGA | TKSQTFTCNVAHPASSTK | | | |
| | | | GCCGGTGACCGTGACCTGGA | VDTAVGFSSDCCKFPKPC | | | |
| | | | ACTCGGGTGCCCTGAAGAGC | VRGPSVFIFPPKPKDTLMI | | | |
| | | | GGCGTGCACACCTTCCCGGCC | TGNPEVTCVVVDVGRDN | | | |
| | | | GTCCTTCAGTCCTCTGGGCTC | PEVQFSWFVGDVEVHTG | | | |
| | | | TACTCTCTCAGCAGCACGGTG | RSKPREEQFNSTYRVVSTL | | | |
| | | | ACCGCGCCCGCCAGCGCCAC | PIQHNDWTGGKEFKCKV | | | |
| | | | AAAAAGCCAGACCTTCACCT | NNKGLPAPIVRTISRTKGQ | | | |
| | | | GCAACGTAGCCCACCCGGCC | AREPQVYVLAPPQEELSK | | | |
| | | | AGCAGCACCAAGGTGGACAC | STVSVTCMVTGFYPDYIA | | | |
| | | | GGCTGTTGGGTTCTCCAGTGA | VEWHRDRQAESEDKYRT | | | |
| | | | CTGCTGCAAGTTTCCTAAGCC | TPPQLDSDGSYFLYSRLKV | | | |
| | | | TTGTGTGAGGGGACCATCTGT | NKNSWQEGGAYTCVVMH | | | |
| | | | CTTCATCTTCCCGCCGAAACC | E (SEQ ID NO: 74) | | | |
| | | | CAAAGACACCCTGATGATCAC | | | | |
| | | | AGGAAATCCCGAGGTCACATG | | | | |
| | | | TGTGGTGGTGGACGTGGGCC | | | | |
| | | | GGGATAACCCCGAGGTGCAG | | | | |
| | | | TTCTCCTGGTTCGTGGGTGAT | | | | |
| | | | GTGGAGGTGCACACGGGCAG | | | | |
| | | | GTCGAAGCCGAGAGAGGAGC | | | | |
| | | | AGTTCAACAGCACCFACCGCG | | | | |
| | | | TGGTCAGCACCCTGCCCATCC | | | | |
| | | | AGCACAATGACTGGACTGGA | | | | |
| | | | GGAAAGGAGITCAAGTGCAA | | | | |
| | | | GGTCAACAACAAAGGCCTCC | | | | |
| | | | CAGCCCCCATCGTGAGGACCA | | | | |
| | | | TCTCCAGGACCAAAGGGCAG | | | | |
| | | | GCCCGGGAGCCGCAGGTGTA | | | | |
| | | | CGTCCTGGCCCCACCCCAGGA | | | | |
| | | | AGAGCTCAGCAAAAGCACGG | | | | |
| | | | TCAGCGTCACTTGCATGGTCA | | | | |
| | | | CTGGCTTCTACCCAGACTACA | | | | |
| | | | TCGCCGTAGAGTGGCATAGAG | | | | |
| | | | ACCGGCAGGCTGAGTCGGAG | | | | |
| | | | GACAAGTACCGCACGACCCC | | | | |
| | | | GCCCCAGCTGGACAGCGATG | | | | |
| | | | GGTCCTACTTCCTGTACAGCA | | | | |
| | | | GGCTCAAGGTGAACAAGAAC | | | | |
| | | | AGCTGGCAAGAAGGAGGCGC | | | | |
| | | | CTACACGTGTGTAGTGATGCA | | | | |
| | | | TGAGGC (SEQ ID NO: 75) | | | | |
| | | IgG3? | GCCTCCACCACAGCCCCGAA | ASTTAPKVYPLASSCGDTS | NW_005784206 | | |
| | | | AGTCTACCCTCTGGCATCCAG | SSTVTLGCLVSSYMPEPVT | | | |
| | | | CTGCGGGGACACGTCCAGCT | VTWNSGALKNGVHTFPA | | | |
| | | | CCACCGTGACCCTGGGCTGCC | VRQSSGLYSLSSMVTMPT | | | |
| | | | TGGTCTCCAGCTACATGCCCG | STAGTQTFTCNVAHPASST | | | |
| | | | AGCCGGTGACCGTGACCTGG | KVDTAVTARHPVPKTPET | | | |
| | | | AACTCGGGTGCCCTGAAGAA | PIHPVKPPTQEPRDEKTPC | | | |
| | | | CGGCGTGCACACCTTCCCGGC | QCPKCPEPLGGLSVFIFPP | | | |
| | | | CGTCCGGCAGTCCTCCGGGCT | KPKDTLTISGTPEVTCVVV | | | |
| | | | CTACTCTCTCAGCAGCATGGT | DVGQDDPEVQFSWFVDD | | | |
| | | | GACCATGCCCACCAGCACCGC | VEVHTARMKPREEQFNST | | | |
| | | | AGGAACCCAGACCTTCACCT | YRVVSALPIQHQDWLREK | | | |
| | | | GCAACGTAGCCCACCCGGCC | EFKCKVNNKGLPAPIVRTI | | | |
| | | | AGCAGCACCAAGGTGGACAC | SRTKGQAREPQVYVLAPP | | | |
| | | | GGCTGTCACTGCAAGGCATCC | REELSKSTLSLTCLITGFYP | | | |
| | | | GGTCCCGAAGACACCAGAGA | EEVDVEWQRNGQPESED | | | |
| | | | CACCTATCCATCCTGTAAAAC | KYHTTPPQLDADGSYFLY | | | |
| | | | CCCCAACCCAGGAGCCCAGA | SRLRVNRSSWQEGDHYTC | | | |
| | | | GATGAAAAGACACCCTGCCA | AVMHEALRNHYKEKPISR | | | |
| | | | GTGTCCCAAATGCCCAGAACC | SPGK* (SEQ ID NO: 76) | | | |
| | | | TCTGGGAGGACTGTCTGTCTT | | | | |
| | | | CATCTTCCCACCGAAACCCAA | | | | |
| | | | GGACACCCTCACAATCTCTGG | | | | |
| | | | AACGCCCGAGGTCACGTGTG | | | | |
| | | | TGGTGGTGGACGTGGGCCAG | | | | |
| | | | GATGACCCCGAAGTGCAGTTC | | | | |
| | | | TCCTGGTTCGTGGATGACGTG | | | | |
| | | | GAGGTGCACACAGCCAGGAT | | | | |
| | | | GAAGCCAAGAGAGGAGCAGT | | | | |
| | | | TCAACAGCACCTACCGCGTGG | | | | |
| | | | TCAGCGCCCTGCCCATCCAGC | | | | |
| | | | ACCAGGACTGGCTGCGGGAA | | | | |
| | | | AAGGAGTTCAAGTGCAAGGT | | | | |
| | | | CAACAACAAAGGCCTCCCGG | | | | |
| | | | CCCCCATCGTGAGGACCATCT | | | | |
| | | | CCAGGACCAAAGGGCAGGCC | | | | |
| | | | CGGGAGCCACAGGTGTATGTC | | | | |
| | | | CTGGCCCCACCCCGGGAAGA | | | | |
| | | | GCTCAGCAAAAGCACGCTCA | | | | |
| | | | GCCTCACCTGCCTAATCACCG | | | | |
| | | | GCTTCTACCCAGAAGAGGTAG | | | | |
| | | | ACGTGGAGTGGCAGAGAAAT | | | | |
| | | | GGGCAGCCTGAGTCAGAGGA | | | | |
| | | | CAAGTACCACACGACCCCAC | | | | |
| | | | CCCAGCTGGACGCTGACGGC | | | | |
| | | | TCCTACTTCCTGTACAGCAGG | | | | |
| | | | CTCAGGGTGAACAGGAGCAG | | | | |
| | | | CTGGCAGGAAGGAGACCACT | | | | |
| | | | ACACGTGTGCAGTGATGCATG | | | | |
| | | | AAGCTTTACGGAATCACTACA | | | | |
| | | | AAGAGAAGCCCATCTCGAGG | | | | |
| | | | TCTCCGGGTAAATGA (SEQ ID | | | | |
| | | | NO: 77) | | | | |
| | Water | Ig | CAGCCCAAGTCCGCACCCTCA | QPKSAPSVTLFPPSTEELS | NW_005690786 | Not | None |
| | buffalo | lambda? | GTCACCCTGTTCCCACCCTCC | ANKATLVCLISDFYPGSMT | | registered | |
| | Ig | | ACGGAGGAGCTCAGCGCCAA | VARKADGSTITRNVETTR | | | |
| | light | | CAAGGCCACCCTGGTGTGTCT | ASKQSNSKYAASSYLSLT | | | |
| | chain | | CATCAGCGACTTCTACCCGGG | GSEWKSKGSYSCEVTHEG | | | |
| | constant | | TAGCATGACCGTGGCCAGGA | STVTKTVKPSECS* (SEQ | | | |
| | region | | AGGCAGACGGCAGCACCATC | ID NO: 78) | | | |
| | (CL) | | ACCCGGAACGTGGAGACCAC | | | | |
| | | | CCGGGCCTCCAAACAGAGCA | | | | |
| | | | ACAGCAAGTACGCGGCCAGC | | | | |
| | | | AGCTACCTGAGCCTGACGGG | | | | |
| | | | CAGCGAGTGGAAATCGAAAG | | | | |
| | | | GCAGTTACAGCTGCGAGGTC | | | | |
| | | | ACGCACGAGGGGAGCACCGT | | | | |
| | | | GACAAAGACAGTGAAGCCCT | | | | |
| | | | CAGAGTGTTCTTAG (SEQ ID | | | | |
| | | | NO: 79) | | | | |
| Human | Human | IgG4 | GAGTCCAAATATGGTCCCCCA | ESKYGPPCPSCPAPEFLGG | K01316 | http://ww | Ellison J. et al., |
| (Scienti- | Ig | variant | TGCCCATCATGCCCAGCACCT | PSVFLFPPKPKDTLMISRT | | w.imgt.or | DNA, 1, 11-18 |
| fic | heavy | 1 | GAGTTCCTGGGGGGACCATCA | PEVTCVVVDVSQEDPEVQ | | g/IMGTre | (1981). PMID: |
| Name: | chain | | GTCTTCCTGTTCCCCCCAAAA | FNWYNDGVEVHNAKTKP | | pertoire/i | 6299662 |
| Homo | constant | | CCCAAGGACACTCTCATGATC | REEQFNSTYRVVSVLTVL | | ndex.php | |
| sapiens) | region | | TCCCGGACCCCTGAGGTCACG | HQDWLNGKEYKCKVSNK | | ?section = | |
| | (CH1- | | TGCGTGGTGGTGGACGTGAG | GLPSSIEKTISKAKGQPRE | | LocusGe | |
| | CH3) | | CCAGGAAGACCCCGAGGTCC | PQVYTLPPSQEEMTKNQV | | nes & repe | |
| | | | AGTTCAACTGGTACGTGGATG | SLTCLVKGFYPSDIAVEWE | | rtoire = ge | |
| | | | GCGTGGAGGTGCATAATGCCA | SNGQPENNYKTTPPVLDS | | netable & s | |
| | | | AGACAAAGCCGCGGGAGGAG | DGSFFLYSRLTVDKSRWQ | | pecies = hu | |
| | | | CAGTTCAACAGCACGTACCGT | EGNVFSCSVMHEALHNH | | man & gro | |
| | | | GTGGTCAGCGTCCTCACCGTC | YTQKSLSLSLGK* (SEQ ID | | up = IGHC | |
| | | | CTGCACCAGGACTGGCTGAA | NO: 12) | | | |
| | | | CGGCAAGGAGTACAAGTGCA | | | | |
| | | | AGGTCTCCAACAAAGGCCTC | | | | |
| | | | CCGTCCTCCATCGAGAAAACC | | | | |
| | | | ATCTCCAAAGCCAAAGGGCA | | | | |
| | | | GCCCCGAGAGCCACAGGTGT | | | | |
| | | | ACACCCTGCCCCCATCCCAGG | | | | |
| | | | AGGAGATGACCAAGAACCAG | | | | |
| | | | GTCAGCCTGACCTGCCTGGTC | | | | |
| | | | AAAGGCTTCTACCCCAGCGAC | | | | |
| | | | ATCGCCGTGGAGTGGGAGAG | | | | |
| | | | CAATGGGCAGCCGGAGAACA | | | | |
| | | | ACTACAAGACCACGCCTCCCG | | | | |
| | | | TGCTGGACTCCGACGGCTCCT | | | | |
| | | | TCTTCCTCTACAGCAGGCTAA | | | | |
| | | | CCGTGGACAAGAGCAGGTGG | | | | |
| | | | CAGGAGGGGAATGTCTTCTCA | | | | |
| | | | TGCTCCGTGATGCATGAGGCT | | | | |
| | | | CTGCACAACCACTACACACA | | | | |
| | | | GAAGAGCCTCTCCCTGTCTCT | | | | |
| | | | GGGTAAATGA (SEQ ID NO: 14) | | | | |
| | | 1gG4 | GAGTCCAAATATGGTCCCCCG | ESKYGPPCPSCPAPEFLGG | AJ001563 | | Brusco A. et al., |
| | | variant | TGCCCATCATGCCCAGCACCT | PSVFLFPPKPKDTLMISRT | | | Eur. J. |
| | | 2 | GAGTTCCTGGGGGGACCATCA | PEVTCVVVDVSQEDPEVQ | | | Immunogenet., |
| | | | GTCTTCCTGTTCCCCCCAAAA | FNWYVDGVEVHNAKTKP | | | 25, 349-355 |
| | | | CCCAAGGACACTCTCATGATC | REEQFNSTYRVVSVLTVV | | | (1998). PMID: |
| | | | TCCCGGACCCCTGAGGTCACG | HQPWLNGKEYKCKVSNK | | | 9805657 |
| | | | TGCGTGGTGGTGGACGTGAG | GLPSSIEKTISKAKGQPRE | | | |
| | | | CCAGGAAGACCCCGAGGTCC | PQVYTLPPSQEEMTKNQV | | | |
| | | | AGTTCAACTGGTACGTGGATG | SLTCLVKGFYPSDIAVEWE | | | |
| | | | GCGTGGAGGTGCATAATGCCA | SNGQPENNYKTTPPVLDS | | | |
| | | | AGACAAAGCCGCGGGAGGAG | DGSFFLYSRLTVDKSRWQ | | | |
| | | | CAGTTCAACAGCACGTACCGT | EGNVFSCSVMHEALHNH | | | |
| | | | GTGGTCAGCGTCCTCACCGTC | YTQKSLSLSEGK (SEQ ID | | | |
| | | | GTGCACCAGGACTGGCTGAA | NO: 80) | | | |
| | | | CGGCAAGGAGTACAAGTGCA | | | | |
| | | | AGGTCTCCAACAAAGGCCTC | | | | |
| | | | CCGTCCTCCATCGAGAAAACC | | | | |
| | | | ATCTCCAAAGCCAAAGGGCA | | | | |
| | | | GCCCCGAGAGCCACAGGTGT | | | | |
| | | | ACACCCTGCCCCCATCCCAGG | | | | |
| | | | AGGAGATGACCAAGAACCAG | | | | |
| | | | GTCAGCCTGACCTGCCTGGTC | | | | |
| | | | AAAGGCTTCTACCCCAGCGAC | | | | |
| | | | ATCGCCGTGGAGTGGGAGAG | | | | |
| | | | CAATGGGCAGCCGGAGAACA | | | | |
| | | | ACTACAAGACCACGCCTCCCG | | | | |
| | | | TGCTGGACTCCGACGGCTCCT | | | | |
| | | | TCTTCCTCTACAGCAGGCTAA | | | | |
| | | | CCGTGGACAAGAGCAGGTGG | | | | |
| | | | CAGGAGGGGAATGTCTTCTCA | | | | |
| | | | TGCTCCGTGATGCATGAGGCT | | | | |
| | | | CTGCACAACCACTACACCXA | | | | |
| | | | GAAGAGCCTCTCCCTGTCTCT | | | | |
| | | | GGGTAAATGA (SEQ ID NO: 81) | | | | |
| | | IgG4 | GCACCTGAGTTCCTGGGGGG | APEFLGGSVFLFPPKPKD | AJ001564 | | |
| | | variant | ACCATCAGTCTTCCTGTTCCC | TLMISKTPEVTCVVVDVS | | | |
| | | 3 | CCCAAAACCCAAGGACACTC | QEDPEVQFNWYVDGVEV | | | |
| | | | TCATGATCTCCCGGACCCCTG | HNAKTKPREEQFNSTYRV | | | |
| | | | AGGTCACGTGCGTGGTGGTG | VSVLTVLHQDWLNGKEY | | | |
| | | | GACGTGAGCCAGGAAGACCC | KCKVSNKGLPSSIEKTISK | | | |
| | | | CGAGGTCCAGTTCAACTGGTA | AKGQPREPQVYTLPPSQE | | | |
| | | | CGTGGATGGCGTGGAGGTGCA | EMTKNQVSLTCLVKGFYP | | | |
| | | | TAATGCCAAGACAAAGCCGC | SDIAVEWESNGQPENNYK | | | |
| | | | GGGAGGAGCAGTTCAACAGC | TTPPVLDSDGSFFLYSKLT | | | |
| | | | ACGTACCGTGTGGTCAGCGTC | VDKSRWQEGNVFSCSVM | | | |
| | | | CTCACCGTCCTGCACCAGGAC | HEALHNHYTQKSLSLSLG | | | |
| | | | TGGCTGAACGGCAAGGAGTA | K* (SEQ ID NO: 82) | | | |
| | | | CAAGTGCAAGGTCTCCAACA | | | | |
| | | | AAGGCCTCCCGTCCTCCATCG | | | | |
| | | | AGAAAACCATCTCCAAAGCC | | | | |
| | | | AAAGGGCAGCCCCGAGAGCC | | | | |
| | | | ACAGGTGTACACCCTGCCCCC | | | | |
| | | | ATCCCAGGAGGAGATGACCA | | | | |
| | | | AGAACCAGGTCAGCCTGACC | | | | |
| | | | TGCCTGGTCAAAGGCTTCTAC | | | | |
| | | | CCCAGCGACATCGCCGTGGA | | | | |
| | | | GTGGGAGAGCAATGGGCAGC | | | | |
| | | | CGGAGAACAACTACAAGACC | | | | |
| | | | ACGCCTCCCGTGCTGGACTCC | | | | |
| | | | GACGGCTCCTTCTTCCTCTAC | | | | |
| | | | AGCAAGCTCACCGTGGACAA | | | | |
| | | | GAGCAGGTGGCAGGAGGGGA | | | | |
| | | | ACGTCTTCTCATGCTCCGTGA | | | | |
| | | | TGCATGAGGCTCTGCACAACC | | | | |
| | | | ACTACACGCAGAAGAGCCTC | | | | |
| | | | TCCCTGTCTCTGGGTAAATGA | | | | |
| | | | (SEQ ID NO: 83) | | | | |
| | Human | Ig | ACTGTGGCTGCACCATCTGTC | TVAAPSVFIFPPSDEQLKS | X96754 | http://ww | None |
| | Ig | kappa | TTCATCTTCCCGCCATCTGATG | GTASVVCLLNNFYPREAK | | w.imgt.or | |
| | light | (CK) | AGCAGTTGAAATCTGGAACTG | VQWKVDNALQSGNSQES | | g/IMGTre | |
| | chain | | CCTCTGTTGTGTGCCTGCTGA | VTEQDSKDSTYSLSSTLTL | | perioire/i | |
| | constant | | ATAACTTCTATCCCAGAGAGG | SKADYEKHKVYACEVTH | | ndex.php | |
| | region | | CCAAAGTACAGTGGAAGGTG | QGLSSPVTKSFNRGEC* | | ?section = | |
| | | | GATAACGCCCTCCAATCGGGT | (SEQ ID NO: 11) | | LocusGe | |
| | | | AACTCCCAGGAGAGTGYCAC | | | nes & repe | |
| | | | AGAGCAGGACAGCAAGGACA | | | rtoire = ge | |
| | | | GCACCTACAGCCTCAGCAGC | | | netable & s | |
| | | | ACCCTGACGCTGAGCAAAGC | | | pecies = hu | |
| | | | AGACTACGAGAAACACAAAG | | | man & gro | |
| | | | TCTACGCCTGCGAAGTCACCC | | | up = IGKC | |
| | | | ATCAGGGCCTGAGCTCGCCCG | | | | |
| | | | TCACAAAGAGCTTCAACAGG | | | | |
| | | | GGAGAGTGTTAG (SEQ ID NO: | | | | |
| | | | 13) | | | | |
| Bovine | Bovine | IgG1 | GCCTCCACCACAGCCCCGAA | ASTTAPKVYPLSSCCGDK | X62916 | hap://ww | Symons D.B. et |
| (Scienti- | Ig | variant | AGTCTACCCTCTGAGTTCTTG | SSSTVTLGCLVSSYMPEPV | | w.imgt.or | al. J. |
| fic | heavy | 1 | CTGCGGGGACAAGTCCAGCT | TVTWNSGALKSGVHTFPA | | g/IMGTre | Immunogenet., |
| Name: | chain | | CCACCGTGACCCTGGGCTGCC | VLQSSGLYSLSSMVTVPG | | pertoire/i | 14, 273-283 |
| Bos | constant | | TGGTCTCCAGCTACATGCCCG | STSGQTFTCNVAHPASSTK | | ndex.php | (1987). PMID: |
| taurus) | region | | AGCCGGTGACCGTGACCTGG | VDKAVDPTCKPSPCDCCP | | ?section = | 3141517 |
| | (CH1- | | AACTCGGGTGCCCTGAAGAG | PPELPGGPSVFIFPPKPKDT | | LocusGe | Symons D.B. et |
| | CH3) | | CGGCGTGCACACCTTCCCGGC | LTISGTPEVTCVVVDVGH | | nes & repe | al., Mol. |
| | | | TGTCCTTCAGTCCTCCGGGCT | DDPEVKFSWFVDDVEVNT | | rtoire = ge | Immunol., 26, |
| | | | GTACTCTCTCAGCAGCATGGT | ATTKPREEQFNSTYRVVS | | netable & s | 841-850 (1989). |
| | | | GACCGTGCCCGGCAGCACCT | ALRIQHQDWTGGKEFKC | | pecies = bo | PMID: 2513487 |
| | | | CAGGACAGACCTTCACCTGC | KVHNEGLPARIVRTISRTK | | vine & gro | Kacskovics I. and |
| | | | AACGTAGCCCACCCGGCCAG | GPAREPQVYVLAPPQEEL | | up = IGHC | Butler J.E., Mot |
| | | | CAGCACCAAGGTGGACAAGG | SKSTVSLTCMVTSFYPDYI | | | immunol., 33, |
| | | | CTGTTGATCCCACATGCAAAC | AVEWQRNGQPESEDKYG | | | 189-195 (1996). |
| | | | CATCACCCTGTGACTGTTGCC | TTPPQLDADSSYFLYSKLR | | | PMID: 8649440 |
| | | | CACCCCCTGAGCTCCCCGGAG | VDRNSWQEGDTYTCVVM | | | Rabbani H. et al., |
| | | | GACCCTCTGTCTTCATCTTCCC | HEALHNHYTQKSTSKSAG | | | Immunogenetics, |
| | | | ACCGAAACCCAAGGACACCC | K (SEQ ID NO: 84) | | | 46, 326-331 |
| | | | TCACAATCTCGGGAACGCCCG | | | | (1997). PMID: |
| | | | AGGTCACGTGTGTGGTGGTG | | | | 9218535 |
| | | | GACGTGGGCCACGATGACCC | | | | Saini S.S. et al., |
| | | | CGAGGTGAAGTTCTCCTGGTT | | | | Scand. J. |
| | | | CGTGGACGACGTGGAGGTAA | | | | Immunol. 65, 32- |
| | | | ACACAGCCACGACGAAGCCG | | | | 8 (2007). PMID: |
| | | | AGAGAGGAGCAGTTCAACAG | | | | 17212764 |
| | | | CACCTACCGCGTGGTCAGCGC | | | | |
| | | | CCTGCGCATCCAGCACCAGGA | | | | |
| | | | CTGGACTGGAGGAAAGGAGT | | | | |
| | | | TCAAGTGCAAGGTCCACAAC | | | | |
| | | | GAAGGCCTCCCGGCCCCCATC | | | | |
| | | | GTGAGGACCATCTCCAGGACC | | | | |
| | | | AAAGGGCCGGCCCGGGAGCC | | | | |
| | | | GCAGGTGTATGTCCTGGCCCC | | | | |
| | | | ACCCCAGGAAGAGCTCAGCA | | | | |
| | | | AAAGCACGGTCAGCCTCACC | | | | |
| | | | TGCATGGTCACCAGCTTCTAC | | | | |
| | | | CCAGACTACATCGCCGTGGAG | | | | |
| | | | TGGCAGAGAAACGGGCAGCC | | | | |
| | | | TGAGTCGGAGGACAAGTACG | | | | |
| | | | GCACGACCCCGCCCCAGCTG | | | | |
| | | | GACGCCGACAGCTCCTACTTC | | | | |
| | | | CTGTACAGCAAGCTCAGGGT | | | | |
| | | | GGACAGGAACAGCTGGCAGG | | | | |
| | | | AAGGAGACACCTACACGTGT | | | | |
| | | | GTGGTGATGCACGAGGCCCTG | | | | |
| | | | CACAATCACTACACGCAGAA | | | | |
| | | | GTCCACCTCTAAGTCTGCGGG | | | | |
| | | | TAAATGA (SEQ ID NO: 92) | | | | |
| | | IgG1 | GCCTCCACCACAGCCCCGAA | ASTTAPKVYPLSSCCGDK | X16701 | | |
| | | variant | AGTCTACCCTCTGAGTTCTTG | SSSTVTLGCLVSSYMPEPV | (M25278) | | |
| | | 2 | CTGCGGGGACAAGTCCAGCT | TVTWNSGALKSGVHTFPA | | | |
| | | | CCACCGTGACCCTGGGCTGCC | VLQSSGLYSLSSMVTVPG | | | |
| | | | TGGTCTCCAGCTACATGCCCG | STSGQTFTCNVAHPASSTK | | | |
| | | | AGCCGGTGACCGTGACCTGG | VDKAVDPTCKPSPCDCCP | | | |
| | | | AACTCGGGTGCCCTGAAGAG | PPELPGGPSVFIFPPKPKDT | | | |
| | | | CGGCGTGCACACCTTCCCGGC | LTISGTPEVTCVVVDVGH | | | |
| | | | CGTCCTTCAGTCCTCCGGGCT | DDPEVKFSWFVDDVEVNT | | | |
| | | | GTACTCTCTCAGCAGCATGGT | ATTKPREEQFNSTYRVVS | | | |
| | | | GACCGTGCCCGGCAGCACCT | ALRIQHQDWTGGKEFKC | | | |
| | | | CAGGACAGACCTTCACCTGC | KVHNEGLPAPIVRTISRTK | | | |
| | | | AACGTAGCCCACCCGGCCAG | GPAREPQVYVLAPPQEEL | | | |
| | | | CAGCACCAAGGTGGACAAGG | SKSTVSLTCMVTSFYPDYI | | | |
| | | | CTGTTGATCCCACATGCAAAC | AVEWQRNGQPESEDKYG | | | |
| | | | CATCACCCTGTGACTGTTGCC | TTPPQLDADSSYFLYSKLR | | | |
| | | | CACCCCCTGAGCTCCCCGGAG | VDRNSWQEGDTYTCVVM | | | |
| | | | GACCCTCTGTCTTCATCTTCCC | HEALHNHYTQKSTSKSAG | | | |
| | | | ACCGAAACCCAAGGACACCC | K* (SEQ ID NO: 85) | | | |
| | | | TCACAATCTCGGGAACGCCCG | | | | |
| | | | AGGTCACGTGTGTGGTGGTG | | | | |
| | | | GACGTGGGCCACGATGACCC | | | | |
| | | | CGAGGTGAAGTTCTCCTGGTT | | | | |
| | | | CGTGGACGACGTGGAGGTAA | | | | |
| | | | ACACAGCCACGACGAAGCCG | | | | |
| | | | AGAGAGGAGCAGTTCAACAG | | | | |
| | | | CACCTACCGCGTGGTCAGCGC | | | | |
| | | | CCTGCGCATCCAGCACCAGGA | | | | |
| | | | CTGGACTGGAGGAAAGGAGT | | | | |
| | | | TCAAGTGCAAGGTCCACAAC | | | | |
| | | | GAAGGCCTCCCGGCCCCCATC | | | | |
| | | | GTGAGGACCATCTCCAGGACC | | | | |
| | | | AAAGGGCCGGCCCGGGAGCC | | | | |
| | | | GCAGGTGTATGTCCTGGCCCC | | | | |
| | | | ACCCCAGGAAGAGCTCAGCA | | | | |
| | | | AAAGCACGGTCAGCCATCACC | | | | |
| | | | TGCATGGTCACCAGCTTCTAC | | | | |
| | | | CCAGACTACATCGCCGTGGAG | | | | |
| | | | TGGCAGAGAAACGGGCAGCC | | | | |
| | | | TGAGTCGGAGGACAAGTACG | | | | |
| | | | GCACGACCCCGCCCCAGCTG | | | | |
| | | | GACGCCGACAGCTCCTACTTC | | | | |
| | | | CTGTACAGCAAGCTCAGGGT | | | | |
| | | | GGACAGGAACAGCTGGCAGG | | | | |
| | | | AAGGAGACACCTACACGTGT | | | | |
| | | | GTGGTGATGCACGAGGCCCTG | | | | |
| | | | CACAATCACTACACGCAGAA | | | | |
| | | | GTCCACCTCTAAGTCTGCGGG | | | | |
| | | | TAAATGA (SEQ ID NO: 93) | | | | |
| | | IgG1 | GCCTCCACCACAGCCCCGAA | ASTTAPKVYPLSSCCGDK | S82409 | | |
| | | variant | AGTCTACCCTCTGAGTTCTTG | SSSTVTLGCLVSSYMPEPV | | | |
| | | 3 | CTGCGGGGACAAGTCCAGCT | TVTWNSGALKSGVHTFPA | | | |
| | | | CCACCGTGACCCTGGGCTGCC | VLQSSGLYSLSSMVTVPG | | | |
| | | | TGGTCTCCAGCTACATGCCCG | STSGTQTFTCNVAHPASST | | | |
| | | | AGCCGGTGACCGTGACCTGG | KVDKAVDPRCKTTCDCCP | | | |
| | | | AACTCGGGTGCCCTGAAGAG | PPELPGGPSVFIFPRKPKDT | | | |
| | | | CGGCGTGCACACCTTCCCGGC | LTISGTPEVTCVVVDVGH | | | |
| | | | CGTCCTTCAGTCCTCCGGGCT | DDPEVKFSWFVDDVEVNT | | | |
| | | | CTACTCTCTCAGCAGCATGGT | ATTKPREEQFNSTYRVVS | | | |
| | | | GACCGTGCCCGGCAGCACCT | ALRIQHQDWTGGKEFKC | | | |
| | | | CAGGAACCCAGACCTTCACCT | KVHNEGLPARIVRTISRTK | | | |
| | | | GCAACGTAGCCCACCCGGCC | GPAREPQVYVLAPPQEEL | | | |
| | | | AGCAGCACCAAGGTGGACAA | SKSTVSLTCMVTSFYPDYI | | | |
| | | | GGCTGTTGATCCCAGATGCAA | AVEWQRNGQPESEDKYG | | | |
| | | | AACAACCTGTGACTGTTGCCC | TIPPQLDADGSYFLYSRLR | | | |
| | | | ACCGCCTGAGCTCCCTGGAG | VDRNSWQEGDTYTCVVM | | | |
| | | | GACCCTGTCTTCATCTTCCC | HEALHNHYTQKSTSKSAG | | | |
| | | | ACCGAAACCCAAGGACACCC | K* (SEQ ID NO: 86) | | | |
| | | | TCACAATCTCGGGAACGCCCG | | | | |
| | | | AGGTCACGTGTGTGGTGGTG | | | | |
| | | | GACGTGGGCCACGATGACCC | | | | |
| | | | CGAGGTGAAGTTCTCCTGGTT | | | | |
| | | | CGTGGACGACGTGGAGGTAA | | | | |
| | | | ACACAGCCACGACGAAGCCG | | | | |
| | | | AGAGAGGAGCAGTTCAACAG | | | | |
| | | | CACCTACCGCGTGGTCAGCGC | | | | |
| | | | CCTGCGCATCCAGCACCAGGA | | | | |
| | | | CTGGACTGGAGGAAAGGAGT | | | | |
| | | | TCAAGTGCAAGGTCCACAAC | | | | |
| | | | GAAGGCCTCCCAGCCCCCATC | | | | |
| | | | GTGAGGACCATCTCCAGGACC | | | | |
| | | | AAAGGGCCGGCCCGGGAGCC | | | | |
| | | | GCAGGTGTATGTCCTGGCCCC | | | | |
| | | | ACCCCAGGAAGAGCTCAGCA | | | | |
| | | | AAAGCACGGTCAGCCTCACC | | | | |
| | | | TGCATGGTCACCAGCTTCTAC | | | | |
| | | | CCAGACTACATCGCCGTGGAG | | | | |
| | | | TGGCAGAGAAATGGGCAGCC | | | | |
| | | | TGAGTCAGAGGACAAGTACG | | | | |
| | | | GCACGACCCCTCCCCAGCTGG | | | | |
| | | | ACGCCGACGGCTCCTACTTCC | | | | |
| | | | TGTACAGCAGGCTCAGGGTG | | | | |
| | | | GACAGGAACAGCTGGCAGGA | | | | |
| | | | AGGAGACACCTACACGTGTG | | | | |
| | | | TGGTGATGCACGAGGCCCTGC | | | | |
| | | | ACAATCACTACACGCAGAAGT | | | | |
| | | | CCACCTCTAAGTCTGCGGGTA | | | | |
| | | | AATGA (SEQ ID NO: 94) | | | | |
| | | IgG2 | GCCTCCACCACAGCCCCGAA | ASTTAPKVYPLASSCGDTS | S82407 | | |
| | | variant | AGTCTACCCTCTGGCATCCAG | SSTVTLGCLVSSYMPEPVT | | | |
| | | 1 | CTGCGGAGACACATCCAGCTC | VTWNSGALKSGVHTFPAV | | | |
| | | | CACCGTGACCCTGGGCTGCCT | LQSSGLYSLSSMVTVPASS | | | |
| | | | GGTGTCCAGCTACATGCCCGA | SGQTFTCNVAHPASSTKV | | | |
| | | | GCCGGTGACCGTGACCTGGA | DKAVGVSIDCSKCHNQPC | | | |
| | | | ACTCGGGTGCCCTGAAGAGC | VREPSVFIFPPKPKDTLMI | | | |
| | | | GGCGTGCACACCTTCCCGGCT | TGTPEVTCVVVNVGHDN | | | |
| | | | GTCCTTCAGTCCTCCGGGCTC | PEVQFSWFVDDVEVHTAR | | | |
| | | | TACTCTCTCAGCAGCATGGTG | SKPREEQFNSTYRVVSALP | | | |
| | | | ACCGTGCCCGCCAGCAGCTC | IQHQDWTGGKEFKCKVN | | | |
| | | | AGGACAGACCTTCACCTGCA | NKGLSAPIVRIISRSKGPAR | | | |
| | | | ACGTAGCCCACCCGGCCAGC | EPQVYVLDPPKEELSKSTL | | | |
| | | | AGCACCAAGGTGGACAAGGC | SVTCMVTGFYPEDVAVEW | | | |
| | | | TGTTGGGGTCTCCATTGACTG | QRNRQTESEDKYRTTPPQ | | | |
| | | | CTCCAAGTGTCATAACCAGCC | LDTDRSYFLYSKLRVDRN | | | |
| | | | TTGCGTGAGGGAACCATCTGT | SWQEGDAYTCVVMHEAL | | | |
| | | | CTTCATCTTCCCACCGAAACC | HNHYMQKSTSKSAGK* | | | |
| | | | CAAAGACACCCTGATGATCAC | (SEQ ID NO: 87) | | | |
| | | | AGGAACGCCCGAGGTCACGT | | | | |
| | | | GTGTGGTGGTGAACGTGGGC | | | | |
| | | | CACGATAACCCCGAGGTGCA | | | | |
| | | | GTTCTCCTGGTTCGTGGATGA | | | | |
| | | | CGTGGAGGTGCACACGGCCA | | | | |
| | | | GGTCGAAGCCAAGAGAGGAG | | | | |
| | | | CAGTTCAACAGCACGTACCGC | | | | |
| | | | GTGGTCAGCGCCCTGCCCATC | | | | |
| | | | CAGCACCAGGACTGGACTGG | | | | |
| | | | AGGAAAGGAGTTCAAGTGCA | | | | |
| | | | AGGTCAACAACAAAGGCCTC | | | | |
| | | | TCGGCCCCCATCGTGAGGATC | | | | |
| | | | ATCTCCAGGAGCAAAGGGCC | | | | |
| | | | GGCCCGGGAGCCGCAGGTGT | | | | |
| | | | ATGTCCTGGACCCACCCAAGG | | | | |
| | | | AAGAGCTCAGCAAAAGCACG | | | | |
| | | | CTCAGCGTCACCTGCATGGTC | | | | |
| | | | ACCGGCTTCTACCCAGAAGAT | | | | |
| | | | GTAGCCGTGGAGTGGCAGAG | | | | |
| | | | AAACCGGCAGACTGAGTCGG | | | | |
| | | | AGGACAAGTACCGCACGACC | | | | |
| | | | CCGCCCCAGCTGGACACCGA | | | | |
| | | | CCGCTCCTACTTCCTGTACAG | | | | |
| | | | CAAGCTCAGGGTGGACAGGA | | | | |
| | | | ACAGCTGGCAGGAAGGAGAC | | | | |
| | | | GCCTACACGTGTGTGGTGATG | | | | |
| | | | CACGAGGCCCTGCACAATCAC | | | | |
| | | | TACATGCAGAAGTCCACCTCT | | | | |
| | | | AAGTCTGCGGGTAAATGA | | | | |
| | | | (SEQ ID NO: 95) | | | | |
| | | IgG2 | GCCTCCACCACAGCCCCGAA | ASTTAPKVYPLSSCCGDK | M36946 | | |
| | | variant | AGTCTACCCTCTGAGTTCTTG | SSSTVTLGCLVSSYMPEPV | (X06703) | | |
| | | 2 | CTGCGGGGACAAGTCCAGCT | TVTWNSGALKSGVHTFPA | | | |
| | | | CCACCGTGACCCTGGGCTGCC | VLQSSGLYSLSSMVTVPG | | | |
| | | | TGGTGTCCAGCTACATGCCCG | STSGQTFTCNVAHPASSTK | | | |
| | | | AGCCGGTGACCGTGACCTGG | VDKAVGVSSDCSKPNNQ | | | |
| | | | AACTCGGGTGCCCTGAAGAG | HCVREPSVFIFPPKPKDTL | | | |
| | | | CGGCGTGCACACCTTCCCGGC | MITGTPEVTCVVVNVGHD | | | |
| | | | CGTCCTTCAGTCCTCCGGGCT | NPEVQFSWFVDDVEVHTA | | | |
| | | | CTACTCTCTCAGCAGCATGGT | RTKPREEQFNSTYRVVSA | | | |
| | | | GACCGTGCCCGGCAGCACCT | LPIQHQDWTGGKEFKCKV | | | |
| | | | CAGGACAGACCTTCACCTGC | NIKGLSASINRIIRSKGPA | | | |
| | | | AACGTAGCCCACCCGGCCAG | REPQVYVLDPPKEELSKS | | | |
| | | | CAGCACCAAGGTGGACAAGG | TVSVTCMVIGFYPEDVDV | | | |
| | | | CTGTTGGGGTCTCCAGTGACT | EWQRDRQTESEDKYRTTP | | | |
| | | | GCTCCAAGCCTAATAACCAGC | PQLDADRSYFLYSKLRVD | | | |
| | | | ATTGCGTGAGGGAACCATCTG | RNSWQRGDTYTCVVMHE | | | |
| | | | TCTTCATCTTCCCACCGAAAC | ALHNHYMQKSTSKSAGK | | | |
| | | | CCAAAGACACCCTGATGATCA | * (SEQ ID NO: 88) | | | |
| | | | CAGGAACGCCCGAGGTCACG | | | | |
| | | | TGTGTGGTGGTGAACGTGGG | | | | |
| | | | CCACGATAACCCCGAGGTGCA | | | | |
| | | | GTTCTCCTGGTTCGTGGACGA | | | | |
| | | | CGTGGAGGTGCACACGGCCA | | | | |
| | | | GGACGAAGCCGAGAGAGGAG | | | | |
| | | | CAGTTCAACAGCACGTACCGC | | | | |
| | | | GTGGTCAGCGCCCTGCCCATC | | | | |
| | | | CAGCACCAGGACTGGACTGG | | | | |
| | | | AGGAAAGGAGTTCAAGTGCA | | | | |
| | | | AGGTCAACATCAAAGGCCTCT | | | | |
| | | | CGGCCTCCATCGTGAGGATCA | | | | |
| | | | TCTCCAGGAGCAAAGGGCCG | | | | |
| | | | GCCCGGGAGCCGCAGGTGTAT | | | | |
| | | | GTCCTGGACCCACCCAAGGA | | | | |
| | | | AGAGCTCAGCAAAAGCACGG | | | | |
| | | | TCAGCGTCACCTGCATGGTCA | | | | |
| | | | TCGGCTTCTACCCAGAAGATG | | | | |
| | | | TAGACGTGGAGTGGCAGAGA | | | | |
| | | | GACCGGCAGACTGAGTCGGA | | | | |
| | | | GGACAAGTACCGCACGACCC | | | | |
| | | | CGCCCCAGCTGGACGCCGAC | | | | |
| | | | CGCTCCTACTTCCTGTACAGC | | | | |
| | | | AAGCTCAGGGTGGACAGGAA | | | | |
| | | | CAGCTGGCAGAGAGGAGACA | | | | |
| | | | CCTACACGTGTGTGGTGATGC | | | | |
| | | | ACGAGGCCCTGCACAATCACT | | | | |
| | | | ACATGCAGAAGTCCACCTCTA | | | | |
| | | | AGTCTGCGGGTAAATGA (SEQ | | | | |
| | | | ID NO: 96) | | | | |
| | | IgG2 | GCCTCCACCACAGCCCCGAA | ASTTAPKVYPLSSCCGDK | X16702 | | |
| | | variant | AGTCTACCCTCTGAGTTCTTG | SSSGVTLGCLVSSYMPEPV | (M25279) | | |
| | | 3 | CTGCGGGGACAAGTCCAGCT | TVTWNSGALKSGVHTFPA | | | |
| | | | CGGGGGTGACCCTGGGCTGC | VLQSSGLYSLSSMVTVPAS | | | |
| | | | CTGGTCTCCAGCTACATGCCC | SSGTQTFTCNVAHPASSTK | | | |
| | | | GAGCCGGTGACCGTGACCTG | VDKAVGVSSDCSKPNNQ | | | |
| | | | GAACTCGGGTGCCCTGAAGA | HCVREPSVFIFPPKPKDTL | | | |
| | | | GCGGCGTGCACACCTTCCCGG | MITGTPEVTCVVVNVGHD | | | |
| | | | CCGTCCTTCAGTCCTCCGGGC | NPEVQFSWFVDDVEVHTA | | | |
| | | | TCTACTCTCTCAGCAGCATGG | RTKPREEQFNSTYRVVSA | | | |
| | | | TGACCGTGCCCGCCAGCAGCT | LPIQHQDWTGGKEEKCKV | | | |
| | | | CAGGAACCCAGACCTTCACCT | NIKGLSASIVRIISRSKGPA | | | |
| | | | GCAACGTAGCCCACCCGGCC | REPQVYVLDPPKEELSKS | | | |
| | | | AGCAGCACCAAGGTGGACAA | TVSLTCMVIGFYPEDVDV | | | |
| | | | GGCTGTTGGGGTCTCCAGTGA | EWQRDRQTESEDKYRTTP | | | |
| | | | CTGCTCCAAGCCTAATAACCA | PQLDADRSYFLYSKLRVD | | | |
| | | | GCATTGCGTGAGGGAACCATC | RNSWQRGDTYTCVVMHE | | | |
| | | | TGTCTTCATCTTCCCACCGAA | ALHNHYMQKSTSKSAGK | | | |
| | | | ACCCAAAGACACCCTGATGAT | * (SEQ ID NO: 89) | | | |
| | | | CACAGGAACGCCCGAGGTCA | | | | |
| | | | CGTGTGTGGTGGTGAACGTG | | | | |
| | | | GGCCACGATAACCCCGAGGT | | | | |
| | | | GCAGTTCTCCTGGTTCGTGGA | | | | |
| | | | CGACGTGGAGGTGCACACGG | | | | |
| | | | CCAGGACGAAGCCGAGAGAG | | | | |
| | | | GAGCAGTTCAACAGCACGTA | | | | |
| | | | CCGCGTGGTCAGCGCCCTGCC | | | | |
| | | | CATCCAGCACCAGGACTGGA | | | | |
| | | | CTGGAGGAAAGGAGTTCAAG | | | | |
| | | | TGCAAGGTCAACATCAAAGG | | | | |
| | | | CCTCTCGGCCTCCATCGTGAG | | | | |
| | | | GATCATCTCCAGGAGCAAAGG | | | | |
| | | | GCCGGCCCGGGAGCCGCAGG | | | | |
| | | | TGTATGTCCTGGACCCACCCA | | | | |
| | | | AGGAAGAGCTCAGCAAAAGC | | | | |
| | | | ACGGTCAGCCTCACCTGCATG | | | | |
| | | | GTCATCGGCTTCTACCCAGAA | | | | |
| | | | GATGTAGACGTGGAGTGGCA | | | | |
| | | | GAGAGACCGGCAGACTGAGT | | | | |
| | | | CGGAGGACAAGTACCGCACG | | | | |
| | | | ACCCCGCCCCAGCTGGACGC | | | | |
| | | | CGACCGCTCCTACTTCCTGTA | | | | |
| | | | CAGCAAGCTCAGGGTGGACA | | | | |
| | | | GGAACAGCTGGCAGAGAGGA | | | | |
| | | | GACACCTACACGTGTGTGGTG | | | | |
| | | | ATGCACGAGGCCCTGCACAAT | | | | |
| | | | CACTACATGCAGAAGTCCACC | | | | |
| | | | TCTAAGTCTGCGGGTAAATGA | | | | |
| | | | (SEQ ID NO: 97) | | | | |
| | | IgG3 | GCCTCCACCACAGCCCCGAA | ASTTAPKVYPLASSCGDTS | U63638 | | |
| | | variant | AGTCTACCCTCTGGCATCCAG | SSTVTLGCLVSSYMPEPVT | | | |
| | | 1 | CTGCGGAGACACATCCAGCTC | VTWNSGALKSGVHTFPAV | | | |
| | | | CACCGTGACCCTGGGCTGCCT | RQSSGLYSLSSMVTVPASS | | | |
| | | | GGTCTCCAGCTACATGCCCGA | SETQTFTCNVAHPASSTKV | | | |
| | | | GCCGGTGACCCGTGACCTGGA | DKAVTARRPVPTTPKTTIP | | | |
| | | | ACTCGGGTGCCCTGAAGAGC | PGKPTTPKSEVEKTPCQCS | | | |
| | | | GGCGTGCACACCTTCCCGGCC | KCPEPLGGLSVFIFPPKPK | | | |
| | | | GTCCGGCAGTCCTCTGGGCTG | DILTISGTPEVTCVVVDV | | | |
| | | | TACTCTCTCAGCAGCATGGTG | GQDDPEVQFSWFVDDVE | | | |
| | | | ACTGTGCCCGCCAGCAGCTCA | VHTARTKPREEQFNSTYR | | | |
| | | | GAAACCCAGACCTTCACCTGC | VVSALRIQHQDWLQGKEF | | | |
| | | | AACGTAGCCCACCCGGCCAG | KCKVNNKGLPAPIVRTISR | | | |
| | | | CAGCACCAAGGTGGACAAGG | TKGQAREPQVYVLAPPRE | | | |
| | | | CTGTCACTGCAAGGCGTCCAG | ELSKSTLSLTCLITGFYPEE | | | |
| | | | TCCCGACGACGCCAAAGACA | IDVEWQRNGQPESEDKYH | | | |
| | | | ACTATCCCTCCTGGAAAACCC | TTAPQLDADGSYFLYSKL | | | |
| | | | ACAACCCCAAAGTCTGAAGT | RVNKSSWQEGDHYTCAV | | | |
| | | | TGAAAAGACACCCTGCCAGT | MHEALRNHYKEKSISRSP | | | |
| | | | GTTCCAAATGCCCAGAACCTC | GK* (SEQ ID NO: 90) | | | |
| | | | TGGGAGGACTGTCTGTCTTCA | | | | |
| | | | TCTTCCCACCGAAACCCAAGG | | | | |
| | | | ACACCCTCACAATCTCGGGAA | | | | |
| | | | CGCCCGAGGTCACGTGTGTG | | | | |
| | | | GTGGTGGACGTGGGCCAGGA | | | | |
| | | | TGACCCCGAGGTGCAGTTCTC | | | | |
| | | | CTGGTTCGTGGACGACGTGG | | | | |
| | | | AGGTGCACACGGCCAGGACG | | | | |
| | | | AAGCCGAGAGAGGAGCAGTT | | | | |
| | | | CAACAGCACCTACCGCGTGGT | | | | |
| | | | CAGCGCCCTGCGCATCCAGCA | | | | |
| | | | CCAGGACTGGCTGCAGGGAA | | | | |
| | | | AGGAGTTCAAGTGCAAGGTC | | | | |
| | | | AACAACAAAGGCCTCCCGGC | | | | |
| | | | CCCCATTGTGAGGACCATCTC | | | | |
| | | | CAGGACCAAAGGGCAGGCCC | | | | |
| | | | GGGAGCCGCAGGTGTATGTCC | | | | |
| | | | TGGCCCCACCCCGGGAAGAG | | | | |
| | | | CTCAGCAAAAGCACGCTCAG | | | | |
| | | | CCTCACCTGCCTGATCACCGG | | | | |
| | | | TTTCTACCCAGAAGAGATAGA | | | | |
| | | | CGTGGAGTGGCAGAGAAATG | | | | |
| | | | GGCAGCCTGAGTCGGAGGAC | | | | |
| | | | AAGTACCACACGACCGCACC | | | | |
| | | | CCAGCTGGAIGCTGACGGCTC | | | | |
| | | | CTACTTCCTGTACAGCAAGCT | | | | |
| | | | CAGGGTGAACAAGAGCAGCT | | | | |
| | | | GGCAGGAAGGAGACCACTAC | | | | |
| | | | ACGTGTGCAGTGATGCACGA | | | | |
| | | | AGCTTTACGGAATCACTACAA | | | | |
| | | | AGAGAAGTCCATCTCGAGGTC | | | | |
| | | | TCCGGGTAAATGA (SEQ ID | | | | |
| | | | NO: 98) | | | | |
| | | IgG3 | GCCTCCACCACAGCCCCGAA | ASTTAPKVYPLASRCGDT | 1163639 | | |
| | | variant | AGTCTACCCTCTGGCATCCCG | SSSTVTLGCLVSSYMPEPV | | | |
| | | 2 | CTGCGGAGACACATCCAGCTC | TVTWNSGALKSGVHTFPA | | | |
| | | | CACCGTGACCCTGGGCTGCCT | VLQSSGLYSLSSMVTVPAS | | | |
| | | | GGTCTCCAGCTACATGCCCGA | TSETQTFTCNVAHPASSTK | | | |
| | | | GCCGGTGACCGTGACCTGGA | VDKAVTARRPVPTTPKTTI | | | |
| | | | ACTCGGGTGCCCTGAAGAGT | PPGKPTTQESEVEKTPCQC | | | |
| | | | GGCGTGCACACCTTCCCGGCC | SKCPEPLGGLSVFIFPPKP | | | |
| | | | GTCCTTCAGTCCTCCGGGCTG | KDTLTISGTPEVTCVVVD | | | |
| | | | TACTCTCTCAGCAGCATGGTG | VGQDDPEVQFSWFVDDV | | | |
| | | | ACCGTGCCCGCCAGCACCTCA | EVHTARTKPREEQFNSTY | | | |
| | | | GAAACCCAGACCTTCACCTGC | RVVSALRIQHQDWLQGKE | | | |
| | | | AACGTAGCCCACCCGGCCAG | FKCKVNNKGLPAPIVRTIS | | | |
| | | | CAGCACCAAGGTGGACAAGG | RTKGQAREPQVYVLAPPR | | | |
| | | | CTGTCCTGCAAGGCGTCCAG | EELSKSTLSLTCLITGFYPE | | | |
| | | | TCCCGACGACGCCAAAGACA | EIDVEWQRNGQPESEDKY | | | |
| | | | ACCATCCCTCCTGGAAAACCC | HTFAPQLDADGSYFLYSR | | | |
| | | | ACAACCCAGGAGTCTGAAGT | LRVNKSSWQEGDHYTCA | | | |
| | | | TGAAAAGACACCCTGCCAGT | VMHEALRNHYKEKSISRS | | | |
| | | | GTTCCAAATGCCCAGAACCTC | PGK* (SEQ ID NO: 91) | | | |
| | | | TGGGAGGACTGTCTGTCTTCA | | | | |
| | | | TCTTCCCACCGAAACCCAAGG | | | | |
| | | | ACACCCTCACAATCTCGGGAA | | | | |
| | | | CGCCCGAGGTCACGTGTGTG | | | | |
| | | | GTGGTGGACGTGGGCCAGGA | | | | |
| | | | TGACCCCGAGGTGCAGTTCTC | | | | |
| | | | CTGGTTCGTGGACGACGTGG | | | | |
| | | | AGGTGCACACGGCCAGGACG | | | | |
| | | | AAGCCGAGAGAGGAGCAGTT | | | | |
| | | | CAACAGCACCTACCGCGTGGT | | | | |
| | | | CAGCGCCCTGCGCATCCAGCA | | | | |
| | | | CCAGGACTGGCTGCAGGGAA | | | | |
| | | | AGGAGTTCAAGTGCAAGGTC | | | | |
| | | | AACAACAAAGGCCTCCCGGC | | | | |
| | | | CCCCATTGTGAGGACCATCTC | | | | |
| | | | CAGGACCAAAGGGCAGGCCC | | | | |
| | | | GGGAGCCCGCAGGTGTATGTCC | | | | |
| | | | TGGCCCCACCCCGGGAAGAG | | | | |
| | | | CTCAGCAAAAGCACGCTCAG | | | | |
| | | | CCTCACCTGCCTGATCACCGG | | | | |
| | | | TTTCTACCCAGAAGAGATAGA | | | | |
| | | | CGTGGAGTGGCAGAGAAATG | | | | |
| | | | GGCAGCCTGAGTCGGAGGAC | | | | |
| | | | AAGTACCACACGACCGCACC | | | | |
| | | | CCAGCTGGATGCTGACGGCTC | | | | |
| | | | CTACTTCCTGTACAGCAGGCT | | | | |
| | | | CAGGGTGAACAAGAGCAGCT | | | | |
| | | | GGCAGGAAGGAGACCACTAC | | | | |
| | | | ACGTGTGCAGTGATGCATGAA | | | | |
| | | | GCTTTACGGAATCACTACAAA | | | | |
| | | | GAGAAGTCCATCTCGAGGTCT | | | | |
| | | | CCGGGTAAATGA (SEQ ID NO: | | | | |
| | | | 99) | | | | |
| | Bovine, | Ig | CAGCCCAAGTCCCCACCCTCG | QPKSPPSVTLFPPSTEELN | X62917 | Not | Chen L. et al., |
| | Ig light | lambda | GTCACCCTGTTCCCGCCCTCC | GNKATLVCLISDFYPGSVT | | registered | Vet. Immunol. |
| | chain | | ACGGAGGAGCTCAACGGCAA | VVWKADGSTITRNVETTR | | | Immunopathol, |
| | constant | | CAAGGCCACCCTGGTGTGTCT | ASKQSNSKYAASSYLSLTS | | | 124, 284-294 |
| | region | | CATCAGCGACTTCTACCCGGG | SDWKSKGSYSCEVTHEGS | | | (2008). PMID: |
| | (CL) | | TAGCGTGACCGTGGTCTGGAA | TVTKTVKPSECS* (SEQ ID | | | 18538861 |
| | | | GGCAGACGGCAGCACCATCA | NO: 100) | | | |
| | | | CCCGCAACGTGGAGACCACC | | | | |
| | | | CGGGCCTCCAAACAGAGCAA | | | | |
| | | | CAGCAAGTACGCGGCCAGCA | | | | |
| | | | GCTACCTGAGCCTGACGAGC | | | | |
| | | | AGCGACTGGAAATCGAAAGG | | | | |
| | | | CAGTTACAGCTGCGAGGTCAC | | | | |
| | | | GCACGAGGGGAGCACCGTGA | | | | |
| | | | CGAAGACAGTGAAGCCCTCA | | | | |
| | | | GAGTGTTCTTAG (SEQ ID NO: | | | | |
| | | | 101) | | | | |

In the anti-PD-1 antibody of the present disclosure, the VL and VH may be derived from rat. For example, the VL may be the VL of a rat anti-bovine PD-1 antibody, and the VH may be the VH of the rat anti-bovine PD-1 antibody.

The amino acid sequence of the VL and the amino acid sequence of the VH of the rat anti-bovine PD-1 antibody are shown in SEQ ID NOS: 1 and 2, respectively. The amino acid sequences as shown in SEQ ID NOS: 1 and 2 may have deletion(s), substitution(s) or addition(s) of one or several (e.g., up to five, about 10 at the most) amino acids. Even when such mutations are introduced, the resulting amino acid sequences may be capable of having the function as VL or VH of the PD-1 antibody.

The VL and VH of an antibody of an animal other than rat may be derived from an animal which produces a PD-1 that cross-reacts with rat anti-bovine PD-1 antibody 5D2.

There are two types of immunoglobulin light chain, which are called Kappa chain (κ) and Lambda chain (λ). In the anti-PD-1 antibody of the present disclosure, the light chain constant region (CL) of an antibody of an animal other than rat may have the amino acid sequence of the constant region of either Kappa chain or Lambda chain. However, the relative abundance of Lambda chain is higher in bovine, ovine, feline, canine and equine, and that of Kappa chain is higher in mouse, rat, human and porcine. Since a chain with a higher relative abundance is considered to be preferable, a bovine, ovine, feline, canine or equine antibody preferably has the amino acid sequence of the constant region of Lambda chain whereas a mouse, rat, human or porcine antibody preferably has the amino acid sequence of the constant region of Kappa chain.

The heavy chain constant region (CH) of an antibody of an animal other than rat may have the amino acid sequence of the constant region of an immunoglobulin equivalent to human IgG4. Immunoglobulin heavy chain is classified into γ chain, μ chain, α chain, δ chain and E chain depending on the difference in constant region. According to the type of heavy chain present, five classes (isotypes) of immunoglobulin are formed; they are IgG, IgM, IgA, IgD and IgE.

Immunoglobulin G (IgG) accounts for 70-75% of human immunoglobulins and is the most abundantly found monomeric antibody in plasma. IgG has a four-chain structure consisting of two light chains and two heavy chains. Human IgG1, IgG2 and IgG4 have a molecular weight of about 146,000, whereas human IgG3 has a long hinge region that connects Fab region and Fc region and has a larger molecular weight of 170,000. Human IgG1 accounts for about 65% of human IgG, human IgG2 about 25%, human IgG3 about 7%, and human IgG4 about 3%. They are uniformly distributed inside and outside of blood vessels. Having a strong affinity for Fc receptors and complement factors on effector cell surfaces, human IgG1 induces antibody-dependent cell cytotoxicity (ADCC) and also activates complements to induce complement-dependent cell cytotoxicity (CDC). Human IgG2 and IgG4 are low at ADCC and CDC activities because their affinity for Fc receptors and complement factors is low.

Immunoglobulin M (IgM), which accounts for about 10% of human immunoglobulins, is a pentameric antibody consisting of five basic four-chain structures joined together. It has a molecular weight of 970,000. Usually occurring only in blood, IgM is first produced against infectious microorganisms and takes charge of early stage immunity.

Immunoglobulin A (IgA) accounts for 10-15% of human immunoglobulins. It has a molecular weight of 160,000. Secreted IgA is a dimeric antibody consisting of two IgA molecules joined together. IgA1 is found in serum, nasal discharge, saliva and breast milk. In intestinal juice, IgA2 is found abundantly.

Immunoglobulin D (IgD) is a monomeric antibody accounting for no more than 1% of human immunoglobulins. IgD is found on B cell surfaces and involved in induction of antibody production.

Immunoglobulin E (IgE) is a monomeric antibody that occurs in an extremely small amount, accounting for only 0.001% or less of human immunoglobulins. Immunoglobulin E is considered to be involved in immune response to parasites but in advanced countries where parasites are rare, IgE is largely involved in bronchial asthma and allergy among other things.

In canine, sequences of IgG-A (equivalent to human IgG2), IgG-B (equivalent to human IgG1), IgG-C (equivalent to human IgG3) and IgG-D (equivalent to human IgG4) have been identified as the heavy chain of IgG. In the antibody of the present disclosure, an IgG's heavy chain constant region with neither ADCC activity nor CDC activity is preferable (IgG4 in human). In the case where the constant region of an immunoglobulin equivalent to human IgG4 has not been identified, one may use a constant region that has lost both ADCC activity and CDC activity as a result of introducing mutations into the relevant region of an immunoglobulin equivalent to human IgG4.

In bovine, sequences of IgG1, IgG2 and IgG3 have been identified as the heavy chain of IgG. In the antibody of the present disclosure, an IgG's heavy chain constant region with neither ADCC activity nor CDC activity is preferable (IgG4 in human). Although the constant region of wild-type human IgG1 has ADCC activity and CDC activity, it is known that these activities can be reduced by introducing amino acid substitutions or deletions into specific sites. In bovine, the constant region of an immunoglobulin equivalent to human IgG4 has not been identified, so mutations may be added to the relevant region of an immunoglobulin equivalent to human IgG1 and the resultant constant region then used. As one example, the amino acid sequence of the CH of a bovine antibody (IgG1 chain, GenBank: X62916) having mutations introduced into CH2 domain and a nucleotide sequence for such amino acid sequence (after codon optimization) are shown in SEQ ID NOS: 4 and 8, respectively.

An anti-PD-1 antibody is preferable in which (i) the CL of a bovine antibody has the amino acid sequence of the constant region of Lambda chain and (ii) the CH of the bovine antibody has mutations introduced thereinto that reduce ADCC activity and/or CDC activity.

The anti-PD-1 antibody of the present disclosure encompasses rat-bovine chimeric antibodies, bovinized antibodies and complete bovine-type antibodies. However, the animal is not limited to bovine and may be exemplified by human, canine, porcine, simian, mouse, feline, equine, goat, ovine, water buffalo, rabbit, hamster, guinea pig and the like.

For example, the anti-PD-1 antibody of the present disclosure may be an anti-PD-1 antibody in which the CL of a bovine antibody has the amino acid sequence as shown in SEQ ID NO: 151 and the CH of the bovine antibody has the amino acid sequence as shown in SEQ ID NO: 152.

The amino acid sequences as shown in SEQ ID NOS: 151 and 152 may have deletion(s), substitution(s) or addition(s) of one or several (e.g., up to five, about 10 at the most) amino acids. Even when such mutations are introduced, the resulting amino acid sequences may be capable of having the function as CL or CH of the PD-1 antibody.

The anti-PD-1 antibody of the present disclosure may have a four-chain structure comprising two light chains and two heavy chains.

The anti-PD-1 antibody of the present disclosure may be prepared as described below. Briefly, an artificial gene is synthesized which comprises (i) the identified variable region sequences of a rat anti-bovine PD-1 antibody and (ii) the constant region sequences of an antibody of an animal other than rat (e.g., bovine) (preferably, an immunoglobulin equivalent to human IgG1, in which mutations have been introduced into the relevant region to reduce ADCC activity and/or CDC activity). The resultant gene is inserted into a vector (e.g., plasmid), which is then introduced into a host cell (e.g., mammal cell such as CHO cell). The host cell is cultured, and the antibody of interest is collected from the resultant culture.

The amino acid sequence and the nucleotide sequence of the VL of the rat anti-bovine PD-1 antibody identified by the present inventors are shown in SEQ ID NOS: 149 and 153, respectively. Further, the nucleotide sequence after codon optimization is shown in SEQ ID NO: 159.

The amino acid sequence and the nucleotide sequence of the VH of the rat anti-bovine PD-1 antibody identified by the present inventors are shown in SEQ ID NOS: 150 and 154, respectively. Further, the nucleotide sequence after codon optimization is shown in SEQ ID NO: 160.

The amino acid sequence and the nucleotide sequence of the CL (Lambda chain, GenBank: X62917) of a bovine antibody are shown in SEQ ID NOS: 151 and 155, respectively. Further, the nucleotide sequence after codon optimization is shown in SEQ ID NO: 161.

The amino acid sequence and the nucleotide sequence (after codon optimization) of the CH (IgG1 chain, modified from GenBank: X62916) of a bovine antibody are shown in SEQ ID NOS: 152 and 156, respectively.

Further, SEQ ID NO: 157 shows the amino acid sequence of a chimeric light chain consisting of the VL of the rat anti-bovine PD-1 antibody and the CL (Lambda chain, GenBank: X62917) of the bovine antibody. The nucleotide sequence (after codon optimization) of the chimeric light chain consisting of the VL of the rat anti-PD-1 antibody and the CL (Lambda chain, GenBank: X62917) of the bovine antibody is shown in SEQ ID NO: 162.

SEQ ID NO: 158 shows the amino acid sequence of a chimeric heavy chain consisting of the VH of the rat anti-bovine PD-1 antibody and the CH (IgG1 chain, modified from GenBank: X62916) of the bovine antibody. The nucleotide sequence (after codon optimization) of the chimeric heavy chain consisting of the VH of the rat anti-bovine PD-1 antibody and the CH (IgG1 chain, modified from GenBank: X62916) of the bovine antibody is shown in SEQ ID NO: 163.

Amino acid sequences and nucleotide sequences of CLs and CHs of various animals other than rat may be obtained from known databases for use in the present disclosure.

Amino acid sequences and nucleotide sequences of bovine CL and CH are summarized in the table below.

TABLE

| | | | | | GenBank | | |
|---|---|---|---|---|---|---|---|
| | | | | | Accession | IMGT | |
| Species | Ig Domain | | Nucleotide Sequence | Amino Acid Sequence | No. | Database | Reference |
| Bovine | Bovine | IgG1 | GCCTCCACCACAGCCCCGAAAGTCTACCCTCTGAGTTCTTG | ASTTAPKVYPLSSCCGDKSSSTVTLGCLVSSYM | X62916 | http://www. | Symons D.B. |
| (Scientific | Ig | variant | CTGCGGGGACAAGTCCAGCTCCACCGTGACCCTGGGCTGCC | PEPVTVTWNSGALKSGVHTFPAVLQSSGLYSLS | | imgt.org/ | et al., J. |
| Name: | heavy | 1 | TGGTCTCCAGCTACATGCCCGAGCCGGTGACCGTGACCTGG | SMVTVPGSTSGQTFTCNVAHPASSTKVDKAVDP | | IMGTreper- | Immunogenet., |
| Bos | chain | | AACTCGGGTGCCCTGAAGAGCGGCGTGCACACCTTCCCGG | TCKPSPCDCCPPPELPGGPSVFIFPPKPKDTLT | | toire/ | 14, 273- |
| taurus) | con- | | CTGTCCTTCAGTCCTCCGGGCTGTACTCTCTCAGCAGCATG | ISGTPEVTCVVVDVGHDDPEVKFSWFVDDVEVN | | index.php? | 283 (1987). |
| | stant | | GTGACCGTGCCCGGCAGCACCTCAGGACAGACCTTCACCTG | TATTKPREEQFNSTYRVVSALRIQHQDWTGGKE | | section= | PMID: |
| | region | | CAACGTAGCCCACCCGGCCAGCAGCACCAAGGTGGACAAGG | FKCKVHNEGLPAPIVRTISRTKGPAREPQVYVL | | Locus | 3141517 |
| | (CH1 | | CTGTTGATCCCACATGCAAACCATCACCCTGTGACTGTTGC | APPQEELSKSTVSLTCMVTSFYPDYIAVEWQRN | | Genes& | Symons D.B. |
| | ~CH3) | | CCACCCCCTGAGCTCCCCGGAGGACCCTCTGTCTTCATCTT | GQPESEDKYGTTPPQLDADSSYFLYSKLRVDRN | | repertoire= | et al., Mol. |
| | | | CCCACCGAAACCCAAGGACACCCTCACAATCTCGGGAACGC | SWQEGDTYTCVVMHEALHNHYTQKSTSKSAGK | | genetable& | Immunol., 26, |
| | | | CCGAGGTCACGTGTGTGGTGGTGGACGTGGGCCACGATGAC | * | | species= | 841-850 |
| | | | CCCGAGGTGAAGTTCTCCTGGTTCGTGGACGACGTGGAGGT | (SEQ ID NO: 169) | | bovine& | (1989). PMID: |
| | | | AAACACAGCCACGACGAAGCCGAGAGAGGAGCAGTTCAAC | | | group=IGHC | 2513487 |
| | | | AGCACCTACCGCGTGGTCAGCGCCCTGCGCATCCAGCACCA | | | | Kacskovics I. |
| | | | GGACTGGACTGGAGGAAAGGAGTTCAAGTGCAAGGTCCA | | | | and Butler |
| | | | CAACGAAGGCCTCCCGGCCCCCATCGTGAGGACCATCTCCA | | | | J.E., Mol. |
| | | | GGACCAAAGGGCCGGCCCGGGAGCCGCAGGTGTATGTCCT | | | | Immunol., 33, |
| | | | GGCCCCACCCCAGGAAGAGCTCAGCAAAAGCACGGTCAGC | | | | 189-195 |
| | | | CTCACCTGCATGGTCACCAGCTTCTACCCAGACTACATCGC | | | | (1996). PMID: |
| | | | CGTGGAGTGGCAGAGAAACGGGCAGCCTGAGTCGGAGGAC | | | | 8649440 |
| | | | AAGTACGGCACGACCCCGCCCCAGCTGGACGCCGACAGCT | | | | Rabbani H. |
| | | | CCTACTTCCTGTACAGCAAGCTCAGGGTGGACAGGAACAG | | | | et al., |
| | | | CTGGCAGGAAGGAGACACCTACACGTGTGTGGTGATGCAC | | | | Immuno- |
| | | | GAGGCCCTGCACAATCACTACACGCAGAAGTCCACCTCTAA | | | | genetics, |
| | | | GTCTGCGGGTAAATGA | | | | 46, 326- |
| | | | (SEQ ID NO: 177) | | | | 331 (1997). |
| | | IgG1 | GCCTCCACCACAGCCCCGAAAGTCTACCCTCTGAGTTCTTG | ASTTAPKVYPLSSCCGDKSSSTVTLGCLVSSYM | X16701 | | PMID: |
| | | variant | CTGCGGGGACAAGTCCAGCTCCACCGTGACCCTGGGCTGCC | PEPVTVTWNSGALKSGVHTFPAVLQSSGLYSLS | (M25278) | | 9218535 |
| | | 2 | TGGTCTCCAGCTACATGCCCGAGCCGGTGACCGTGACCTGG | SMVTVPGSTSGQTFTCNVAHPASSTKVDKAVDP | | | Saini S.S. et |
| | | | AACTCGGGTGCCCTGAAGAGCGGCGTGCACACCTTCCCGG | TCKPSPCDCCPPPELPGGPSVFIFPPKPKDTLT | | | al., Scand. |
| | | | CCGTCCTTCAGTCCTCCGGGCTGTACTCTCTCAGCAGCATG | ISGTPEVTCVVVDVGHDDPEVKFSWFVDDVEVN | | | J. |
| | | | GTGACCGTGCCCGGCAGCACCTCAGGACAGACCTTCACCTG | TATTKPREEQFNSTYRVVSALRIQHQDWTGGKE | | | Immunol. 65, |
| | | | CAACGTAGCCCACCCGGCCAGCAGCACCAAGGTGGACAAG | FKCKVHNEGLPAPIVRTISRTKGPAREPQVYVL | | | 32-8 (2007). |
| | | | GCTGTTGATCCCACATGCAAACCATCACCCTGTGACTGTTG | APPQEELSKSTVSLTCMVTSFYPDYIAVEWQRN | | | PMID: |
| | | | CCCACCCCCTGAGCTCCCCGGAGGACCCTCTGTCTTCATCT | QGPESEDKYGTTPPQLDADSSYFLYSKLRVDRN | | | 17212764 |
| | | | TCCCACCGAAACCCAAGGACACCCTCACAATCTCGGGAACG | SWQEGDTYTCVVMHEALHNHYTQKSTSKSAGK | | | |
| | | | CCCGAGGTCACGTGTGTGGTGGTGGACGTGGGCCACGATGA | * | | | |
| | | | CCCCGAGGTGAAGTTCTCCTGGTTCGTGGACGACGTGGAG | (SEQ ID NO: 170) | | | |
| | | | GTAAACACAGCCACGACGAAGCCGAGAGAGGAGCAGTTCA | | | | |
| | | | ACAGCACCTACCGCGTGGTCAGCGCCCTGCGCATCCAGCAC | | | | |
| | | | CAGGACTGGACTGGAGGAAAGGAGTTCAAGTGCAAGGTC | | | | |
| | | | CACAACGAAGGCCTCCCGGCCCCCATCGTGAGGACCATCTC | | | | |
| | | | CAGGACCAAAGGGCCGGCCCGGGAGCCGCAGGTGTATGT | | | | |
| | | | CCTGGCCCCACCCCAGGAAGAGCTCAGCAAAAGCACGGTC | | | | |
| | | | AGCCTCACCTGCATGGTCACCAGCTTCTACCCAGACTACAT | | | | |
| | | | CGCCGTGGAGTGGCAGAGAAACGGGCAGCCTGAGTCGGAG | | | | |
| | | | GACAAGTACGGCACGACCCCGCCCCAGCTGGACGCCGACA | | | | |
| | | | GCTCCTACTTCCTGTACAGCAAGCTCAGGGTGGACAGGAAC | | | | |
| | | | AGCTGGCAGGAAGGAGACACCTACACGTGTGTGGTGATGC | | | | |
| | | | ACGAGGCCCTGCACAATCACTACACGCAGAAGTCCACCTCT | | | | |
| | | | AAGTCTGCGGGTAAATGA | | | | |
| | | | (SEQ ID NO: 178) | | | | |
| | | IgG1 | GCCTCCACCACAGCCCCGAAAGTCTACCCTCTGAGTTCTTG | ASTTAPKVYPLSSCCGDKSSSTVTLGCLVSSYM | S82409 | | |
| | | variant | CTGCGGGGACAAGTCCAGCTCCACCGTGACCCTGGGCTGCC | PEPVTVTWNSGALKSGVHTFPAVLQSSGLYSLS | | | |
| | | 3 | TGGTCTCCAGCTACATGCCCGAGCCGGTGACCGTGACCTGG | SMVTVPGSTSGTQTFTCNVAHPASSTKVDKAVD | | | |
| | | | AACTCGGGTGCCCTGAAGAGCGGCGTGCACACCTTCCCGG | PRCKTTCDCCPPPELPGGPSVFIFPPKPKDTLT | | | |
| | | | CCGTCCTTCAGTCCTCCGGGCTCTACTCTCTCAGCAGCATG | ISGTPEVTCVVVDVGHDDPEVKFSWFVDDVEVN | | | |
| | | | GTGACCGTGCCCGGCAGCACCTCAGGAACCCAGACCTTCAC | TATTKPREEQFNSTYRVVSALRIQHQDWTGGKE | | | |
| | | | CTGCAACGTAGCCCACCCGGCCAGCAGCACCAAGGTGGACA | FKCKVHNEGLPAPIVRTISRTKGPAREPQVYVL | | | |
| | | | AGGCTGTTGATCCCAGATGCAAAACAACCTGTGACTGTTGC | APPQEELSKSTVSLTCMVTSFYPDYIAVEWQRN | | | |
| | | | CCACCGCCTGAGCTCCCTGGAGGACCCTCTGTCTTCATCTT | GQPESEDKYGTTPPQLDADGSYFLYSRLRVDRN | | | |
| | | | CCCACCGAAACCCAAGGACACCCTCACAATCTCGGGAACGC | SWQEGDTYTCVVMHEALHNHYTQKSTSKSAGK | | | |
| | | | CCGAGGTCACGTGTGTGGTGGTGGACGTGGGCCACGATGAC | * | | | |
| | | | CCCGAGGTGAAGTTCTCCTGGTTCGTGGACGACGTGGAGG | (SEQ ID NO: 171) | | | |
| | | | TAAACACAGCCACGACGAAGCCGAGAGAGGAGCAGTTCAA | | | | |
| | | | CAGCACCTACCGCGTGGTCAGCGCCCTGCGCATCCAGCACC | | | | |
| | | | AGGACTGGACTGGAGGAAAGGAGTTCAAGTGCAAGGTCC | | | | |
| | | | ACAACGAAGGCCTCCCAGCCCCCATCGTGAGGACCATCTCC | | | | |
| | | | AGGACCAAAGGGCCGGCCCGGGAGCCGCAGGTGTATGTC | | | | |
| | | | CTGGCCCCACCCCAGGAAGAGCTCAGCAAAAGCACGGTCA | | | | |
| | | | GCCTCACCTGCATGGTCACCAGCTTCTACCCAGACTACATC | | | | |
| | | | GCCGTGGAGTGGCAGAGAAATGGGCAGCCTGAGTCAGAG | | | | |
| | | | GACAAGTACGGCACGACCCCTCCCCAGCTGGACGCCGACG | | | | |
| | | | GCTCCTACTTCCTGTACAGCAGGCTCAGGGTGGACAGGAA | | | | |
| | | | CAGCTGGCAGGAAGGAGACACCTACACGTGTGTGGTGATG | | | | |
| | | | CACGAGGCCCTGCACAATCACTACACGCAGAAGTCCACCTC | | | | |
| | | | TAAGTCTGCGGGTAAATGA | | | | |
| | | | (SEQ ID NO: 179) | | | | |
| | | IgG2 | GCCTCCACCACAGCCCCGAAAGTCTACCCTCTGGCATCCAG | ASTTAPKVYPLASSCGDTSSSTVTLGCLVSSYM | S82407 | | |
| | | variant | CTGCGGAGACACATCCAGCTCCACCGTGACCCTGGGCTGCC | PEPVTVTWNSGALKSGVHTFPAVLQSSGLYSLS | | | |
| | | 1 | TGGTGTCCAGCTACATGCCCGAGCCGGTGACCGTGACCTG | SMVTVPASSSGQTFTCNVAHPASSTKVDKAVGV | | | |
| | | | GAACTCGGGTGCCCTGAAGAGCGGCGTGCACACCTTCCCG | SIDCSKCHNQPCVREPSVFIFPPKPKDTLMITG | | | |
| | | | GCTGTCCTTCAGTCCTCCGGGCTCTACTCTCTCAGCAGCAT | TPEVTCVVVNVGHDNPEVQFSWFVDDVEVHTA | | | |
| | | | GGTGACCGTGCCCGCCAGCAGCTCAGGACAGACCTTCACCT | RSKPREEQFNSTYRVVSALPIQHQDWTGGKEFK | | | |
| | | | GCAACGTAGCCCACCCGGCCAGCAGCACCAAGGTGGACAAG | CKVNNKGLSAPIVRIISRSKGPAREPQVYVLDP | | | |
| | | | GCTGTTGGGGTCTCCATTGACTGCTCCAAGTGTCATAACCA | PKEELSKSTLSVTCMVTGFYPEDVAVEWQRNRQ | | | |
| | | | GCCTTGCGTGAGGGAACCATCTGTCTTCATCTTCCCACCGA | TESEDKYRTTPPQLDTDRSYFLYSKLRVDRNSW | | | |
| | | | AACCCAAAGACACCCTGATGATCACAGGAACGCCCGAGGT | QEGDAYTCVVMHEALHNHYMQKSTSKSAGK* | | | |
| | | | CACGTGTGTGGTGGTGAACGTGGGCCACGATAACCCCGAG | (SEQ ID NO: 24) | | | |
| | | | GTGCAGTTCTCCTGGTTCGTGGATGACGTGGAGGTGCACA | | | | |
| | | | CGGCCAGGTCGAAGCCAAGAGAGGAGCAGTTCAACAGCA | | | | |
| | | | CGTACCGCGTGGTCAGCGCCCTGCCCATCCAGCACCAGGAC | | | | |
| | | | TGGACTGGAGGAAAGGAGTTCAAGTGCAAGGTCAACAACA | | | | |
| | | | AAGGCCTCTCGGCCCCCATCGTGAGGATCATCTCCAGGAGC | | | | |
| | | | AAAGGGCCGGCCCGGGAGCCGCAGGTGTATGTCCTGGACC | | | | |
| | | | CACCCAAGGAAGAGCTCAGCAAAAGCACGCTCAGCGTCAC | | | | |
| | | | CTGCATGGTCACCGGCTTCTACCCAGAAGATGTAGCCGTGG | | | | |
| | | | AGTGGCAGAGAAACCGGCAGACTGAGTCGGAGGACAAGT | | | | |
| | | | ACCGCACGACCCCGCCCCAGCTGGACACCGACCGCTCCTAC | | | | |
| | | | TTCCTGTACAGCAAGCTCAGGGTGGACAGGAACAGCTGGC | | | | |
| | | | AGGAAGGAGACGCCTACACGTGTGTGGTGATGCACGAGG | | | | |
| | | | CCCTGCACAATCACTACATGCAGAAGTCCACCTCTAAGTCT | | | | |
| | | | GCGGGTAAATGA | | | | |
| | | | (SEQ ID NO: 32) | | | | |
| | | IgG2 | GCCTCCACCACAGCCCCGAAAGTCTACCCTCTGAGTTCTTG | ASTTAPKVYPLSSCCGDKSSSTVTLGCLVSSYM | M36946 | | |
| | | variant | CTGCGGGGACAAGTCCAGCTCCACCGTGACCCTGGGCTGCC | PEPVTVTWNSGALKSGVHTFPAVLQSSGLYSLS | (X06703) | | |
| | | 2 | TGGTGTCCAGCTACATGCCCGAGCCGGTGACCGTGACCTG | SMVTVPGSTSGQTFTCNVAHPASSTKVDKAVGV | | | |
| | | | GAACTCGGGTGCCCTGAAGAGCGGCGTGCACACCTTCCCG | SSDCSKPNNQHCVREPSVFIFPPKPKDTLMITG | | | |
| | | | GCCGTCCTTCAGTCCTCCGGGCTCTACTCTCTCAGCAGCAT | TPEVTCVVVNVGHDNPEVQFSWFVDDVEVHTA | | | |
| | | | GGTGACCGTGCCCGGCAGCACCTCAGGACAGACCTTCACCT | RTKPREEQFNSTYRVVSALPIQHQDWTGGKEFK | | | |
| | | | GCAACGTAGCCCACCCGGCCAGCAGCACCAAGGTGGACAAG | CKVNIKGLSASIVRIISRSKGPAREPQVYVLDP | | | |
| | | | GCTGTTGGGGTCTCCAGTGACTGCTCCAAGCCTAATAACCA | PKEELSKSTVSVTCMVIGFYPEDVDVEWQRDRQ | | | |
| | | | GCATTGCGTGAGGGAACCATCTGTCTTCATCTTCCCACCGA | TESEDKYRTTPPQLDADRSYFLYSKLRVDRNSW | | | |
| | | | AACCCAAAGACACCCTGATGATCACAGGAACGCCCGAGGT | QRGDTYTCVVMHEALHNHYMQKSTSKSAGK* | | | |
| | | | CACGTGTGTGGTGGTGAACGTGGGCCACGATAACCCCGAG | (SEQ ID NO: 173) | | | |
| | | | GTGCAGTTCTCCTGGTTCGTGGACGACGTGGAGGTGCACA | | | | |
| | | | CGGCCAGGACGAAGCCGAGAGAGGAGCAGTTCAACAGCA | | | | |
| | | | CGTACCGCGTGGTCAGCGCCCTGCCCATCCAGCACCAGGAC | | | | |
| | | | TGGACTGGAGGAAAGGAGTTCAAGTGCAAGGTCAACATCA | | | | |
| | | | AAGGCCTCTCGGCCTCCATCGTGAGGATCATCTCCAGGAGC | | | | |
| | | | AAAGGGCCGGCCCGGGAGCCGCAGGTGTATGTCCTGGACC | | | | |
| | | | CACCCAAGGAAGAGCTCAGCAAAAGCACGGTCAGCGTCAC | | | | |
| | | | CTGCATGGTCATCGGCTTCTACCCAGAAGATGTAGACGTGG | | | | |
| | | | AGTGGCAGAGAGACCGGCAGACTGAGTCGGAGGACAAGT | | | | |
| | | | ACCGCACGACCCCGCCCCAGCTGGACGCCGACCGCTCCTAC | | | | |
| | | | TTCCTGTACAGCAAGCTCAGGGTGGACAGGAACAGCTGGC | | | | |
| | | | AGAGAGGAGACACCTACACGTGTGTGGTGATGCACGAGGC | | | | |
| | | | CCTGCACAATCACTACATGCAGAAGTCCACCTCTAAGTCTG | | | | |
| | | | CGGGTAAATGA | | | | |
| | | | (SEQ ID NO: 181) | | | | |
| | | IgG2 | GCCTCCACCACAGCCCCGAAAGTCTACCCTCTGAGTTCTTG | ASTTAPKVYPLSSCCGDKSSSGVTLGCLVSSYM | X16702 | | |
| | | variant | CTGCGGGGACAAGTCCAGCTCGGGGGTGACCCTGGGCTGCC | PEPVTVTWNSGALKSGVHTFPAVLQSSGLYSLS | (M25279) | | |
| | | 3 | TGGTCTCCAGCTACATGCCCGAGCCGGTGACCGTGACCTGG | SMVTVPASSSGTQTFTCNVAHPASSTKVDKAVG | | | |
| | | | AACTCGGGTGCCCTGAAGAGCGGCGTGCACACCTTCCCGG | VSSDCSKPNNQHCVREPSVFIFPPKPKDTLMIT | | | |
| | | | CCGTCCTTCAGTCCTCCGGGCTCTACTCTCTCAGCAGCATG | GTPEVTCVVVNVGHDNPEVQFSWFVDDVEVHT | | | |
| | | | GTGACCGTGCCCGCCAGCAGCTCAGGAACCCAGACCTTCAC | ARTKPREEQFNSTYRVVSALPIQHQDWTGGKEF | | | |
| | | | CTGCAACGTAGCCCACCCGGCCAGCAGCACCAAGGTGGACA | KCKVNIKGLSASIVRIISRSKGPAREPQVYVLD | | | |
| | | | AGGCTGTTGGGGTCTCCAGTGACTGCTCCAAGCCTAATAAC | PPKEELSKSTVSLTCMVIGFYPEDVDVEWQRDR | | | |
| | | | CAGCATTGCGTGAGGGAACCATCTGTCTTCATCTTCCCACC | QTESEDKYRTTPPQLDADRSYFLYSKLRVDRNS | | | |
| | | | GAAACCCAAAGACACCCTGATGATCACAGGAACGCCCGAG | WQRGDTYTCVVMHEALHNHYMQKSTSKSAGK* | | | |
| | | | GTCACGTGTGTGGTGGTGAACGTGGGCCACGATAACCCCG | (SEQ ID NO: 26) | | | |
| | | | AGGTGCAGTTCTCCTGGTTCGTGGACGACGTGGAGGTGCA | | | | |
| | | | CACGGCCAGGACGAAGCCGAGAGAGGAGCAGTTCAACAG | | | | |
| | | | CACGTACCGCGTGGTCAGCGCCCTGCCCATCCAGCACCAGG | | | | |
| | | | ACTGGACTGGAGGAAAGGAGTTCAAGTGCAAGGTCAACAT | | | | |
| | | | CAAAGGCCTCTCGGCCTCCATCGTGAGGATCATCTCCAGGA | | | | |
| | | | GCAAAGGGCCGGCCCGGGAGCCGCAGGTGTATGTCCTGG | | | | |
| | | | ACCCACCCAAGGAAGAGCTCAGCAAAAGCACGGTCAGCCT | | | | |
| | | | CACCTGCATGGTCATCGGCTTCTACCCAGAAGATGTAGACG | | | | |
| | | | TGGAGTGGCAGAGAGACCGGCAGACTGAGTCGGAGGACA | | | | |
| | | | AGTACCGCACGACCCCGCCCCAGCTGGACGCCGACCGCTCC | | | | |
| | | | TACTTCCTGTACAGCAAGCTCAGGGTGGACAGGAACAGCT | | | | |
| | | | GGCAGAGAGGAGACACCTACACGTGTGTGGTGATGCACGA | | | | |
| | | | GGCCCTGCACAATCACTACATGCAGAAGTCCACCTCTAAGT | | | | |
| | | | CTGCGGGTAAATGA | | | | |
| | | | (SEQ ID NO: 34) | | | | |
| | | IgG3 | GCCTCCACCACAGCCCCGAAAGTCTACCCTCTGGCATCCAG | ASTTAPKVYPLASSCGDTSSSTVTLGCLVSSYM | U63638 | | |
| | | variant | CTGCGGAGACACATCCAGCTCCACCGTGACCCTGGGCTGCC | PEPVTVTWNSGALKSGVHTFPAVRQSSGLYSLS | | | |
| | | 1 | TGGTCTCCAGCTACATGCCCGAGCCGGTGACCGTGACCTGG | SMVTVPASSSETQTFTCNVAHPASSTKVDKAVT | | | |
| | | | AACTCGGGTGCCCTGAAGAGCGGCGTGCACACCTTCCCGG | ARRPVPTTPKTTIPPGKPTTPKSEVEKTPCQCS | | | |
| | | | CCGTCCGGCAGTCCTCTGGGCTGTACTCTCTCAGCAGCATG | KCPEPLGGLSVFIFPPKPKDTLTISGTPEVTCV | | | |
| | | | GTGACTGTGCCCGCCAGCAGCTCAGAAACCCAGACCTTCAC | VVDVGQDDPEVQFSWFVDDVEVHTARTKPREEQ | | | |
| | | | CTGCAACGTAGCCCACCCGGCCAGCAGCACCAAGGTGGAC | FNSTYRVVSALRIQHQDWLQGKEFKCKVNNKGL | | | |
| | | | AAGGCTGTCACTGCAAGGCGTCCAGTCCCGACGACGCCAA | PAPIVRTISRTKGQAREPQVYVLAPPREELSKS | | | |
| | | | AGACAACTATCCCTCCTGGAAAACCCACAACCCCAAAGTCT | TLSLTCLITGFYPEEIDVEWQRNGQPESEDKYH | | | |
| | | | GAAGTTGAAAAGACACCCTGCCAGTGTTCCAAATGCCCAG | TTAPQLDADGSYFLYSKLRVNKSSWQEGDHYTC | | | |
| | | | AACCTCTGGGAGGACTGTCTGTCTTCATCTTCCCACCGAAA | AVMHEALRNHYKEKSISRSPGK* | | | |
| | | | CCCAAGGACACCCTCACAATCTCGGGAACGCCCGAGGTCA | (SEQ ID NO: 175) | | | |
| | | | CGTGTGTGGTGGTGGACGTGGGCCAGGATGACCCCGAGGT | | | | |
| | | | GCAGTTCTCCTGGTTCGTGGACGACGTGGAGGTGCACACG | | | | |
| | | | GCCAGGACGAAGCCGAGAGAGGAGCAGTTCAACAGCACC | | | | |
| | | | TACCGCGTGGTCAGCGCCCTGCGCATCCAGCACCAGGACT | | | | |
| | | | GGCTGCAGGGAAAGGAGTTCAAGTGCAAGGTCAACAACAA | | | | |
| | | | AGGCCTCCCGGCCCCCATTGTGAGGACCATCTCCAGGACCA | | | | |
| | | | AAGGGCAGGCCCGGGAGCCGCAGGTGTATGTCCTGGCCCC | | | | |
| | | | ACCCCGGGAAGAGCTCAGCAAAAGCACGCTCAGCCTCACC | | | | |
| | | | TGCCTGATCACCGGTTTCTACCCAGAAGAGATAGACGTGGA | | | | |
| | | | GTGGCAGAGAAATGGGCAGCCTGAGTCGGAGGACAAGTA | | | | |
| | | | CCACACGACCGCACCCCAGCTGGATGCTGACGGCTCCTACT | | | | |
| | | | TCCTGTACAGCAAGCTCAGGGTGAACAAGAGCAGCTGGCA | | | | |
| | | | GGAAGGAGACCACTACACGTGTGCAGTGATGCACGAAGCT | | | | |
| | | | TTACGGAATCACTACAAAGAGAAGTCCATCTCGAGGTCTCC | | | | |
| | | | GGGTAAATGA | | | | |
| | | | (SEQ ID NO: 183) | | | | |
| | | IgG3 | GCCTCCACCACAGCCCCGAAAGTCTACCCTCTGGCATCCCG | ASTTAPKVYPLASRCGDTSSSTVTLGCLVSSYM | U63639 | | |
| | | variant | CTGCGGAGACACATCCAGCTCCACCGTGACCCTGGGCTGCC | PEPVTVTWNSGALKSGVHTFPAVLQSSGLYSLS | | | |
| | | 2 | TGGTCTCCAGCTACATGCCCGAGCCGGTGACCGTGACCTGG | SMVTVPASTSETQTFTCNVAHPASSTKVDKAVT | | | |
| | | | AACTCGGGTGCCCTGAAGAGTGGCGTGCACACCTTCCCGG | ARRPVPTTPKTTIPPGKPTTQESEVEKTPCQCS | | | |
| | | | CCGTCCTTCAGTCCTCCGGGCTGTACTCTCTCAGCAGCATG | KCPEPLGGLSVFIFPPKPKDTLTISGTPEVTCV | | | |
| | | | GTGACCGTGCCCGCCAGCACCTCAGAAACCCAGACCTTCAC | VVDVGQDDPEVQFSWFVDDVEVHTARTKPREEQ | | | |
| | | | CTGCAACGTAGCCCACCCGGCCAGCAGCACCAAGGTGGAC | FNSTYRVVSALRIQHQDWLQGKEFKCKVNNKGL | | | |
| | | | AAGGCTGTCACTGCAAGGCGTCCAGTCCCGACGACGCCAA | PAPIVRTISRTKGQAREPQVYVLAPPREELSKS | | | |
| | | | AGACAACCATCCCTCCTGGAAAACCCACAACCCAGGAGTCT | TLSLTCLITGFYPEEIDVEWQRNGQPESEDKYH | | | |
| | | | GAAGTTGAAAAGACACCCTGCCAGTGTTCCAAATGCCCAG | TTAPQLDADGSYFLYSRLRVNKSSWQEGDHYTC | | | |
| | | | AACCTCTGGGAGGACTGTCTGTCTTCATCTTCCCACCGAAA | AVMHEALRNHYKEKSISRSPGK* | | | |
| | | | CCCAAGGACACCCTCACAATCTCGGGAACGCCCGAGGTCA | (SEQ ID NO: 176) | | | |
| | | | CGTGTGTGGTGGTGGACGTGGGCCAGGATGACCCCGAGGT | | | | |
| | | | GCAGTTCTCCTGGTTCGTGGACGACGTGGAGGTGCACACG | | | | |
| | | | GCCAGGACGAAGCCGAGAGAGGAGCAGTTCAACAGCACC | | | | |
| | | | TACCGCGTGGTCAGCGCCCTGCGCATCCAGCACCAGGACT | | | | |
| | | | GGCTGCAGGGAAAGGAGTTCAAGTGCAAGGTCAACAACAA | | | | |
| | | | AGGCCTCCCGGCCCCCATTGTGAGGACCATCTCCAGGACCA | | | | |
| | | | AAGGGCAGGCCCGGGAGCCGCAGGTGTATGTCCTGGCCCC | | | | |
| | | | ACCCCGGGAAGAGCTCAGCAAAAGCACGCTCAGCCTCACC | | | | |
| | | | TGCCTGATCACCGGTTTCTACCCAGAAGAGATAGACGTGGA | | | | |
| | | | GTGGCAGAGAAATGGGCAGCCTGAGTCGGAGGACAAGTA | | | | |
| | | | CCACACGACCGCACCCCAGCTGGATGCTGACGGCTCCTACT | | | | |
| | | | TCCTGTACAGCAGGCTCAGGGTGAACAAGAGCAGCTGGCA | | | | |
| | | | GGAAGGAGACCACTACACGTGTGCAGTGATGCATGAAGCT | | | | |
| | | | TTACGGAATCACTACAAAGAGAAGTCCATCTCGAGGTCTCC | | | | |
| | | | GGGTAAATGA | | | | |
| | | | (SEQ ID NO: 184) | | | | |
| | Bovine | Ig | CAGCCCAAGTCCCCACCCTCGGTCACCCTGTTCCCGCCCTC | QPKSPPSVTLFPPSTEELNGNKATLVCLISDFY | X62917 | Not | Chen L. et |
| | Ig | lambda | CACGGAGGAGCTCAACGGCAACAAGGCCACCCTGGTGTGTC | PGSVTVVWKADGSTITRNVETTRASKQSNSKYA | | registered | al., Vet. |
| | light | | TCATCAGCGACTTCTACCCGGGTAGCGTGACCGTGGTCTGG | ASSYLSLTSSDWKSKGSYSCEVTHEGSTVTKTV | | | Immunol. |
| | chain | | AAGGCAGACGGCAGCACCATCACCCGCAACGTGGAGACCA | KPSECS* | | | Immuno- |
| | con- | | CCCGGGCCTCCAAACAGAGCAACAGCAAGTACGCGGCCAG | (SEQ ID NO: 151) | | | pathol., |
| | stant | | CAGCTACCTGAGCCTGACGAGCAGCGACTGGAAATCGAAA | | | | 124, 284-294 |
| | region | | GGCAGTTACAGCTGCGAGGTCACGCACGAGGGGAGCACC | | | | (2008). PMID: |
| | (CL) | | GTGACGAAGACAGTGAAGCCCTCAGAGTGTTCTTAG | | | | 18538861 |
| | | | (SEQ ID NO: 155) | | | | +A2:H11 |

Amino acid sequences and nucleotide sequences of ovine, water buffalo and human CL and CH are summarized in the table below.

TABLE

| | | | | | GenBank | | |
|---|---|---|---|---|---|---|---|
| | | | | | Accession | IMGT | |
| Species | Ig Domain | | Nucleotide Sequence | Amino Acid Sequence | No. | Database | Reference |
| Human | Human | IgG4 | GAGTCCAAATATGGTCCCCCATGCCCATCATGC | ESKYGPPCPSCPAPEFLGGPSVFLFPPKPKD | K01316 | http://www. | Ellison J. |
| (Scientific | Ig | variant | CCAGCACCTGAGTTCCTGGGGGGACCATCAGT | TLMISRTPEVTCVVVDVSQEDPEVQFNWY | | imgt.org/ | et al., |
| Name: | heavy | 1 | CTTCCTGTTCCCCCCAAAACCCAAGGACACTCT | VDGVEVHNAKTKPREEQFNSTYRVVSVLT | | IMGTrepertoire/ | DNA, 1, |
| Homo | chain | | CATGATCTCCCGGACCCCTGAGGTCACGTGCG | VLHQDWLNGKEYKCKVSNKGLPSSIEKTIS | | index.php? | 11-18 |
| sapiens) | con- | | TGGTGGTGGACGTGAGCCAGGAAGACCCCGA | KAKGQPREPQVYTLPPSQEEMTKNQVSLT | | section= | (1981). PMID: |
| | stant | | GGTCCAGTTCAACTGGTACGTGGATGGCGTGG | CLVKGFYPSDIAVEWESNGQPENNYKTTP | | LocusGenes& | 6299662 |
| | region | | AGGTGCATAATGCCAAGACAAAGCCGCGGGA | PVLDSDGSFFLYSRLTVDKSRWQEGNVFS | | repertoire= | Brusco A. et |
| | (CH1 | | GGAGCAGTTCAACAGCACGTACCGTGTGGTCA | CSVMHEALHNHYTQKSLSLSLGK* | | genetable& | al., Eur. J. |
| | ~CH3) | | GCGTCCTCACCGTCCTGCACCAGGACTGGCTG | (SEQ ID NO: 201) | | species= | Immunogenet., |
| | | | AACGGCAAGGAGTACAAGTGCAAGGTCTCCA | | | human&group= | 25, 349-355 |
| | | | ACAAAGGCCTCCCGTCCTCCATCGAGAAAACC | | | IGHC | (1998). PMID: |
| | | | ATCTCCAAAGCCAAAGGGCAGCCCCGAGAGCC | | | | 9805657 |
| | | | ACAGGTGTACACCCTGCCCCCATCCCAGGAGG | | | | |
| | | | AGATGACCAAGAACCAGGTCAGCCTGACCTGC | | | | |
| | | | CTGGTCAAAGGCTTCTACCCCAGCGACATCGC | | | | |
| | | | CGTGGAGTGGGAGAGCAATGGGCAGCCGGA | | | | |
| | | | GAACAACTACAAGACCACGCCTCCCGTGCTGG | | | | |
| | | | ACTCCGACGGCTCCTTCTTCCTCTACAGCAGGC | | | | |
| | | | TAACCGTGGACAAGAGCAGGTGGCAGGAGGG | | | | |
| | | | GAATGTCTTCTCATGCTCCGTGATGCATGAGG | | | | |
| | | | CTCTGCACAACCACTACACACAGAAGAGCCTCT | | | | |
| | | | CCCTGTCTCTGGGTAAATGA | | | | |
| | | | (SEQ ID NO: 202) | | | | |
| | | IgG4 | GAGTCCAAATATGGTCCCCCGTGCCCATCATG | ESKYGPPCPSCPAPEFLGGPSVFLFPPKPKD | AJ001563 | | |
| | | variant | CCCAGCACCTGAGTTCCTGGGGGGACCATCAG | TLMISRTPEVTCVVVDVSQEDPEVQFNWY | | | |
| | | 2 | TCTTCCTGTTCCCCCCAAAACCCAAGGACACTC | VDGVEVHNAKTKPREEQFNSTYRVVSVLT | | | |
| | | | TCATGATCTCCCGGACCCCTGAGGTCACGTGC | VVHQDWLNGKEYKCKVSNKGLPSSIEKTIS | | | |
| | | | GTGGTGGTGGACGTGAGCCAGGAAGACCCCG | KAKGQPREPQVYTLPPSQEEMTKNQVSLT | | | |
| | | | AGGTCCAGTTCAACTGGTACGTGGATGGCGTG | CLVKGFYPSDIAVEWESNGQPENNYKTTP | | | |
| | | | GAGGTGCATAATGCCAAGACAAAGCCGCGGG | PVLDSDGSFFLYSRLTVDKSRWQEGNVFS | | | |
| | | | AGGAGCAGTTCAACAGCACGTACCGTGTGGTC | CSVMHEALHNHYTQKSLSLSLGK* | | | |
| | | | AGCGTCCTCACCGTCGTGCACCAGGACTGGCT | (SEQ ID NO: 203) | | | |
| | | | GAACGGCAAGGAGTACAAGTGCAAGGTCTCC | | | | |
| | | | AACAAAGGCCTCCCGTCCTCCATCGAGAAAAC | | | | |
| | | | CATCTCCAAAGCCAAAGGGCAGCCCCGAGAGC | | | | |
| | | | CACAGGTGTACACCCTGCCCCCATCCCAGGAG | | | | |
| | | | GAGATGACCAAGAACCAGGTCAGCCTGACCTG | | | | |
| | | | CCTGGTCAAAGGCTTCTACCCCAGCGACATCG | | | | |
| | | | CCGTGGAGTGGGAGAGCAATGGGCAGCCGGA | | | | |
| | | | GAACAACTACAAGACCACGCCTCCCGTGCTGG | | | | |
| | | | ACTCCGACGGCTCCTTCTTCCTCTACAGCAGGC | | | | |
| | | | TAACCGTGGACAAGAGCAGGTGGCAGGAGGG | | | | |
| | | | GAATGTCTTCTCATGCTCCGTGATGCATGAGG | | | | |
| | | | CTCTGCACAACCACTACACGCAGAAGAGCCTC | | | | |
| | | | TCCCTGTCTCTGGGTAAATGA | | | | |
| | | | (SEQ ID NO: 204) | | | | |
| | | IgG4 | GCACCTGAGTTCCTGGGGGGACCATCAGTCTT | APEFLGGPSVFLFPPKPKDTLMISRTPEVTC | AJ001564 | | |
| | | variant | CCTGTTCCCCCCAAAACCCAAGGACACTCTCAT | VVVDVSQEDPEVQFNWYVDGVEVHNAK | | | |
| | | 3 | GATCTCCCGGACCCCTGAGGTCACGTGCGTGG | TKPREEQFNSTYRVVSVLTVLHQDWLNGK | | | |
| | | | TGGTGGACGTGAGCCAGGAAGACCCCGAGGT | EYKCKVSNKGLPSSIEKTISKAKGQPREPQV | | | |
| | | | CCAGTTCAACTGGTACGTGGATGGCGTGGAG | YTLPPSQEEMTKNQVSLTCLVKGFYPSDIA | | | |
| | | | GTGCATAATGCCAAGACAAAGCCGCGGGAGG | VEWESNGQPENNYKTTPPVLDSDGSFFLY | | | |
| | | | AGCAGTTCAACAGCACGTACCGTGTGGTCAGC | SKLTVDKSRWQEGNVFSCSVMHEALHNH | | | |
| | | | GTCCTCACCGTCCTGCACCAGGACTGGCTGAA | YTQKSLSLSLGK* | | | |
| | | | CGGCAAGGAGTACAAGTGCAAGGTCTCCAACA | (SEQ ID NO: 205) | | | |
| | | | AAGGCCTCCCGTCCTCCATCGAGAAAACCATCT | | | | |
| | | | CCAAAGCCAAAGGGCAGCCCCGAGAGCCACA | | | | |
| | | | GGTGTACACCCTGCCCCCATCCCAGGAGGAGA | | | | |
| | | | TGACCAAGAACCAGGTCAGCCTGACCTGCCTG | | | | |
| | | | GTCAAAGGCTTCTACCCCAGCGACATCGCCGT | | | | |
| | | | GGAGTGGGAGAGCAATGGGCAGCCGGAGAA | | | | |
| | | | CAACTACAAGACCACGCCTCCCGTGCTGGACT | | | | |
| | | | CCGACGGCTCCTTCTTCCTCTACAGCAAGCTCA | | | | |
| | | | CCGTGGACAAGAGCAGGTGGCAGGAGGGGA | | | | |
| | | | ACGTCTTCTCATGCTCCGTGATGCATGAGGCTC | | | | |
| | | | TGCACAACCACTACACGCAGAAGAGCCTCTCC | | | | |
| | | | CTGTCTCTGGGTAAATGA | | | | |
| | | | (SEQ ID NO: 206) | | | | |
| | Human | Ig | ACTGTGGCTGCACCATCTGTCTTCATCTTCCCG | TVAAPSVFIFPPSDEQLKSGTASVVCLLNNF | X96754 | http://www. | None |
| | Ig | kappa | CCATCTGATGAGCAGTTGAAATCTGGAACTGC | YPREAKVQWKVDNALQSGNSQESVTEQD | | imgt.org/ | |
| | light | (CK) | CTCTGTTGTGTGCCTGCTGAATAACTTCTATCC | SKDSTYSLSSTLTLSKADYEKHKVYACEVTH | | IMGTrepartoire/ | |
| | chain | | CAGAGAGGCCAAAGTACAGTGGAAGGTGGAT | QGLSSPVTKSFNRGEC* | | index.php? | |
| | con- | | AACGCCCTCCAATCGGGTAACTCCCAGGAGAG | (SEQ ID NO: 207) | | section= | |
| | stant | | TGTCACAGAGCAGGACAGCAAGGACAGCACC | | | LocusGenes& | |
| | region | | TACAGCCTCAGCAGCACCCTGACGCTGAGCAA | | | repertoire= | |
| | | | AGCAGACTACGAGAAACACAAAGTCTACGCCT | | | genetable& | |
| | | | GCGAAGTCACCCATCAGGGCCTGAGCTCGCCC | | | species= | |
| | | | GTCACAAAGAGCTTCAACAGGGGAGAGTGTTA | | | human&group= | |
| | | | G | | | IGKC | |
| | | | (SEQ ID NO: 208) | | | | |

The amino acid sequences as shown in SEQ ID NOS: 4, 3, 42, 44, 46, 48, 50, 52, 54, 56, 58, 60, 62, 64, 66, 68, 70, 72, 74, 76, 78, 12, 80, 82, 84-91, 100, 102, 11, 151, 169-76, 185, 187, 189, 191, 193, 196, 197, 199, 201, 203, 205 and 207 may have deletion(s), substitution(s) or addition(s) of one or several (e.g., up to five, about 10 at the most) amino acids. Even when such mutations are introduced, the resulting amino acid sequences may be capable of having the function as a constant region of Ig heavy chain or light chain.

Although the constant region of wild-type human IgG1 has ADCC activity and CDC activity, it is known that these activities can be reduced by introducing amino acid substitutions and deletions into specific sites. In the case of animals other than human where the constant region of an immunoglobulin equivalent to human IgG4 has not been identified, mutations may be introduced into the relevant region of an immunoglobulin equivalent to human IgG1 so that the resultant constant region with reduced ADCC activity and CDC activity can be used.

Certain embodiments of the present disclosure provides an artificial genetic DNA comprising (a′) a DNA encoding a light chain comprising a light chain variable region containing CDR1 having the amino acid sequence of QSLEYSDGYTY (SEQ ID NO: 164), CDR2 having the amino acid sequence of GVS and CDR3 having the amino acid sequence of FQATHDPDT (SEQ ID NO: 165) and the light chain constant region of an antibody of an animal other than rat and (b′) a DNA encoding a heavy chain comprising a heavy chain variable region containing CDR1 having the amino acid sequence of GFSLTSYY (SEQ ID NO: 166), CDR2 having the amino acid sequence of IRSGGST (SEQ ID NO: 167) and CDR3 having the amino acid sequence of ARTSSGYEGGFDY (SEQ ID NO: 168) and the heavy chain constant region of an antibody of an animal other than rat. The present disclosure also provides a DNA encoding a light chain comprising a light chain variable region containing CDR1 having the amino acid sequence of QSLEYSDGYTY (SEQ ID NO: 164), CDR2 having the amino acid sequence of GVS and CDR3 having the amino acid sequence of FQATHDPDT (SEQ ID NO: 165) and the light chain constant region of an antibody of an animal other than rat. Further, the present disclosure also provides a DNA encoding a heavy chain comprising a heavy chain variable region containing CDR1 having the amino acid sequence of GFSLTSYY (SEQ ID NO: 166), CDR2 having the amino acid sequence of IRSGGST (SEQ ID NO: 167) and CDR3 having the amino acid sequence of ARTSSGYEGGFDY (SEQ ID NO: 168) and the heavy chain constant region of an antibody of an animal other than rat.

For (a) a light chain comprising a light chain variable region containing CDR1 having the amino acid sequence of QSLEYSDGYTY (SEQ ID NO: 164), CDR2 having the amino acid sequence of GVS and CDR3 having the amino acid sequence of FQATHDPDT (SEQ ID NO: 165) and the light chain constant region of an antibody of an animal other than rat; and (b) a heavy chain comprising a heavy chain variable region containing CDR1 having the amino acid sequence of GFSLTSYY (SEQ ID NO: 166), CDR2 having the amino acid sequence of IRSGGST (SEQ ID NO: 167) and CDR3 having the amino acid sequence of ARTSSGYEGGFDY (SEQ ID NO: 168) and the heavy chain constant region of an antibody of an animal other than rat, reference should be had to the foregoing description. The DNA of (a′) is a DNA (gene) encoding the light chain of (a); and the DNA of (b′) is a DNA (gene) encoding the heavy chain of (b). An artificial genetic DNA comprising the DNA of (a′) and the DNA of (′b) may be synthesized on commercial synthesizer. Restriction enzyme recognition sites, KOZAK sequences, poly-A addition signal sequences, promoter sequences, intron sequences or the like may be added to the artificial genetic DNA.

Certain embodiments of the present disclosure also provides a vector comprising the above-mentioned artificial genetic DNA.

As the vector, Escherichia coli-derived plasmids (e.g., pBR322, pBR325, pUC12 or pUC13); Bacillus subtilis-derived plasmids (e.g., pUB110, pTP5 or pC194), yeast-derived plasmids (e.g., pSH19 or pSH15); bacteriophages such as λ phage; animal viruses such as retrovirus or vaccinia virus; or insect pathogen viruses such as baculovirus may be used. In the Examples described later, pDN112 (Marzi A, Yoshida R, Miyamoto H, Ishijima M, Suzuki Y, Higuchi M, Matsuyama Y, Igarashi M, Nakayama E, Kuroda M, Saijo M, Feldmann F, Brining D, Feldmann H, TakadaA. PLoS One, 7:e36192, Apr. 27, 2012; Ikebuchi R, Konnai S, Okagawa T, Yokoyama K, Nakajima C, Suzuki Y, Murata S, Ohashi K. Immunology, 142(4):551-561, August 2014) was used.

The vector may also comprise promoters, enhancers, splicing signals, poly-A addition signals, intron sequences, selection markers, SV40 replication origins, and so forth.

The present disclosure also provides a host cell transformed by the above vector. It is possible to prepare the anti-PD-1 antibody of the invention by culturing the host cell and collecting the antibody of interest from the resultant culture. Therefore, the present disclosure also provides a method of preparing an antibody, comprising culturing the above-described host cell and collecting the anti-PD-1 antibody of the invention from the culture. In the method of the present disclosure for preparing an antibody, a vector incorporating an artificial genetic DNA comprising a DNA encoding the light chain and a DNA encoding the heavy chain may be transfected into a host cell. Alternatively, a vector incorporating a DNA encoding the light chain and a vector incorporating a DNA encoding the heavy chain may be co-transfected into a host cell.

Examples of the host cell include, but are not limited to, bacterial cells (such as Escherichia bacteria, Bacillus bacteria or Bacillus subtilis), fungal cells (such as yeast or Aspergillus), insect cells (such as S2 cells or Sf cells), animal cells (such as CHO cells, COS cells, HeLa cells, C127 cells, 3T3 cells, BHK cells or HEK 293 cells) and plant cells. Among these, CHO-DG44 cell (CHO-DG44(dfhr^{-/-})) which is a dihydrofolate reductase deficient cell is preferable.

Introduction of a recombinant vector into a host cell may be performed by the methods disclosed in Molecular Cloning 2nd Edition, J. Sambrook et al., Cold Spring Harbor Lab. Press, 1989 (e.g., the calcium phosphate method, the DEAE-dextran method, transfection, microinjection, lipofection, electroporation, transduction, scrape loading, the shotgun method, etc.) or by infection.

The resultant transformant may be cultured in a medium, followed by collection of the anti-PD-1 antibody of the present disclosure from the culture. When the antibody is secreted into the medium, the medium may be recovered, followed by isolation and purification of the antibody from the medium. When the antibody is produced within the transformed cells, the cells may be lysed, followed by isolation and purification of the antibody from the cell lysate.

Examples of the medium include, but are not limited to, OptiCHO medium, Dynamis medium, CD CHO medium, ActiCHO medium, FortiCHO medium, Ex-Cell CD CHO medium, BalanCD CHO medium, ProCHO 5 medium and Cellvento CHO-100 medium.

The pH of the medium varies depending on the cell to be cultured. Generally, a pH range from 6.8 to 7.6 is used; mostly, a pH range from 7.0 to 7.4 is appropriate.

When the cell to be cultured is CHO cells, culture may be performed by methods known to those skilled in the art. For example, it is usually possible to perform culturing in a gas-phase atmosphere having a CO₂ concentration of 0-40%, preferably 2-10%, at 30-39° C., preferably around 37° C.

The appropriate period of culture is usually from one day to three months, preferably from one day to three weeks.

Isolation and purification of the antibody may be performed by known methods.

Known isolation/purification methods which may be used in the present disclosure include, but are not limited to, methods using difference in solubility (such as salting-out or solvent precipitation); methods using difference in molecular weight (such as dialysis, ultrafiltration, gel filtration or SDS-polyacrylamide gel electrophoresis); methods using difference in electric charge (such as ion exchange chromatography); methods using specific affinity (such as affinity chromatography); methods using difference in hydrophobicity (such as reversed phase high performance liquid chromatography); and methods using difference in isoelectric point (such as isoelectric focusing).

The pharmaceutical composition in certain embodiments of the present disclosure may be used for prevention and/or treatment of cancers and/or infections. Examples of cancer and/or infection include, but are not limited to, neoplastic diseases (e.g., malignant melanoma, lung cancer, gastric cancer, renal cancer, breast cancer, bladder cancer, esophageal cancer, ovarian cancer and the like), leukemia, Johne's disease, anaplasmosis, bacterial mastitis, mycotic mastitis, mycoplasma infections (such as mycoplasma mastitis, mycoplasma pneumonia or the like), tuberculosis, Theileria orientalis infection, cryptosporidiosis, coccidiosis, trypanosomiasis and leishmaniasis.

The pharmaceutical composition in certain embodiments of the present disclosure comprises a COX-2 inhibitor and is administered before, after or simultaneously with the administration of an inhibitor targeting PD-1/PD-L1.

In the pharmaceutical composition in certain embodiments of the present disclosure, an inhibitor targeting PD-1/PD-L1 and a COX-2 inhibitor may be used in combination or may be formulated as a single dosage.

In the pharmaceutical composition in certain embodiments of the present disclosure comprises an anti-PD-1 antibody as an antibody drug for animals or human.

When an inhibitor targeting PD-1/PD-L1 and a COX-2 inhibitor are used in combination, the inhibitor targeting PD-1/PD-L1 and the COX-2 inhibitor may be administered separately.

When an inhibitor targeting PD-1/PD-L1 and a COX-2 inhibitor are formulated as a single dosage, a combination drug containing the inhibitor targeting PD-1/PD-L1 and the COX-2 inhibitor may be prepared.

The pharmaceutical composition of the present disclosure can be administered to human or animal subjects systemically or locally by an oral or parenteral route.

The inhibitor targeting PD-1/PD-L1 and/or the anti-PD-1 antibody of the present disclosure may be dissolved in buffers such as PBS, physiological saline or sterile water, optionally filter- or otherwise sterilized before being administered to animal subjects (including human) by injection. To the solution of inhibitors targeting PD-1/PD-L1 or the solution of the anti-PD-1 antibody, additives such as coloring agents, emulsifiers, suspending agents, surfactants, solubilizers, stabilizers, preservatives, antioxidants, buffers, isotonizing agents, pH adjusters and the like may be added. As routes of administration, intravenous, intramuscular, intraperitoneal, subcutaneous or intradermal administration may be selected. Transnasal or oral administration may also be selected.

The content of the inhibitor targeting PD-1/PD-L1 in a preparation varies with the type of the preparation and is usually 1-100% by weight, preferably 50-100% by weight. Such a preparation may be formulated into a unit dosage form.

The dose and the number of times and frequency of administration of the inhibitor targeting PD-1/PD-L1 (e.g., PD-L1 antibody) may vary with the symptoms, age and body weight of the human or animal subject, the method of administration, dosage form and so on. For example, 0.1-100 mg/kg body weight, preferably 1-10 mg/kg body weight, may usually be administered per adult at least once at a frequency that enables obtainment of the desired effect.

The dose and the number of times and frequency of administration of the anti-PD-1 antibody of the present disclosure may vary depending on the symptoms, age and body weight of the animal subject, the method of administration, the dosage form and so on. For example, 0.1-100 mg/kg body weight, preferably 1-10 mg/kg body weight, per adult animal may usually be administered at least once at such a frequency that enables confirmation of desired effect.

A COX-2 inhibitor may be contained in a preparation comprising an inhibitor targeting PD-1/PD-L1. Alternatively, the COX-2 inhibitor either alone or in admixture with an excipient or carrier may be formulated into tablets, capsules, powders, granules, liquids, syrups, aerosols, suppositories, injections or the like. The excipient or carrier may be of any type that is routinely used in the art and pharmaceutically acceptable, with their type and composition being appropriately changed. As a liquid carrier, for example, water, plant oil or the like may be used. As a solid carrier, saccharides such as lactose, sucrose or glucose, starches such as potato starch or corn starch, cellulose derivatives such as microcrystalline cellulose, and the like may be used. Lubricants such as magnesium stearate, binders such as gelatin or hydroxypropyl cellulose, and disintegrants such as carboxymethyl cellulose, and the like may be added. What is more, antioxidants, coloring agents, flavoring agents, preservatives, and the like may also be added.

The COX-2 inhibitor may be administered via various routes such as oral, transnasal, rectal, transdermal, subcutaneous, intravenous or intramuscular route.

The content of the COX-2 inhibitor in a preparation varies with the type of the preparation and is usually 1-100% by weight, preferably 50-100% by weight. In the case of a liquid, for example, the content of the COX-2 inhibitor in the preparation is preferably 1-100% by weight. In the case of a capsule, tablet, granule or powder, the content of the COX-2 inhibitor in the preparation is usually 10-100% by weight, preferably 50-100% by weight, with the balance being the carrier. The preparation may be formulated into a unit dosage form.

The dose and the number of times and frequency of administration of the COX-2 inhibitor may vary with the symptoms, age and body weight of the animal or human subject, the method of administration, dosage form and so on. For example, in terms of the amount of the active ingredient, 0.05 to 20 mg (or ml)/kg body weight may usually be administered per adult at least once at a frequency that enables confirmation of the desired effect.

The ratio (in mass) of inhibitor targeting PD-1/PD-L1 to COX-2 inhibitor is appropriately from 1:100 to 1000:1, preferably from 1:10 to 100:1.

The present disclosure provides a method of preventing and/or treating cancer and/or infection, comprising administering to a human or animal subject a pharmaceutically effective amount of a COX-2 inhibitor before, after or simultaneously with the administration of an inhibitor targeting PD-1/PD-L1.

Further, the present disclosure provides use of a COX-2 inhibitor for preventing and/or treating cancer and/or infection, wherein the COX-2 inhibitor is administered before, after or simultaneously with the administration of an inhibitor targeting PD-1/PD-L1.

Still further, the present disclosure provides use of a COX-2 inhibitor for use in a method of preventing and/or treating cancer and/or infection, wherein the COX-2 inhibitor is administered before, after or simultaneously with the administration of an inhibitor targeting PD-1/PD-L1.

The immunostimulatory effect of an inhibitor targeting PD-1/PD-L1 can be enhanced by using a COX-2 inhibitor in combination. Therefore, the present disclosure provides a potentiator for the immunostimulatory effect of an inhibitor targeting PD-1/PD-L1, which comprises a COX-2 inhibitor.

The potentiator may be used in combination with an inhibitor targeting PD-1/PD-L1 or formulated together with an inhibitor targeting PD-1/PD-L1 into a combination drug. Combined use of an inhibitor targeting PD-1/PD-L1 and a COX-2 inhibitor, as well as formulating them together as a single dosage are as described above. The potentiator may be used as an experimental reagent in addition to its application as a pharmaceutical.

While the pharmaceutical composition according to certain embodiments of the present disclosure may be used alone, it may be used in combination with surgical operations, radiation therapies, other immunotherapies such as cancer vaccine, or molecular target drugs.

Synergistic effect can be expected from such combinations.

### EXAMPLES

Hereinbelow, the present disclosure will be described in more detail with reference to the following Examples. However, the present disclosure is not limited to these Examples.

### Example 1

### Examination of Combined Effect of Anti-PD-L1 Antibody and COX-2 Inhibitor in Dogs

### 1. Introduction

The interaction between PD-1 and PD-L1 is one of the major molecular mechanisms through which tumors evade immune responses. It has been reported that inhibition of the above interaction by using an antibody which specifically binds to either of those molecules can produce antitumor effects. In the subject Example, toward establishment of a novel control method for canine tumors, the present inventors have confirmed in in vitro tests an immunostimulatory effect induced by a COX-2 inhibitor and enhancement of that effect when the inhibitor is used in combination with anti-PD-L1 antibody.

### 2. Materials and Methods, as Well as Experimental Results

2.1. PGE₂ Production from Canine Tumor Cell Lines

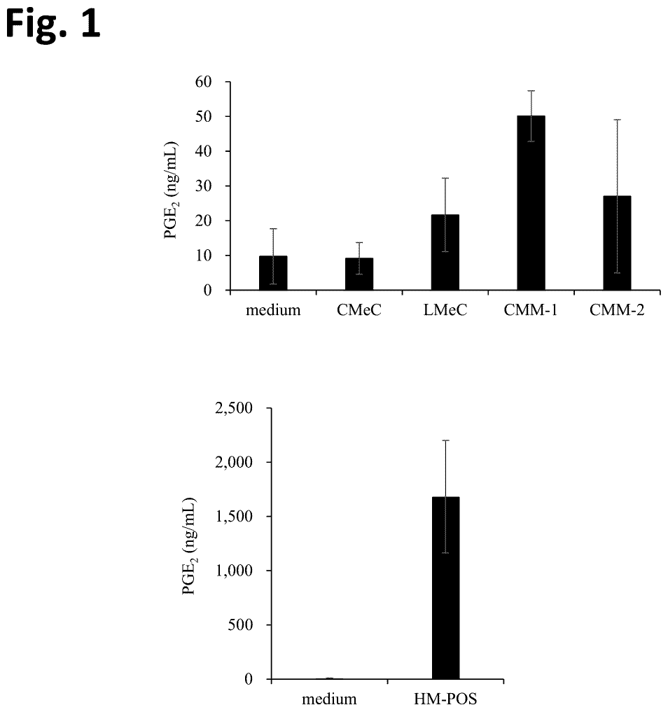

Canine melanoma-derived cell lines of CMeC and LMeC (Ohashi E, Inoue K, Kagechika H, Hong S H, Nakagawa T, et al: Effect of natural and synthetic retinoids on the proliferation and differentiation of three canine melanoma cell lines. J Vet Med Sci 64: 169-172, 2002) as well as CMM-1 and CMM-2 (Ohashi E, Hong S H, Takahashi T, Nakagawa T, Mochizuki M, et al.: Effect of retinoids on growth inhibition of two canine melanoma cell lines. J Vet Med Sci 63: 83-86, 2001) were cultured in RPMI 1640 medium (Sigma) supplemented with 2-mercaptoethanol 2×10⁻⁵ M, 10% inactivated fetal bovine serum (Valley Biomedical), antibiotics (streptomycin 100 μg/ml, penicillin 100 U/ml) (Invitrogen) and 2 mM L-glutamine (Invitrogen) at 37° C. in the presence of 5% CO₂. A canine osteosarcoma-derived cell line HM-POS (Barroga E F, Kadosawa T, Okumura M, Fujinaga T: Establishment and characterization of the growth and pulmonary metastasis of a highly lung metastasizing cell line from canine osteosarcoma in nude mice. J Vet Med Sci 61: 361-367, 1999) was cultured in Dulbecco's Modified Eagle Medium (D-MEM; Invitrogen) supplemented with 10% inactivated fetal bovine serum (Valley Biomedical), antibiotics (streptomycin 100 μg/ml, penicillin 100 U/ml) (Invitrogen) and 2 mM L-glutamine (Invitrogen) at 37° C. in the presence of 5% CO₂. Cells adjusted to a density of 5×10⁵ cells/mL were cultured for 24 hours. The amount of PGE₂ in the culture supernatant was quantified with Prostaglandin E2 Express EIA Kit (Cayman Chemical). As a result, CMM-1 and HM-POS showed a relatively high PGE₂ production (FIG. 1).

### 2.2. COX2 Expression in Canine Tumor Cell Lines

From the canine tumor-derived cell lines cultured as described in section 2.1. of Materials and Methods, RNA was extracted with TRI reagent (Molecular Research Center) and the concentration thereof was measured with NanoDrop8000 (Thermo Scientific). RNA samples were stored at −80° C. until use in experiments.

To 1 μg of the thus obtained RNA, DNase I Reaction buffer and 1 U DNase I Amplification Grade (Invitrogen) were added. Then, deionized distilled water was added to make a 10 μl solution, which was subjected to DNase I treatment at room temperature for 15 mn. Subsequently, 25 nmol ethylenediaminetetraacetic acid (EDTA) was added and the resultant mixture was treated at 65° C. for 10 min. Then, 200 pmol oligo-dT primer was added to the reaction mixture which was treated at 65° C. for 5 min. Thereafter, reverse transcription reaction solution [PrimeScript Buffer (TaKaRa), 7.5 nmol dNTPs, 20 U RNase Plus RNase inhibitor (Promega), 100 U PrimeScript RTase (TaKaRa)] was added to give a final volume of 20 μl. Reverse transcription reaction was carried out at 42° C. for 60 min to thereby synthesize a single-stranded cDNA.

Primers (canine COX2 rt F and canine COX2 rt R; canine HPRT1 rt F and canine HPRT1 rt R) were designed based on the nucleotide sequences of canine COX2 (NM_001003354.1) and canine HPRT1 (AY283372.1) registered at the National Center for Biotechnology Information (NCBI), and real-time PCR was performed. Using 1 μl of the cDNA of each tumor-derived cell line as a template, real-time PCR was performed with LightCycler480 System II (Roche) in a PCR reaction mixture containing 0.3 μl each of primers canine COX2 rt F and canine COX2 rt R or canine HPRT1 rt F and canine HPRT1 rt R (each of which had been adjusted to a concentration of 10 pmol/μl), 5 μl of SYBR Premix DimerEraser (TaKaRa) and 3.4 μl of DDW under the conditions described below.

Primer (canine COX2 rt F):
(SEQ ID NO: 108)
AAGCTTCGATTGACCAGAGCAG
Primer (canine COX2 rt R):
(SEQ ID NO: 109)
TCACCATAAAGGGCCTCCAAC
Primer (canine HPRT1 rt F):
(SEQ ID NO: 110)
TGGCGTCGTGATTAGTGATGA
Primer (canine HPRT1 rt R):
(SEQ ID NO: 111)
CAGAGGGCTACGATGTGATGG

### (PCR Reaction Conditions)

1. Pre incubation 95° C. for 30 sec
2. Quantification 50 cycles each consisting of the following 3 steps:
   I. Thermal denaturation 95° C. for 5 sec
   II. Annealing 58° C. for 30 sec
   III. Extension 72° C. for 30 sec
3. Melting curve
   I. 95° C. for 1 sec
   II. 65° C. for 15 sec
   III. 95° C. continue
4. Cooling 40° C.

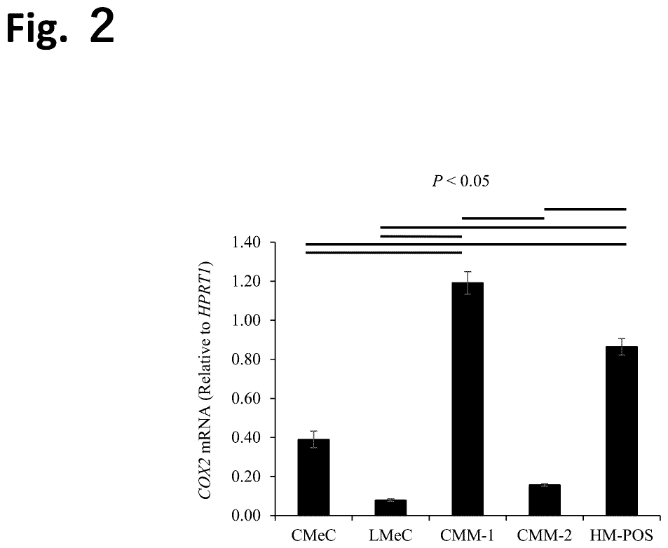

The resultant COX2 mRNA expression level was divided by the expression level of internal control gene HPRT1 mRNA, and the thus obtained value was taken as COX2 expression level. As it turned out, COX2 expression level was high in CMM-1 and HM-POS, consistent with the results of PGE₂ production in culture supernatant (FIG. 2; Tukey's multiple comparison test; P<0.05).

2.3. Effect of PGE₂ on Cytokine Production from Canine Peripheral Blood Mononuclear Cells

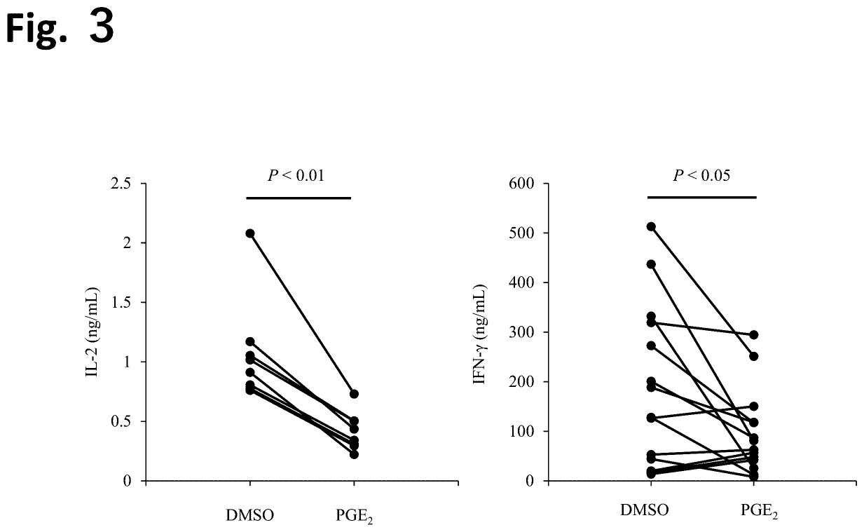

Peripheral blood mononuclear cells (PBMCs) were isolated from heparinized canine peripheral blood samples collected from healthy beagles and mixed breed dogs by density gradient centrifugation using Percoll (GE Healthcare). The resultant PBMCs were cultured in RPMI 1640 medium (Sigma), supplemented with 10% inactivated fetal bovine serum (Valley Biomedical), antibiotics (streptomycin 100 μg/ml, penicillin 100 U/ml) (Invitrogen) and 2 mM L-glutamine (Invitrogen) and further supplemented with 5 μg/ml of Staphylococcal Enterotoxin B (SEB) (Sigma) and 1 μg/ml of Anti-Canine CD28 (eBioscience), at 37° C. in the presence of 5% CO₂ for 3 days. Production of interleukin 2 (IL-2) and interferon γ (IFN-γ) into the culture supernatant upon addition of prostaglandin E2 (Cayman Chemical) at a final concentration of 2.5 μM was measured with Canine IL-2 DuoSet ELISA (R&D systems) and Canine IFN-gamma DuoSet ELISA (R&D systems). PGE₂ significantly decreased IL-2 and IFN-γ productions from canine PBMCs (FIG. 3; Wilcoxon signed-rank test; P<0.01 and P<0.05).

### 2.4. PGE₂ Production Inhibitory Effect of COX-2 Inhibitor

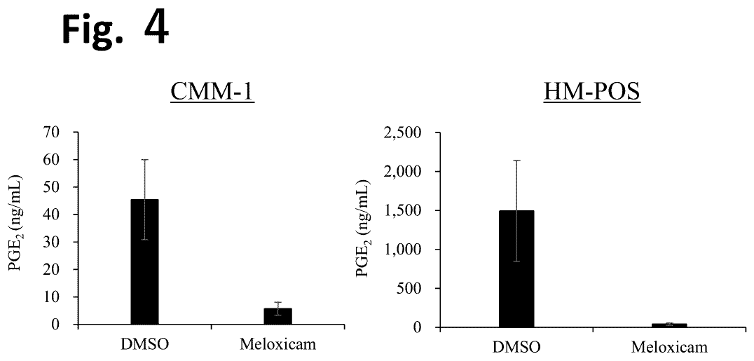

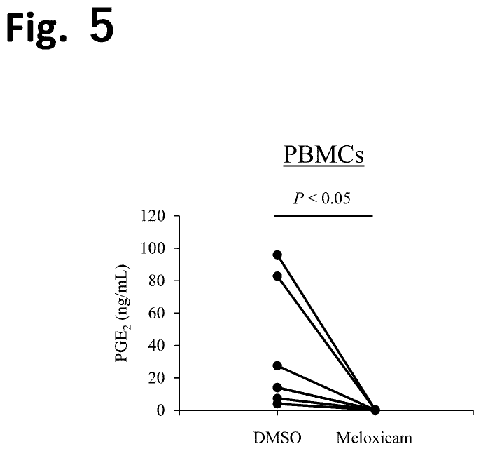

Canine tumor cell lines CMM-1 and HM-POS were cultured as described in section 2.1 of Materials and Methods, and meloxicam (Sigma) was added to give a final concentration of 5 μM. PGE₂ production from each tumor cell line was quantified by ELISA. PGE₂ production showed a tendency to decrease as a result of addition of meloxicam (FIG. 4). Further, canine PBMCs were cultured as described in section 2.3 of Materials and Methods, and meloxicam (Sigma) was added to give a final concentration of 5 μM. As a result, PGE₂ production from PBMCs decreased significantly (FIG. 5; Wilcoxon signed-rank test; P<0.05).

2.5. Effect of COX-2 Inhibitor on Cytokine Production from Canine Peripheral Blood Mononuclear Cells

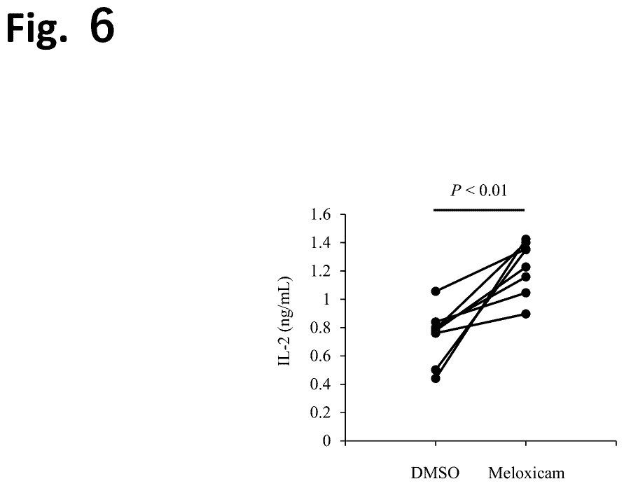

Canine PBMCs were cultured as described in section 2.3 of Materials and Methods, and meloxicam (Sigma) was added to give a final concentration of 5 μM. Then, IL-2 concentration in the culture supernatant was quantified by ELISA. IL-2 production from canine PBMCs was increased significantly as a result of addition of meloxicam (FIG. 6; Wilcoxon signed-rank test; P<0.01).

### 2.6. Enhancement of Canine PBMC Activation Effect by Combined Use of Anti-PD-L1 Antibody and COX-2 Inhibitor

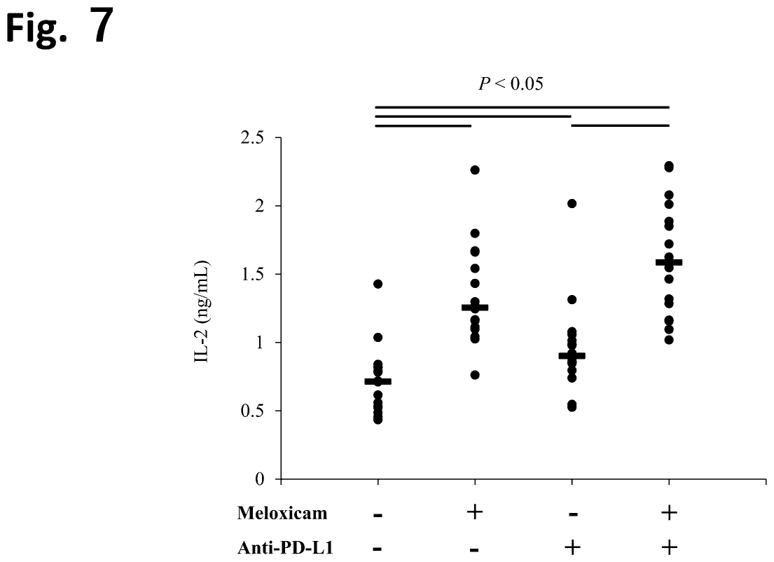

Canine PBMCs were cultured as described in section 2.3 of Materials and Methods. To the resultant PBMCs, rat-canine chimeric anti-PD-L1 antibody c4G12 (Maekawa et al., data in submission; see Reference Example 1 described below) and meloxicam (Sigma) were added to give final concentrations of 20 μg/mL and 5 μM, respectively. Subsequently, IL-2 concentration in the culture supernatant was quantified by ELISA. Although the PD-L1 antibody taken alone increased IL-2 production, combined use of meloxicam further increased IL-2 production (FIG. 7; Steel-Dwass test; P<0.05).

### Reference Example 1

### Rat-Canine Chimeric Anti-PD-L1 Antibody

### 1. Introduction

Programmed cell death 1 (PD-1), an immunoinhibitory receptor, and its ligand programmed cell death ligand 1 (PD-L1) are molecules identified by Prof Tasuku Honjo et al., Kyoto University, as factors which inhibit excessive immune response and are deeply involved in immunotolerance. Recently, it has been elucidated that these molecules are also involved in immunosuppression in tumors. In the subject Example, for the purpose of establishing a novel therapy for canine neoplastic diseases, a chimeric antibody gene was prepared in which a variable region gene of a rat anti-bovine PD-L1 monoclonal antibody (4G12) capable of inhibiting the binding of canine PD-1 to PD-L1 was linked to a constant region gene of a canine immunoglobulin (IgG4). The resultant chimeric antibody gene was introduced into Chinese hamster ovary cells (CHO cells), which were cultured to produce a rat-canine chimeric anti-PD-L1 antibody c4G12. The effect of this chimeric antibody was confirmed in vitro and in vivo.

### 2. Materials and Methods

### 2.1 Bovine PD-L1 Monoclonal Antibody Producing Cells

The nucleotide sequence of bovine PD-L1 was identified (Ikebuchi R, Konnai S, Shirai T, Sunden Y, Murata S, Onuma M, Ohashi K. Vet Res. 2011 Sep. 26; 42:103). Based on the sequence information, a recombinant bovine PD-L1 was prepared. Rat was immunized with this recombinant protein to prepare a rat anti-bovine PD-L1 antibody (Ikebuchi R, Konnai S, Okagawa T, Yokoyama K, Nakajima C, Suzuki Y, Murata S, Ohashi K. Immunology. 2014 August; 142(4):551-61; Clone 4G12 which would later serve as the variable region of the canine chimeric antibody of interest is described in this article.)

### 2.2 Identification of Full-Length Canine PD-1 and PD-L1 Genes

To determine the full lengths of canine PD-1 and PD-L1 cDNAs, PCR primers were first designed based on the putative nucleotide sequences of canine PD-1 and PD-L1 already registered at The National Center for Biotechnology Information (NCBI) (GenBank accession number; XM_543338 and XM_541302). Briefly, primers to amplify the inner sequence of the open reading frame (ORF) of each gene were designed (cPD-1 inner F and R, cPD-L1 inner F and R), and PCR was performed. For the amplified products, nucleotide sequences were determined with a capillary sequencer according to conventional methods. Further, to determine the nucleotide sequences of full-length PD-1 and PD-L1 cDNA, primers (cPD-1 5′ GSP and 3′ GSP; cPD-L1 5′ GSP and 3′GSP) were designed based on the canine PD-1 and PD-L1 cDNA sequences determined above. 5′-RACE and 3′-RACE were then performed using the 5′-RACE system for rapid amplification of cDNA ends and 3′-RACE system for rapid amplification of cDNA ends (Invitrogen), respectively. The resultant gene fragments of interest were sequenced as described (Maekawa N, Konnai S, Ikebuchi R, Okagawa T, Adachi M, Takagi S, Kagawa Y, Nakajima C, Suzuki Y, Murata S, Ohashi K. PLoS One. 2014 Jun. 10; 9(6):e98415).

Primer (cPD-1 inner F):
(SEQ ID NO: 21)
AGGATGGCTCCTAGACTCCC
Primer (cPD-1 inner R):
(SEQ ID NO: 22)
AGACGATGGTGGCATACTCG
Primer (cPD-L1 inner F):
(SEQ ID NO: 23)
ATGAGAATGTTTAGTGTCTT
Primer (cPD-L1 inner R):
(SEQ ID NO: 24)
TTATGTCTCTTCAAATTGTATATC
Primer (cPD-1 5′GSP):
(SEQ ID NO: 25)
GTTGATCTGTGTGTTG
Primer (cPD-1 3′GSP):
(SEQ ID NO: 26)
CGGGACTTCCACATGAGCAT
Primer (cPD-L1 5′GSP):
(SEQ ID NO: 27)
TTTTAGACAGAAAGTGA
Primer (cPD-L1 3′GSP):
(SEQ ID NO: 28)
GACCAGCTCTTCTTGGGGAA

### 2.3 Construction of Canine PD-1 and PD-L1 Expressing COS-7 Cells

For preparing canine PD-1-EGFP and PD-L1-EGFP expression plasmids, PCR was performed using a synthesized beagle PBMC-derived cDNA as a template and primers designed by adding XhoI and BamHI recognition sites (PD-1) and BglII and EcoRI recognition sites (PD-L1) on the 5′ side (cPD-1-EGFP F and R; cPD-L1-EGFP F and R). The resultant PCR products were digested with XhoI (Takara) and BamHI (Takara) (PD-1) and with BglII (New England Biolabs) and EcoRI (Takara) (PD-L1), and then purified with FastGene Gel/PCR Extraction Kit (NIPPON Genetics), followed by cloning into pEGFP-N2 vector (Clontech) treated with restriction enzymes in the same manner. The resultant expression plasmids of interest were extracted with QIAGEN Plasmid Midi kit (Qiagen) and stored at −30° C. until use in experiments. Hereinafter, the thus prepared expression plasmids are designated as pEGFP-N2-cPD-1 and pEGFP-N2-cPD-L1.

Primer (cPD-1-EGFP F):
(SEQ ID NO: 29)
CCGCTCGAGATGGGGAGCCGGCGGGGGCC
Primer (cPD-1-EGFP R):
(SEQ ID NO: 30)
CGCGGATCCTGAGGGGCCACAGGCCGGGTC
Primer (cPD-L1-EGFP F):
(SEQ ID NO: 31)
GAAGATCTATGAGAATGTTTAGTGTC
Primer (cPD-L1-EGFP R):
(SEQ ID NO: 32)
GGAATTCTGTCTCTTCAAATTGTATATC

COS-7 cells were subcultured at a density of 5×10⁴ cells/cm² in 6-well plates, and then cultured overnight in RPMI 1640 medium containing 10% inactivated fetal bovine serum and 0.01% L-glutamine at 37° C. in the presence of 5% CO₂. The pEGFP-N2-cPD-1, pEGFP-N2-cPD-L1 or pEGFP-N2 (negative control) was introduced into COS-7 cells at 0.4 μg/cm² using Lipofectamine 2000 (Invitrogen). The cells were cultured for 48 hours (cPD-1-EGFP expressing cell and cPD-L1-EGFP expressing cell). In order to confirm the expression of canine PD-1 and PD-L1 in the thus prepared expressing cells, intracellular localization of enhanced green fluorescent protein (EGFP) was visualized with an inverted confocal laser microscope LSM700 (ZEISS) (Maekawa N, Konnai S, Ikebuchi R, Okagawa T, Adachi M, Takagi S, Kagawa Y, Nakajima C, Suzuki Y, Murata S, Ohashi K. PLoS One. 2014 Jun. 10; 9(6):e98415).

### 2.4 Construction of Recombinant Canine PD-1, PD-L1 and CD80

In order to amplify the extracellular regions of canine PD-1, PD-L1 and CD80 estimated from their putative amino acid sequences, primers were designed. Briefly, primers having an NheI or EcoRV recognition sequence (PD-1 and PD-L1) added on the 5′ side (cPD-1-Ig F and R; cPD-L1-Ig F and R) or having an EcoRV or KpnI (CD80) recognition sequence added on the 5′ side (cCD80-Ig F and R) were designed. PCR was performed using a synthesized beagle PBMC-derived cDNA as a template. The PCR products were digested with NheI (Takara) and EcoRV (Takara) or with EcoRV (Takara) and KpnI (New England Biolabs) and purified with FastGene Gel/PCR Extraction Kit (NIPPON Genetics). The thus purified DNAs were individually cloned into pCXN2.1-Rabbit IgG Fc vector (Niwa et al., 1991; Zettlmeissl et al., 1990; kindly provided by Dr. T. Yokomizo, Juntendo University Graduate School of Medicine, and modified in the inventors' laboratory) treated with restriction enzymes in the same manner. The expression plasmids were purified with QIAGEN Plasmid Midi kit (Qiagen) and stored at −30° C. until use in experiments. Hereinafter, the thus prepared expression plasmids are designated as pCXN2.1-cPD-1-Ig, pCXN2.1-cPD-L1-Ig and pCXN2.1-cCD80-Ig, respectively.

Primer (cPD-1-Ig F):
(SEQ ID NO: 33)
CGCGGCTAGCATGGGGAGCCGGCGGGGGCC
Primer (cPD-1-Ig R):
(SEQ ID NO: 34)
CGCGGATATCCAGCCCCTGCAACTGGCCGC
Primer (cPD-L1-Ig F):
(SEQ ID NO: 35)
CGCGGCTAGCATGAGAATGTTTAGTGTCTT
Primer (c-PD-L1-Ig R):
(SEQ ID NO: 36)
CGCGGATATCAGTCCTCTCACTTGCTGGAA
Primer (cCD80-Ig F):
(SEQ ID NO: 104)
CGCGGATATCATGGATTACACAGCGAAGTG
Primer (cCD80-Ig R):
(SEQ ID NO: 105)
CGGGGTACCCCAGAGCTGTTGCTGGTTAT

These expression vectors were individually transfected into Expi293F cells (Life Technologies) to obtain a culture supernatant containing a recombinant Ig fusion protein. The recombinant protein produced was purified from the supernatant with Ab Capcher Extra (Protein A mutant; ProteNova). After buffer exchange with phosphate-buffered physiological saline (PBS; pH 7.4) using PD-MidiTrap G-25 (GE Healthcare), each recombinant protein was stored at −30° C. until use in experiments (cPD-1-Ig, cPD-L1-Ig and cCD80-Ig). The concentration of each protein was measured with Pierce BCA Protein Assay Kit (Thermo Fisher Scientific) before use in subsequent experiments.

2.5 Identification of Rat Anti-Bovine PD-L1 Monoclonal Antibody Showing Cross-Reactivity with Canine PD-L1

In order to identify rat anti-bovine PD-L1 monoclonal antibody showing cross-reactivity with canine PD-L1, flow cytometry was performed using the anti-bovine PD-L1 antibody prepared in 2.1 above. The anti-bovine PD-L1 antibody (10 μg/ml) was reacted with 2×10⁵-1×10⁶ cells at room temperature for 30 min. After washing, the anti-bovine PD-L1 antibody was detected with allophycocyanine-labeled anti-rat Ig goat antibody (Beckman Coulter). FACS Verse (Becton, Dickinson and Company) was used for analysis. As negative controls, rat IgG2a (κ) isotype control (BD Biosciences), rat IgG1 (κ) isotype control (BD Biosciences) and rat IgM (κ) isotype control (BD Biosciences) were used. For every washing operation and dilution of antibodies, 10% inactivated goat serum-supplemented PBS was used (MaekawaN, Konnai S, Ikebuchi R, Okagawa T, Adachi M, Takagi S, KagawaY, Nakajima C, Suzuki Y, Murata S, Ohashi K. PLoS One. 2014 Jun. 10; 9(6):e98415 which is an article describing the use of three bovine PD-L1 monoclonal antibodies: 4G12 (Rat IgG2a (κ)), 5A2 (Rat IgG1 (κ)) and 6G7 (Rat IgM (κ)).

### 2.6 Selection Test of Variable Region for Establishment of Rat-Canine Chimeric Anti-PD-L1 Antibody

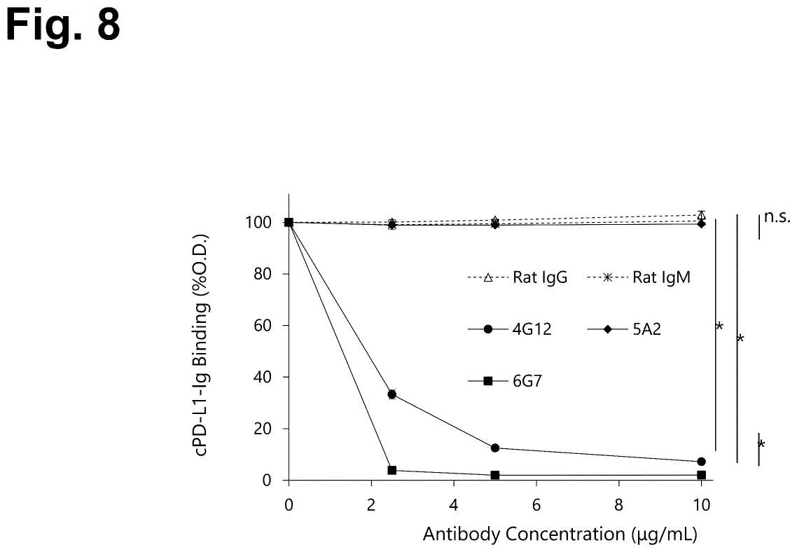

Out of 10 clones of rat anti-bovine PD-L1 monoclonal antibody which showed cross-reactivity with canine PD-L1, 4G12 (Rat IgG2a (κ)), 5A2 (Rat IgG1 (κ)) and 6G7 (Rat IgM (κ)) were selected and check was made to see whether these antibodies would inhibit canine PD-1/PD-L1 binding. Briefly, canine PD-1-Ig (prepared in 2.4 above) was immobilized on flat bottomed 96-well plates and blocked with 1% BSA and 0.05% Tween 20-containing PBS. Canine PD-L1-Ig (prepared in 2.4 above) was biotinylated using Lightning-Link Biotin Conjugation Kit (Innova Bioscience) and reacted with various concentrations (0, 2.5, 5 and 10 μg/ml) of rat anti-bovine PD-L1 antibodies 4G12, 5A2 and 6G7 at 37° C. for 30 min, followed by addition to the 96-well plates. The binding of cPD-L1-Ig to cPD-1-Ig was measured by color reaction using Neutravidin-HRP (Thermo Fisher Scientific) and TMB one component substrate (Bethyl Laboratories). As a result, rat anti-bovine PD-L1 monoclonal antibodies 4G12 and 6G7 showed a good inhibitory activity against canine PD-1/PD-L1 binding, whereas 5A2 showed no binding inhibitory activity (FIG. 8).

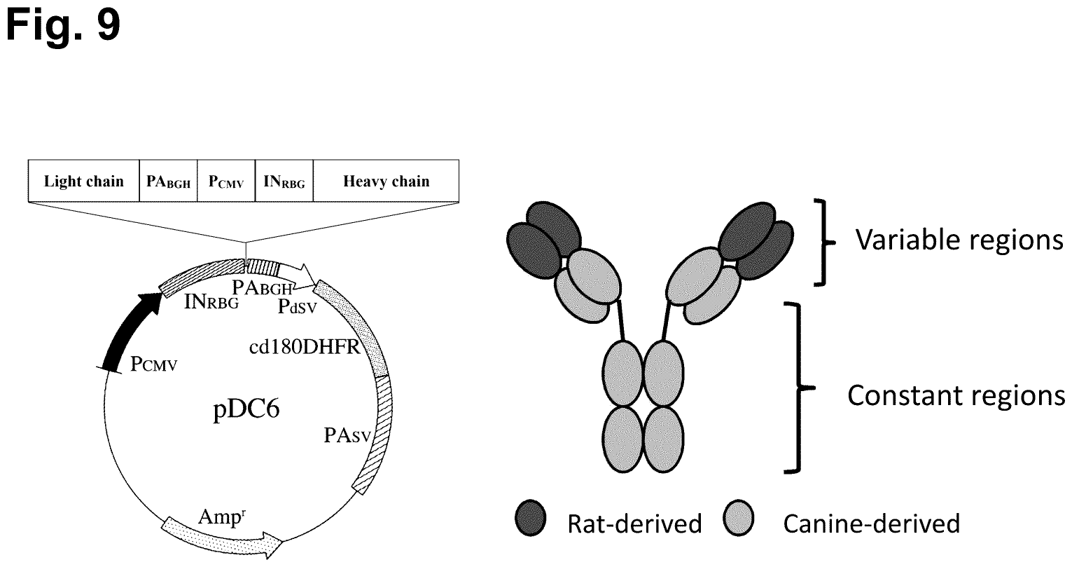

### 2.7 Preparation of Rat-Canine Chimeric Anti-PD-L1 Antibody Expressing Vector (FIG. 9)

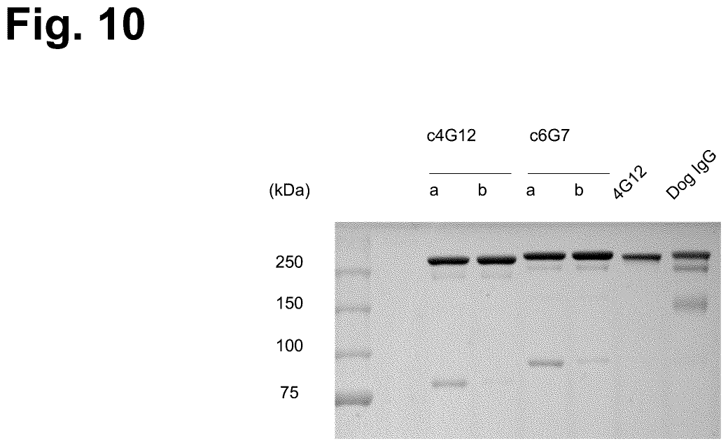

Using rat anti-bovine PD-L1 monoclonal antibodies 4G12 and 6G7 which showed a good inhibitory activity against canine PD-1/PD-L1 binding (FIG. 1) as the variable region, two types of rat-canine chimeric anti-PD-L1 antibodies were established. Briefly, heavy chain and light chain variable region genes were identified from hybridomas producing rat anti-bovine PD-L1 monoclonal antibodies 4G12 and 6G7. Further, the heavy chain and light chain variable region genes of the above rat antibodies were linked to the constant region of heavy chain IgG4 and the constant region of light chain Lambda of a known canine antibody, respectively, to prepare nucleotide sequences, followed by codon optimization (SEQ ID NOS: 9 and 10 (amino acid sequences), SEQ ID NOS: 19 and 20 (nucleotide sequences after codon optimization). Then, synthesis of genes was performed so that Nod restriction enzyme recognition site, KOZAK sequence, chimeric antibody's light chain sequence, poly-A addition signal sequence (PABGH), promoter sequence (PCMV), Sad restriction enzyme recognition site, intron sequence (INRBG), KOZAK sequence, chimeric antibody's heavy chain sequence and XbaI restriction enzyme recognition site would be located in this order. The synthesized gene strands were individually incorporated into the cloning site (Nod and XbaI restriction enzyme recognition sequences downstream of PCMV and between INRBG and PABGH) of expression vector pDC6 (kindly provided by Prof. S. Suzuki, Hokkaido University Research Center for Zoonosis Control) using restriction enzyme recognition sequences so that the above-listed sequences would be located in the above-mentioned order (FIG. 9). Thus, rat-canine chimeric anti-PD-L1 antibody expressing vectors were constructed. Each of the expression vectors was transfected into Expi293F cells (Life Technologies) to obtain a culture supernatant containing a chimeric antibody. The chimeric antibody was purified from the supernatant with Ab Capcher Extra (Protein A mutant; ProteNova) and further purified by gel filtration chromatography. SDS-PAGE was performed under non-reducing conditions using 10% acrylamide gel. Bands were stained with Quick-CBB kit (Wako Pure Chemical) and decolorized in distilled water. Although contaminant proteins were observed after protein A purification alone, a highly purified antibody could be obtained by performing gel filtration chromatography (FIG. 10). It was confirmed by flow cytometry that the resultant purified antibodies specifically bound to canine PD-L1 expressing cells (data not shown). When the inhibitory activity of the two chimeric antibodies against canine PD-1/PD-L1 binding was examined by the method described in 2.6 above, rat-canine chimeric anti-PD-L1 antibody c4G12 showed a binding inhibitory activity similar to that of its original rat anti-bovine PD-L1 monoclonal antibody 4G12, whereas binding inhibition capacity was clearly attenuated in rat-canine chimeric anti-PD-L1 antibody c6G7 (FIG. 4) Therefore, rat-canine chimeric anti-PD-L1 antibody c4G12 was selected as a therapeutic antibody, which incorporated the variable region sequences of rat anti-bovine PD-L1 monoclonal antibody 4G12 (SEQ ID NOS: 2 and 1 (amino acid sequences) and SEQ ID NOS: 16 and 15 (nucleotide sequences after codon optimization)). The amino acid sequence and the nucleotide sequence (after codon optimization) of the light chain of c4G12 are shown in SEQ ID NOS: 9 and 19, and the amino acid sequence and the nucleotide sequence (after codon optimization) of the heavy chain of c4G12 are shown in SEQ ID NOS: 10 and 20.

2.8 Expression of Rat-Canine Chimeric Anti-PD-L1 Antibody c4G12

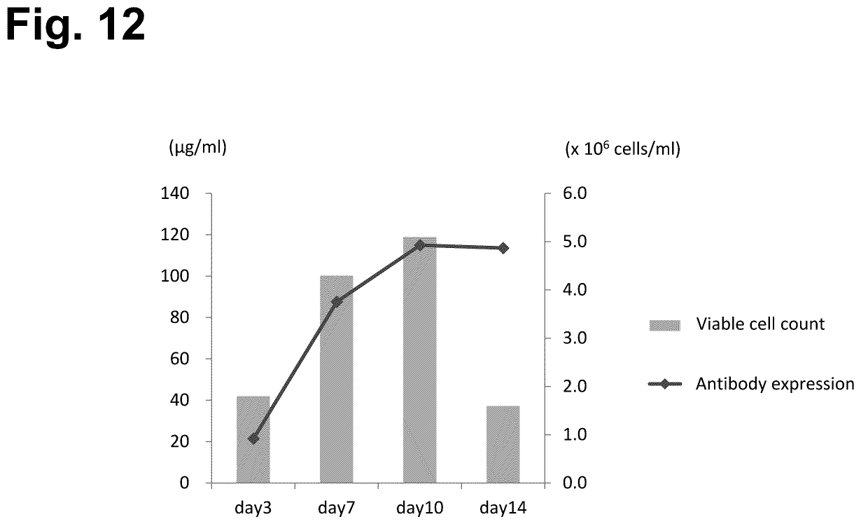

Rat-canine chimeric anti-PD-L1 antibody c4G12 expressing vector pDC6 as used in 2.7 above was transfected into CHO-DG44 cells (CHO-DG44(dfhr^{-/-})) which were dihydrofolate reductase deficient cells and high expression clones were selected by dot blotting. Further, gene amplification treatment was performed by adding load on cells in a medium containing 60 nM methotrexate (Mtx). Cells stably expressing rat-canine chimeric anti-PD-L1 antibody c4G12 (clone name: 4.3F1) after gene amplification were transferred to Mtx-free Opti-CHO medium and cultured under shaking for 14 days (125 rpm, 37° C., 5% CO₂). Cell survival rate was calculated by trypan blue staining (FIG. 12). Chimeric antibody production in the culture supernatant was measured by ELISA (FIG. 12). The culture supernatant at day 14 was centrifuged at 10,000 g for 10 min to remove cells, then passed through a 0.22 μm filter before the process proceeded to purification steps for the antibody.

It should be noted that by exchanging the medium with Dynamis medium and doing appropriate feeding, antibody production was improved about two-fold compared to the conventional production (data not shown).

2.9 Purification of Rat-Canine Chimeric Anti-PD-L1 Antibody c4G12

The culture supernatant provided as described above was purified with Ab Capcher Extra (ProteNova). An open column method was used for binding to resin; PBS pH 7.4 was used as equilibration buffer and wash buffer. As elution buffer, IgG Elution Buffer (Thermo Scientific) was used. As neutralization buffer, 1 M Tris was used. The purified antibody was concentrated and buffer-exchanged with PBS by ultrafiltration using Amicon Ultra-15 (50 kDa, Millipore). The resultant antibody was passed through a 0.22 μm filter for use in respective experiments.

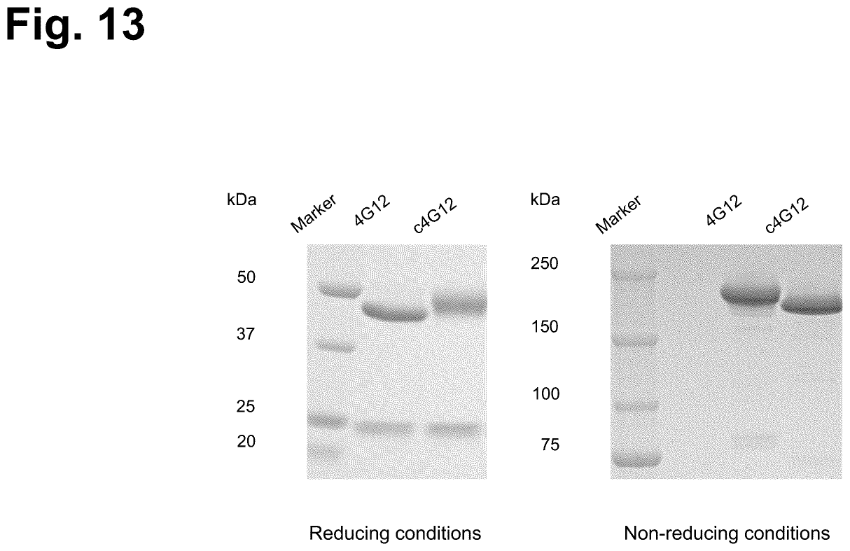

2.10 Confirmation of Purification of Rat-Canine Chimeric Anti-PD-L1 Antibody c4G12 (FIG. 13)

In order to confirm the purity of the purified antibody, antibody proteins were detected by SDS-PAGE and CBB staining. Using SuperSep Ace 5-20% (Wako) gradient gel, rat anti-bovine PD-L1 monoclonal antibody 4G12 and rat-canine chimeric anti-PD-L1 antibody c4G12 were electrophoresed under reducing conditions and non-reducing conditions. Bands were stained with Quick-CBB kit (Wako) and decolored in distilled water. Bands were observed at positions of molecular weights corresponding to antibodies. No bands of contaminant proteins were recognized visually.

2.11 Measurement of Binding Avidities to cPD-L1-His of Rat Anti-Bovine PD-L1 Monoclonal Antibody 4G12 and Rat-Canine Chimeric Anti-PD-L1 Antibody c4G12

In order to amplify the extracellular region of canine PD-L1 estimated from its putative amino acid sequence, primers were designed. Briefly, a primer having an NheI recognition sequence added on the 5′ side (cPD-L1-His F) and a primer having an EcoRV recognition sequence and 6xHis tag sequence added on the 5′ side (cPD-L1-His R) were designed. PCR was performed using a synthesized beagle PBMC-derived cDNA as a template. The PCR products were digested with NheI (Takara) and EcoRV (Takara) and purified with FastGene Gel/PCR Extraction Kit (NIPPON Genetics). The thus purified DNA was cloned into pCXN2.1 vector (Niwa et al., 1991; kindly provided by Dr. T. Yokomizo, Juntendo University Graduate School of Medicine) treated with restriction enzymes in the same manner. The expression plasmids were purified with QIAGEN Plasmid Midi kit (Qiagen) and stored at −30° C. until use in experiments. Hereinafter, the thus prepared expression plasmid is designated as CXN2.1-cPD-L1-His.

Primer (cPD-L1-His F):
(SEQ ID NO: 106)
CGCGGCTAGCATGAGAATGTTTAGTGTCTT
Primer (cPD-L1-His R):
(SEQ ID NO: 107)
CGCGGATATCTTAATGGTGATGGTGATGGT
GAGTCCTCTCACTTGCTGG

The expression vector was transfected into Expi293F cells (Life Technologies) to obtain a culture supernatant containing a recombinant protein. The recombinant protein produced was purified from the supernatant using TALON Metal Affinity Resin (Clontech), and the buffer was exchanged with PBS using Amicon Ultra-4 Ultracel-3 (Merck Millipore). The thus obtained recombinant protein was stored at 4° C. until use in experiments (cPD-L1-His). The protein concentration was measured with Pierce BCA Protein Assay Kit (Thermo Fisher Scientific) for use in subsequent experiments.

Using a biomolecular interaction analyzer (Biacore X100), the binding avidities to cPD-L1-His of rat anti-bovine PD-L1 monoclonal antibody 4G12 and rat-canine chimeric anti-PD-L1 antibody c4G12 were assessed. Briefly, anti-histidine antibody was fixed on CM5 censor chip, followed by capturing of cPD-L1-His. Subsequently, monoclonal antibodies were added as analyte to observe specific binding. Both antibodies exhibited specific binding and their avidities were almost comparable (Table 1). Further, the binding avidities of canine PD-1-Ig and CD80-Ig to cPD-L1-His were measured in the same manner and found to be clearly lower than that of rat-canine chimeric anti-PD-L1 antibody c4G12 (Table 1).

TABLE 1
Binding Avidity of Each Antibody and
Recombinant Protein to Canine PD-L1-His

| | ka (×10⁶/Ms) | kd (×10⁻³/s) | KD (nM) |
|---|---|---|---|
| 4G12 | 2.42 ± 0.10 | 4.54 ± 0.19 | 1.88 ± 0.06 |
| c4G12 | 3.14 ± 0.19 | 7.19 ± 0.26 | 2.30 ± 0.07 |
| cPD-1 | | | 25.4 ± 4.89 |
| cCD80 | | | 24.3 ± 0.89 |

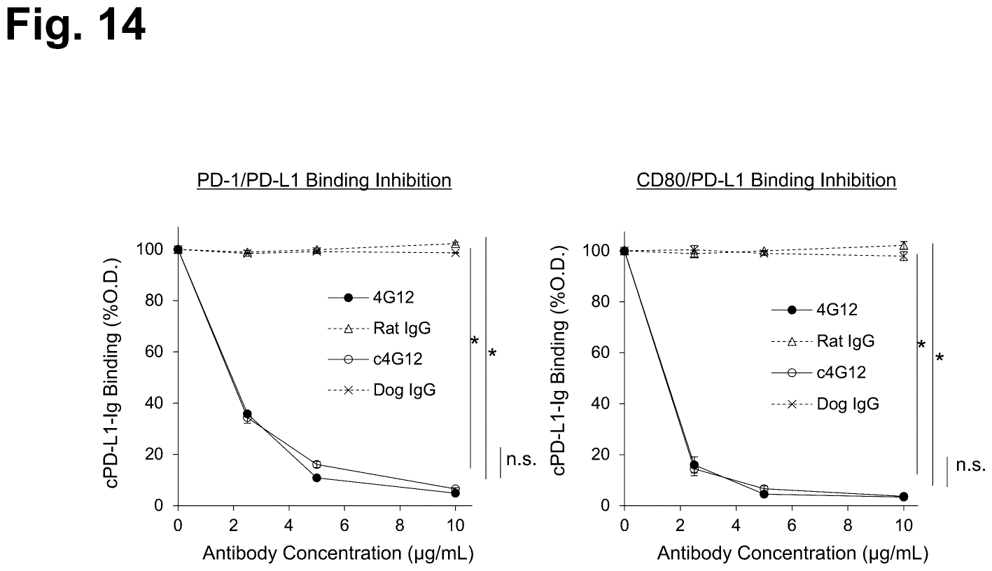

2.12 Inhibitory Activity of Rat-Canine Chimeric Anti-PD-L1 Antibody c4G12 against Canine PD-1/PD-L1 Binding and CD80/PD-L1 Binding (FIG. 14)

Using the canine PD-1-Ig, PD-L1-Ig and CD80-Ig (described above), anti-PD-L1 antibody was tested for its ability to inhibit canine PD-1/PD-L1 binding and CD80/PD-L1 binding. Briefly, canine PD-1-Ig or CD80-Ig was immobilized on flat-bottom 96-well plates. Canine PD-L1-Ig was reacted with various concentrations (0, 2.5, 5 and 10 μg/ml) of rat anti-bovine PD-L1 antibody 4G12 or rat-canine chimeric anti-PD-L1 antibody c4G12 according to the same procedures as described in 2.6 above, and the binding of canine PD-L1-Ig was assessed. No change in binding inhibition activity was observed due to the chimerization of antibody.

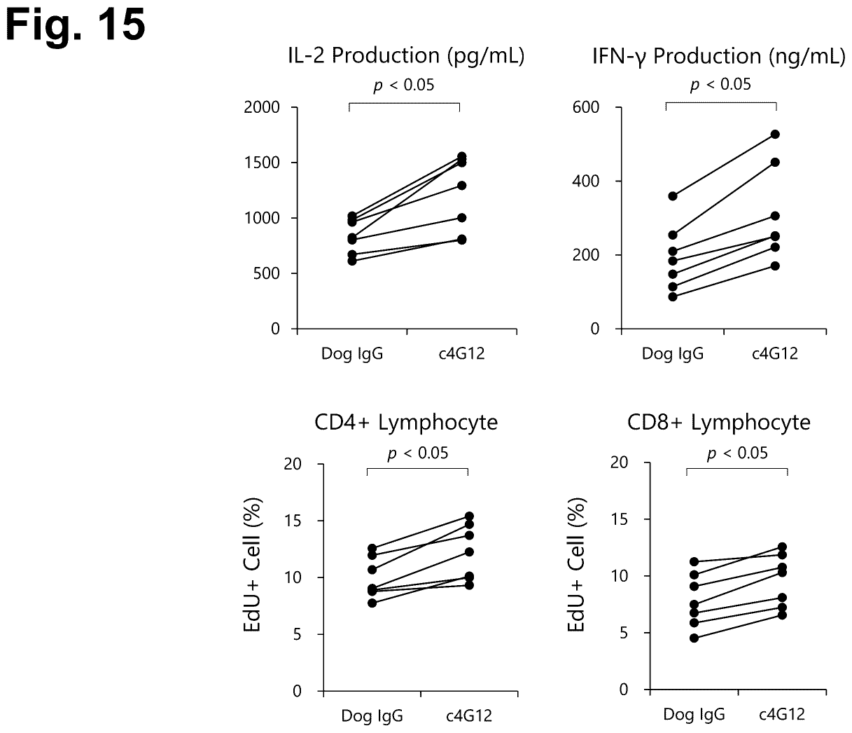

2.13. Canine Immune Cell Activating Effect of Rat-Canine Chimeric Anti-PD-L1 Antibody c4G12 (FIG. 15)

Canine PBMCs were cultured under stimulation with a superantigen Staphylococcal Enterotoxin B (SEB) for three days, and changes in cytokine production by addition of rat-canine chimeric anti-PD-L1 antibody c4G12 were measured by ELISA using Duoset ELISA canine IL-2 or IFN-γ (R&D systems). Rat-canine chimeric anti-PD-L1 antibody c4G12 increased the production of IL-2 and IFN-γ from canine PBMCs. Further, nucleic acid analogue EdU was added to the culture medium at day 2 of the culture under SEB stimulation. Two hours later, uptake of EdU was measured by flow cytometry using Click-iT Plus EdU flow cytometry assay kit (Life Technologies). As a result, EdU uptake in canine CD4⁺ and CD8⁺ lymphocytes was enhanced by addition of rat-canine chimeric anti-PD-L1 antibody c4G12, indications an elevated cell proliferation capacity.

### 2.14 Selection of Tumor-Affected Dogs to be Used in Canine Inoculation Test

Since the subject treatment is expected to manifest a higher efficacy when PD-L1 is being expressed in tumors, PD-L1 expression analysis at the tumor site of dogs was performed by immunohistochemical staining. Briefly, tumor tissue samples fixed with formaldehyde and embedded in paraffin were sliced into 4 μm thick sections with a microtome, attached to and dried on silane-coated slide glass (Matsunami Glass) and deparaffinized with xylene/alcohol. While the resultant sections were soaked in citrate buffer [citric acid (Wako Pure Chemical) 0.37 g, trisodium citrate dihydrate (Kishida Chemical) 2.4 g, distilled water 1000 ml], antigen retrieval treatment was performed for 10 min with microwave, followed by staining using a Nichirei automatic immuno-staining device. As pretreatment, sample sections were soaked in 0.3% hydrogen peroxide-containing methanol solution at room temperature for 15 min and washed with PBS. Then, anti-bovine PD-L1 monoclonal antibody was added and reaction was conducted at room temperature for 30 min. After washing with PBS, histofine simple stain MAX-PO (Rat) (Nichirei Bioscience) was added and reaction was carried at room temperature for 30 min, followed by coloring with 3,3′-diaminobenzidine tetrahydrocholride and observation with a light microscope. Dogs with oral melanoma or undifferentiated sarcoma in which tumor cells were PD-L1 positive were used in the following inoculation test (clinical trial). Anti-bovine PD-L1 monoclonal antibody was established from a rat anti-bovine PD-L1 monoclonal antibody producing hybridoma (Ikebuchi R, Konnai S, Okagawa T, Yokoyama K, Nakajima C, Suzuki Y, Murata S, Ohashi K. Immunology. 2014 August; 142(4):551-61).

### 2.15 Inoculation Test on Dogs

With respect to the rat-canine chimeric anti-PD-L1 antibody c4G12 to be inoculated into dogs in the clinical trial, the culture supernatant obtained by the procedures described in 2.8 above was purified by affinity chromatography using MabSelect SuRe LX (GE Healthcare) and then by hydroxyapatite chromatography using BioScale CHT20-I prepacked column (Bio-Rad) in order to remove contaminants and polymeric proteins. Aggregate-containing fractions were further purified by anion exchange chromatography using HiScreen Q-Sepharose HP prepacked column (GE Healthcare).

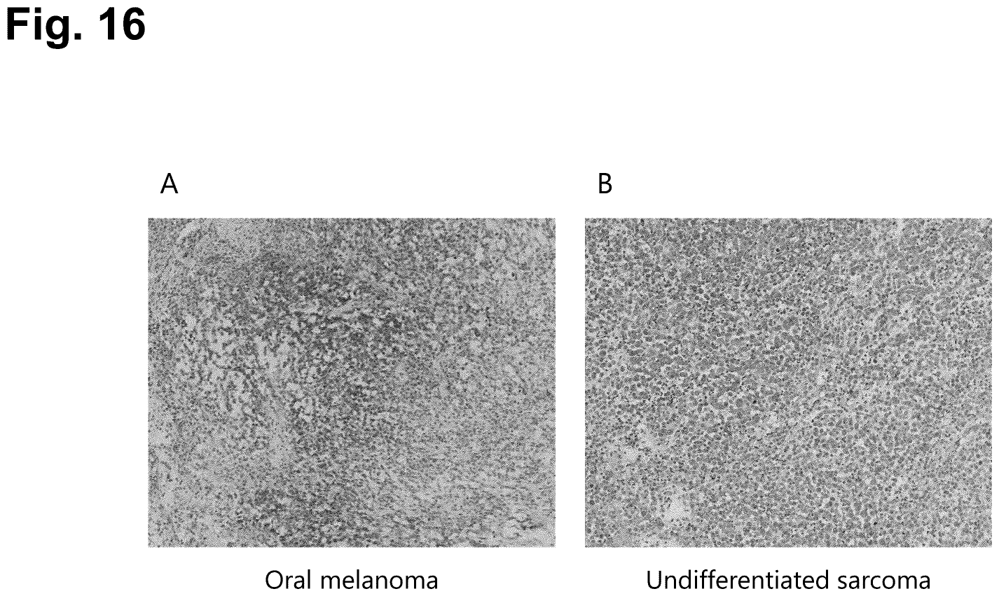

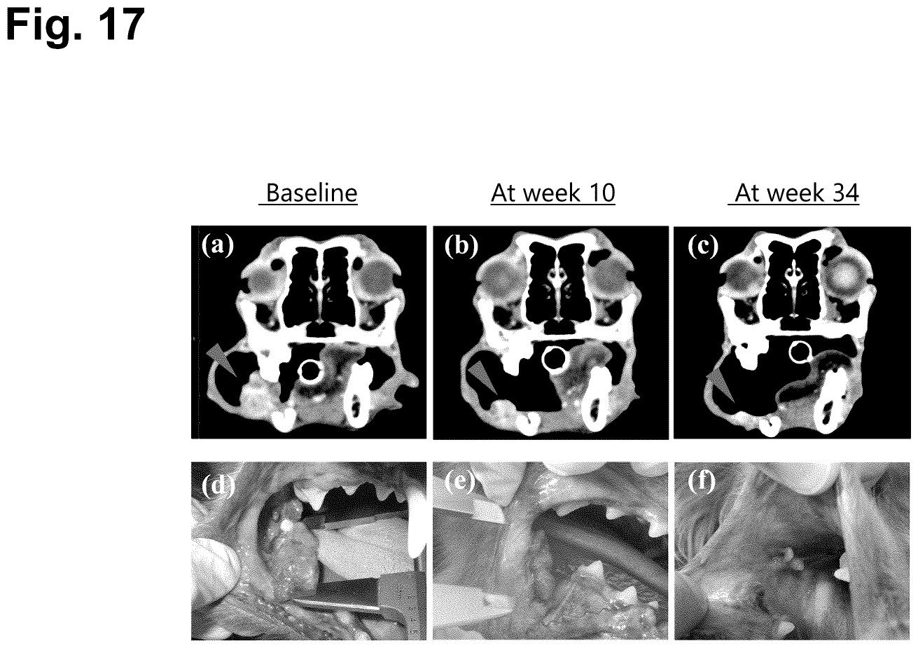

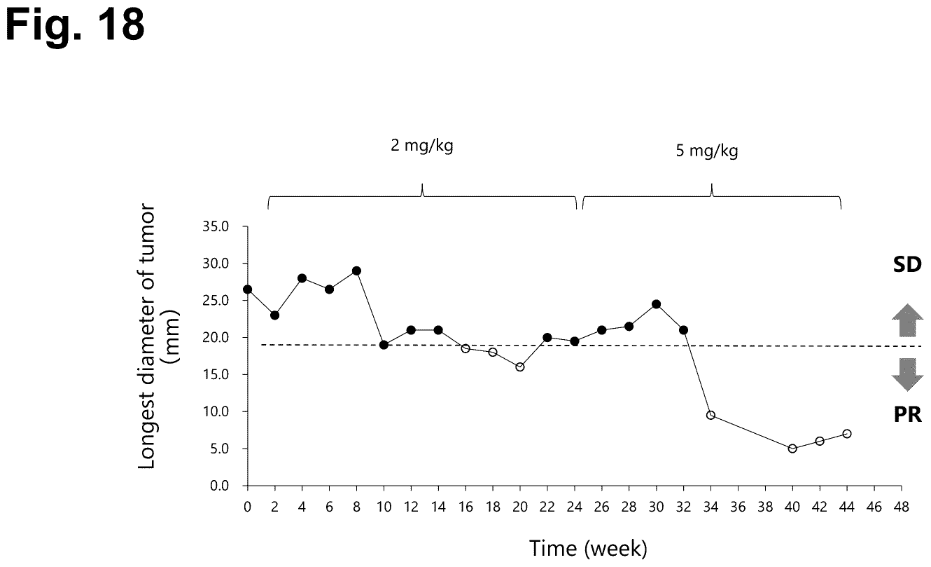

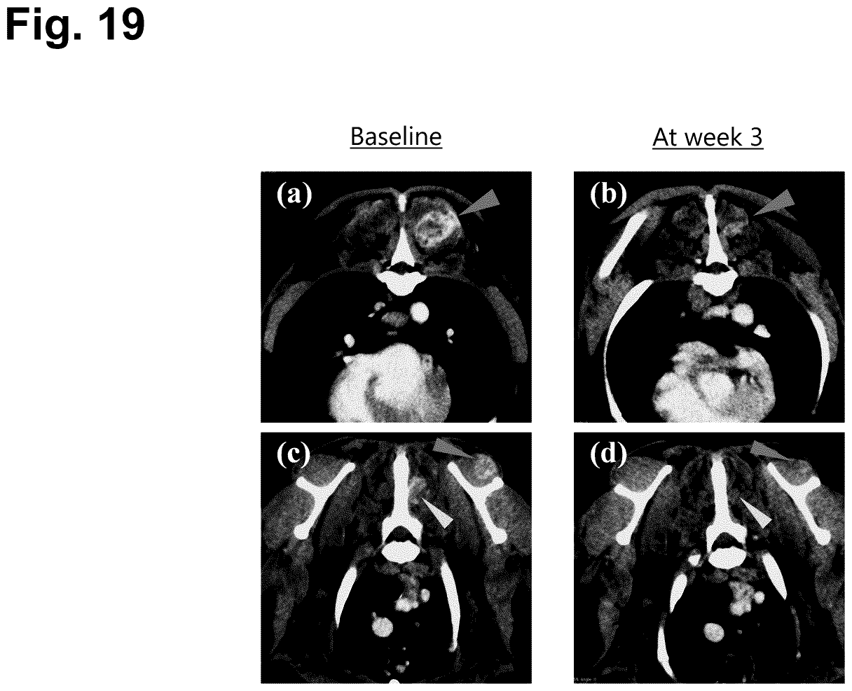

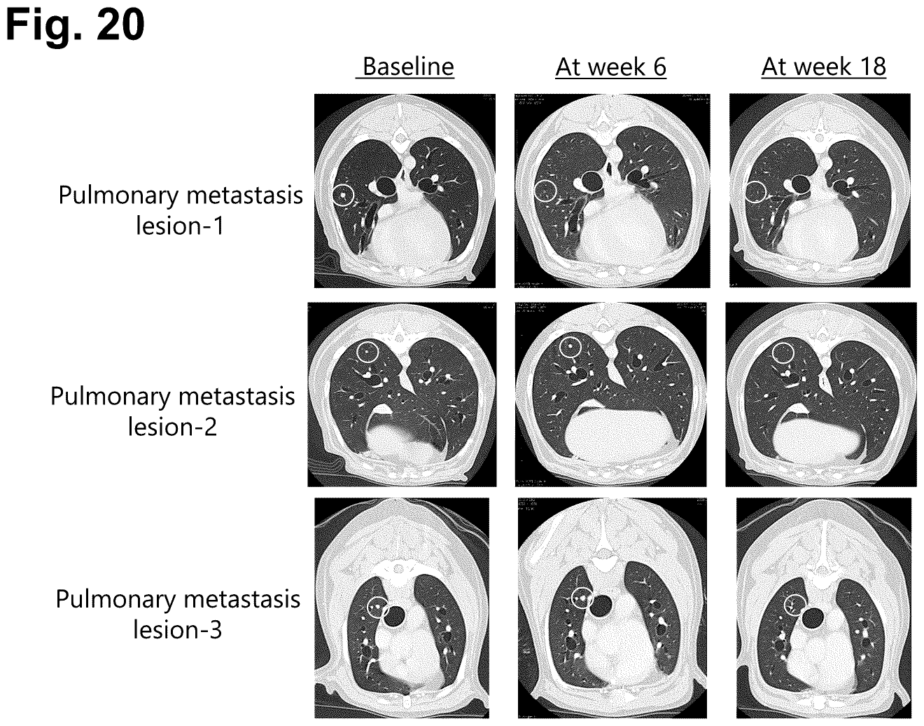

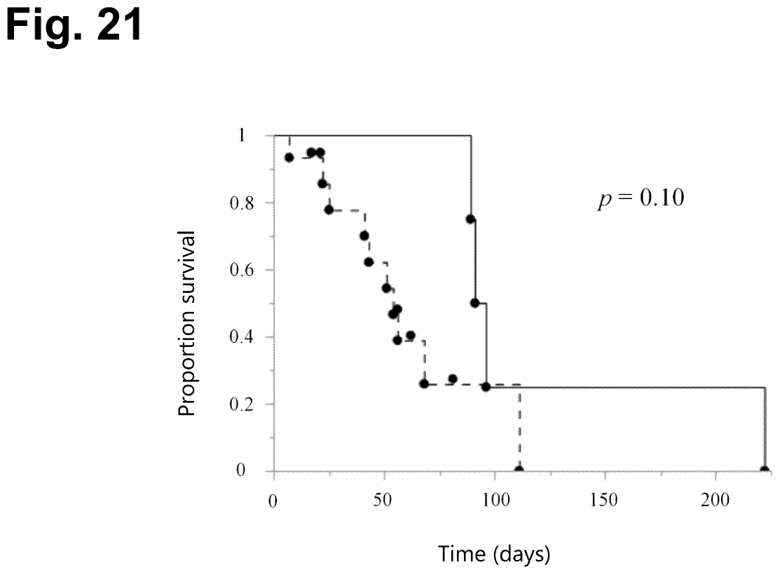

(1) Safety Test: The established rat-canine chimeric anti-PD-L1 antibody c4G12 was administered intravenously into a dog (beagle, spayed female, 13-year-old, about 10 kg in body weight) at 2 mg/kg, every 2 weeks, 3 times in total. There was observed no anaphylaxis or other adverse effects that would cause any trouble in clinical trials. (2) Clinical Trial 1: The established rat-canine chimeric anti-PD-L1 antibody c4G12 was administered intravenously into a PD-L1 positive dog with relapsed oral melanoma (FIG. 16A) (miniature dachshund, male, 11-year-old, about 7.5 kg in body weight) at 2 mg/kg or 5 mg/kg, every 2 weeks, 22 times in total. At week 10 after the start of treatment, a remarkable reduction in tumor size was recognized. At week 34 after the start of treatment, a still further reduction was confirmed (FIG. 10). During the observation period of 44 weeks, no metastases to lymph nodes or the lung were observed. When 30% or more reduction in the longest diameter of tumor compared to that at the baseline is defined as PR (partial response), the criterion of PR was satisfied at weeks 16-20 and at week 34 and thereafter (FIG. 18).
(3) Clinical Trial 2: Rat-canine chimeric anti-PD-L1 antibody c4G12 was administered intravenously into a dog with undifferentiated sarcoma whose primary lesion was PD-L1 positive (FIG. 16B) and who had a plurality of metastatic lesions in muscles throughout the body (west highland white terrier, castrated male, 12-year-old, about 8 kg in body weight) at 5 mg/kg, every 2 weeks, 2 times in total. At week 3 from the start of treatment, a clear regression of tumor was recognized (FIG. 19).
(4) Clinical Trial 3: Rat-canine chimeric anti-PD-L1 antibody c4G12 was administered intravenously into a dog with oral melanoma whose primary lesion had been removed by surgery (beagle, spayed female, 11-year-old, about 10 kg in body weight) at 2 mg/kg or 5 mg/kg, every 2 weeks, 9 times in total. At week 18 after the start of treatment, a plurality of pulmonary metastatic lesions disappeared (FIG. 20),
(5) Clinical Trial 4: Rat-canine chimeric anti-PD-L1 antibody c4G12 was administered intravenously into 4 dogs with oral melanoma with pulmonary metastasis at 2 mg/kg or 5 mg/kg, every 2 weeks. Although no clear reduction in tumor size was observed during the observation period, the duration of the treated dogs' survival after confirmation of pulmonary metastasis tended to be longer than that of a control group (antibody not administered, historical control group: n=15) (FIG. 21). Therefore, the survival duration may have been extended by antibody administration.

### 2.16 CDR Analysis of Anti-PD-L1 Antibody

The complementarity-determining regions (CDRs) of rat anti-bovine PD-L1 antibody 4G12 were determined using NCBI IGBLAST (http://www.ncbi.nlm.nih.gov/igblast/). The results are shown in FIG. 22.

### Example 2

Examination of Combined Effect of Anti-Bovine PD-L1 Antibody and COX-2 Inhibitor in Cattle with Johne's Disease

### 1. Introduction

The interaction between PD-1 and PD-L1 is one of the major molecular mechanisms through which pathogens evade immune responses. It has been reported that inhibition of the above interaction by using an antibody which specifically binds to either of those molecules can produce anti-pathogenic effects. In the subject Example, toward establishment of a novel control method against Johne's disease, the present inventors have confirmed in in vitro tests an immunostimulatory effect induced by a COX-2 inhibitor and enhancement of that effect when the inhibitor is used in combination with anti-bovine PD-L1 antibody.

### 2. Materials and Methods, as Well as Experimental Results

### 2.1. Examination of Immunosuppressive Effects of PGE₂

In order to examine the immunosuppressive effects of PGE₂ in cattle, the present inventors evaluated how PBMCs derived from uninfected cattle stimulated with anti-CD3 monoclonal antibody and anti-CD28 monoclonal antibody changed in proliferation capacity and cytokine production capacity as well as in expression levels of cytokine and transcription factor genes and PD-L1 in the presence of PGE₂.

### (1) Changes in Cell Proliferation Capacity Induced by PGE₂

Peripheral blood mononuclear cells (PBMCs) derived from cattle not infected with Mycobacterium avium subsp. paratuberculosis (hereinafter, referred to as “uninfected cattle”) were seeded in 96-well plates (Corning) at 4×10⁵ cells/well. The cells were cultured in RPMI 1640 medium (Sigma-Aldrich) supplemented with 10% inactivated fetal bovine serum (Thermo Fisher Scientific), antibiotics (streptomycin 100 μg/ml, penicillin 100 U/ml) (Thermo Fisher Scientific) and 2 mM L-glutamine (Thermo Fisher Scientific) for 3 days at 37° C. in the presence of 5% CO₂. The PBMCs were labeled with Carboxyfluorescein Diacetate Succinimidyl Ester (CFSE, Invitrogen). To the medium, 10-fold serially diluted PGE₂ (from 2.5 nM to 2,500 nM) (Cayman Chemical) or, as a negative control, phosphate buffered physiological saline (PBS, pH 7.2, Wako Pure Chemical) was added to give a total volume of 200 μl. As stimulants for T cells, 1 μg/ml of anti-CD3 monoclonal antibody (MM1A; Washington State University Monoclonal Antibody Center) and 1 μg/ml of anti-CD28 monoclonal antibody (CC220; Bio-Rad) were added. After culturing, PBMCs were harvested and analyzed by flow cytometry. In order to prevent non-specific reactions, PBS supplemented with 10% inactivated goat serum (Thermo Fisher Scientific) was added to each well at 100 μl/well, and left stationary at room temperature for 15 min. After washing, Alexa Fluor 647-labeled anti-CD4 monoclonal antibody (CC30; Bio-Rad), peridinin-chlorophyll-protein complex/cyanin 5.5 (PerCp/Cy 5.5)-labeled anti-CD8 monoclonal antibody (CC63; Bio-Rad) and phycoerythrin/cyanin 7 (PE/Cy7)-labeled anti-IgM monoclonal antibody (IL-A30; Bio-Rad) were reacted at room temperature for 20 min. The anti-CD4 monoclonal antibody (CC30) was labeled with Alexa Fluor 647 using Zenon Mouse IgG1 labeling Kits (Thermo Fisher Scientific). The anti-CD8 monoclonal antibody (CC63) and the anti-IgM monoclonal antibody (IL-A30) were labeled with PerCp/Cy5.5 and PE/Cy7, respectively, using Lightning-Link Conjugation Kit (Innova Biosciences). After reaction, washing was performed twice. Then, cell proliferation was analyzed with FACS Verse (BD Biosciences) and FCS Express 4 (De Novo Software). For every washing operation and dilution of antibodies, PBS supplemented with 1% bovine serum albumin (Sigma Aldrich) was used.

### (2) Changes in Cytokine Production Induced by PGE₂

PBMCs derived from uninfected cattle were seeded in 96-well plates at 4×10⁵ cells/well and cultured in the same manner as described in (1) above for 3 days (Note: analysis of TNF-α production was performed only for the cells cultured under stimulation with 2,500 nM PGE₂). After 3 days, the culture supernatant was collected. IFN-γ production was measured with ELISA for Bovine IFN-γ (MABTECH), and TNF-α production was measured with Bovine TNF alpha Do-It-Yourself ELISA (Kingfisher Biotech). For the measurement, absorbance at 450 nm was measured using a microplate reader MTP-900 (Corona Electric).

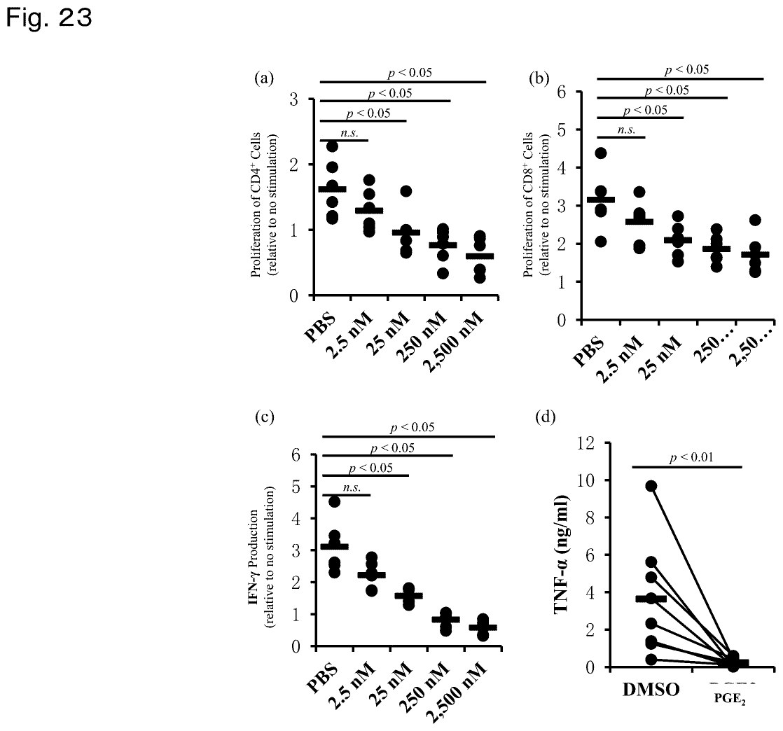

Experimental results of (1) and (2) above are shown in FIG. 23. In groups where PGE₂ was added at 25 nM, 250 nM and 2,500 nM, proliferation of CD4+ cells and CD8+ cells was significantly inhibited (FIGS. 23a and b). Likewise, IFN-γ production was significantly inhibited in groups where PGE₂ was added at 25 nM, 250 nM and 2,500 nM (FIG. 23c). Further, in the group where PGE₂ was added at 2,500 nM, TNF-α production was also significantly inhibited (FIG. 23d). These results demonstrated that PGE₂ has immunosuppressive effects also in cattle.

(3) Changes in mRNA Expression Levels of Cytokines, etc. Induced by PGE₂

PBMCs derived from uninfected cattle were seeded in 96-well plates at 1×10⁶ cells/well and cultured for 3 days in the presence of 2,500 nM PGE₂ or DMSO. Total cellular RNA was extracted from the thus cultured PBMCs using TRI reagent (Molecular Research Center), and cDNA was synthesized with PrimeScript Reverse Transcriptase (TaKaRa) and Oligo-dT primers. Using the synthesized cDNA as a template, real-time PCR was performed with LightCycler480 System II (Roche) in a 10 μl reaction solution containing SYBR Premix DimerEraser (TaKaRa) and 3 pmol each of the primers specific to individual genes. Then, changes in expression levels of individual genes were observed.

Primer (boIL2 F):)
(SEQ ID NO: 119)
TTT TAC GTG CCC AAG GTT AA
Primer (boIL2 R):
(SEQ ID NO: 120)
CGT TTA CTG TTG CAT CAT CA
Primer (boIL10 F):
(SEQ ID NO: 121)
TGC TGG ATG ACT TTA AGG G
Primer (boIL10 R):
(SEQ ID NO: 122)
AGG GCA GAA AGC GAT GAC A
Primer (boIFNγ F):
(SEQ ID NO: 123)
ATA ACC AGG TCA TTC AAA GG
Primer (boIFNγ R):
(SEQ ID NO: 124)
ATT CTG ACT TCT CTT CCG CT
Primer (boTNFα F):
(SEQ ID NO: 125)
TAA CAA GCC AGT AGC CCA CG
Primer (boTNFα R):
(SEQ ID NO: 126)
GCA AGG GCT CTT GAT GGC AGA
Primer (boTGFβ1 F):
(SEQ ID NO: 127)
CTG CTG AGG CTC AAG TTA AAA GTG
Primer (boTGFβ1 R):
(SEQ ID NO: 128)
CAG CCG GTT GCT GAG GTA G
Primer (boFoxp3 F):
(SEQ ID NO: 129)
CAC AAC CTG AGC CTG CAC AA
Primer (boFoxp3 R):
(SEQ ID NO: 130)
TCT TGC GGA ACT CAA ACT CAT C
Primer (boSTAT3 F):
(SEQ ID NO: 131)
ATG GAA ACA ACC AGT CGG TGA
Primer (boSTAT3 R):
(SEQ ID NO: 132)
TTT CTG CAC ATA CTC CAT CGC T
Primer (boACTB F):
(SEQ ID NO: 133)
TCT TCC AGC CTT CCT TCC TG
Primer (boACTB R):
(SEQ ID NO: 134)
ACC GTG TTG GCG TAG AGG TC
Primer (boGAPDH F):
(SEQ ID NO: 135)
GGC GTG AAC CAC GAG AAG TAT AA
Primer (boGAPDH R):
(SEQ ID NO: 136
CCC TCC ACG ATG CCA AAG T

Reaction conditions of the real-time PCR were as described below.
Thermal denaturation 95° C. for 5 sec (30 sec only for the first cycle)
Annealing 60° C. for 30 sec
Extension 72° C. for 30 sec
   After 45 cycles of thermal denaturation, annealing and extension, the temperature was raised from 65° C. to 95° C. at 0.1° C./see for melting curve analysis. The melting temperatures of amplified products were measured to confirm specificity. For each sample, expression levels of genes ACTB and GAPDH were quantified as internal standards.

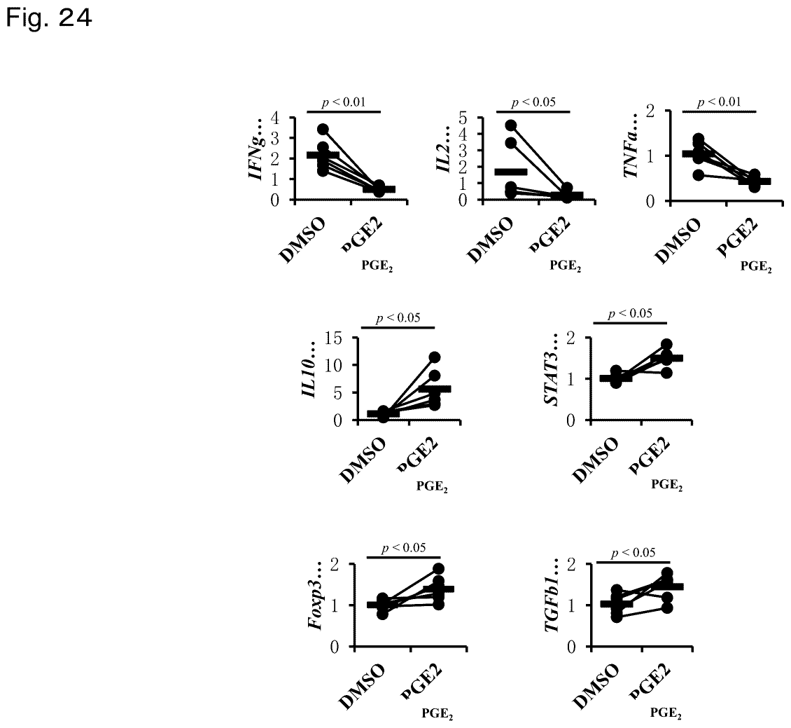

Experimental results of (3) are shown in FIG. 24. Addition of PGE₂ significantly decreased the expression levels of IFNγ, IL-2 and TNFα in PBMCs. On the other hand, PGE₂ significantly increased the expression levels of IL-10, STAT3, Foxp3 and TGFβ1 in PBMCs.

### (4) Changes in PD-L1 Expression Induced by PGE₂

PBMCs derived from uninfected cattle were seeded in 96-well plates at 1×10⁶ cells/well and cultured for 24 hours under the same conditions as described in (1) above. Total cellular RNA was extracted from the cultured PBMCs, and cDNA was synthesized in the same manner as described in (3) above. Then, real-time PCR was performed using PDL1 specific primers. Primer (boPDL1 F): GGG GGT TTA CTG TTG CTT GA (SEQ ID NO: 137) Primer (boPDL1 R): GCC ACT CAG GAC TTG GTG AT (SEQ ID NO: 138) Further, expression of PD-L1 protein in the cultured PBMCs was analyzed by flow cytometry. Briefly, PBS supplemented with 10% inactivated goat serum (Thermo Fisher Scientific) was added to harvested PBMCs at 100 μl/well and left stationary at room temperature for 15 min. After washing, rat anti-bovine PD-L1 antibody (4G12; Rat IgG2a; Ikebuchi R, Konnai S, Okagawa T, Yokoyama K, Nakajima C, Suzuki Y, Murata S, Ohashi K. Immunology 2014 August; 142(4):551-561) or rat IgG2a isotype control (BD Bioscience) was added and reaction was conducted at room temperature for 20 min. After washing twice, allophycocyanin (APC)-labeled anti-rat Ig antibody (Southern Biotech) was added and reaction was conducted at room temperature for 20 min. After washing twice, expression of PD-L1 protein was analyzed with FACS Verse (BD Biosciences) and FCS Express 4 (De Novo Software). For every washing operation and dilution of antibodies, PBS supplemented with 1% bovine serum albumin (Sigma Aldrich) was used.

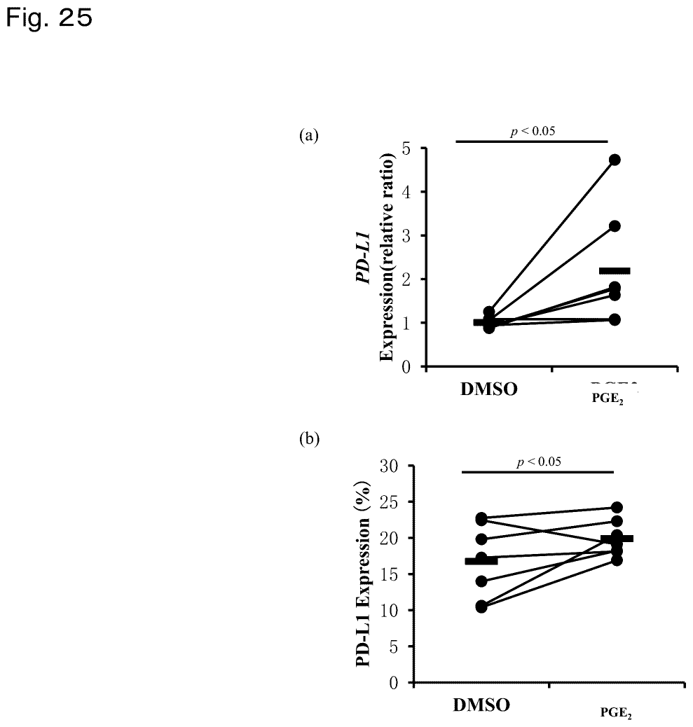

Experimental results of (3) are shown in FIG. 25. Expressions of PD-L1 mRNA (FIG. 25a) and PD-L1 protein (FIG. 25b) were significantly increased in PBMCs derived from the uninfected cattle that were cultured in the presence of added PGE₂. These results show a possibility that PGE₂ affects PD-L1 expression also in cattle.

### 2.2. Examination of Immunostimulatory Effects of COX-2 Inhibitor in Cattle PBMCs

In order to examine immunostimulatory effects of COX-2 inhibitor in cattle, a COX-2 inhibitor meloxicam was added to a PBMC culture test under stimulation with anti-CD3 monoclonal antibody and anti-CD28 monoclonal antibody, followed by evaluation of the proliferation capacity and cytokine production capacity of PBMCs derived from uninfected cattle.

PBMCs derived from uninfected cattle were seeded in 96-well plates at 4×10⁵ cells/well and cultured for 3 days in the presence of 1,000 nM meloxicam (Sigma-Aldrich) or DMSO as a negative control. As stimulants for T cells, 1 μg/ml of anti-CD3 monoclonal antibody (MM1A; Washington State University Monoclonal Antibody Center) and 1 μg/ml of anti-CD28 monoclonal antibody (CC220; Bio-Rad) were added. After 3 days, cell proliferation capacity and cytokine production were evaluated in the same manner as described in (1) and (2) in section 2.1 above.

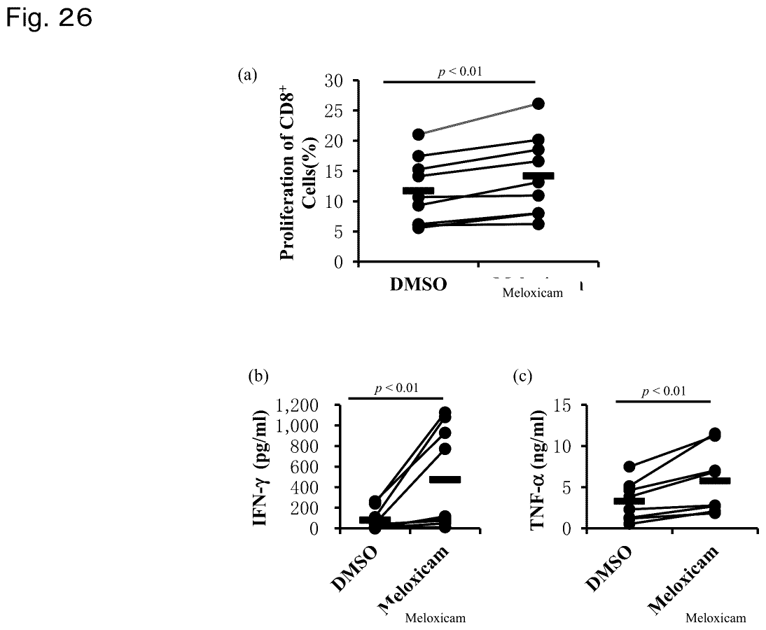

Experimental results are shown in FIG. 26. In the meloxicam-added group, proliferation rate of CD8⁺ cells was increased significantly (FIG. 26a), and so were the production of IFN-γ and TNF-α (FIGS. 26b and 26c). These results demonstrated that COX-2 inhibitor has immunostimulatory effects also in cattle.

2.3. Kinetic Analysis of PGE₂ in Cattle Infected with M. avium Subsp. Paratuberculosis

In order to elucidate the relation between bovine chronic infections and PGE₂, the present inventors performed kinetic analysis of PGE₂ in cattle infected with M. avium subsp. paratuberculosis.

### (1) Measurement of Serum PGE₂

First, PGE₂ contained in serum derived from cattle that developed Johne's disease from natural infection and PGE₂ contained in serum derived from uninfected cattle were quantified by ELISA. Briefly, the amount of PGE₂ contained in serum derived from cattle that naturally developed Johne's disease (kindly provided by Dr. Yasuyuki Mori, National Institute of Animal Health, National Agriculture and Food Research Organization) was measured with Prostaglandin E2 Express ELISA Kit (Cayman Chemical). For the measurement, absorbance at 450 nm was measured with a microplate reader MTP-900 (Corona Electric).

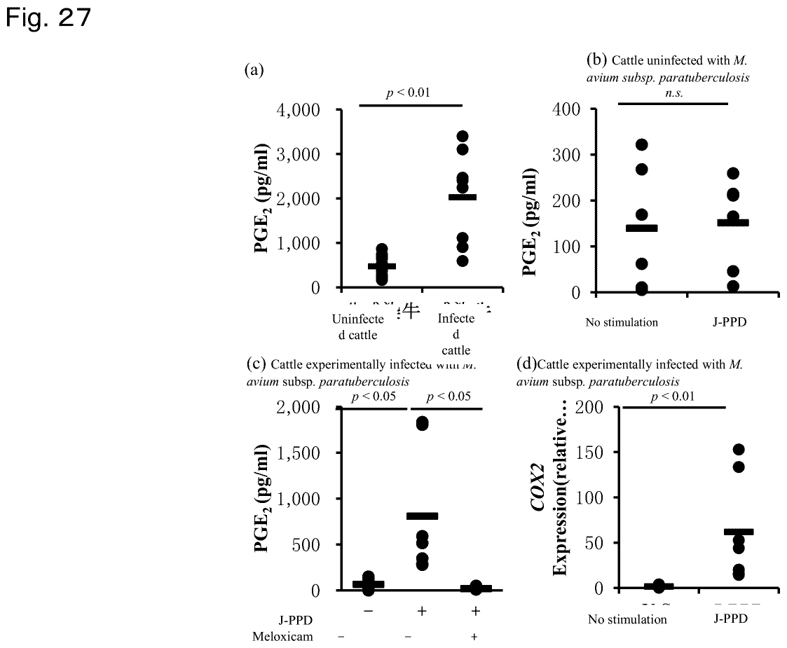

Experimental results of (1) are shown in FIG. 27a. Compared to uninfected cattle, the cattle manifesting Johne's disease showed a significant increase in serum PGE₂.

(2) Changes in PGE₂ Production by M. avium subsp. paratuberculosis Antigen Stimulation

In order to confirm that PGE₂ production is promoted by M. avium subsp. paratuberculosis antigen, PBMCs derived from cattle experimentally infected with M. avium subsp. paratuberculosis and those derived from uninfected cattle were cultured with M. avium subsp. paratuberculosis antigen, and PGE₂ in culture supernatants was quantified by ELISA. Briefly, PBMCs derived from experimentally infected cattle and those from uninfected cattle were seeded in 96-well plates at 4×10⁵ cells/well and cultured for 5 days in the presence of 1 μg/ml of M. avium subsp. paratuberculosis antigen. As the M. avium subsp. paratuberculosis antigen, Johnin Purified Protein Derivative (J-PPD) was used. Further, in order to confirm that PGE₂ production by antigen stimulation is inhibited by COX-2 inhibitor, 1 μg/ml of J-PPD and 1000 nM meloxicam (Signa-Aldrich) were added to the medium. After 5 days, the culture supernatant was collected, and PGE₂ contained therein was quantified with Prostaglandin E2 Express ELISA Kit (Cayman Chemical).

Experimental results of (2) are shown in FIGS. 27b and c. Addition of J-PPD significantly promoted PGE₂ production from PBMCs in experimentally infected cattle (FIG. 27c). On the other hand, no significant difference was observed in uninfected cattle (FIG. 27b). It has been demonstrated that J-PPD-promoted PGE₂ production from experimentally infected cattle is a specific response to M. avium subsp. paratuberculosis. Further, PGE₂ production was inhibited significantly by culture with a COX-2 inhibitor added under the above-described conditions (FIG. 27c).

### (3) Changes in COX2 Expression by J-PPD Stimulation

PBMCs derived from cattle experimentally infected with M. avium subsp. paratuberculosis were seeded in 96-well plates at 1×10⁶ cells/well and cultured in the presence of J-PPD for 24 hours. After culturing, PBMCs were harvested and total cellular RNA was extracted as described above. Then, cDNA was synthesized. Using the synthesized cDNA as a template, real-time PCR was performed with COX2 specific primers in the same manner as described above.

Primer (boCOX2 F):
(SEQ ID NO: 139)
ACG TTT TCT CGT GAA GCC CT
Primer (boCOX2 R):
(SEQ ID NO: 140)
TCT ACC AGA AGG GCG GGA TA

Experimental results of (3) are shown in FIG. 27d. COX2 expression was increased significantly by J-PPD stimulation in cattle experimentally infected with M. avium subsp. paratuberculosis. These results suggested that COX2 expression is increased by J-PPD stimulation in cattle experimentally infected with M. avium subsp. paratuberculosis and that this increase results in promoted PGE₂ production.

### 2.4. Changes in PD-L1 Expression by J-PPD Stimulation

Effects of J-PPD stimulation on PD-L1 expression in cattle infected with M. avium subsp. paratuberculosis were evaluated. PBMCs derived from cattle experimentally infected with M. avium subsp. paratuberculosis and those derived from uninfected cattle were seeded in 96-well plates at 1×10⁶ cells/well and cultured for 24 hours in the presence of J-PPD. Cultured PBMCs were harvested, and then PD-L1 expression on lymphocytes, CD4⁺ T cells, CD8⁺ T cells, IgM⁺ cells and CD14⁺ cells was analyzed by flow cytometry. Briefly, PBS supplemented with 10% inactivated goat serum (Thermo Fisher Scientific) was added to each well in a volume of 100 μl and cells were left stationary at room temperature for 15 min. After washing, rat anti-bovine PD-L1 antibody (4G12; Rat IgG2a; Ikebuchi R, Konnai S, Okagawa T, Yokoyama K, Nakajima C, Suzuki Y, Murata S, Ohashi K. Immunology 2014 August; 142(4):551-561) or rat IgG2a isotype control (BD Bioscience) was added and reaction was conducted at room temperature for 20 min. After washing twice, secondary antibodies were added and reaction was conducted at room temperature for 20 min. As secondary antibodies, phycoerythrin (PE)-labeled anti-CD3 monoclonal antibody (MM1A; Washington State University Monoclonal Antibody Center), fluorescein isothiocyanate (FITC)-labeled anti-CD4 monoclonal antibody (CC8; Bio-Rad), PerCp/Cy 5.5-labeled anti-CD8 monoclonal antibody (CC63; Bio-Rad), PE/Cy7-labeled anti-IgM monoclonal antibody (IL-A30; Bio-Rad) and APC-labeled anti-rat Ig antibody (Southern Biotech) were used for analysis of PD-L1 expression on T cells and IgM⁺ cells. Anti-CD3 monoclonal antibody (MM1A) was labeled with PE using Zenon Mouse IgG1 labeling Kit. For analysis of PD-L1 expression on CD14⁺ cells, PerCp/Cy5.5-labeld anti-CD14 monoclonal antibody (CAM36A; Washington State University Monoclonal Antibody Center) and APC-labeled anti-rat Ig antibody (Southern Biotech) were used. Anti-CD14 monoclonal antibody (CAM36A) was labeled with PerCp/Cy5.5 using Lightning-Link Conjugation Kit. After reaction, washing was conducted twice. Then, PD-L1 expression was analyzed with FACS Verse (BD Biosciences) and FCS Express 4 (De Novo Software). For every washing operation and dilution of antibodies, PBS supplemented with 1% bovine serum albumin (Sigma Aldrich) was used.

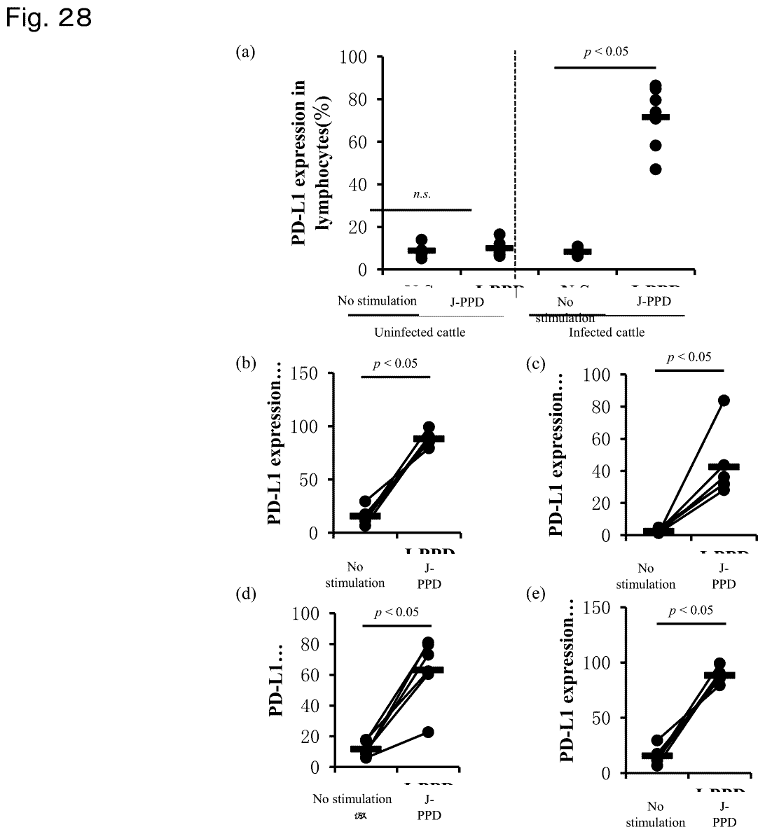

Experimental results are shown in FIG. 28. It was shown that PD-L1 expression rate is significantly increased in J-PPD-added groups of cattle experimentally infected with M. avium subsp. paratuberculosis, as compared to uninfected cattle (FIG. 28a). PD-L1 expression rates were also increased in CD4⁺ T cells, CD8⁺ T cells, IgM⁺ B cells and CD14⁺ cells in cattle infected with M. avium subsp. paratuberculosis (FIG. 28b-e).

### 2.5. Expression Analyses of PGE₂, EP2 and PD-L1 in Johne's Disease Lesions

Subsequently, the present inventors performed expression analyses of PGE₂, EP2 and PD-L1 in Johne's disease lesions by immunohistochemical staining. Briefly, ilium tissue blocks from cattle which naturally developed Johne's disease (#1, presenting clinical symptoms of Johne's disease such as diarrhea and severe emaciation), cattle experimentally infected with M. avium subsp. paratuberculosis (#65, clinical symptoms such as shedding of M. avium subsp. paratuberculosis and diarrhea were observed; Okagawa T, Konnai S, Nishimori A, Ikebuchi R, Mizorogi S, Nagata R, Kawaji S, Tanaka S, KagawaY, Murata S, Mori Y and Ohashi K., Infect Immun, 84:77-89, 2016) and uninfected control cattle (C#6) (those blocks were kindly provided by Dr. Yasuyuki Mori, National Institute of Animal Health, National Agriculture and Food Research Organization) were used for immunohistochemical staining. Samples fixed with 4% paraformaldehyde [paraformaldehyde 20 g, PBS (pH 7.4) 500 ml] and embedded in paraffin were sliced into 4 mm thick sections with a microtome, attached to and dried on silane-coated slide glass (Matsunami Glass) and deparaffinized with xylene/alcohol. While the resultant sections were soaked in citrate buffer (citric acid 0.37 g, trisodium citrate dihydrate 2.4 g, distilled water 1000 ml), antigen retrieval treatment was performed for 10 min with microwave, followed by staining using a Nichirei automatic immuno-staining device. As pretreatment, sample sections were soaked in 0.3% hydrogen peroxide-containing methanol solution at room temperature for 15 min and washed with PBS. Then, anti-PGE₂-polyclonal antibody (Abcam), anti-EP2 monoclonal antibody (EPR8030(B); Abcam) or rat anti-bovine PD-L1 monoclonal antibody (6C11-3A11; Rat IgG2a; Japanese Patent Application No. 2017-61389, Konnai S, Ohashi K, Murata S, Okagawa T, Nishimori A, Maekawa N, Takagi S, Kagawa Y, Suzuki S, Nakajima C, titled “Anti-PD-L1 Antibody for Detecting PD-L1”) was added and reaction was conducted at room temperature for 30 min. After washing with PBS, histofine simple stain MAX-PO (Nichirei Bioscience) was added and reaction was carried out at room temperature for 30 min, followed by coloring with 3,3′-diaminobenzidine tetrahydrocholride and observation with a light microscope.

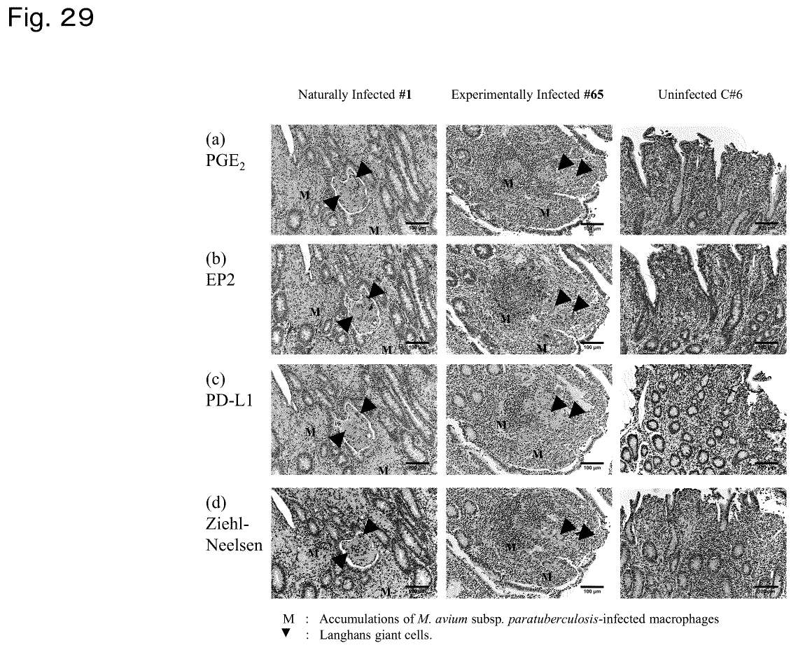

Experimental results are shown in FIG. 29. In ileal lesions of cattle (#1 cattle naturally developing johne's disease and #65 experimentally infected cattle) where M. avium subsp. paratuberculosis was confirmed by Ziehl-Neelsen staining, PGE₂, EP2 and PD-L1 were expressed strongly (FIG. 29a-d). On the other hand, in the ileum of uninfected cattle (C#6), the expression of EP2 was confirmed but PGE₂ and PD-L1 were hardly expressed (FIG. 29a-d). These results indicated a possibility that PGE₂ production and PD-L1 expression are enhanced in Johne's disease lesions.

2.6. Examination of Stimulatory Effects of COX-2 Inhibitor on M. avium subsp. paratuberculosis-Specific Immune Responses

In order to confirm that COX-2 inhibitor has stimulatory effects on M. avium subsp. paratuberculosis-specific immune responses, the present inventors cultured PBMCs derived from cattle experimentally infected with M. avium subsp. paratuberculosis in the presence of added meloxicam and J-PPD and evaluated their proliferation capacity and cytokine production capacity. Briefly, PBMCs derived from cattle experimentally infected with M. avium subsp. paratuberculosis were seeded in 96-well plates at 4×10⁵ cells/well and cultured for 5 days under stimulation in the presence of 1 μg/ml of J-PPD and 1000 nM meloxicam (Sigma-Aldrich). After culturing, cell proliferation capacity and cytokine production capacity were evaluated in the same manner as described above.

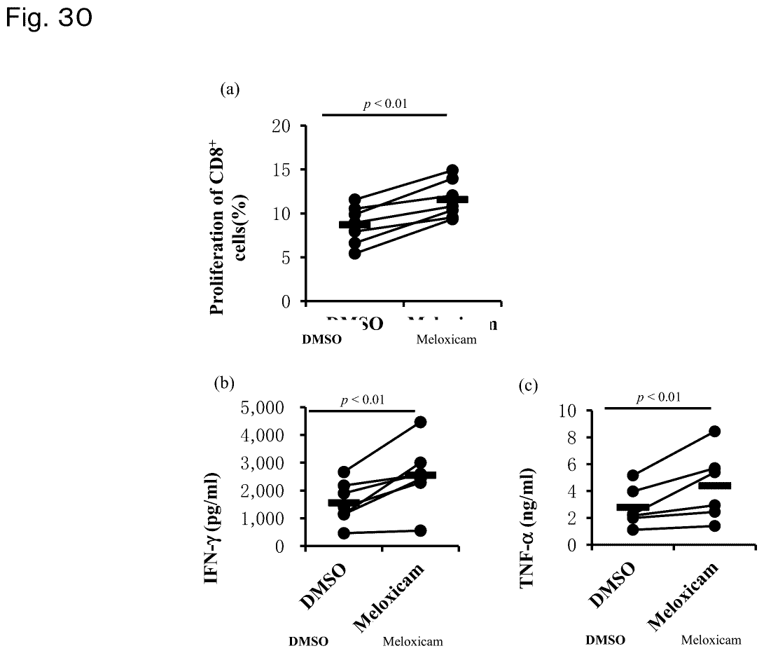

Experimental results are shown in FIG. 30. In cattle experimentally infected with M. avium subsp. paratuberculosis, a significant increase was observed in proliferation rate of CD8⁺ cells (FIG. 30a), IFN-γ production (FIG. 30b) and TNF-α production (FIG. 30c). These results demonstrated that COX-2 inhibitor has stimulatory effects on M. avium subsp. paratuberculosis-specific immune responses.

2.7. Examination of Immunostimulatory Effects of Rat Anti-Bovine PD-L1 Antibody in M avium subsp. paratuberculosis-Infected Cattle

In order to confirm that rat anti-bovine PD-L1 antibody also has immunostimulatory effects in M. avium subsp. paratuberculosis-infected cattle, the present inventors performed a PBMC culture test under stimulation in the presence of rat anti-bovine PD-L1 antibody and evaluated J-PPD-specific T-cell responses. Briefly, PBMCs derived from cattle experimentally infected with M. avium subsp. paratuberculosis were seeded in 96-well plates at 4×10⁵ cells/well and cultured for 5 days under stimulation with 1 μg/ml of J-PPD. At the time of this stimulation culture, 1 μg/ml of rat anti-bovine PD-L1 antibody (4G12; Ikebuchi R, Konnai S, Okagawa T, Yokoyama K, Nakajima C, Suzuki Y, Murata S, Ohashi K. Immunology 2014 August; 142(4):551-561) as a blocking antibody or the same amount of a rat serum-derived IgG (Sigma-Aldrich) as a negative control was added. After culturing, cell proliferation capacity and cytokine production were evaluated in the same manner as described above.

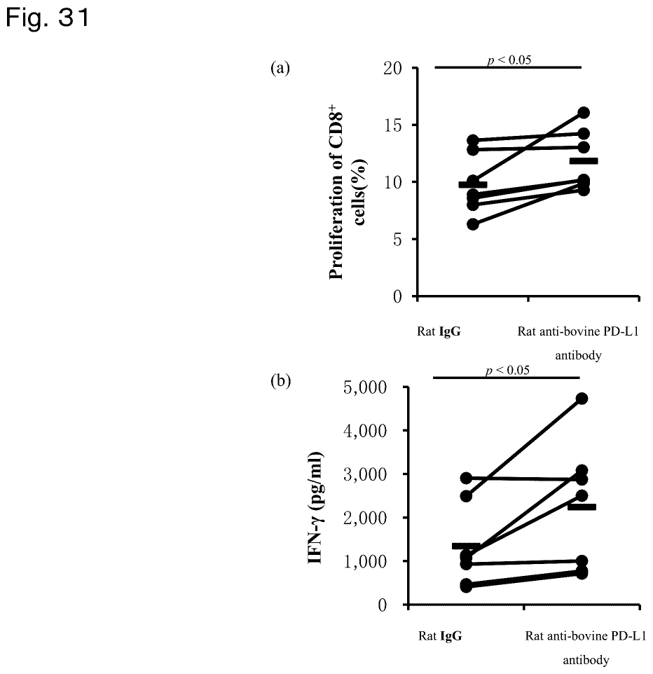

Experimental results are shown in FIG. 31. In the cattle experimentally infected with M. avium subsp. paratuberculosis, a significant increase was observed in the proliferation rate of CD8⁺ cells (FIG. 30a), IFN-γ production (FIG. 30b) and TNF-α production (FIG. 30c) as a result of the addition of rat anti-bovine PD-L1 antibody. These results indicated that PD-1/PD-L1 blockade has stimulatory effects on J-PPD-specific T-cell responses.

### 2.8. Examination of Combined Effects of COX-2 Inhibitor and Rat Anti-Bovine PD-L1 Antibody on Immunostimulation

Subsequently, the present inventors examined combined effects of COX-2 inhibitor and rat anti-bovine PD-L1 antibody on immunostimulation in cattle experimentally infected with M. avium subsp. paratuberculosis. Briefly, PBMCs derived from cattle experimentally infected with M. avium subsp. paratuberculosis were seeded in 96-well plated at 4×10⁵ cells/well and cultured in the presence of J-PPD or a negative control antigen for 5 days. As the negative control antigen, Mycobacterium bovis BCG strain-derived purified protein (B-PPD) was used. To the medium, 1,000 nM meloxicam (Sigma-Aldrich) and 1 μg/ml of rat anti-bovine PD-L1 antibody (4G12; Ikebuchi R, Konnai S, Okagawa T, Yokoyama K, Nakajima C, Suzuki Y, Murata S, Ohashi K. Immunology 2014 August; 142(4):551-561) were added to make a total volume 200 μl. As a negative control for meloxicam, DMSO was used. As a negative control antibody, rat serum-derived IgG (Sigma-Aldrich) was used. After culturing, cell proliferation capacity and cytokine production were evaluated in the same manner as described above.

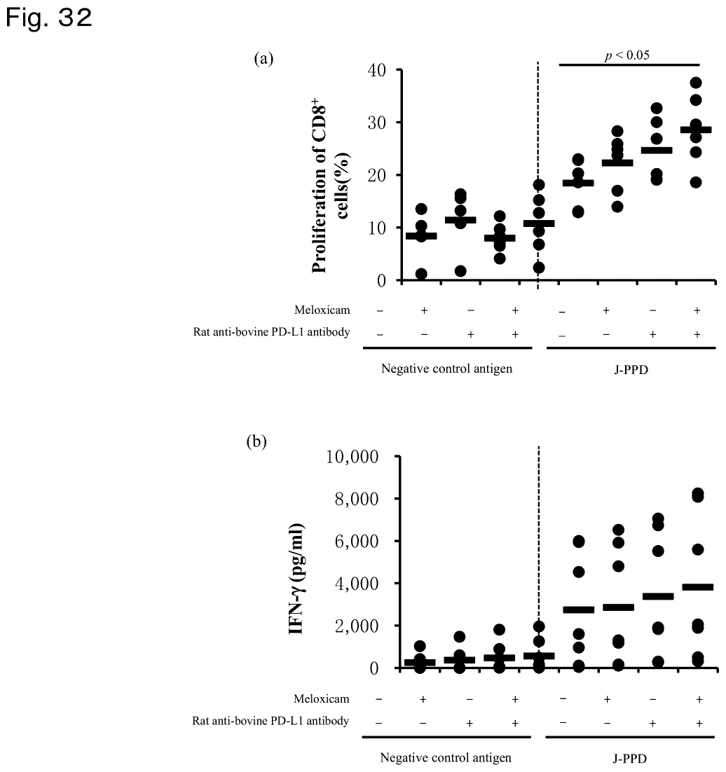

Experimental results are shown in FIG. 32. The proliferation rate of CD8⁺ cells was increased significantly in the groups where meloxicam and rat anti-bovine PD-L1 antibody had been added, as compared to the negative control groups (FIG. 32a). Since no such change was observed in the groups stimulated with the negative control antigen, a possibility was shown that combined use of COX-2 inhibitor and anti-bovine PD-L1 monoclonal antibody stimulates J-PPD-specific CD8⁺ cell responses (FIG. 32a). With respect to IFN-γ production, no significant change was observed whether J-PPD stimulation or negative control antigen stimulation was applied (FIG. 32b). These results showed a possibility that combined use of COX-2 inhibitor and rat anti-bovine PD-L1 antibody will produce a larger immunostimulatory effect in M. avium subsp. paratuberculosis-infected cattle than when the individual agents are used as single dosages.

### 2.9. Examination of Combined Effects of COX-2 Inhibitor and Rat-Bovine Chimeric Anti-PD-L1 Antibody on Immunostimulation

Finally, the present inventors examined combined effects of COX-2 inhibitor and rat-bovine chimeric anti-PD-L1 antibody on immunostimulation in cattle experimentally infected with M. avium subsp. paratuberculosis. Briefly, PBMCs derived from cattle experimentally infected with M. avium subsp. paratuberculosis were seeded in 96-well plated at 4×10⁵ cells/well and cultured in the presence of J-PPD or a negative control antigen for 5 days. As the negative control antigen, B-PPD was used. To the medium, 1,000 nM meloxicam (Sigma-Aldrich) and 1 μg/ml of rat-bovine chimeric anti-PD-L1 antibody (ch4G12; Japanese Patent Application No. 2016-159089, Konnai S, Ohashi K, Murata S, Okagawa T, Nishimori A, Maekawa N, Suzuki S, Nakajima C; Anti-PD-L1 Antibody for Cattle) were added to make a total volume 200 μl. As a negative control for meloxicam, DMSO was used. As a negative control antibody, bovine serum-derived IgG (Sigma-Aldrich) was used. After culturing, cell proliferation capacity and cytokine production were evaluated in the same manner as described above.

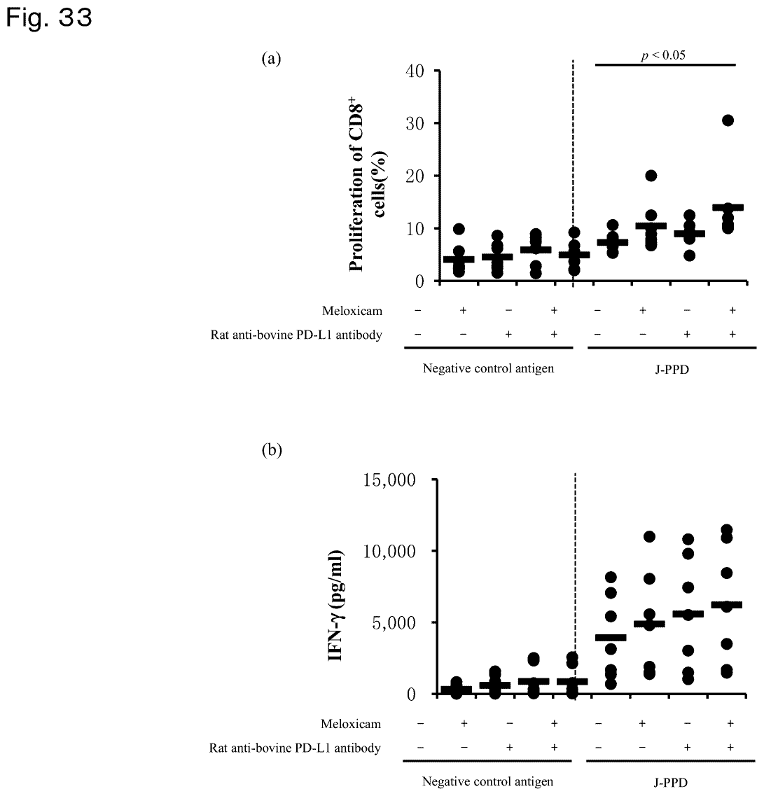

Experimental results are shown in FIG. 33. As a result of evaluation of combined effects m M. avium subsp. paratuberculosis-infected cattle, a possibility was shown that combined use of COX-2inhibitor and rat-bovine chimeric anti-PD-L1 antibody stimulates J-PPD-specific CD8⁺ cell responses (FIG. 33a) as in the case of combined use with rat anti-bovine PD-L1 antibody. With respect to IFN-γ production, no significant change was observed whether J-PPD stimulation or negative control antigen stimulation was applied (FIG. 33b). From these results, it is understood that immunostimulatory effects by combined use of COX-2 inhibitor and PD-1/PD-L1 inhibitor were shown even when rat-bovine chimeric anti-PD-L1 antibody was used in M. avium subsp. paratuberculosis-infected cattle.

### Example 3

### Examination of Combined Effects of Anti-Bovine PD-L1 Antibody and COX-2 Inhibitor in Bovine Leukemia Virus-Infected Cattle

### 1. Introduction

The interaction between PD-1 and PD-L1 is one of the major molecular mechanisms through which pathogens evade immune responses. It has been reported that inhibition of the above interaction by using an antibody which specifically binds to either of those molecules can produce anti-pathogenic effects. In the subject Example, toward establishment of a novel control method against bovine leukemia virus (BLV) infection, the present inventors have confirmed in in vitro tests an immunostimulatory effect induced by a COX-2 inhibitor and enhancement of that effect when the inhibitor is used in combination with anti-bovine PD-L1 antibody.

2. Materials and Methods, as well as Experimental Results

### 2.1. Kinetic Analysis of PGE₂ in BLV-Infected Cattle

In order to elucidate the involvement of PGE₂ in the progression of pathology in BLV infection as a bovine chronic viral infection, the present inventors performed kinetic analysis of PGE₂ in BLV-infected cattle.

(1) Measurement of Plasma PGE₂ and Analysis of Correlation with Other Indicators

First, the amount of PGE₂ contained in the plasma of BLV-infected cattle was quantified with Prostaglandin E2 Express ELISA Kit (Cayman Chemical). For the measurement, absorbance at 450 nm was measured using a microplate reader MTP-900 (Corona Electric). Further, correlation between the amount of plasma PGE₂ and the number of lymphocytes in peripheral blood or PD-L1 expression rate in IgM⁺ cells was examined. Briefly, PBMCs isolated from BLV-infected cattle were analyzed by flow cytometry to quantify the PD-L1 expression on IgM⁺ cells. First, in order to block non-specific reactions of antibody, PBS supplemented with 10% inactivated goat serum (Thermo Fisher Scientific) was added to each well in an amount of 100 μl and left stationary at room temperature for 15 min. After washing, rat anti-bovine PD-L1 antibody (4G12; Rat IgG2a; Ikebuchi R, Konnai S, Okagawa T, Yokoyama K, Nakajima C, Suzuki Y, Murata S, Ohashi K. Immunology 2014 August; 142(4):551-561) or rat IgG2a isotype control (BD Bioscience) was added and reaction was conducted at room temperature for 20 min. After washing twice, PE/Cy7-labeled anti-IgM monoclonal antibody (IL-A30; Bio-Rad) and APC-labeled anti-rat Ig antibody (Southern Biotech) were added and reaction was conducted at room temperature for 20 min. Anti-IgM monoclonal antibody (IL-A30) was labeled with PE/Cy7 using Lightning-Link Conjugation Kit. After the reaction, washing was performed twice. Then, cells were analyzed with FACS Verse (BD Biosciences) and FCS Express 4 (De Novo Software). For every washing operation and dilution of antibodies, PBS supplemented with 1% bovine serum albumin (Sigma Aldrich) was used.

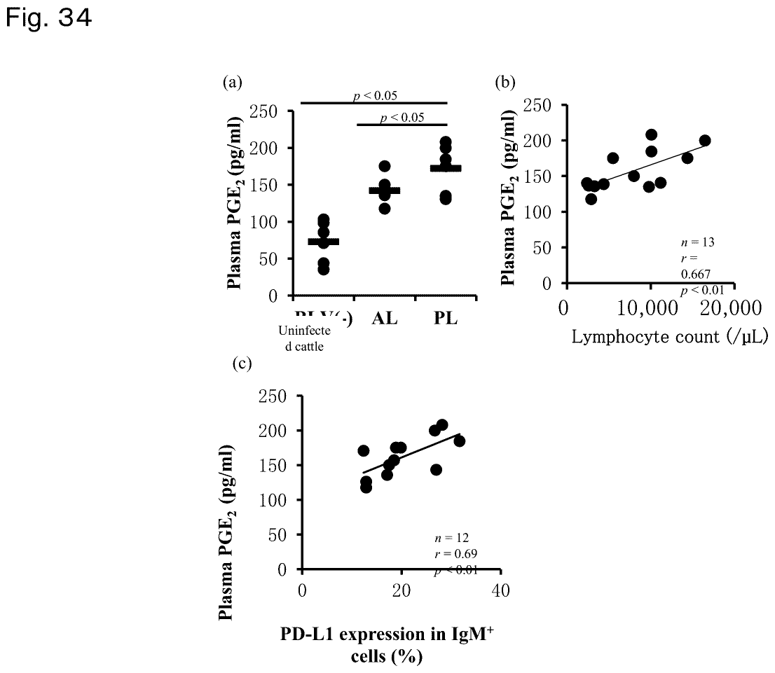

Experimental results of (1) are shown in FIG. 34. As the disease stage of BLV infection progressed (AL: aleukemic, asymptomatic; PL: persistent lymphocytosis), plasma PGE₂ increased significantly (FIG. 34a). As a result of examination of correlation between the number of lymphocytes in peripheral blood (an indicator of the disease progression in BLV infection) and plasma PGE₂, a positive correlation was observed (FIG. 34b). Further, as a result of examination of correlation between plasma PGE₂ and PD-L1 expression rate in IgM⁺ cells, a positive correlation was shown (FIG. 34c). These results suggested that PGE₂ is involved in the disease progression in BLV infection and the resultant immunosuppression.

### (2) Expression Analysis of COX2 and EP4 in BLV-Infected Cattle

For more detailed analysis, in addition to plasma PGE₂, expression levels of COX2 (i.e., involved in PGE₂ synthesis) and the gene of EP4 (a PGE₂ receptor that transmits immunosuppressive signals) were quantified by real-time PCR. Briefly, total cellular RNA was extracted from PBMCs, CD4⁺ cells, CD8⁺ cells, CD14⁺ cells and CD21⁺ cells derived from BLV-infected cattle and uninfected cattle in the same manner as described in (3), section 2.1 of Example 2, and cDNA was synthesized. Using the synthesized cDNA as a template, real-time PCR was performed with COX2-specific primers (already described in (3), section 2.3 of Example 2) and EP4-specific primers in the same manner as described in Example 2.

Primer (boEP4 F):
(SEQ ID NO: 141)
GTG ACC ATC GCC ACC TAC TT
Primer (boEP4 R):
(SEQ ID NO: 142)
CTC ATC GCA CSG ATG ATG CT

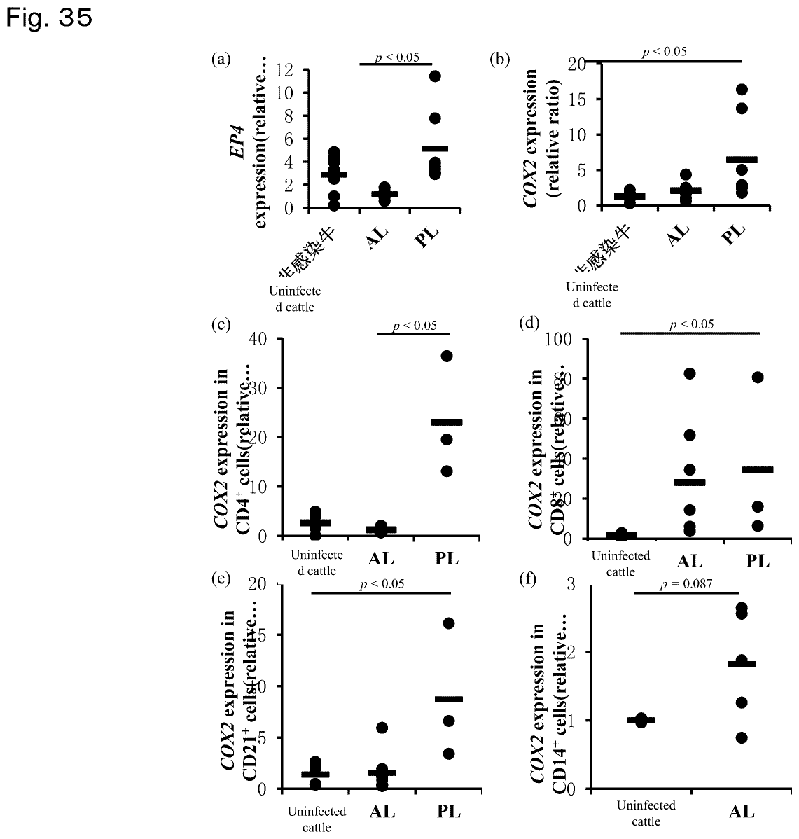

Experimental results of (2) are shown in FIG. 35. It was confirmed that EP4 expression level increased in cattle with PL (an advanced stage of the disease) (hereinafter, referred to as “PL cattle”) (FIG. 35a). The same is true for COX2 expression levels, which also increased in PL cattle (FIG. 35b). In order to examine PGE₂ producing cells, COX2 expression in CD4⁺, CD8⁺, CD14 and CD21⁺ cells was quantified. As it turned out, COX2 expression clearly increased in CD4⁺, CD8⁺ and CD21⁺ cells in PL cattle (FIG. 35c-e). With respect to CD14 cells, evaluation using samples from PL cattle is yet to be performed, but COX2 expression showed a tendency to increase in AL cattle compared to uninfected cattle (FIG. 35f). These results showed that COX2 expression increases in various cell groups as the disease stage of BLV infection progresses.

### (3) Changes in PGE₂ Production by BLV Antigen Stimulation

It has been reported that COX2 expression is increased by antigen stimulation in BLV infection (Pyeon D, Diaz F J, Splitter G A. J Virol. 74:5740-5745, 2000). From this report, it is predicted that PGE₂ production by PBMCs derived from BLV-infected cattle will also be promoted by antigen stimulation. To verify this hypothesis, the present inventors cultured PBMCs with BLV antigen and quantified PGE₂ in the culture supernatant by ELISA. Briefly, PBMCs derived from BLV-infected cattle and those derived from uninfected cattle were seeded in 96-wel plates at 4×10⁵ cells/well and cultured in the presence of BLV antigen (final concentration 2%) for 6 days. As the BLV antigen, a culture supernatant of fetal lamb kidney cells persistently infected with BLV (FLK-BLV) was used after heat treatment at 65° C. In order to confirm that PGE₂ production by antigen stimulation is inhibited by COX-2 inhibitor, cells were also cultured under such conditions that both BLV antigen and 1,000 nM meloxicam (Sigma Aldrich) were added to the medium. After 6 days, culture supernatant was collected and PGE₂ contained in it was quantified with Prostaglandin E2 Express ELISA Kit (Cayman Chemical).

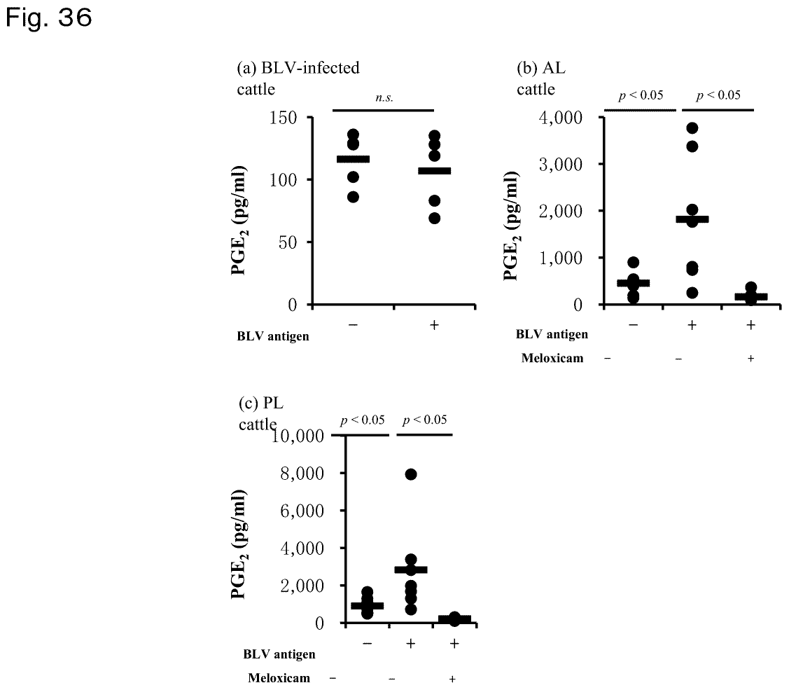

Experimental results of (3) are shown in FIG. 36. In BLV-infected cattle (AL: FIG. 36b; PL: FIG. 36c), PGE₂ production from PBMCs was shown to be significantly promoted by addition of BLV antigen (FIG. 36b, c). No significant differences were observed in uninfected cattle (FIG. 36a). Thus, it has been demonstrated that the induction of PGE₂ production stimulated by the BLV antigen in PBMCs derived from BLV-infected cattle is a response specific to BLV. Further, it was confirmed that PGE₂ production is significantly inhibited by culture in the presence of an added COX-2 inhibitor under the above-described conditions (FIG. 36b, c).

### (4) Effect of PGE₂ on BLV Provirus Load

In various chronic infections, a possibility has been suggested that PGE₂ promotes viral replication (Pyeon D, Diaz F J, Splitter G A. J Virol. 74:5740-5745, 2000; Waris D, Siddiqui A. J Virol. 79:9725-9734, 2005). Then, in order to evaluate the effect of PGE₂ on viral replication in BLV infection, the present inventors cultured PBMCs with PGE₂ and quantified BLV provirus load by real-time PCR. Briefly, PBMCs derived from BLV-infected cattle were seeded in 96-well plates at 1×10⁶ cells/well and cultured in the presence of 2,500 nM PGE₂ or DMSO for 3 days. After culturing, DNA was extracted from harvested PBMCs with Wizard DNA Purification kit (Promega). The concentration of the extracted DNA was quantified by measuring the absorbance (260 nm) with Nanodrop 8000 Spectrophotometer (Thermo Fisher Scientific). For measuring BLV provirus load in PBMCs, real-time PCR was performed using Cycleave PCR Reaction Mix SP (TaKaRa) and Probe/Primer/Positive control for detecting bovine leukemia virus (TaKaRa). LightCycler480 System II (Roche Diagnosis) was used for measurement.

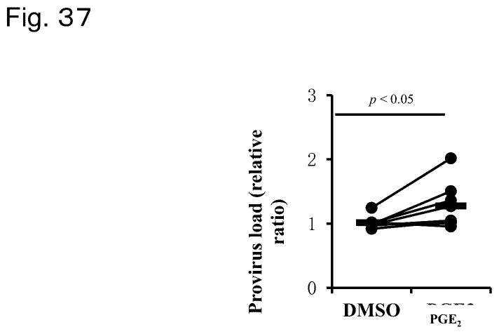

Experimental results of (4) are shown in FIG. 37. Provirus loads were shown to increase significantly in PGE₂-treated group (FIG. 37), suggesting a possibility that PGE₂ promotes BLV replication.

### 2.2. Changes in PD-L1 Expression by BLV Antigen Stimulation

Effects of BLV antigen stimulation on PD-L1 expression in BLV-infected cattle were evaluated. Briefly, PBMCs derived from BLV-infected and those from uninfected cattle were seeded in 96-well plates at 1×10⁶ cells/well and cultured in the presence of BLV antigen (final concentration 2%) for 24 hours. After culturing, PBMCs were harvested, and PD-L1 expression on lymphocytes, CD4⁺ T cells, CD8⁺ T cells, IgM⁺ cells and CD14⁺ cells was analyzed by flow cytometry in the same manner as described in section 2.4 of Example 2.

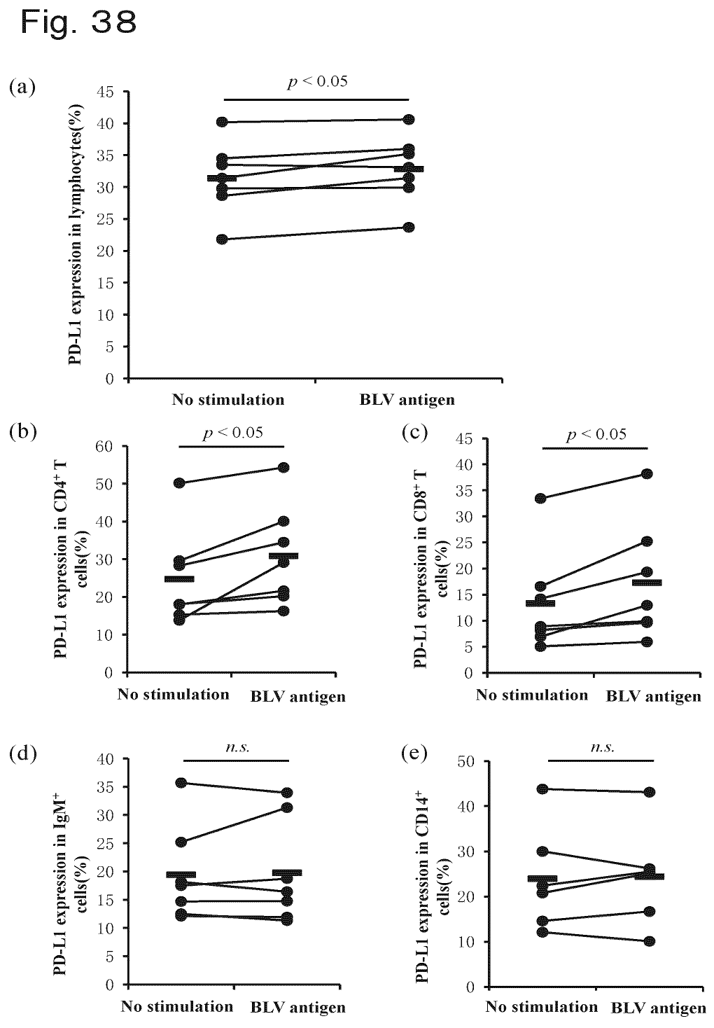

Experimental results of 2.2 are shown in FIG. 38. PD-L1 expression was shown to increase significantly in lymphocytes when PBMCs from BLV-infected cattle were stimulated with BLV antigen (FIG. 38a). Further, changes in PD-L1 expression in individual cell groups (CD4⁺ T cells, CD8⁺ T cells, IgM⁺ cells and CD14⁺ cells) were analyzed to reveal that PD-L1 expression was increased in CD4⁺ cells and CD8⁺ cells by BLV antigen stimulation (FIG. 38b-e).

### 2.3. Examination of Stimulatory Effects of COX-2 Inhibitor on BLV-Specific Immune Responses

In order to confirm that COX-2 inhibitor has stimulatory effects on BLV antigen-specific immune responses, the present inventors evaluated the proliferation capacity and cytokine production capacity of PBMCs that were cultured in the presence of meloxicam and BLV antigen. Briefly, PBMCs derived from BLV-infected cattle were seeded in 96-well plated at 4×10⁵ cells/well and cultured under stimulation in the presence of BLV antigen (final concentration 2%) and 1,000 nM meloxicam (Sigma-Aldrich) for 6 days. After culturing, cell proliferation capacity and cytokine production capacity were evaluated in the same manner as described in Example 2.

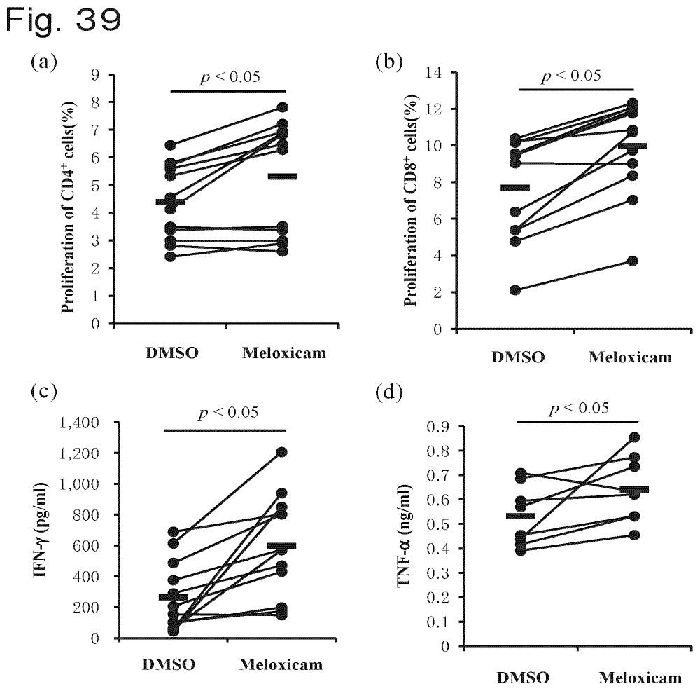

Experimental results are shown in FIG. 39. In the cattle experimentally infected with BLV, a significant increase was observed in proliferation rate of CD4⁺ cells (FIG. 39a), proliferation rate of CD8⁺ cells (FIG. 39b), IFN-γ production (FIG. 39c) and TNF-α production due to the addition of meloxicam. These results indicated that COX-2 inhibitor has stimulatory effects on BLV antigen-specific T cell responses.

### 2.3. Examination of Antiviral Effects of COX-2 Inhibitor in BLV-Infected Cattle

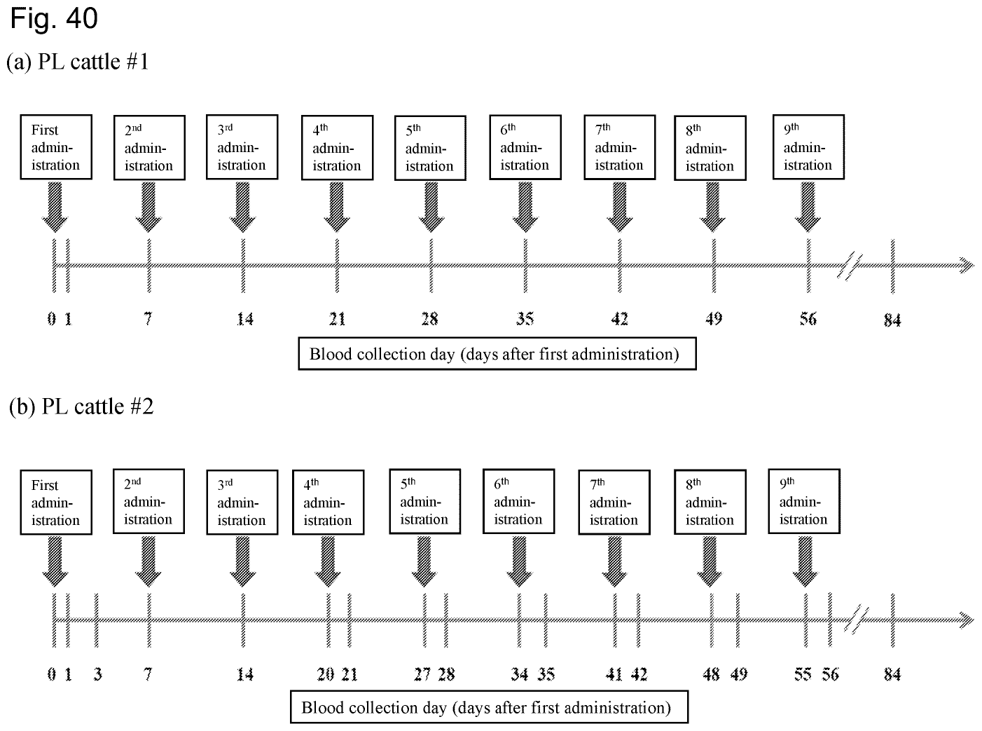

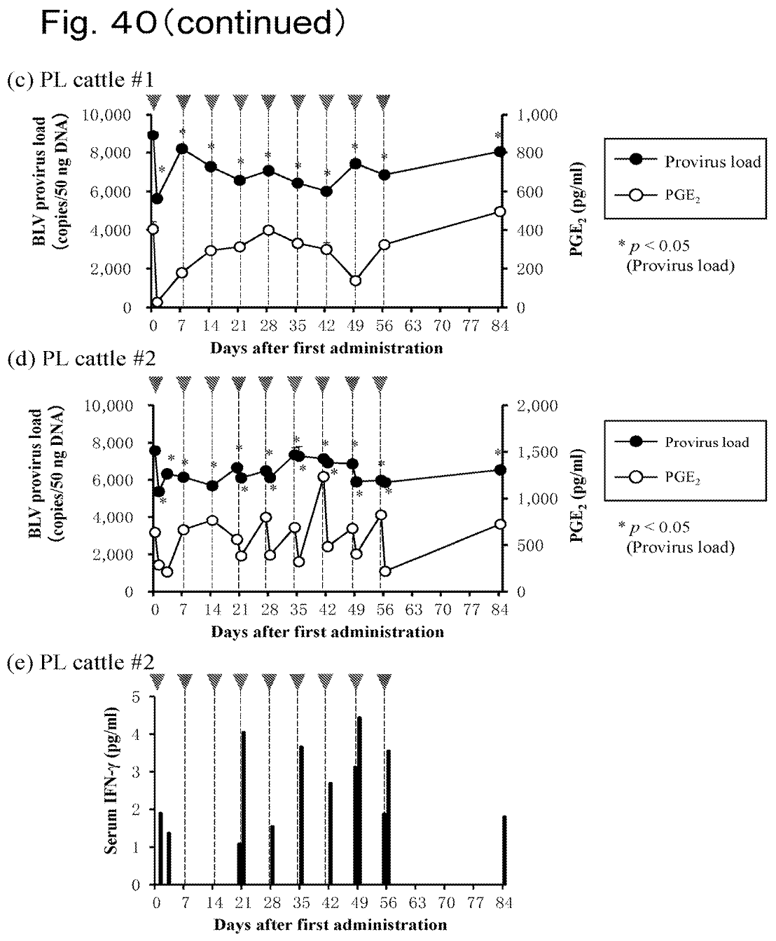

In order to examine the antiviral effects of COX-2 inhibitor in vivo, the present inventors conducted a clinical application test using BLV-infected cattle. Two individuals (#1 and #2) of PL cattle of Holstein species were used in the test. The body weight and age at the beginning of the test were 736 kg and 8 years and 1 month for #1 and 749 kg and 3 years and 7 months for #2. As a COX-2 inhibitor, Metacam™ 2% injection (hereinafter, referred to as “Metacam™”; Kyoritsu Seiyaku) was inoculated subcutaneously at 0.5 mg/kg. In addition to the first inoculation, individual #1 received inoculation 7, 14, 21, 28, 35, 42, 49 and 56 days after the first inoculation; and individual #2 received inoculation 7, 14, 20, 27, 34, 41, 48 and 55 days after the first inoculation (FIG. 40a, b). Blood collection was performed on each inoculation day, and 1 day and 84 days after the first inoculation for individual #1; and for individual #2, each inoculation day, the day after each inoculation, and 3 days and 84 days after the first inoculation (FIG. 40a, b). Blood collection on each inoculation day was conducted before Metacam™ inoculation. Using the collected blood samples, BLV provirus loads, serum PGE₂ concentrations and IFN-γ concentrations were quantified. Provirus loads were quantified in the same manner as described in (4), section 2.1 above. Serum PGE₂ concentrations and IFN-γ concentrations were measured with Prostaglandin E2 Express ELISA Kit (Cayman Chemical) and ELISA for Bovine IFN-γ (MABTECH), respectively.

Experimental results are shown in FIG. 40. As a result of Metacam™ administration, BLV provirus load decreased significantly in both #1 and #2 (FIG. 40c, d). Serum PGE₂ concentration decreased on the day after Metacam™ administration, and provirus load also decreased on the day after Metacam™ administration (FIG. 40c, d). Further, in #2, serum IFN-γ concentration increased on the day after Metacam™ administration (FIG. 40e). In #1, serum IFN-γ concentration was below the measurement limit of ELISA. These results indicated that COX-2 inhibitor has antiviral effects in BLV-infected cattle in vivo.

### 2.4. Examination of Immunostimulatory Effects Due to Combined Use of COX-2 Inhibitor and Rat Anti-Bovine PD-L1 Antibody

Provirus loads in BLV-infected cattle decreased upon administration of a COX-2 inhibitor (FIG. 40c, d). In order to obtain stronger antiviral effects, the present inventors examined immunostimulatory effects of combined use of COX-2 inhibitor and anti-bovine PD-L1 antibody in BLV-infected cattle. Briefly, PBMCs derived from BLV-infected cattle were seeded in 96-well plates at 4×10⁵ cells/well and cultured in the presence of BLV antigen or a negative control antigen for 6 days. As the negative control antigen, a culture supernatant of BLV-uninfected fetal lamb kidney cells (FLK) was used after heat treatment at 65° C. To the medium, 1,000 nM meloxicam (Sigma-Aldrich) and 1 μg/ml of rat anti-bovine PD-L1 antibody (4G12; Ikebuchi R, Konnai S, Okagawa T, Yokoyama K, Nakajima C, Suzuki Y, Murata S, Ohashi K. Immunology 2014 August; 142(4):551-561) were added to make a total volume 200 μl. As a negative control for meloxicam, DMSO was used; and as a negative control antibody, rat serum derived IgG (Sigma-Aldrich) was used. After culturing, cell proliferation capacity and cytokine production capacity were evaluated in the same manner as described above.

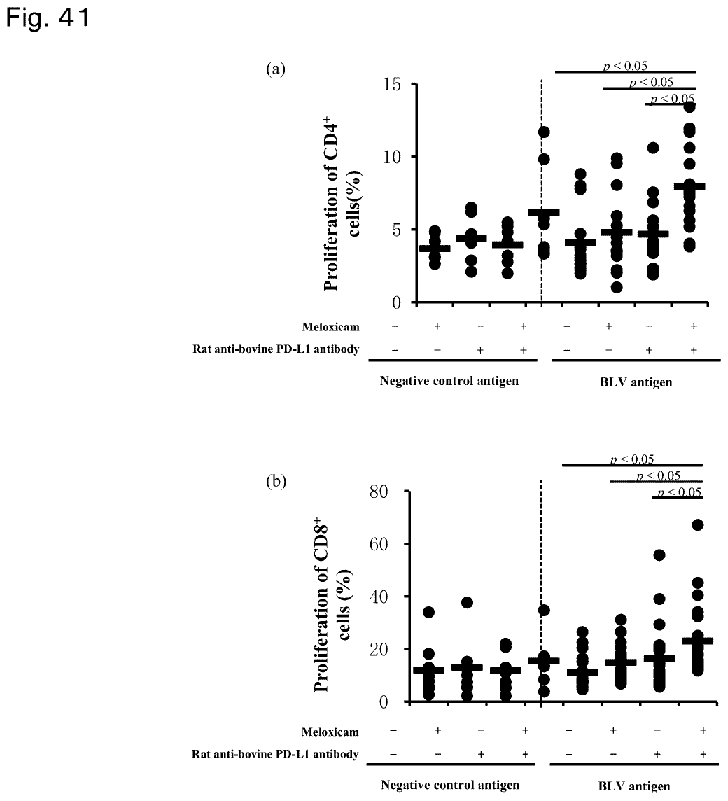

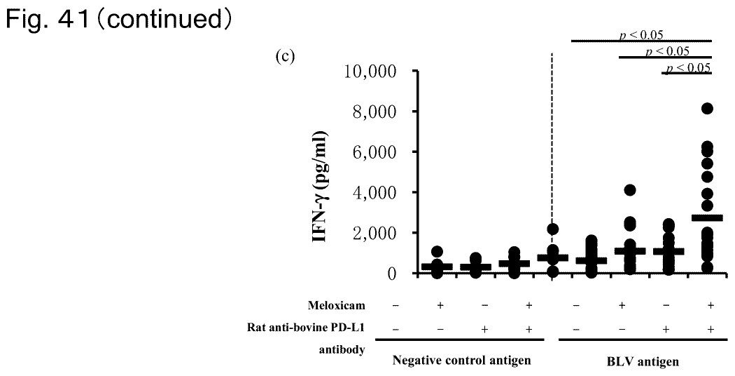

Experimental results are shown in FIG. 41. When PBMCs were stimulated with BLV antigen, the group in which both meloxicam and rat anti-bovine PD-L1 antibody were added showed significant increases in proliferation rate of CD4⁺ cells (FIG. 41a), proliferation rate of CD8⁺ cells (FIG. 41b) and IFN-γ production (FIG. 41c), as compared to the negative control group, the meloxicam alone group and the rat anti-bovine PD-L1 antibody alone group. No such changes were observed when PBMCs were stimulated with the negative control antigen. Therefore, it was suggested that BLV antigen-specific T cell responses are activated by combined use of COX-2 inhibitor and rat anti-bovine PD-L1 antibody (FIG. 41a-c). From these results, a possibility was shown that a greater immunostimulatory effect may be obtained from combined use of COX-2 inhibitor and rat anti-bovine PD-L1 antibody in BLV-infected cattle than when the inhibitor or the antibody is used alone.

### 2.5. Examination of Immunostimulatory Effects due to Combined Use of COX-2 Inhibitor and Rat-Bovine Chimeric Anti-PD-L1 Antibody

Finally, the present inventors examined immunostimulatory effects due to combined use of COX-2 inhibitor and rat-bovine chimeric anti-PD-L1 antibody in BLV-infected cattle. Briefly, PBMCs derived from BLV-infected cattle were seeded in 96-well plates at 4×10⁵ cells/well and cultured in the presence of BLV antigen or a negative control antigen for 6 days. As the negative control antigen, a culture supernatant of BLV-uninfected fetal lamb kidney cells (FLK) was used after heat treatment at 65° C. To the medium, 1,000 nM meloxicam (Sigma-Aldrich) and 1 μg/ml of rat-bovine chimeric anti-PD-L1 antibody (ch4G12; Japanese Patent Application No. 2016-159089, Konnai S, Ohashi K, Murata S, Okagawa T, Nishimori A, Maekawa N, Suzuki S, Nakajima C; Anti-PD-L1 Antibody for Cattle) were added to make a total volume of 200 μl. As a negative control for meloxicam, DMSO was used. As a negative control antibody, bovine serum-derived IgG (Sigma-Aldrich) was used. After culturing, cell proliferation capacity and cytokine production were evaluated in the same manner as described above.

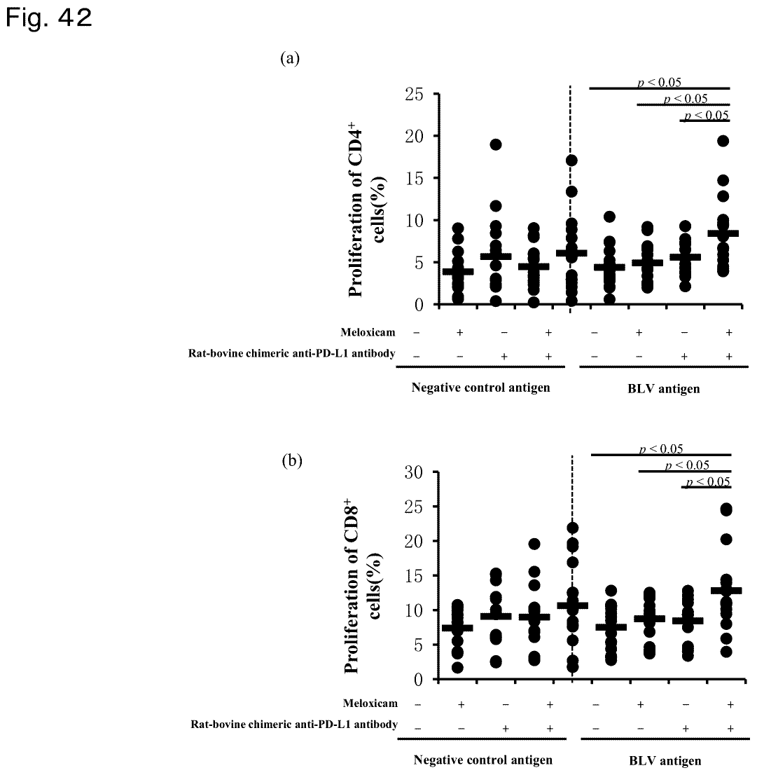

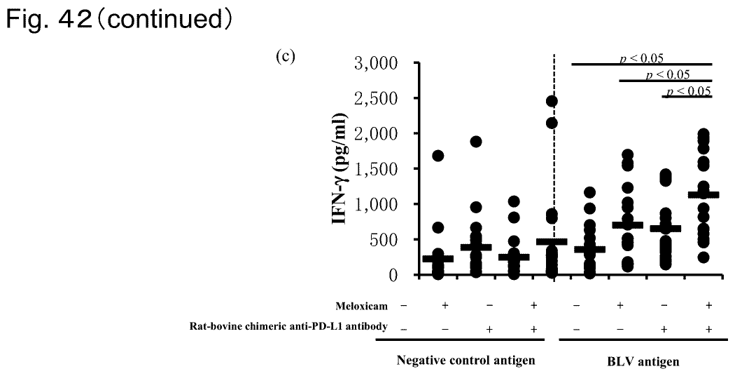

Experimental results are shown in FIG. 42. In BLV-infected cattle, BLV antigen-specific CD4⁺ cell response, CD8⁺ cell response and IFN-γ production were activated by combined use of COX-2 inhibitor and rat-bovine chimeric anti PD-L1 antibody (FIG. 42a-c), as seen in the case where rat anti-bovine PD-L1 antibody was used. Thus, in BLV-infected cattle, the immunostimulatory effect of combined use of COX-2 inhibitor and PD-1/PD-L1 inhibitor was also shown when rat-bovine chimeric anti-PD-L1 antibody was used.

### 2.6. Examination of In Vivo Antiviral Effect Due to Combined Use of COX-2 Inhibitor and Rat-Bovine Chimeric Anti-PD-L1 Antibody

In order to examine the in vivo antiviral effect of combined use of COX-2 inhibitor and PD-1/PD-L1 inhibitor, the present inventors conducted a clinical application test using BLV-infected cattle. Two individuals (#1719 and #2702; Holstein species) of BLV-infected cattle with a high BLV provirus load were used in the test. The body weight and age at the beginning of the test were 799 kg and 7 years and 4 months for #1719 and 799 kg and 4 years and 3 months for #2702. As a COX-2 inhibitor, Metacam™ 20 injection (hereinafter, referred to as “Metacam™”; Kyoritsu Seiyaku) was inoculated subcutaneously at 0.5 mg/kg. As a PD-1/PD-L1 inhibitor, rat-bovine chimeric anti-PD-L1 antibody (ch4G12; WO2018/034225, Konnai S, Ohashi K, Murata S, Okagawa T, Nishimori A, Maekawa N, Suzuki S, Nakajima C; Anti-PD-L1 Antibody for Cattle) was administered intravenously at 1.0 mg/kg. In addition to the first inoculation, Metacam™ was inoculated 7 and 14 days after the first inoculation. Blood collection was performed 7 days before the antibody administration (at −7 day); at the antibody administration day; at day 1, day 3 and day 7 after the antibody administration; and once weekly from day 14 to day 58 after the antibody administration. Blood collection on antibody/Metacam™ administration day (at day 0) and Metacam™ administration days (at day 7 and day 14) was carried out before administration of antibody and Metacam™. Using the collected blood samples, BLV provirus loads were quantified. Provirus loads were quantified in the same manner as described in (4), section 2.1 above.

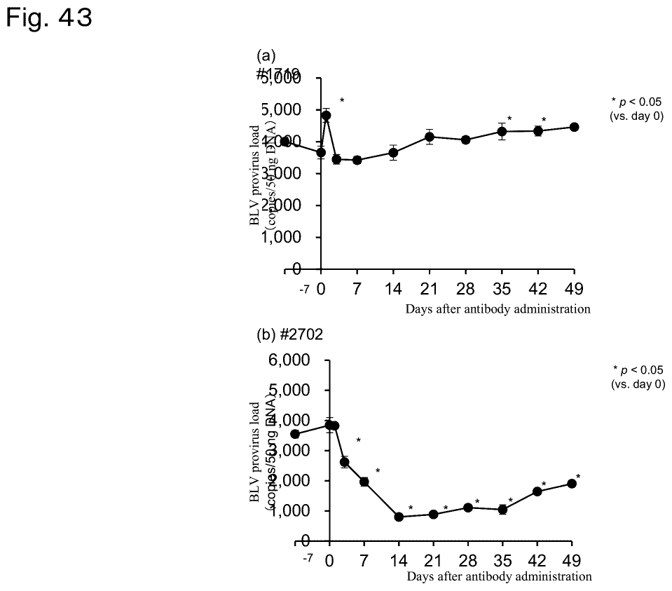

Experimental results are shown in FIG. 43. BLV provirus load on the antibody administration day (immediately before antibody administration) was 3,662 copies/50 ng DNA in #1719 and 3,846 copies/50 ng DNA in #2702. In #1719 which received rat-bovine chimeric anti-PD-L1 antibody alone, no decrease of BLV provirus load was recognized (FIG. 43a), whereas in #2702 which received a combination of Metacam™ and rat-bovine chimeric anti-PD-L1 antibody, significant decreases of BLV provirus load were recognized from day 3 to day 49 after administration (FIG. 43b). These results showed that in vivo antiviral effect is enhanced by combined use of COX-2 inhibitor and PD-1/PD-L1 inhibitor. Such combined effect will be obtained when BLV provirus load is about 3,846 copies/50 ng DNA or less. It should be noted here that this value is based on the BLV provirus load measured in the same manner as described in (4), section 2.1 above.

### Example 4

Examination of Combined Effect of Anti-Bovine PD-L1 Antibody and COX-2 Inhibitor in Mycoplasma bovis-Infected Cattle

### 1. Introduction

The interaction between PD-1 and PD-L1 is one of the major molecular mechanisms through which pathogens evade immune responses. It has been reported that inhibition of the above interaction by using an antibody which specifically binds to either of those molecules can produce anti-pathogenic effects. In the subject Example, toward establishment of a novel control method against bovine mycoplasma infections caused by Mycoplasma bovis, the present inventors have confirmed in in vitro tests, an immunostimulatory effect induced by COX-2 inhibitor and enhancement of that effect when the inhibitor is used in combination with anti-bovine PD-L1 antibody.

### 2. Materials and Methods, as Well as Experimental Results

2.1. Analysis of Serum PGE₂ in M. bovis-Infected Cattle

In order to elucidate the involvement of PGE₂ in the disease progression of bovine mycoplasmosis caused by Mycoplasma bovis, the present inventors performed kinetic analysis of PGE₂ in M. bovis-infected cattle. The amounts of PGE₂ contained in the serum of M. bovis-infected cattle and M. bovis-uninfected cattle (hereinafter, referred to as “uninfected cattle”) were quantified with Prostaglandin E2 Express ELISA Kit (Cayman Chemical). For measurement, absorbance at 450 nm was measured using a microplate reader MTP-900 (Corona Electric).

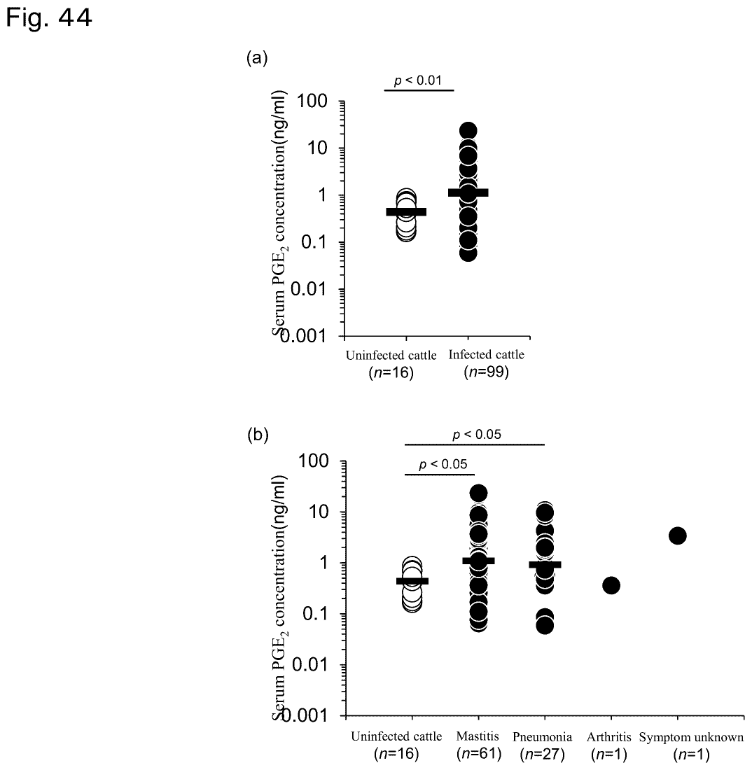

Experimental results are shown in FIG. 44. Serum PGE₂ was significantly higher M bovis-infected cattle than in uninfected cattle (FIG. 44a). Further, M. bovis-infected cattle were classified into groups by clinical symptom, and the concentrations of serum PGE₂ were compared between groups. Those individuals which presented mastitis and pneumonia due to M. bovis showed significantly high PGE₂ levels in serum as compared to the uninfected cattle (FIG. 44b). These results suggested a possibility that PGE₂ is involved in the disease progression of bovine mycoplasmosis.

2.2. Correlation Analysis between Plasma PGE₂ and Indicators of Immune Responses

Subsequently, the present inventors examined correlation between the amount of plasma PGE₂ in M. bovis-infected cattle and M. bovis-specific IFN-γ responses or PD-L1 expression rate in CD14′ cells. First, plasma was isolated from the blood of M. bovis-infected cattle, and the amount of PGE₂ contained in the plasma was measured as described in 2.1 above. Subsequently, peripheral blood mononuclear cells (PBMCs) isolated from the blood of M bovis-infected cattle were suspended in RPMI 1640 medium (Sigma-Aldrich) supplemented with 10% inactivated fetal bovine serum (Thermo Fisher Scientific), antibiotics (streptomycin 100 μg/ml, penicillin 100 U/ml) (Thermo Fisher Scientific) and 2 mM L-glutamine (Thermo Fisher Scientific) and seeded in 96-well plates (Corning) at 4×10⁵ cells/well. Then, 1.5 μg/ml of M. bovis antigen was added and cells were cultured under stimulation at 37° C. in the presence of 5% CO₂ for 5 days. As M. bovis antigen, M. bovis PG45 strain (ATCC 25523; kindly provided by Prof Hidetoshi Higuchi, Rakuno Gakuen University) was used after heat treatment. After 5 days, culture supernatant was collected and IFN-γ production was measured with ELISA for Bovine IFN-γ (MABTECH). For the measurement, absorbance at 450 nm was measured using a microplate reader MTP-900 (Corona Electric).

Further, PD-L1 expression on CD14⁺ cells was measured by flow cytometry analysis of PBMCs derived from M. bovis-infected cattle. First, in order to block non-specific reactions of antibody, PBS supplemented with 10% inactivated goat serum (Thermo Fisher Scientific) was added to each well in an amount of 100 μl and left stationary at room temperature for 15 mn. After washing, rat anti-bovine PD-L1 antibody (4G12; Rat IgG2a; Ikebuchi R, Konnai S, Okagawa T, Yokoyama K, Nakajima C, Suzuki Y, Murata S, Ohashi K. Immunology 2014 August; 142(4):551-561) or rat IgG2a isotype control (BD Bioscience) was added and reaction was conducted at room temperature for 20 min. After washing twice, PerCp/Cy5.5-labeled anti-CD14 monoclonal antibody (CAM36A; Washington State University Monoclonal Antibody Center) and APC-labeled anti-rat Ig antibody (Southern Biotech) were reacted with cells. Anti-CD14 monoclonal antibody (CAM36A) was labeled with PerCp/Cy5.5 using Lightning-Link Conjugation Kit. After the reaction, washing was performed twice. Then, cells were analyzed with FACS Verse (BD Biosciences) and FCS Express 4 (De Novo Software). For every washing operation and dilution of antibodies, PBS supplemented with 1% bovine serum albumin (Sigma Aldrich) was used.

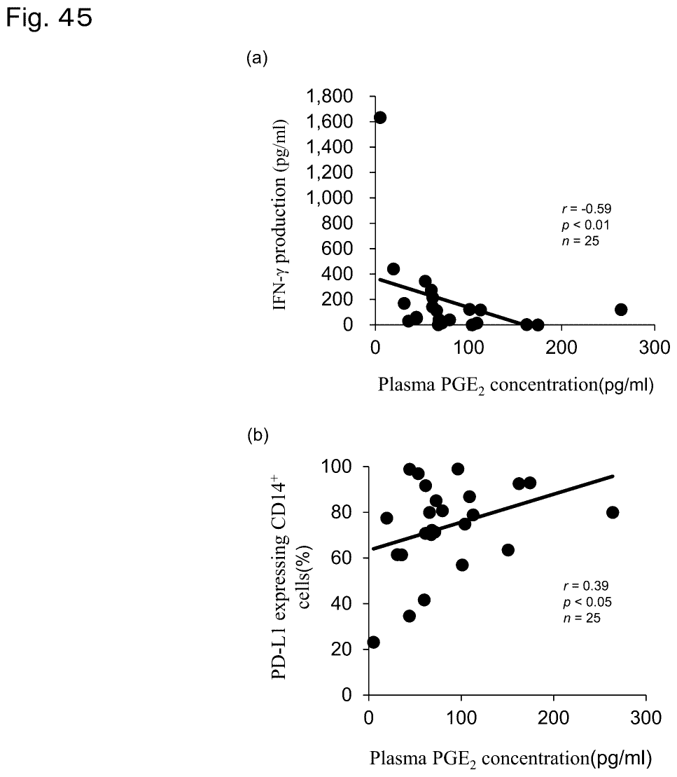

Experimental results are shown in FIG. 45. In M. bovis-infected cattle, a negative correlation was recognized between plasma PGE₂ and M. bovis-specific IFN-γ response (FIG. 45a). Further, positive correlation was observed between plasma PGE₂ and PD-L1 expression in CD14 cells (FIG. 45b). These results suggested that PGE₂ is involved in immunosuppression resulting from bovine mycoplasmosis.

2.3. Expression Analysis of COX2 and EP4 in M. bovis-Infected Cattle

For more detailed analysis, expression levels of COX2 (involved in PGE₂ synthesis) and the gene of EP4 (a PGE₂ receptor that transmits immunosuppressive signals) were quantified by real-time PCR. Briefly, total cellular RNA was extracted from PBMCs derived from M. bovis-infected cattle and those from uninfected cattle in the same manner as described in (3), section 2.1 of Example 2, and cDNA was synthesized. Using the synthesized cDNA as a template, real-time PCR was performed with COX2-specific primers (described in (3), section 2.3 of Example 2) and EP4-specific primers (described in (2), section 2.1 of Example 3) according to the method described in Example 2.

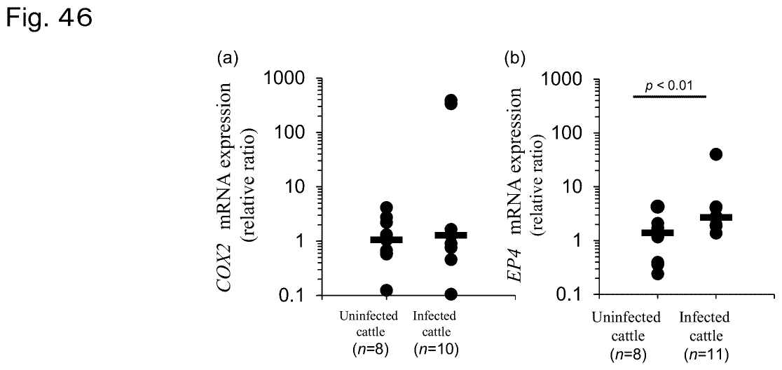

Experimental results are shown in FIG. 46. Although PBMCs from M. bovis-infected cattle showed no significant difference in COX2 expression as compared to those from uninfected cattle (FIG. 46a), they showed significant increases in EP4 expression (FIG. 46b).

2.4. Examination of Immunostimulatory Effects due to Combined Use of COX-2 Inhibitor and Rat Anti-Bovine PD-L1 Antibody in M. bovis-Infected Cattle

Finally, the present inventors examined immunostimulatory effects due to combined use of COX-2 inhibitor and anti-bovine PD-L1 antibody in M. bovis-infected cattle. Briefly, PBMCs derived from M. bovis-infected cattle were seeded in 96-well plates (Corning) at 4×10⁵ cells/well and cultured in the presence of 1.5 μg/ml of M. bovis antigen (as antigen-specific stimulant) or 2 μg/ml each of anti-CD3 monoclonal antibody (MM1A; Washington State University Monoclonal Antibody Center) and anti-CD28 monoclonal antibody (CC220; Bio-Rad) (as T cell stimulants) for 5 days. To the medium, 10 μM meloxicam (Sigma-Aldrich) and 10 μg/ml of rat anti-bovine PD-L1 antibody (4G12; Ikebuchi R, Konnai S, Okagawa T, Yokoyama K, Nakajima C, Suzuki Y, Murata S, Ohashi K. Immunology 2014 August; 142(4):551-561) were added. As a negative control for meloxicam, DMSO was used; and as a negative control antibody, rat serum-derived IgG (Sigma-Aldrich) was used. After culturing, cell proliferation capacity and cytokine production were evaluated in the same manner as described above.

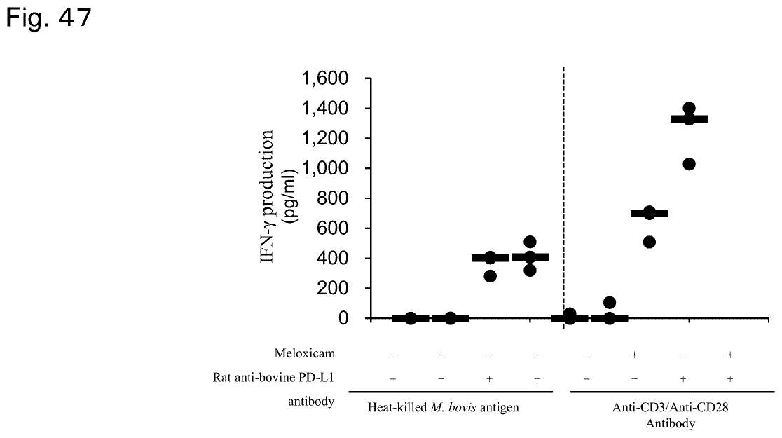

Experimental results are shown in FIG. 47. Under stimulation with M. bovis antigen, IFN-γ production tended to increase in the group which received rat anti-bovine PD-L1 antibody alone or in the group which received the combination of meloxicam and rat anti-bovine PD-L1 antibody, compared to the negative control group and the group that received meloxicam alone (FIG. 47). Under stimulation with anti-CD3 antibody and anti-CD28 antibody, IFN-γ production tended to increase in the group which received rat anti-bovine PD-L1 antibody alone, compared to the negative control group and the group that received meloxicam alone. In the group which received the combination of meloxicam and rat anti-bovine PD-L1 antibody, IFN-γ production was further enhanced although no significant difference was recognized (FIG. 47). These results suggested that immunostimulatory effect is more strongly induced in M. bovis-infected cattle by combined use of COX-2 inhibitor and rat anti-bovine PD-L1 antibody.

### Reference Example 2

### Rat-Bovine Chimeric Anti-PD-L1 Antibody

### 1. Introduction

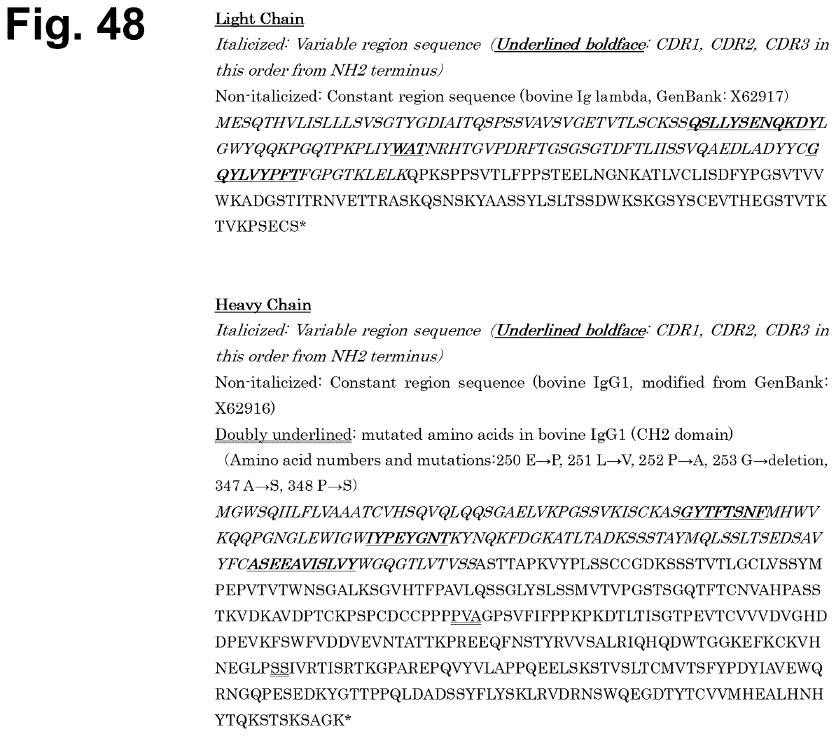

Programmed cell death 1 (PD-1), an immunoinhibitory receptor, and its ligand programmed cell death ligand 1 (PD-L1) are molecules identified by Prof Tasuku Honjo et al., Kyoto University, as factors which inhibit excessive immune response and are deeply involved in immunotolerance. Recently, it has been elucidated that these molecules are also involved in immunosuppression in tumors. In the subject Example, for the purpose of establishing a novel therapy for bovine infections, the present inventors have prepared a chimeric antibody gene by linking the variable region gene of rat anti-bovine PD-L1 monoclonal antibody (4G12) capable of inhibiting the binding of bovine PD-1 and PD-L1 to the constant region gene of a bovine immunoglobulin (IgG1 with mutations having been introduced into the putative binding sites for Fcγ receptors in CH2 domain to inhibit ADCC activity; see FIG. 48 for amino acid numbers and mutations: 250 E->P, 251 L->V, 252 P->A, 253 G->deletion, 347 A->S, 348 P->S; Ikebuchi R, Konnai S, Okagawa T, Yokoyama K, Nakajima C, Suzuki Y, Murata S, Ohashi K. Immunology 2014 August; 142(4):551-561). This chimeric antibody gene was introduced into Chinese hamster ovary cells (CHO cells). By culturing/proliferating the resultant cells, the present inventors have obtained a rat-bovine chimeric anti-bovine PD-L1 antibody (ch4G12) and confirmed its effect in vitro and in vivo.

### 2. Materials and Methods

### 2.1. Construction of Bovine PD-1 and PD-L1 Expressing Cells

The nucleotide sequences of the full length cDNAs of bovine PD-1 gene (GenBank accession number AB510901; Ikebuchi R, Konnai S, Sunden Y, Onuma M, Ohashi K. Microbiol. Immunol. 2010 May; 54(5):291-298) and bovine PD-L1 gene (GenBank accession number AB510902; Ikebuchi R, Konnai S, Shirai T, Sunden Y, Murata S, Onuma M, Ohashi K. Vet. Res. 2011 Sep. 26; 42:103) were determined. Based on the resultant genetic information, bovine PD-1 and bovine PD-L1 membrane expressing cells were prepared. First, for preparing bovine PD-1 or PD-L1 expressing plasmid, PCR was performed using a synthesized bovine PBMC-derived cDNA as a template and designed primers having Nod and HindIII (bovine PD-1) recognition sites and NheI and XhoI (bovine PD-L1) recognition sites on the 5′ side (boPD-1-myc F and R; boPD-L1-EGFP F and R). The PCR products were digested with Nod (Takara) and HindIII (Takara; bovine PD-1), NheI (Takara) and XhoI (Takara; bovine PD-L1), purified with FastGene Gel/PCR Extraction Kit (NIPPON Genetics) and cloned into pCMV-Tag1 vector (Agilent Technologies; bovine PD-1) or pEGFP-N2 vector (Clontech; bovine PD-L1) treated with restriction enzymes in the same manner. The resultant expression plasmid of interest was extracted with QIAGEN Plasmid Midi kit (Qiagen) and stored at −30° C. until use in experiments. Hereinafter, the thus prepared expression plasmid is designated as pCMV-Tag1-boPD-1.

Primer (boPD-1-myc F):
(SEQ ID NO: 143)
ATATGCGGCCGCATGGGGACCCCGCGGGCGCT
Primer (boPD-1-myc R):
(SEQ ID NO: 144)
GCGCAAGCTTTCAGAGGGGCCAGGAGCAGT
Primer (boPD-L1-EGFP F):
(SEQ ID NO: 145)
CTAGCTAGCACCATGAGGATATATAGTGTCTTAAC
Primer (boPD-L1-EGFP R):
(SEQ ID NO: 146)
CAATCTCGAGTTACAGACAGAAGATGACTGC

Bovine PD-1 membrane expressing cells were prepared by the procedures described below. First, 2.5 μg of pCMV-Tag1-boPD-1 was introduced into 4×10⁶ CHO-DG44 cells using Lipofectamine LTX (Invitrogen). Forty-eight hours later, the medium was exchanged with CD DG44 medium (Life Technologies) containing 800 μg/ml G418 (Enzo Life Science), 20 ml/L GlutaMAX supplement (Life Technologies), and 18 ml/L 10% Pluronic F-68 (Life Technologies), followed by selection. The resultant expression cells were reacted with rat anti-bovine PD-1 antibody 5D2 at room temperature. After washing, the cells were further reacted with anti-rat IgG microbeads-labeled antibody (Miltenyi Biotec) at room temperature. Cells expressing bovine PD-1 at high levels were isolated with Auto MACS (Miltenyi Biotec). Subsequently, re-isolation was performed in the same manner to obtain still higher purity. The resultant expression cells were subjected to cloning by limiting dilution to thereby obtain a CHO DG44 cell clone expressing bovine PD-1 at high level (bovine PD-1 expressing cells).

Bovine PD-L1 membrane expressing cells were prepared by the procedures described below. First, 2.5 μg of pEGFP-N2-boPD-L1 or pEGFP-N2 (negative control) was introduced into 4×10⁶ CHO-DG44 cells using Lipofectamine LTX (Invitrogen). Forty-eight hours later, the medium was exchanged with CD DG44 medium (Life Technologies) containing G418 (Enzo Life Science) 800 μg/ml, GlutaMAX supplement (Life Technologies) 20 ml/L, and 10% Pluronic F-68 (Life Technologies) 18 ml/L, followed by selection and cloning by limiting dilution (bovine PD-L1 expressing cell clone). In order to confirm the expression of bovine PD-L1 in the thus prepared expressing cell clone, intracellular localization of EGFP was visualized with an inverted confocal laser microscope LSM700 (ZEISS).

### 2.2. Construction of Soluble Bovine PD-1 and PD-L1

Bovine PD-1-Ig expressing plasmid was constructed by the procedures described below. Briefly, the signal peptide and the extracellular region of bovine PD-1 (GenBank accession number AB510901) were linked to the Fc domain of the constant region of a known bovine IgG1 (GenBank accession number X62916) to prepare a gene sequence. After codons were optimized for CHO cells, gene synthesis was performed in such a manner that NotI recognition sequence, KOZAK sequence, bovine PD-1 signal peptide sequence, bovine PD-1 gene extracellular region sequence, bovine IgG1 Fc region sequence, and XbaI recognition sequence would be located in the gene in this order. It should be noted here that bovine IgG1 was mutated to inhibit ADCC activity; more specifically, mutations were introduced into the putative binding sites for Fcγ receptors of CH2 domain (sites of mutation: 185 E->P, 186 L->V, 187 P->A, 189 G->deletion, 281 A->S, 282 P->S; Ikebuchi R, Konnai S, Okagawa T, Yokoyama K, Nakajima C, Suzuki Y, Murata S, Ohashi K. Immunology 2014 August; 142(4):551-561; the amino acid sequence of PD-1-Ig and the sites of mutation are disclosed in FIG. 2 of this article). The synthesized gene strand was digested with Nod (Takara) and XbaI (Takara), purified with FastGene Gel/PCR Extraction Kit (NIPPON Genetics), and incorporated into the cloning site (Nod and XbaI restriction enzyme recognition sequences downstream of PCMV and between INRBG and PABGH) of expression vector pDN11 (kindly provided by Prof. S. Suzuki, Hokkaido University Research Center for Zoonosis Control) treated with restriction enzymes in the same manner, whereby bovine PD-1-Ig expressing vector was constructed. The expression plasmid was purified with QIAGEN Plasmid Midi kit (Qiagen) and stored at −30° C. until use in experiments. Hereinafter, the thus prepared expression plasmid is designated as pDN11-boPD-1-Ig.

Bovine PD-L1-Ig expressing plasmid was constructed by the procedures described below. In order to amplify the signal peptide and the extracellular region of bovine PD-L1 (GenBank accession number AB510902), primers were designed that had NheI and EcoRV recognition sites added on the 5′ side (boPD-L1-Ig F and R). PCR was performed using a synthesized bovine PBMC-derived cDNA as a template. The PCR products were digested with NheI (Takara) and EcoRV (Takara), purified with FastGene Gel/PCR Extraction Kit (NIPPON Genetics) and cloned into pCXN2.1-Rabbit IgG1 Fc vector (Niwa et al., 1991; Zettlmeissl et al., 1990; kindly provided by Dr. T. Yokomizo, Juntendo University Graduate School of Medicine, and modified in the inventors' laboratory) treated with restriction enzymes in the same manner. The expression plasmid was purified with QIAGEN Plasmid Midi kit (Qiagen) or FastGene Xpress Plasmid PLUS Kit (NIPPON Genetics) and stored at −30° C. until use in experiments. Hereinafter, the thus prepared expression plasmid is designated as pCXN2.1-boPD-L1-Ig.

Primer (boPD-L1-Ig F):
(SEQ ID NO: 147)
GCTAGCATGAGGATATATAGTGTCTTAAC
Primer (boPD-L1-Ig R):
(SEQ ID NO: 148)
GATATCATTCCTCTTTTTTGCTGGAT

Soluble bovine PD-1-Ig expressing cells were prepared by the procedures described below. Briefly, 2.5 μg of pDN11-boPD-1-Ig was introduced into 4×10⁶ CHO-DG44 cells using Lipofectamine LTX (Invitrogen). Forty-eight hours later, the medium was exchanged with OptiCHO AGT medium (Life Technologies) containing 800 μg/ml G418 (Enzo Life Science) and 20 ml/L GlutaMAX supplement (Life Technologies). After cultured for 3 weeks, the cells were subjected to selection. Briefly, the concentrations of the Fc fusion recombinant protein in the culture supernatants of the resultant cell clones were measured by ELISA using anti-bovine IgG F(c) rabbit polyclonal antibody (Rockland) to thereby select those cell clones that express the Fc fusion recombinant protein at high levels. The resultant highly expressing cell clone was transferred to a G418-free medium and cultured under shaking for 14 days, followed by collection of a culture supernatant. The culture supernatant containing the Fc fusion recombinants protein was ultrafiltered with Centricon Plus-70 (Millipore). Then, the Fc fusion recombinant protein was purified with Ab-Capcher Extra (ProteNova). After purification, the buffer was exchanged with phosphate-buffered physiological saline (PBS; pH 7.4) using PD-10 Desalting Column (GE Healthcare). The resultant protein was stored at −30° C. until use in experiments (bovine PD-1-Ig). The concentration of the purified bovine PD-1-Ig was measured by ELISA using IgG F(c) rabbit polyclonal antibody (Rockland). For each washing operation in ELISA, Auto Plate Washer BIO WASHER 50 (DS Pharma Biomedical) was used. Absorbance was measured with Microplate Reader MTP-650FA (Corona Electric).

Soluble bovine PD-L1-Ig expressing cells were prepared by the procedures described below. Briefly, 30 μg of pCXN2.1-boPD-L1-Ig was introduced into 7.5×10⁷ Expi293F cells (Life Technologies) using Expifectamine (Life Technologies). After 7-day culture under shaking, the culture supernatant was collected. The recombinant protein was purified from the supernatant using Ab-Capcher Extra (ProteNova; bovine PD-L1-Ig). After purification, the buffer was exchanged with PBS (pH 7.4) using PD MiniTrap G-25 (GE Healthcare). The resultant protein was stored at −30° C. until use in experiments (bovine PD-L1-Ig). The concentration of the purified bovine PD-L1-Ig was measured using Rabbit IgG ELISA Quantitation Set (Bethyl). For each washing operation in ELISA, Auto Plate Washer BIO WASHER 50 (DS Pharma Biomedical) was used. Absorbance was measured with Microplate Reader MTP-650FA (Corona Electric).

### 2.3. Preparation of Rat Anti-Bovine PD-L1 Monoclonal Antibody Producing Cells

Rat was immunized in the footpad with bovine PD-L1-Ig (Ikebuchi R, Konnai S, Okagawa T, Yokoyama K, Nakajima C, Suzuki Y, Murata S, Ohashi K. Immunology 2014 August; 142(4):551-561; bovine PD-L1-Ig was prepared by the method disclosed in this article and used for immunization). Hybridomas were established by the iliac lymph node method to thereby obtain rat anti-bovine PD-L1 monoclonal antibody producing hybridoma 4G12. With respect to the method of establishment of rat anti-bovine PD-L1 monoclonal antibody, details are disclosed in the following non-patent document (Ikebuchi R, Konnai S, Okagawa T, Yokoyama K, Nakajima C, Suzuki Y, Murata S, Ohashi K. Vet. Res. 2013 Jul. 22; 44:59; Ikebuchi R, Konnai S, Okagawa T, Yokoyama K, Nakajima C, Suzuki Y, Murata S, Ohashi K. Immunology 2014 August; 142(4):551-561).

### 2.4. Preparation of Rat-Bovine Chimeric Anti-Bovine PD-L1 Antibody Expressing Vector

Rat-bovine chimeric anti-bovine PD-L1 antibody ch4G12 was established by fusing the antibody constant regions of bovine IgG1 and Igλ with rat anti-bovine PD-L1 antibody 4G12 being used as an antibody variable region.

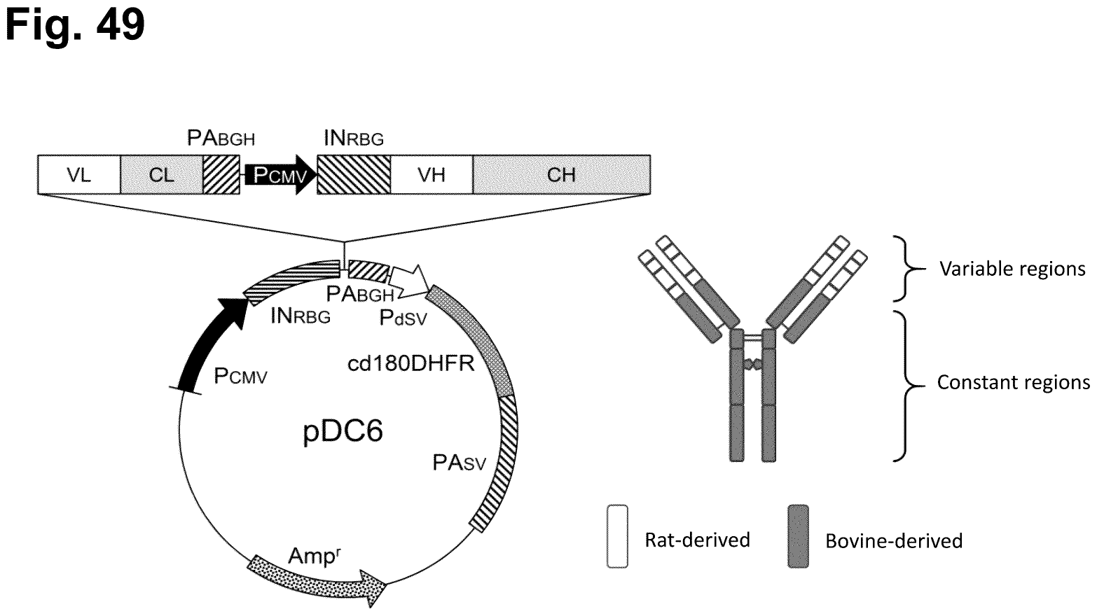

First, the genes of heavy chain and light chain variable regions were identified from a hybridoma that would produce rat anti-bovine PD-L1 antibody 4G12. Subsequently, a gene sequence was prepared in which the heavy chain and the light chain variable regions of the antibody 4G12 were linked to known constant regions of bovine IgG1 (heavy chain; modified from GenBank Accession number X62916) and bovine Igλ (light chain; GenBank Accession number X62917), respectively, and codon optimization was carried out [rat-bovine chimeric anti-bovine PD-L1 antibody ch4G12: SEQ ID NOS: 115 and 116 (amino acid sequences), SEQ ID NOS: 117 and 118 (nucleotide sequences after codon optimization)]. It should be noted that in order to suppress the ADCC activity of bovine IgG1, mutations were added to the putative binding sites of Fcγ receptors in CH2 domain (See FIG. 48 for amino acid numbers and mutations: 250 E->P, 251 L->V, 252 P->A, 253 G->deletion, 347 A->S, 348 P->S; Ikebuchi R, Konnai S, Okagawa T, Yokoyama K, Nakajima C, Suzuki Y, Murata S, Ohashi K. Immunology 2014 August; 142(4):551-561). Then, the gene was artificially synthesized in such a manner that Nod recognition sequence, KOZAK sequence, chimeric antibody light chain sequence, poly-A addition signal sequence (PABGH), promoter sequence (PCMV), Sad recognition sequence, intron sequence (INRBG), KOZAK sequence, chimeric antibody heavy chain sequence and XbaI recognition sequence would be located in this order. The synthesized gene strand was digested with Nod (Takara) and XbaI (Takara), purified with FastGene Gel/PCR Extraction Kit (NIPPON Genetics) and cloned into the cloning site (Nod and XbaI restriction enzyme recognition sequences downstream of PCMV and between INRBG and PABGH) of expression plasmid pDC6 (kindly provided by Prof. S. Suzuki, Hokkaido University Research Center for Zoonosis Control) treated with restriction enzymes in the same manner (FIG. 49). The resultant plasmid was extracted with QIAGEN Plasmid Midi kit (Qiagen) and stored at −30° C. until use in experiments. Hereinafter, the thus prepared expression plasmid is designated as pDC6-boPD-L1ch4G12.

### 2.5. Expression of Rat-Bovine Chimeric Anti-Bovine PD-L1 Antibody

The pDC6-boPD-L1ch4G12 was transfected into CHO-DG44 cells (CHO-DG44 (dfhr^{-/-})) which were a dihydrofolate reductase deficient cell. Forty-eight hours later, the medium was exchanged with OptiCHO AGT medium (Life Technologies) containing 20 ml/L GlutaMAX supplement (Life Technologies). After cultured for 3 weeks, the cells were subjected to selection and cloning by limiting dilution. Subsequently, the concentrations of the chimeric antibody in the culture supernatants were measured by dot blotting and ELISA using anti-bovine IgG F(c) rabbit polyclonal antibody (Rockland) to thereby select high expression clones. Further, the selected clones expressing rat-bovine chimeric anti-bovine PD-L1 antibody at high levels were subjected to gene amplification treatment by adding a load with 60 nM methotrexate (Mtx)-containing medium. The thus established cell clone stably expressing rat-bovine chimeric anti-bovine PD-L1 antibody was transferred into Mtx-free Opti-CHO AGT medium and cultured under shaking for 14 days (125 rpm, 37° C., 5% CO₂). Chimeric antibody production in the culture supernatant was measured by ELISA using anti-bovine IgG F(c) rabbit polyclonal antibody (Rockland). For each washing operation in ELISA, Auto Plate Washer BIO WASHER 50 (DS Pharma Biomedical) was used. Absorbance was measured with Microplate Reader MTP-650FA (Corona Electric). The culture supernatant at day 14 was centrifuged at 10,000 g for 10 min to remove cells, and the centrifugal supernatant was passed through a Steritop-GP 0.22 μm filter (Millipore) for sterilization and then stored at 4° C. until it was subjected to purification.

### 2.6. Purification of Rat-Bovine Chimeric Anti-Bovine PD-L1 Antibody

From the culture supernatant prepared as described above, each chimeric antibody was purified using Ab Capcher Extra (ProteNova). An open column method was used for binding to resin; PBS pH 7.4 was used as an equilibration buffer and a wash buffer. As an elution buffer, IgG Elution Buffer (Thermo Fisher Scientific) was used. As a neutralization buffer, 1M Tris (pH 9.0) was used. The purified antibody was subjected to buffer exchange with PBS (pH 7.4) using PD-10 Desalting Column (GE Healthcare) and concentrated using Amicon Ultra-15 (50 kDa, Millipore). The thus purified chimeric antibody was passed through a 0.22 μm syringe filter (Millipore) for sterilization and stored at 4° C. until use in experiments.

### 2.7. Confirmation of the Purity of Purified Rat-Bovine Chimeric Anti-Bovine PD-L1 Antibody

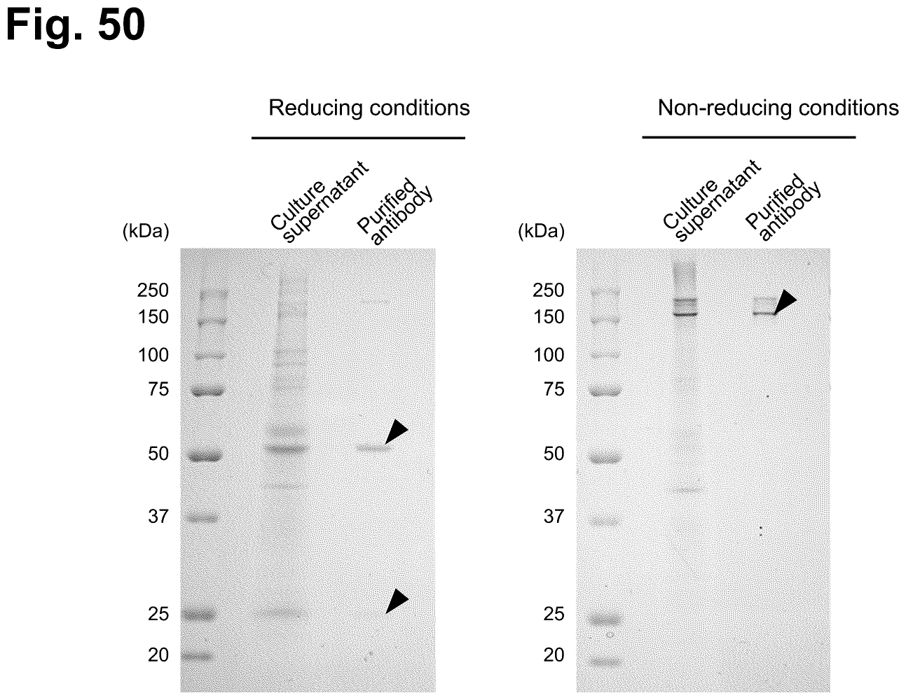

In order to confirm the purity of purified rat-bovine chimeric anti-bovine PD-L1 antibody, antibody proteins were detected by SDS-PAGE and CBB staining. Using 10% acrylamide gel, the purified rat-bovine chimeric antibody was electrophoresed under reducing conditions (reduction with 2-mercaptoethanol from Sigma-Aldrich) and non-reducing conditions. Bands were stained with Quick-CBB kit (Wako) and decolored in distilled water. The results are shown in FIG. 50. Bands were observed at predicted positions, that is, at 25 kDa and 50 kDa under reducing conditions and at 150 kDa under non-reducing conditions.

### 2.8. Binding Specificity of Rat-Bovine Chimeric Anti-Bovine PD-L1 Antibody

It was confirmed by flow cytometry that the rat-bovine chimeric anti-bovine PD-L1 antibody specifically binds to the bovine PD-L1 expressing cells (described above). First, rat anti-bovine PD-L1 antibody 4G12 or rat-bovine chimeric anti-bovine PD-L1 antibody ch4G12 was reacted with bovine PD-L1 expressing cells at room temperature for 30 min. After washing, APC-labeled anti-rat Ig goat antibody (Southern Biotech) or Alexa Fluor 647—labeled anti-bovine IgG (H+L) goat F(ab′)2 (Jackson ImmunoResearch) was reacted at room temperature for 30 min. As negative control antibody, rat IgG2a (κ) isotype control (BD Biosciences) or bovine IgG1 antibody (Bethyl) was used. After washing, each rat antibody or rat-bovine chimeric antibody bound to cell surfaces was detected by FACS Verse (BD Biosciences). For every washing operation and dilution of antibody, PBS supplemented with 1% bovine serum albumin (Sigma-Aldrich) was used.

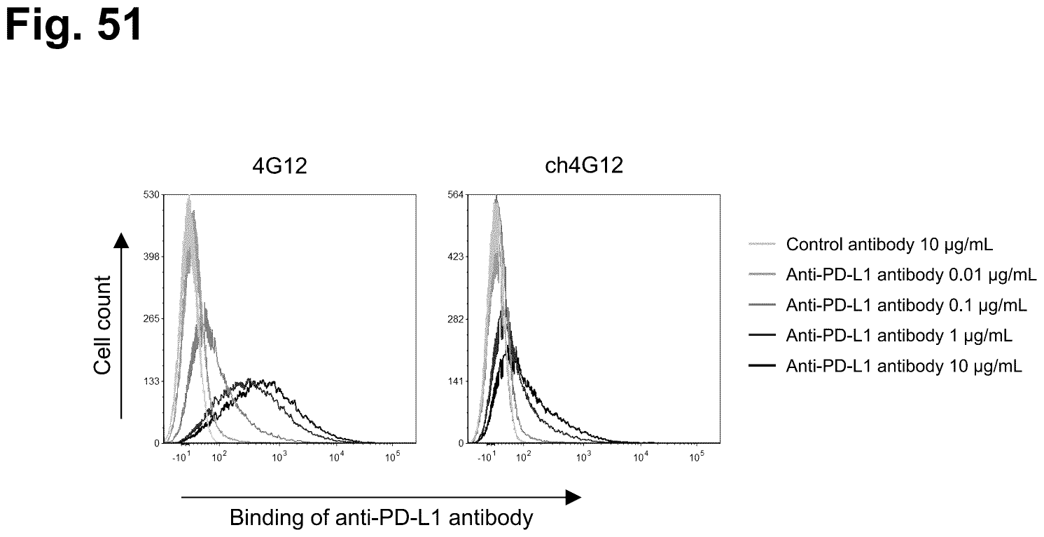

The experimental results are shown in FIG. 51. It was revealed that rat-bovine chimeric anti-bovine PD-L1 antibody ch4G12 binds to bovine PD-L1 expressing cells in the same manner as rat anti-bovine PD-L1 antibody 4G12.

### 2.9. Inhibitory Activity of Rat-Bovine Chimeric Anti-PD-L1 Antibody Against Bovine PD-1/PD-L1 Binding

### (1) Binding Inhibition Test on Bovine PD-L1 Expressing Cells and Soluble Bovine PD-1

Using bovine PD-L1 expressing cells (described above) and bovine PD-1-Ig (described above), bovine PD-1/PD-L1 binding inhibition by anti-bovine PD-L1 antibody was tested. First, 2×10⁵ bovine PD-L1 expressing cells were reacted with various concentrations (0, 0.32, 0.63, 1.25, 2.5, 5 or 10 μg/ml) of rat anti-bovine PD-L1 antibody 4G12 or rat-bovine chimeric anti-bovine PD-L1 antibody ch4G12 at room temperature for 30 min. As negative control antibody, rat IgG2a (κ) isotype control (BD Biosciences) or bovine IgG1 antibody (Bethyl) was used. After washing, bovine PD-1-Ig labeled with biotin using Lightning-Link Type A Biotin Labeling Kit (Innova Bioscience) was added to a final concentration of 2 μg/ml, followed by reaction for another 30 min at room temperature. Subsequently, after washing, bovine PD-1-Ig bound to cell surfaces was detected with APC-labeled streptavidin (BioLegend). For analysis, FACS Verse (BD Biosciences) was used. For every washing operation and dilution of antibody, PBS supplemented with 1% bovine serum albumin (Sigma-Aldrich) was used. Taking the proportion of PD-1-Ig bound cells without antibody addition as 100%, the proportion of PD-1-Ig bound cells at each antibody concentration was shown as relative value.

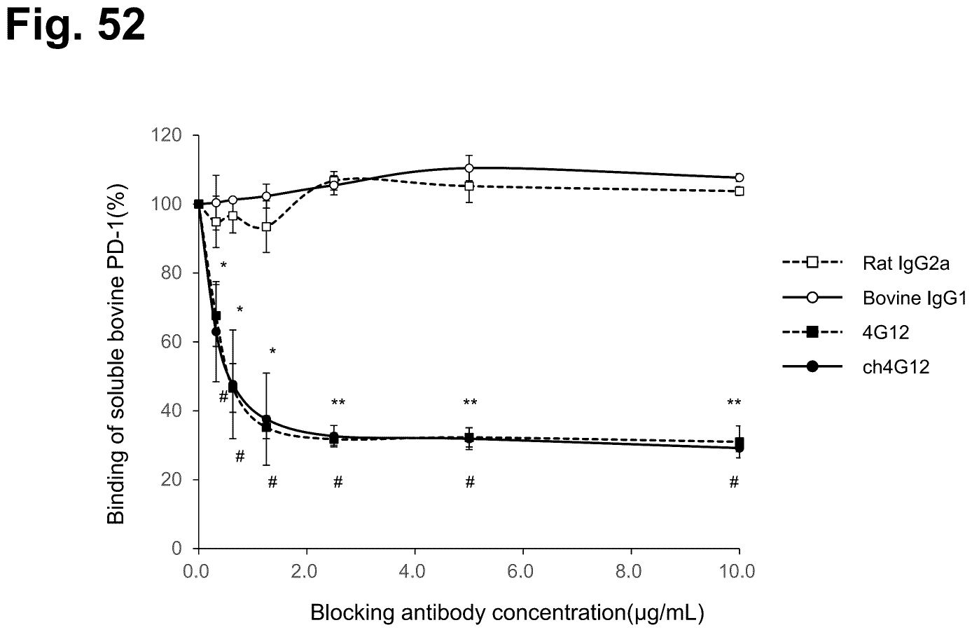

The experimental results are shown in FIG. 52. It was revealed that like rat anti-bovine PD-L1 antibody 4G12, rat-bovine chimeric anti-bovine PD-L1 antibody ch4G12 is capable of inhibiting bovine PD-1/PD-L1 binding in a concentration dependent manner.

### (2) Binding Inhibition Test on Bovine PD-1 Expressing Cells and Soluble Bovine PD-L1

Using bovine PD-1 expressing cells (described above) and bovine PD-L1-Ig (described above), bovine pD-1/PD-L1 binding inhibition by anti-bovine PD-L1 antibody was tested. First, rat anti-bovine PD-L1 antibody 4G12 or rat-bovine chimeric anti-bovine PD-L1 antibody ch4G12 at a final concentration of 0, 0.32, 0.63, 1.25, 2.5, 5 or 10 μg/ml and bovine PD-L1-Ig at a final concentration of 1 μg/ml were placed in 96-well plates, where they were reacted at room temperature for 30 min. The resultant mixture was reacted with 2×10⁵ bovine PD-1 expressing cells at room temperature for 30 min. As negative control antibody, rat IgG2a (κ) isotype control (BD Biosciences) or bovine IgG1 antibody (Bethyl) was used. After washing, Alexa Fluor 647-labeled anti-rabbit IgG (H+L) goat F(ab′)2 (Life Technologies) was reacted at room temperature for 30 min to thereby detect bovine PD-L1-Ig bound to cell surfaces. For analysis, FACS Verse (BD Biosciences) was used. For every washing operation and dilution of antibody, PBS supplemented with 1% bovine serum albumin (Sigma-Aldrich) was used. Taking the proportion of PD-L1-Ig bound cells without antibody addition as 100%, the proportion of PD-L1-Ig bound cells at each antibody concentration was shown as relative value.

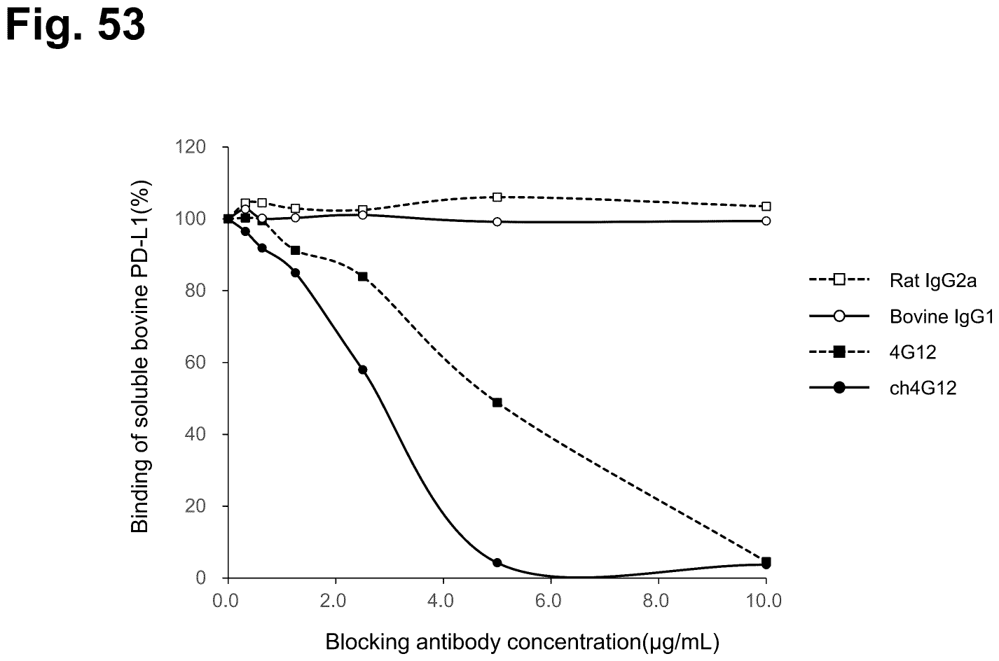

The experimental results are shown in FIG. 53. It was revealed that like rat anti-bovine PD-L1 antibody 4G12, rat-bovine chimeric anti-bovine PD-L1 antibody ch4G12 is capable of inhibiting bovine PD-1/PD-L1 binding in a concentration dependent manner.

### 2.10. Biological Activity Test Using Rat-Bovine Chimeric Anti-Bovine PD-L1 Antibody

### (1) Effect on Cell Proliferation

In order to confirm that bovine PD-1/PD-L1 binding inhibition by rat-bovine chimeric anti-PD-L1 antibody activates lymphocytes, a biological activity test was performed using cell proliferation as an indicator. Briefly, bovine PBMCs isolated from peripheral blood of healthy cattle were suspended in PBS to give a concentration of 10×10⁶ cells/ml, and reacted with carboxyfluorescein succinimidyl ester (CFSE) at room temperature for 20 min. After washing twice with RPMI 1640 medium (Sigma-Aldrich) containing 10% inactivated fetal bovine serum (Cell Culture Technologies), antibiotics (streptomycin 200 μg/ml, penicillin 200 U/ml) (Life Technologies) and 0.01% L-glutamine (Life Technologies), the PBMCs were reacted with anti-bovine CD3 mouse antibody (WSU Monoclonal Antibody Center) at 4° C. for 30 min. After washing, the PBMCs were reacted with anti-mouse IgG1 microbeads (Miltenyi Biotec) at 4° C. for 15 min, followed by isolation of CD3-positive T cells using autoMACS™ Pro(Miltenyi Biotec). To the isolated CD3-positive T cells, anti-bovine CD3 mouse antibody (WSU Monoclonal Antibody Center) and anti-bovine CD28 mouse antibody (Bio-Rad) were added. Then, the cells were co-cultured with bovine PD-L1 expressing cells (CD3-positive T cells: bovine PD-L1 expressing cells =10:1) in the presence or absence of 10 μg/ml of rat-bovine chimeric anti-bovine PD-L1 antibody ch4G12. As a control for antibodies, serum-derived bovine IgG (Sigma-Aldrich) was used; as a control for PD-L1 expressing cells, EGFP expressing cells transfected with pEGFP-N2 were used. After a 6-day coculture, cells were harvested and reacted with anti-bovine CD4 mouse antibody and anti-bovine CD8 mouse antibody (Bio-Rad) at room temperature for 30 min. For the labeling of antibodies, Zenon Mouse IgG1 Labeling Kits (Life Technologies) or Lightning-Link Kit (Innova Biosciences) was used. For analysis, FACS Verse (BD Biosciences) was used. For washing operation after culturing and dilution of antibody, PBS supplemented with 1% bovine serum albumin (Sigma-Aldrich) was used.

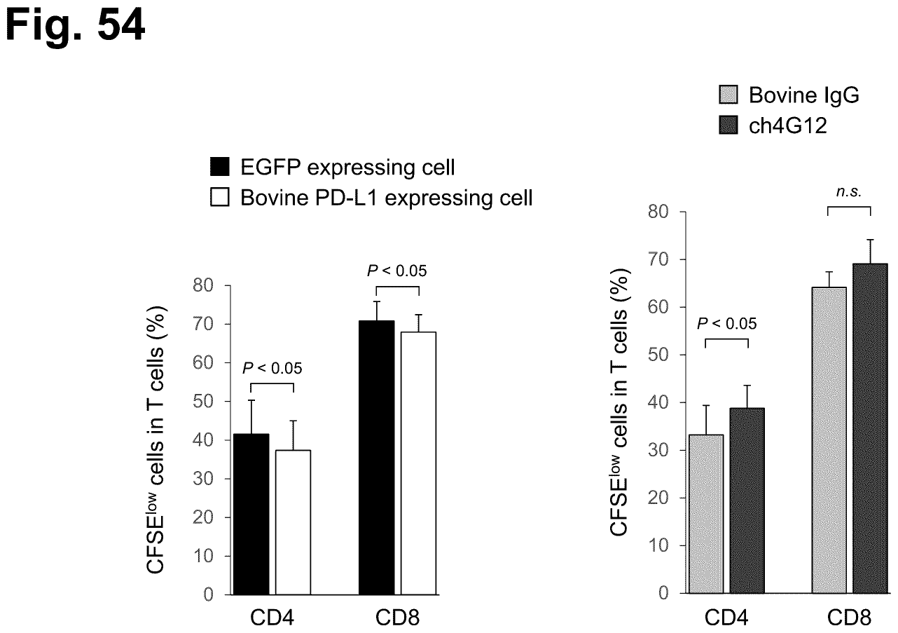

The experimental results are shown in FIG. 54. Proliferation of CD4-positive and CD8-positive T cells was significantly suppressed by co-culture with bovine PD-L1 expressing cells. It was revealed that rat-bovine chimeric anti-bovine PD-L1 antibody ch4G12 inhibits this suppression in CD4-positive T cells.

### (2) Effect on IFN-γ Production

In order to confirm that bovine PD-1/PD-L1 binding inhibition by rat-bovine chimeric anti-PD-L1 antibody activates lymphocytes, a biological activity test was performed using IFN-γ production as an indicator. Briefly, PBMCs isolated from peripheral blood of BLV-infected cattle were suspended in RPMI medium (Sigma-Aldrich) containing 10% inactivated fetal bovine serum (Cell Culture Technologies), antibiotics (streptomycin 200 μg/ml, penicillin 200 U/ml) (Life Technologies) and 0.01% L-glutamine (Life Technologies) to give a concentration of 4×10⁶ cells/ml. To the PBMCs, 10 μg/ml of rat anti-bovine PD-L1 antibody 4G12 or rat-bovine chimeric anti-bovine PD-L1 antibody ch4G12, and 2% BLV-infected fetal lamp kidney cell (FLK-BLV) culture supernatant were added; culturing was then performed at 37° C. under 5% CO₂ for 6 days. As control antibodies, serum-derived rat IgG (Sigma-Aldrich) and serum-derived bovine IgG (Sigma-Aldrich) were used. After a 6-day culture, a culture supernatant was collected, and IFN-γ production was measured with Bovine IFN-γ ELISA Kit (BETYL). For each washing operation in ELISA, Auto Plate Washer BIO WASHER 50 (DS Pharma Biomedical) was used. Absorbance was measured with Microplate Reader MTP-650FA (Corona Electric).

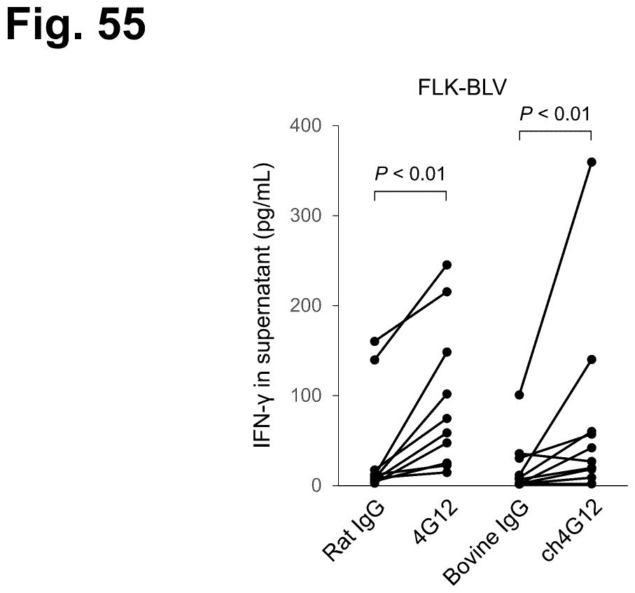

The experimental results are shown in FIG. 55. It was revealed that rat-bovine chimeric anti-bovine PD-L1 antibody ch4G12 increases bovine PBMCs' IFN-γ response to BLV antigen in the same manner as rat anti-bovine PD-L1 antibody 4G12 (n=10).

### 2.11. Inoculation Test on Cattle

Established rat-bovine chimeric anti-bovine PD-L1 antibody ch4G12 (about 260 mg; 1 mg/kg) was intravenously administered into experimentally BLV-infected calf (Holstein, male, 7 months old, 267 kg). Blood samples were collected chronologically from the infected calf, followed by isolation of PBMCs by density gradient centrifugation.

### (1) Cell Proliferation Response of T Cells to BLV Antigen

Bovine PBMCs were suspended in PBS and reacted with CFSE at room temperature for 20 min. After washing twice with RPMI 1640 medium (Sigma-Aldrich) containing 10% inactivated fetal bovine serum (Cell Culture Technologies), antibiotics (streptomycin 200 μg/ml, penicillin 200 U/ml) (Life Technologies) and 0.01% L-glutamine (Life Technologies), the cell concentration was adjusted to 4×10⁶ cells/ml using the same medium. Culture supernatant of 2% BLV-infected fetal lamp kidney cells (FLK-BLV) was added to the PBMCs, which were then cultured at 37° C. under 5% CO₂ for 6 days. As a control, culture supernatant of 2% BLV-not-infected fetal lamp kidney cells (FLK) was used. After a 6-day culture, PBMCs were collected and reacted with anti-bovine CD4 mouse antibody, anti-bovine CD8 mouse antibody and anti-bovine IgM mouse antibody (Bio-Rad) at 4° C. for 20 min. For the labeling of antibodies, Zenon Mouse IgG1 Labeling Kits (Life Technologies) or Lightning-Link Kit (Innova Biosciences) was used. For analysis, FACS Verse (BD Biosciences) was used. For every washing operation and dilution of antibody, PBS supplemented with 1% bovine serum albumin (Sigma-Aldrich) was used.

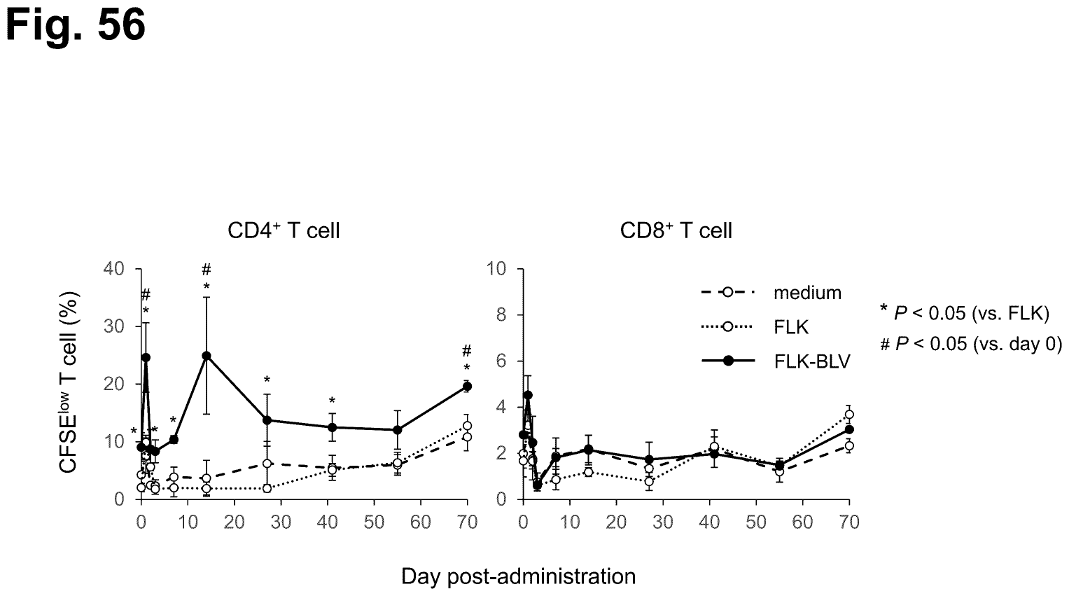

The experimental results are shown in FIG. 56. As a result of antibody administration, BLV-specific cell proliferation response of CD4-positive T cells increased compared to the response before administration.

### (2) Changes in the BLV Provirus Load

DNA was extracted from isolated bovine PBMCs using Wizard DNA Purification kit (Promega). The concentration of the extracted DNA was quantitatively determined, taking the absorbance (260 nm) measured with Nanodrop 8000 Spectrophotometer (ThermoFisher Scientific) as a reference. In order to measure the BLV provirus load in PBMCs, real time PCR was performed using Cycleave PCR Reaction Mix SP (TaKaRa) and Probe/Primer/Positive control for bovine leukemia virus detection (TaKaRa). Light Cycler 480 System II (Roche Diagnosis) was used for measurement.

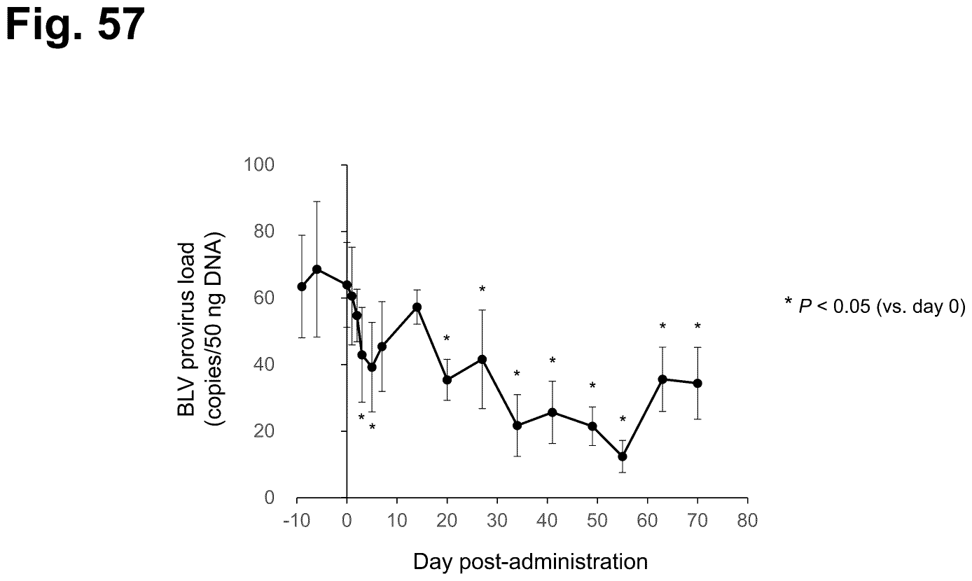

The experimental results are shown in FIG. 57. The BLV provirus load significantly decreased until the end of test period compared to the load before administration.

### [Example 5] Establishment of Rat-Bovine Chimeric Anti-Bovine PD-1 Antibody

### 1. Introduction

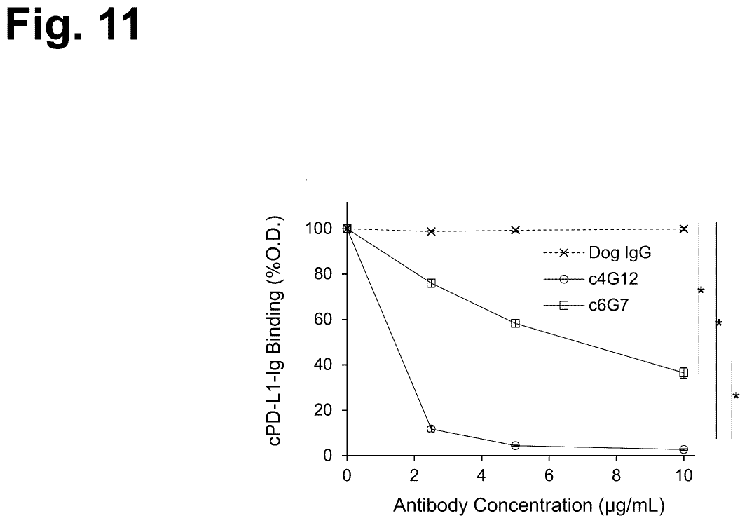

Programmed cell death 1 (PD-1), an immunoinhibitory receptor, and its ligand programmed cell death ligand 1 (PD-L1) are molecules identified by Prof Tasuku Honjo et al., Kyoto University, as factors which inhibit excessive immune response and are deeply involved in immunotolerance. Recently, it has been elucidated that these molecules are also involved in immunosuppression in tumors. In the subject Example, for the purpose of establishing a novel therapy for bovine infections, a chimeric antibody gene was prepared in which variable region genes of rat anti-bovine PD-1 monoclonal antibody 5D2 capable of inhibiting the binding of bovine PD-1 to PD-L1 were linked to constant region genes of bovine immunoglobulins (bovine IgG1 and Igλ, with mutations having been introduced into the putative binding sites of Fcγ receptors in bovine IgG1's CH2 domain to inhibit ADCC activity; see FIGS. 1 and 11 for amino acid numbers and mutations: 250 E->P, 251 L->V, 252 P->A, 253 G->deletion, 347 A->S, 348 P->S; Ikebuchi R, Konnai S, Okagawa T, Yokoyama K, Nakajima C, Suzuki Y, Murata S, Ohashi K. Immunology, 142(4):551-561, August 2014). The resultant chimeric antibody gene was introduced into Chinese hamster ovary cells (CHO cells), which were cultured and proliferated to produce a rat-bovine chimeric anti-bovine PD-1 antibody ch5D2. The effect of this chimeric antibody was confirmed in vitro and in vivo.

### 2. Materials, Methods and Experimental Results

### 2.1. Construction of Bovine PD-1 and PD-L1 Expressing Cells

The nucleotide sequences of the full length cDNAs of bovine PD-1 gene (GenBank accession number AB510901; Ikebuchi R, Konnai S, Sunden Y, Onuma M, Ohashi K. Microbiol. Immunol., 54(5):291-298; May 2010) and bovine PD-L1 gene (GenBank accession number AB510902; Ikebuchi R, Konnai S, Shirai T, Sunden Y, Murata S, Onuma M, Ohashi K. Vet. Res., 42:103, Sep. 26, 2011) were determined. Based on the resultant genetic information, bovine PD-1 and bovine PD-L1 expressing cells were prepared. First, for preparing bovine PD-1 or PD-L1 expressing plasmid, PCR was performed using a synthesized bovine peripheral blood mononuclear cell (PBMC)-derived cDNA as a template and designed primers having Nod and HindIII (bovine PD-1) recognition sites or NheI and XhoI (bovine PD-L1) recognition sites on the 5′ side (boPD-1-myc F and R; or boPD-L1-EGFP F and R). The PCR products were digested with Nod (Takara) and HindIII (Takara; bovine PD-1) or NheI (Takara) and XhoI (Takara; bovine PD-L1), purified with FastGene Gel/PCR Extraction Kit (NIPPON Genetics) and cloned into pCMV-Tag1 vector (Agilent Technologies; bovine PD-1) or pEGFP-N2 vector (Clontech; bovine PD-L1) treated with the restriction enzymes in the same manner. The resultant expression plasmid of interest was extracted with QIAGEN Plasmid Midi kit (Qiagen) and stored at −30° C. until use in experiments. Hereinafter, the thus prepared expression plasmid is designated as pCMV-Tag1-boPD-1 or pEGFP-N2-boPD-L1.

Primer (boPD-1-myc F):
(SEQ ID NO: 209)
ATATGCGGCCGCATGGGGACCCCGCGGGCGCT
Primer (boPD-1-myc R):
(SEQ ID NO: 210)
GCGCAAGCTTTCAGAGGGGCCAGGAGCAGT
Primer (boPD-L1-EGFP F):
(SEQ ID NO: 211)
CTAGCTAGCACCATGAGGATATATAGTGTCTTAAC
Primer (boPD-L1-EGFP R):
(SEQ ID NO: 212)
CAATCTCGAGTTACAGACAGAAGATGACTGC

Bovine PD-1 expressing cells were prepared by the procedures described below. First, 2.5 μg of pCMV-Tag1-boPD-1 was introduced into 4×10⁶ CHO-DG44 cells using Lipofectamine LTX (Invitrogen). Forty-eight hours later, the medium was exchanged with CD DG44 medium (Life Technologies) containing 800 μg/ml G418 (Enzo Life Science), 20 ml/L GlutaMAX supplement (Life Technologies), and 18 ml/L 10% Pluronic F-68 (Life Technologies), followed by selection. The resultant expression cells were reacted with rat anti-bovine PD-1 antibody 5D2 at room temperature. After washing, the cells were further reacted with anti-rat IgG microbead-labeled antibody (Miltenyi Biotec) at room temperature. Cells expressing bovine PD-1 at high levels were isolated with Auto MACS (Miltenyi Biotec). Subsequently, re-isolation was performed in the same manner to obtain still higher purity. The resultant expression cells were subjected to cloning by limiting dilution to thereby obtain a CHO DG44 cell clone expressing bovine PD-1 at high level (bovine PD-1 expressing cells).

Bovine PD-L1 membrane expressing cells were prepared by the procedures described below. First, 2.5 μg of pEGFP-N2-boPD-L1 or pEGFP-N2 (negative control) was introduced into 4×10⁶ CHO-DG44 cells using Lipofectamine LTX (Invitrogen). Forty-eight hours later, the medium was exchanged with CD DG44 medium (Life Technologies) containing 800 μg/ml G418 (Enzo Life Science), 20 ml/L GlutaMAX supplement (Life Technologies) and 18 ml/L 10% Pluronic F-68 (Life Technologies), followed by selection and cloning by limiting dilution (bovine PD-L1 expressing cell clone). In order to confirm the expression of bovine PD-L1 in the thus prepared cell clone, intracellular localization of EGFP was visualized with an inverted confocal laser microscope LSM700 (ZEISS).

### 2.2 Construction of Soluble Bovine PD-1

Bovine PD-1-Ig expressing plasmid was constructed by the procedures described below. Briefly, the signal peptide and the extracellular region of bovine PD-1 (GenBank accession number AB510901) were linked to the constant region of a known bovine IgG1 (GenBank accession number X62916) to prepare a gene sequence. After codons were optimized for CHO cells, gene synthesis was performed in such a manner that Nod recognition sequence, KOZAK sequence, bovine PD-1 signal peptide sequence, bovine PD-1 gene extracellular region sequence, bovine IgG1 Fc region sequence, and XbaI recognition sequence would be located in the gene in this order. It should be noted here that bovine IgG1 was mutated to inhibit ADCC activity; more specifically, mutations were introduced into the putative binding sites for Fcγ receptors of CH2 domain (sites of mutation: 185 E->P, 186 L-V, 187 P->A, 189 G->deletion, 281 A->S, 282 P->S; Ikebuchi R, Konnai S, Okagawa T, Yokoyama K, Nakajima C, Suzuki Y, Murata S, Ohashi K. Immunology, 142(4):551-561, August 204; the amino acid sequence of PD-1-Ig and the sites of mutation are disclosed in FIG. 2 of this article). The synthesized gene strand was digested with Nod (Takara) and XbaI (Takara), purified with FastGene Gel/PCR Extraction Kit (NIPPON Genetics), and incorporated into the cloning site (Nod and XbaI restriction enzyme recognition sequences downstream of PCMV and between INRBG and PABGH) of expression vector pDN11 (kindly provided by Prof. S. Suzuki, Hokkaido University Research Center for Zoonosis Control) treated with the restriction enzymes in the same manner, whereby bovine PD-1-Ig expressing vector was constructed. The expression plasmid was purified with QIAGEN Plasmid Midi kit (Qiagen) and stored at −30° C. until use in experiments. Hereinafter, the thus prepared expression plasmid is designated as pDN11-boPD-1-1g.

Bovine PD-1-His expressing plasmid was prepared by the procedures described below. Briefly, for the purpose of amplifying the signal peptide and the extracellular region of bovine PD-1 (GenBank accession number AB510901), primers were designed in which NotI and XhoI recognition sites were added on the 5′ side (boPD-1-His F and R). A genetic sequence encoding a 6xHis tag was added to the reverse primer. PCR was performed using a synthesized bovine PBMC-derived cDNA as a template. The respective PCR products were digested with Nod (Takara) and XhoI (Takara), purified with FastGene Gel/PCR Extraction Kit (NIPPON Genetics) and cloned into pCXN2.1(+) vector (Niwa H, Yamamura K, Miyazaki J. Gene, 108(2):193-199; Dec. 15, 1991; kindly provided by Dr. T. Yokomizo, Juntendo University Graduate School of Medicine) treated with the restriction enzymes in the same manner. The resultant expression plasmid was purified with FastGene Xpress Plasmid PLUS Kit (NIPPON Genetics) and stored at −30° C. until use in experiments. Hereinafter, the thus prepared expression plasmid is designated as pCXN2.1-boPD-1-His.

Primer (boPD-1-His F):
(SEQ ID NO: 213)
ATAAGAATGCGGCCGCCACCATGGGGACCCCGCGGGCGCT
Primer (boPD-1-His R):
(SEQ ID NO: 214)
GCCCTCGAGTTAATGGTGATGGTGATGGTGGATGACCAGGCTCTGCATCT

Soluble bovine PD-1-Ig expressing cells were prepared by the procedures described below. Briefly, 2.5 μg of pDN11-boPD-1-Ig was introduced into 4×10⁶ CHO-DG44 cells using Lipofectamine LTX (Invitrogen). Forty-eight hours later, the medium was exchanged with CD OptiCHO medium (Life Technologies) containing 800 μg/ml G418 (Enzo Life Science) and 20 ml/L GlutaMAX supplement (Life Technologies). After cultured for 3 weeks, the cells were subjected to selection. Briefly, the concentrations of the Fc fusion recombinant protein in the culture supernatants of the resultant cell clones were measured by ELISA using anti-bovine IgG F(c) rabbit polyclonal antibody (Rockland) to thereby select those cell clones that express the Fc fusion recombinant protein at high levels. The resultant highly expressing cell clone was transferred to a G418-free medium and cultured under shaking for 14 days, followed by collection of a culture supernatant. The culture supernatant containing the Fc fusion recombinants protein was ultrafiltered with Centricon Plus-70 (Millipore). Then, the Fc fusion recombinant protein was purified with Ab-Capcher Extra (ProteNova). After purification, the buffer was exchanged with phosphate-buffered physiological saline (PBS; pH 7.4) using PD-10 Desalting Column (GE Healthcare). The resultant protein was stored at −30° C. until use in experiments (bovine PD-1-Ig). The concentration of the purified bovine PD-1-Ig was measured by ELISA using IgG F(c) rabbit polyclonal antibody (Rockland). For each washing operation in ELISA, Auto Plate Washer BIO WASHER 50 (DS Pharma Biomedical) was used. Absorbance was measured with Microplate Reader MTP-650FA (Corona Electric).

Soluble bovine PD-1-His expressing cells were prepared by the procedures described below. Briefly, 30 μg of pCXN2.1-boPD-1-His was introduced into 7.5×10⁷ Expi293F cells (Life Technologies) using Expifectamine (Life Technologies). After a 7-day culture under shaking, the culture supernatant was collected. The recombinant protein of interest was purified from the culture supernatant using TALON Metal Affinity Resin (Clontech; bovine PD-1-His). After purification, the buffer was exchanged with PBS (pH 7.4) using PD MiniTrap G-25 (GE Healthcare). The resultant protein was stored at −30° C. until use in experiments (bovine PD-1-His). The concentration of purified bovine PD-1-His was quantitatively determined in terms of the absorbance (280 nm) measured with Nanodrop8000 Spectrophotometer (Thermo Fisher Scientific).

### 2.3. Preparation of Rat Anti-Bovine PD-1 Monoclonal Antibody Producing Cells

Rat was immunized in the footpad with bovine PD-1-Ig (described above). Hybridomas were established by the iliac lymph node method to thereby obtain rat anti-bovine PD-1 monoclonal antibody producing hybridoma 5D2. With respect to the method of establishment of rat anti-bovine PD-1 monoclonal antibody, details are disclosed in the following non-patent document (Ikebuchi R, Konnai S, Okagawa T, Yokoyama K, Nakajima C, Suzuki Y, Murata S, Ohashi K. Vet. Res. 44:59; Jul. 22, 2013).

### 2.4. Preparation of Rat-Bovine Chimeric Anti-Bovine PD-1 Antibody Expressing Vector

Rat-bovine chimeric anti-bovine PD-1 antibody ch5D2 was established by fusing the antibody constant regions of bovine IgG1 and Igλ, with rat anti-bovine PD-1 antibody 5D2 being used as antibody variable regions.

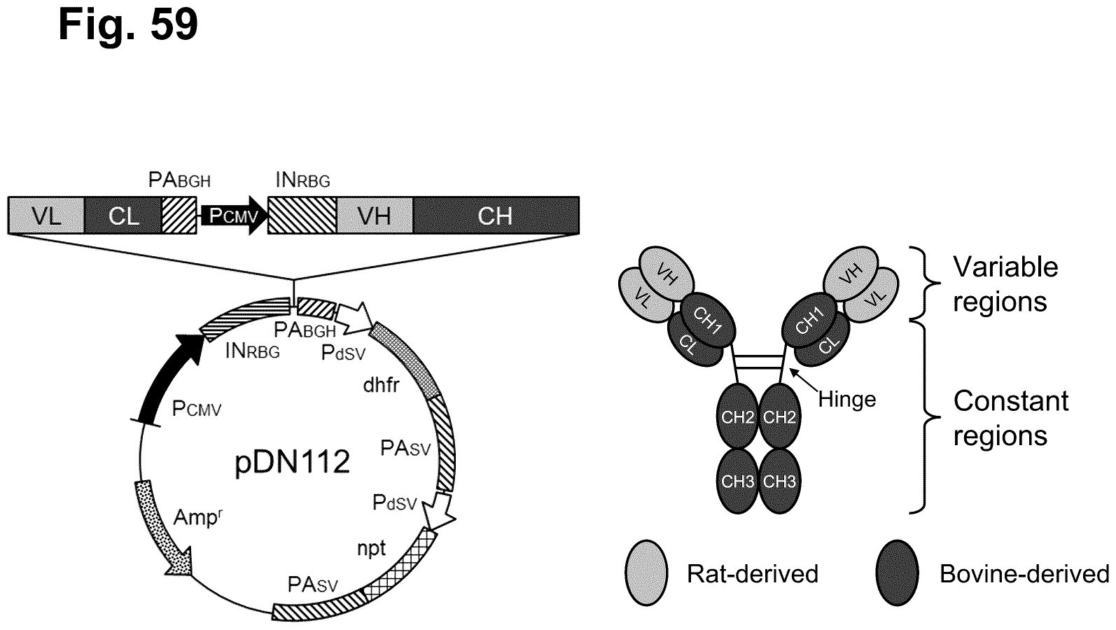

First, the genes of heavy chain and light chain variable regions were identified by the RACE method from a hybridoma that would produce rat anti-bovine PD-1 antibody 5D2. Subsequently, a gene sequence was prepared in which the heavy chain and the light chain variable regions of the rat anti-bovine PD-1 antibody 5D2 were linked to known constant regions of bovine IgG1 (heavy chain, modified from GenBank Accession number X62916) and bovine Igλ (light chain; GenBank Accession number X62917), respectively. Then, codon optimization was carried out (SEQ ID NOS: 157 and 158 (amino acid sequences); SEQ ID NOS: 162 and 163 (nucleotide sequences after codon optimization)). It should be noted that bovine IgG1 had mutations added to the putative binding sites of Fcγ receptors in CH2 domain in order to suppress ADCC activity (See FIGS. 1 and 11 for amino acid numbers and mutations: 251 E->P, 252 L->V, 253 P->A, 254 G->deletion, 348 A->S, 349 P->S; Ikebuchi R, Konnai S Okagawa, T, Yokoyama K, Nakajima C, Suzuki Y, Murata S, Ohashi K. Immunology, 142(4):551-561; August 2014). Then, the gene was artificially synthesized in such a manner that Nod recognition sequence, KOZAK sequence, chimeric antibody light chain sequence, poly-A addition signal sequence (PABGH), promoter sequence (PCMV), Sad recognition sequence, intron sequence (INRBG), KOZAK sequence, chimeric antibody heavy chain sequence and XbaI recognition sequence would be located in this order. The synthesized gene strand was digested with Nod (Takara) and XbaI (Takara), purified with FastGene Gel/PCR Extraction Kit (NIPPON Genetics) and cloned into the cloning site (Nod and XbaI restriction enzyme recognition sequences downstream of PCMV and between INRBG and PABGH) of expression plasmid pDN112 (kindly provided by Prof S. Suzuki, Hokkaido University Research Center for Zoonosis Control) treated with the restriction enzymes in the same manner (FIG. 59). The resultant plasmid of interest was extracted with QIAGEN Plasmid Midi kit (Qiagen) and stored at −30° C. until use in experiments. Hereinafter, the thus prepared expression plasmid is designated as pDN112-boPD-1ch5D2.

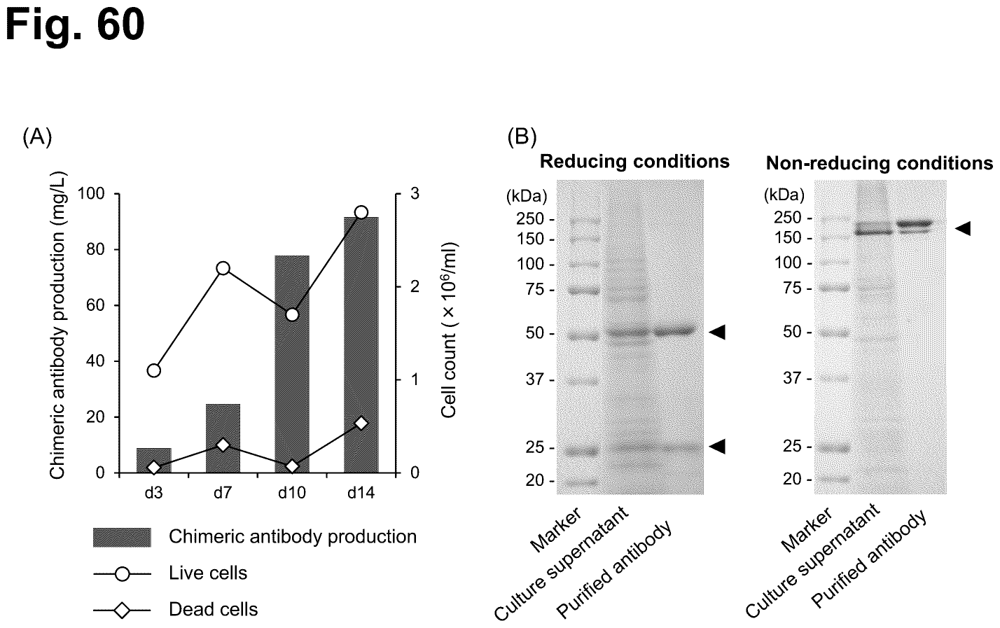

### 2.5. Expression of Rat-Bovine Chimeric Anti-Bovine PD-1 Antibody (FIG. 60)

The pDN112-boPD-1ch5D2 prepared above was transfected into CHO-DG44 cells (CHO-DG44(dfhr^{-/-})) which were a dihydrofolate reductase deficient cell. Forty-eight hours later, the medium was exchanged with CD OptiCHO medium (Life Technologies) containing 2 mM GlutaMAX supplement (Life Technologies) and 800 μg/ml G418 sulfate (Enzo Life Science). After cultured for 3 weeks, the expression cells were subjected to selection and cloning by limiting dilution. Subsequently, the concentrations of the chimeric antibody in the culture supernatants were measured by dot blotting and ELISA using anti-bovine IgG F(c) rabbit polyclonal antibody (Rockland) to thereby select high expression clones. Further, to the selected clones expressing rat-bovine chimeric anti-bovine PD-1 antibody at high levels were subjected to gene amplification treatment by adding a load with 60 nM methotrexate (Mtx; Wako)-containing medium. The thus established cell clone stably expressing rat-bovine chimeric anti-bovine PD-1 antibody was transferred into Mtx-free CD Opti-CHO medium and cultured under shaking for 14 days (125 rpm, 37° C., 5% CO₂). Chimeric antibody production in the culture supernatant was measured by ELISA using anti-bovine IgG F(c) rabbit polyclonal antibody (Rockland). For each washing operation in ELISA, Auto Plate Washer BIO WASHER 50 (DS Pharma Biomedical) was used. Absorbance was measured with Microplate Reader MTP-650FA (Corona Electric). The culture supernatant at day 14 was centrifuged at 10,000 g for 10 min to remove cells, and the centrifugal supernatant was passed through a Steritop-GP 0.22 μm filter (Millipore) for sterilization and then stored at 4° C. until it was subjected to purification.

The results are shown in FIG. 60A. Among the rat-bovine chimeric anti-bovine PD-1 antibody expressing cell clones, the most productive clone secreted 91.7 mg/l of the chimeric antibody into the culture supernatant during the 14-day culture under shaking.

### 2.6. Purification of Rat-Bovine Chimeric Anti-Bovine PD-1 Antibody

From the culture supernatant prepared as described above, each chimeric antibody was purified using Ab Capcher Extra (ProteNova). An open column method was used for binding to resin; 1.5 M Glycine/3 M NaCl (pH 8.0) was used as equilibration buffer and wash buffer. As elution buffer, 0.1 M Glycine-HCl (pH 2.8) was used. As neutralization buffer, 1M Tris (pH 9.0) was used. The purified antibody was subjected to buffer exchange with PBS (pH 7.4) using PD-10 Desalting Column (GE Healthcare) and concentrated using Amicon Ultra-15 (50 kDa, Millipore). The thus purified chimeric antibody was passed through a 0.22 μm syringe filter (Pall Life Sciences) for sterilization and stored at 4° C. until use in experiments.

### 2.7. Confirmation of the Purity of Purified Rat-Bovine Chimeric Anti-Bovine PD-1 Antibody (FIG. 60)

In order to confirm the purity of purified rat-bovine chimeric anti-bovine PD-1 antibody, antibody proteins were detected by SDS-PAGE and CBB staining. Purified rat-bovine chimeric anti-bovine PD-1 antibody ch5D2 was suspended in Laemmli Sample Buffer (Bio-Rad) and denatured at 95° C. for 5 min under reducing conditions (reduced with 2-mercaptoethaanol; Sigma-Aldrich) or under non-reducing conditions. The thus prepared samples were electrophoresed using 10% polyacrylamide gel. As molecular weight markers, Precision Plus Protein All Blue Standards (Bio-Rad) were used. After electrophoresis, the gel was stained with Quick-CBB kit (Wako) and subsequently decolored in distilled water.

The results are shown in FIG. 60B. Bands of rat-bovine chimeric anti-bovine PD-1 antibody were observed at predicted positions, that is, at 25 kDa (light chain) and 50 kDa (heavy chain) under reducing conditions and at 150 kDa under non-reducing conditions.

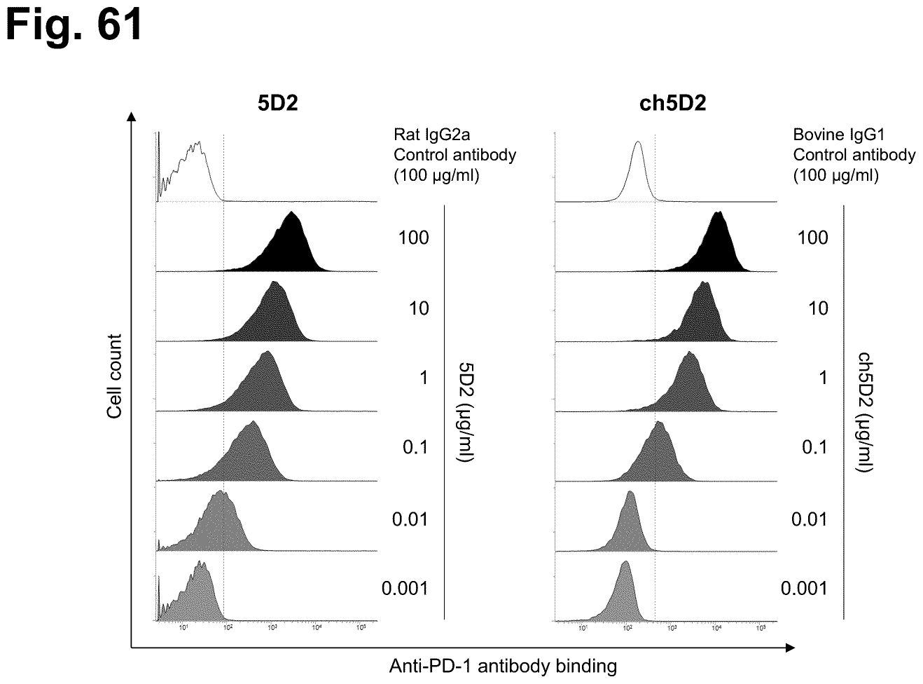

### 2.8. Binding Specificity of Rat-Bovine Chimeric Anti-Bovine PD-1 Antibody (FIG. 61)

It was confirmed by flow cytometry that rat-bovine chimeric anti-bovine PD-1 antibody specifically binds to bovine PD-1 expressing cells (described above). First, rat anti-bovine PD-1 antibody 5D2 or rat-bovine chimeric anti-bovine PD-1 antibody ch5D2 was reacted with bovine PD-1 expressing cells at room temperature for 30 min. After washing, Allophycocyanine (APC)-labeled anti-rat Ig goat antibody (Southern Biotech) or Alexa Fluor 647-labeled anti-bovine IgG (H+L) goat F(ab′)2 (Jackson ImmunoResearch) was reacted at room temperature for 30 min. As negative control antibody, rat IgG2a (κ) isotype control (BD Biosciences) or bovine IgG1 antibody (Bethyl) was used. After washing, each rat antibody or rat-bovine chimeric antibody bound to cell surfaces was detected by FACS Verse (BD Biosciences). For every washing operation and dilution of antibodies, PBS supplemented with 1% bovine serum albumin (Sigma-Aldrich) was used.

The experimental results are shown in FIG. 61. It was revealed that rat-bovine chimeric anti-bovine PD-1 antibody ch5D2 binds to bovine PD-1 expressing cells in the same manner as rat anti-bovine PD-1 antibody 5D2.

### 2.9. PD-1 Binding Avidity of Rat-Bovine Chimeric Anti-Bovine PD-1 Antibody

The binding avidities to bovine PD-1 of rat anti-bovine PD-1 antibody 5D2 and rat-bovine chimeric anti-bovine PD-1 antibody ch5D2 were measured by surface plasmon resonance using a biomolecular interaction analyzer (Biacore X100). Briefly, bovine PD-1-His (described above) was immobilized on a CM5 sensor chip (GE Healthcare) as a ligand. Subsequently, rat anti-bovine PD-1 antibody 5D2 or rat-bovine chimeric anti-bovine PD-1 antibody ch5D2 was reacted as an analyte, followed by single kinetics analysis. The experiment was repeated 3 times under the same conditions. Binding constant (kd value) and dissociation constant (ka value) were determined in each experiment, and binding avidity (KD value) was obtained.

The experimental results are shown in the table below. The binding avidity of rat-bovine chimeric anti-bovine PD-1 antibody for PD-1 protein was similar to that of rat anti-bovine PD-1 antibody 5D2, with no statistical difference observed (p>0.05; Welch's t-test).

| Anti-Bovine | | | |
|---|---|---|---|
| PD-1 | | | |
| Antibody | ka (1/Ms) | kd (1/s) | KD (M) |
| 5D2 | 1.84 × 10⁴ ± 0.27 | 2.15 × 10⁻⁴ ± 0.44 | 1.22 × 10⁻⁸ ± 0.39 |
| ch5D2 | 2.07 × 10⁴ ± 0.06 | 2.16 × 10⁻⁴ ± 1.12 | 1.05 × 10⁻⁸ ± 0.58 |

### 2.10. Blockade of Bovine PD-1/PD-L1 Binding by Rat-Bovine Chimeric Anti-Bovine PD-1 Antibody (FIG. 52)

Using bovine PD-L1 expressing cells (described above) and bovine PD-1-Ig (described above), bovine PD-1/PD-L1 binding inhibition by anti-PD-1 antibodies was tested. First, rat anti-bovine PD-1 antibody 5D2 or rat-bovine chimeric anti-bovine PD-1 antibody ch5D2 (final concentration: 0, 0.39, 0.78, 1.56, 3.12, 6.25, 12.5, 25 or 50 μg/ml) and bovine PD-1-Ig (final concentration: 5 μg/ml) labeled with biotin using Lightning-Link Type A Biotin Labeling Kit (Innova Biosciences) were added to 96-well plates, followed by reaction at 37° C. for 30 min. The resultant mixture was reacted with 1×10⁵ bovine PD-L1 expressing cells at 37° C. for 30 mn. After washing, the reaction mixture was reacted with APC-labeled streptavidin (BioLegend) at room temperature for 30 min to thereby detect bovine PD-1-Ig bound to cell surfaces. For analysis, FACS Verse (BD Biosciences) was used. For every washing operation and dilution of antibodies, PBS supplemented with 1% bovine serum albumin (Sigma-Aldrich) was used. Taking the proportion of bovine PD-1-Ig-bound cells without addition of antibodies as 100%, the proportion of bovine PD-1-Ig-bound cells at each antibody concentration was shown as a relative value.

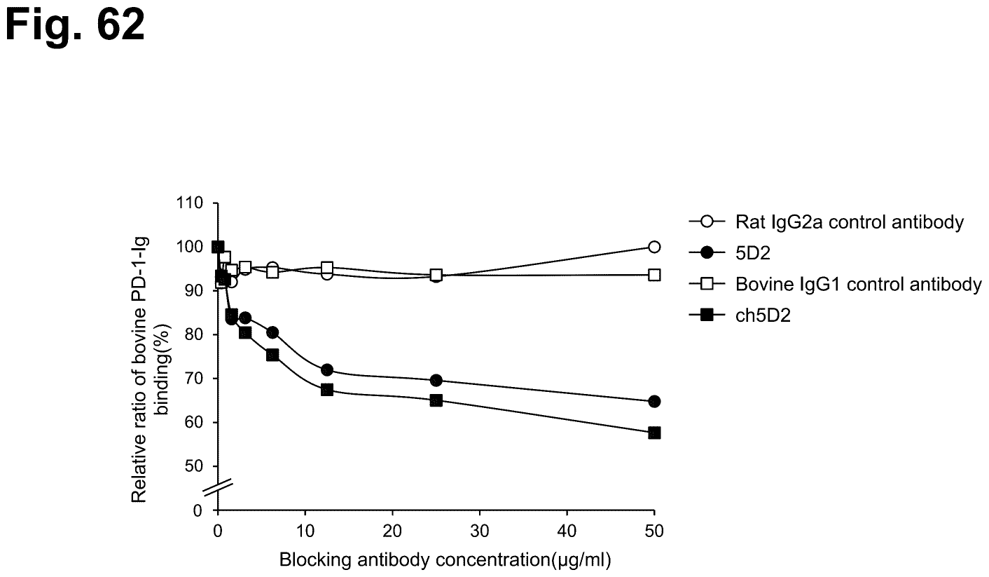

The experimental results are shown in FIG. 62. Rat-bovine chimeric anti-bovine PD-1 antibody ch5D2 inhibited the binding of PD-1-Ig to PD-L1 expressing cells by the same degree as rat anti-bovine PD-1 antibody 5D2 did.

### 2.11. CDR Analysis of Rat Anti-Bovine PD-1 Antibody

The complementarity-determining regions (CDRs) of rat anti-bovine PD-1 antibody 5D2 were determined using NCBI IGBLAST (http://www.ncbi.nlm.nih.2ov/i2blast/). The results are shown in FIG. 58.

2.12. Inoculation Test on Cattle with Rat-Bovine Chimeric Anti-Bovine PD-1 Antibody

Established rat-bovine chimeric anti-bovine PD-1 antibody ch5D2 (14 mg; 0.08 mg/kg) was intravenously administrated into an experimentally BLV-infected calf (Holstein, male, 4 months old, 173.5 kg). Blood samples were collected chronologically from the infected calf, followed by collection of blood (with heparin sodium (Ajinomoto) used as anticoagulant) and serum. Peripheral blood mononuclear cells (PBMCs) were isolated from the blood by density gradient centrifugation using Percoll (GE Healthcare).

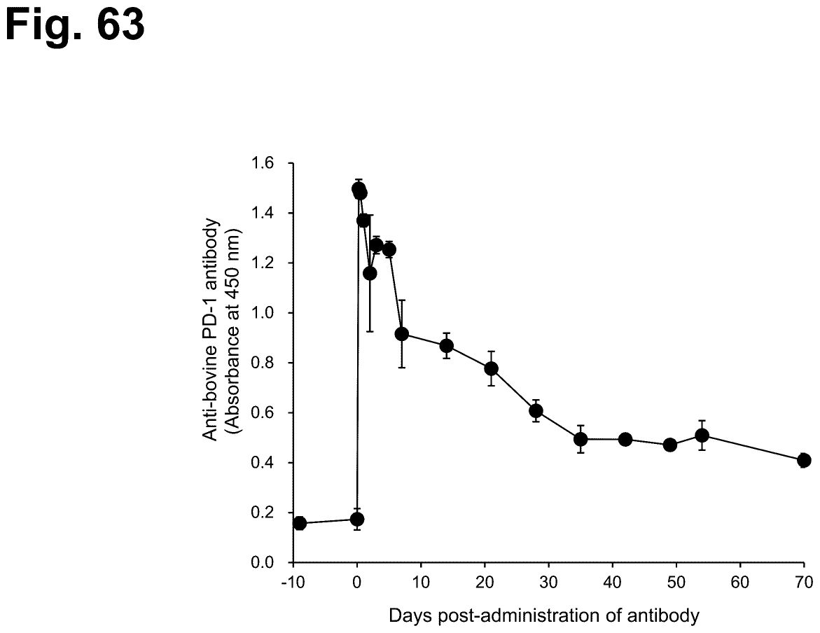

### 2.13. Kinetics of Administered Rat-Bovine Chimeric Anti-Bovine PD-1 Antibody in Blood (FIG. 63)

Bovine PD-1-His (described above) was immobilized on ELISA plates (H type, Sumitomo Bakelite) at a final concentration of 10 μg/ml at 4° C. overnight. Subsequently, each well was washed with 200 μl of 0.05% Tween 20-supplemented Tris-buffered saline (TBS-T) five times, followed by blocking with 1% skim milk-supplemented TBS-T at room temperature for 1 hr. Another washing was carried out in the same manner. The serum collected from the test calf was added to each well and reacted at room temperature for 1 hr. After washing, horseradish peroxidase-labeled anti-bovine IgG F(c) rabbit polyclonal antibody (Rockland) was reacted at room temperature for 1 hr. Each well was washed again and then TMB One Component Substrate (Bethyl) was added for coloring. The enzyme reaction was terminated with 0.18 M dilute sulfuric acid. Absorbance (450 nm) was measured with Microplate Reader MTP-650FA (Corona Electric). For every plate washing operation, Auto Plate Washer BIO WASHER 50 (DS Pharma Biomedical) was used.

The experimental results are shown in FIG. 63. Rat-bovine chimeric anti-bovine PD-1 antibody was detected in the serum of the test calf until 70 days after administration (at the end of the clinical test). The antibody retained particularly high concentrations for one week after administration.

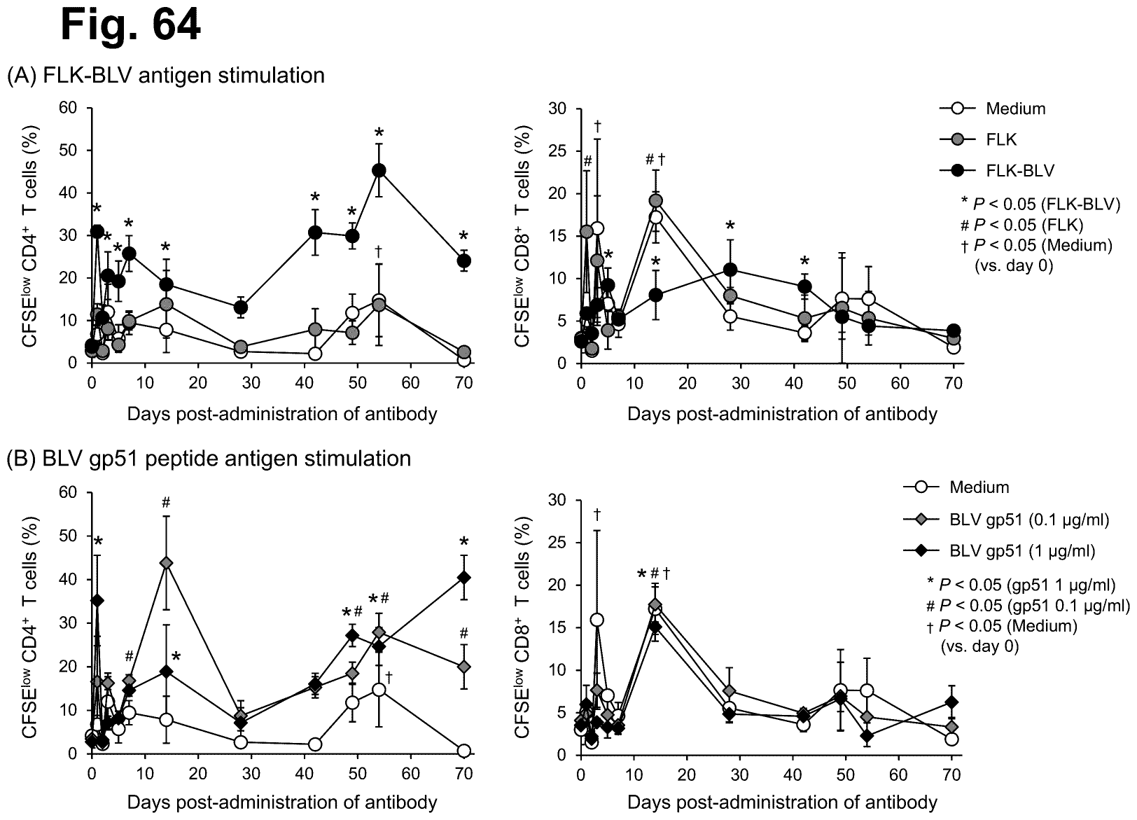

### 2.14. Cell Proliferation Response of T Cells to BLV Antigen (FIG. 64)

Bovine PBMCs were suspended in PBS and reacted with carboxyfluorescein succinimidyl ester (CFSE; Invitrogen) at room temperature for 20 min for labeling. After washing twice with RPMI 1640 medium (Sigma-Aldrich) containing 10% inactivated fetal bovine serum (Cell Culture Technologies), penicillin 200 U/ml, streptomycin 200 μg/ml and 0.01% L-glutamine (Life Technologies), cell concentration was adjusted to 4×10⁶ cells/ml using the same medium. To the PBMCs, culture supernatant of 2% BLV-infected fetal lamb kidney cell (FLK-BLV), culture supernatant of fetal lamb kidney cell (FLK) not infected with 2% BLV, or BLV gp51 peptide mix 0.1 μg/ml or 1 μg/ml was added, followed by a 6-day culture at 37° C. under 5% CO₂. After 6 days, PBMCs were recovered and reacted with Alexa Fluor 647-labeled mouse anti-bovine CD4 antibody (CC30, AbD Serotec), Peridinin-chlorophyll-protein complex/cyanin 5.5-labeled mouse anti-bovine CD8 antibody (CC63, AbD Serotec) and R-Phycoerythrin/cyanin 7 (PE/Cy7)-labeled anti-bovine IgM mouse antibody (IL-A30, AbD Serotec) at 4° C. for 20 min. For labeling antibodies, Zenon Mouse IgG1 Labeling Kits (Life Technologies) or Lightning-Link Kits (Innova Biosciences) was used. For analysis, FACS Verse (BD Biosciences) was used. For every washing operation and dilution of antibodies, PBS supplemented with 1% bovine serum albumin (Sigma-Aldrich) was used. With respect to the proportion of proliferated T cells (CFSE^{low} cells), statistical test was performed using the method of Dunnett.

The experimental results are shown in FIG. 64. Upon administration of rat-bovine chimeric anti-bovine PD-1 antibody, BLV-specific cell proliferative response in CD4⁺ T cells showed a statistically significant increase immediately after the administration, compared to the response before administration.

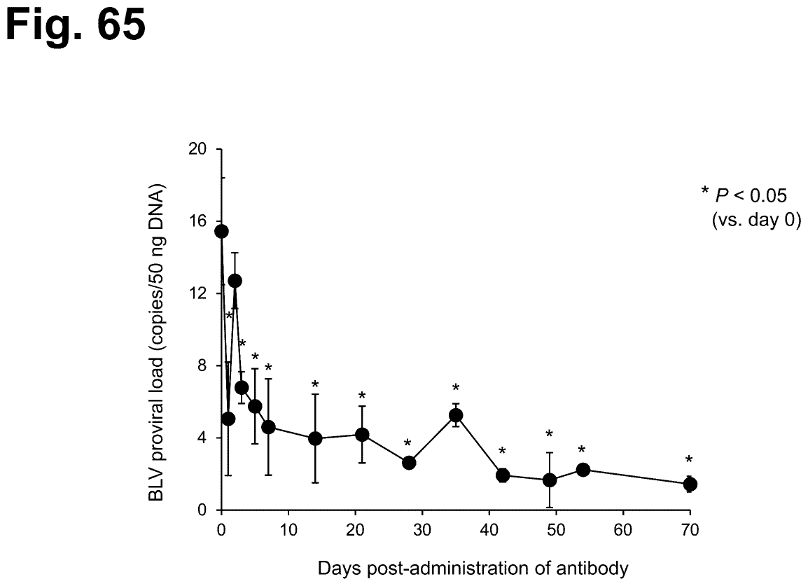

### 2.15. Transition in BLV Proviral Load (FIG. 65)

DNA was extracted from isolated bovine PBMCs using Wizard DNA Purification kit (Promega). The concentration of the extracted DNA was quantitatively determined based on the absorbance (260 nm) measured with Nanodrop 8000 Spectrophotometer (Thermo Fisher Scientific). For measuring BLV proviral load in PBMCs, real time PCR was performed using Cycleave PCR Reaction Mix SP (Takara) and Probe/Primer/Positive control (Takara) for bovine leukemia virus detection. LightCycler480 System II (Roche Diagnosis) was used for the measurement. With respect to the measured proviral load, statistical test was performed by the method of Dunnett.

The experimental results are shown in FIG. 65. Upon administration of rat-bovine chimeric anti-bovine PD-1 antibody, BLV proviral load in PBMCs showed a statistically significant decrease immediately after the administration, compared to the load before administration. The BLV proviral load remained at low levels until the end of the clinical test (day 70).

### [Example 6] Application of Anti-PD-1 Antibody to Other Animal Species

### 1. Materials, Methods and Experimental Results

### 1.1. Identification of Ovine and Water Buffalo PD-1 Genes

In order to determine the full-lengths of the coding sequences (CDSs) of ovine and water buffalo PD-1 cDNAs, primers for amplifying the full lengths of CDSs were first designed (ovPD-1 CDS F and R; buPD-1 CDS F1, R1, F2 and R2) based on the nucleotide sequences of ovine and water buffalo PD-1 genes (GenBank accession numbers BC123854 and XM_012176227), and then PCR was performed using a synthesized ovine or water buffalo PBMC-derived cDNA as a template. For the resultant amplified products, nucleotide sequences were determined with a capillary sequencer according to conventional methods (Mingala C N, Konnai S, Ikebuchi R, Ohashi K. Comp. Immunol. Microbiol. Infect. Dis., 34(1):55-63; January 2011; Water buffalo PD-1 gene was identified in this article).

Primer (ovPD-1 CDS F):
(SEQ ID NO: 215)
ATGGGGACCCCGCGGGCGCC
Primer (ovPD-1 CDS R):
(SEQ ID NO: 216)
TCAGAGGGGCCAGGAGCAGTGTCCA
Primer (buPD-1 CDS F1):
(SEQ ID NO: 217)
ATGGGGACCCCGCGGGCGCT
Primer (buPD-1 CDS R1):
(SEQ ID NO: 218)
GATGACCAGGCTCTGCATCT
Primer (buPD-1 CDS F2):
(SEQ ID NO: 219)
AATGACAGCGGCGTCTACTT
Primer (buPD-1 CDS R2):
(SEQ ID NO: 220)
TCAGAGGGGCCAGGAGCAGT

### 1.2. Construction of Ovine PD-1 Expressing COS-7 Cells

In order to prepare ovine PD-1 expression plasmid, PCR was performed using a synthesized ovine PBMC-derived cDNA as a template and primers designed by adding BglII and SmaI recognition sites on the 5′ side (ovPD-1-EGFP F and R). The resultant PCR products were digested with BglII (Takara) and SmaI (Takara), purified with FastGene Gel/PCR Extraction Kit (NIPPON Genetics) and cloned into pEGFP-N2 vector (Clontech) treated with the restriction enzymes in the same manner. The expression plasmid of interest was extracted using FastGene Xpress Plasmid PLUS Kit (NIPPON Genetics) and stored at −30° C. until use in experiments. Hereinafter, the thus prepared plasmid is designated as pEGFP-N2-ovPD-1.

Primer (ovPD-1-EGFP F):
(SEQ ID NO: 221)
GAAGATCTATGGGGACCCCGCGGGCGCCG
Primer (ovPD-1-EGFP R):
(SEQ ID NO: 222)
GACCCGGGGAGGGGCCAGGAGCAGTGTCC

COS-7 cells were subcultured at a density of 5×10⁴ cells/cm² in 6-well plates, and then cultured overnight in RPMI 1640 medium containing 10% inactivated fetal bovine serum (Invitrogen) and 0.01% L-glutamine (Life Technologies) at 37° C. in the presence of 5% CO₂. The pEGFP-N2-ovPD-1 or pEGFP-N2 (negative control) was introduced into COS-7 cells at 0.4 μg/cm² using Lipofectamine 2000 (Invitrogen). The cells were cultured for 48 hours (ovPD-1-EGFP expressing cells). In order to confirm the expression of ovine PD-1 in the thus prepared expressing cells, intracellular localization of EGFP was visualized with an all-in-one fluorescence microscope BZ-9000 (KEYENCE).

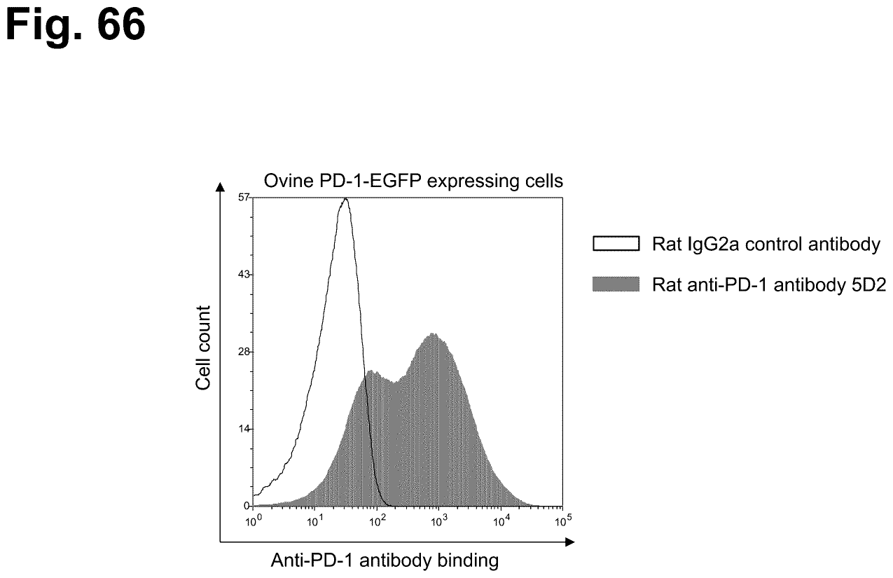

1.3. Reactivity of Rat Anti-Bovine PD-1 Antibody 5D2 with Ovine PD-1 (FIG. 66)

It was confirmed by flow cytometry that rat anti-bovine PD-1 monoclonal antibody cross-reacts with ovine PD-1. Ovine PD-1-EGFP expressing COS-7 cells were blocked with 10% inactivated goat serum (Invitrogen)-supplemented PBS at room temperature for 15 min and reacted with 10 μg/ml of rat anti-bovine PD-1 antibody 5D2 at room temperature for 30 min. After washing, the cells were reacted with APC-labeled anti-rat Ig goat antibody (Beckman Coulter) at room temperature for 30 min. As a negative control antibody, rat IgG2a (κ) isotype control (BD Bioscience) was used. For analysis, FACS Verse (BD Bioscience) was used. For every washing operation and dilution of antibodies, 1% bovine serum albumin (Sigma-Aldrich)-supplemented PBS was used.

The experimental results are shown in FIG. 66. It was confirmed that rat anti-bovine PD-1 antibody 5D2 binds to ovine PD-1 expressing cells.

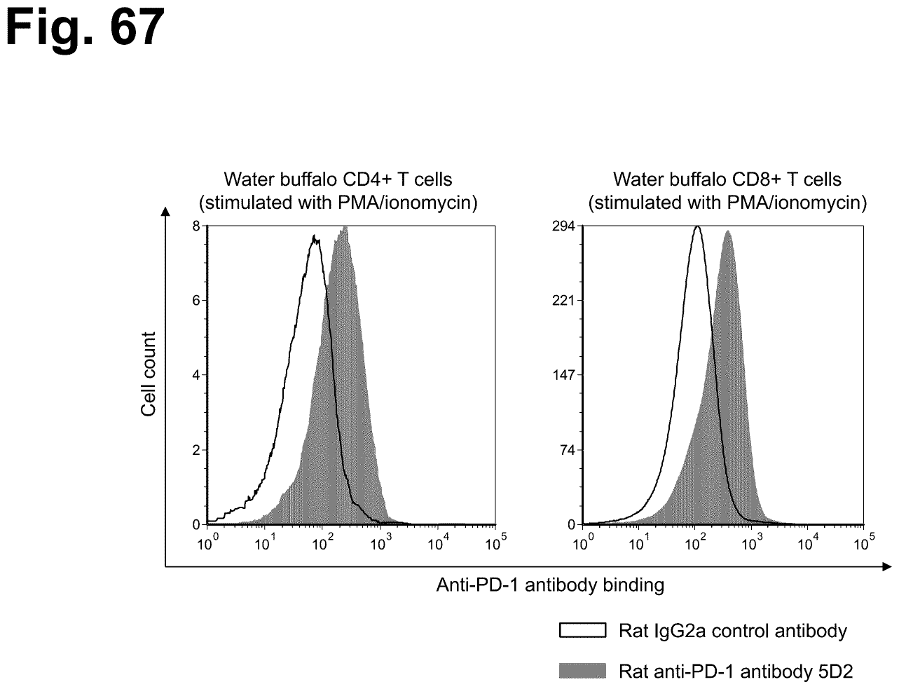

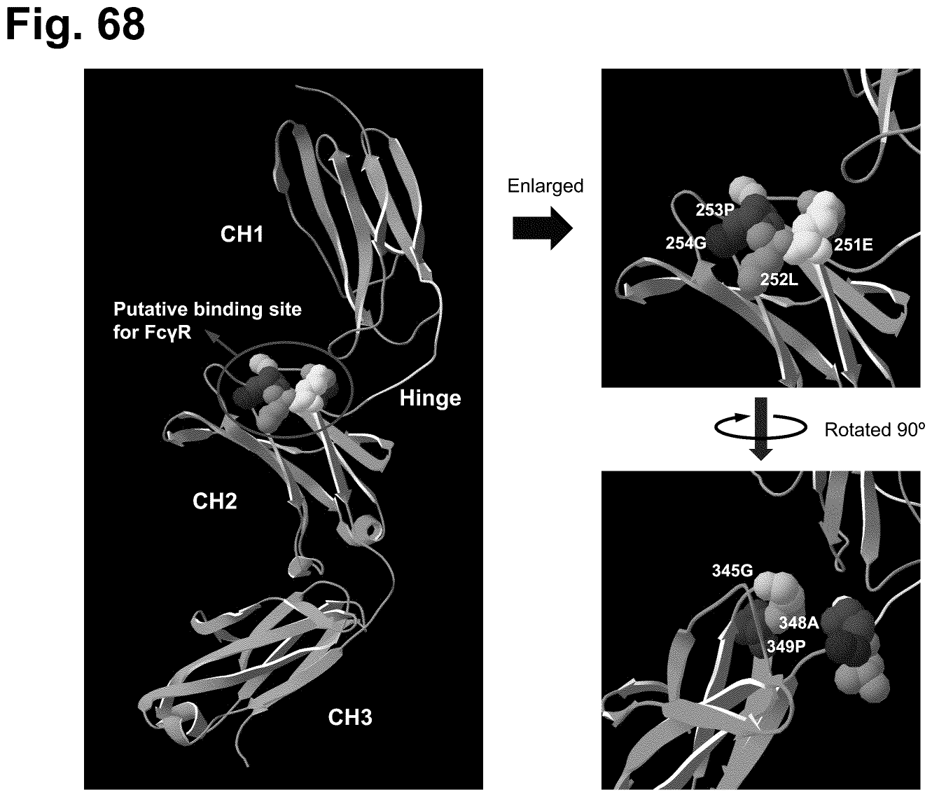

1.4. Reactivity of Rat Anti-Bovine PD-1 Antibody 5D2 with Water Buffalo Lymphocytes (FIG. 67)

Peripheral blood mononuclear cells (PBMCs) were isolated from peripheral blood of water buffalo (Bubalus ubalis; Asian water buffalo) by density gradient centrifugation using Percoll C Healthcare). The isolated water buffalo PBMCs were suspended in RPMI 1640 medium (Sigma-Aldrich) containing 10% inactivated fetal bovine serum (Cell Culture Technologies), penicillin 200 U/ml, streptomycin 200 μg/ml and 0.01% L-glutamine (Life Technologies). Cell density was adjusted to 2×10⁶ cells/ml. To these PBMCs, phorbol 12-myristate acetate (PMA) 20 ng/ml and ionomycin 1 μg/ml (Sigma-Aldrich) were added, followed by a 2-day culture at 37° C. under 5% CO₂. Cultured PBMCs were harvested and blocked with 10% inactivated goat serum (Invitrogen)-supplemented PBS at room temperature for 15 min. Then, rat anti-bovine PD-1 antibody 5D2 and mouse anti-bovine CD8 antibody (38.65, AbD Serotec) were reacted at room temperature for 30 min. As a negative control, rat IgG2a (κ) isotype control (BD Bioscience) was used. After washing, APC-labeled goat anti-rat Ig antibody (Beckman Coulter) and PE-labeled goat anti mouse IgG antibody (Beckman Coulter) were reacted at room temperature for 30 min. After further washing, Alexa Flour488-labeled mouse anti-bovine CD4 antibody (CC30, AbD Serotec) and PE/Cy7-labeled anti-bovine IgM mouse antibody (IL-A30, AbD Serotec) were reacted at room temperature for 30 min. For antibody labeling, Zenon Mouse IgG1 Labeling Kits (Life Technologies) or Lightning-Link Kits (Innova Biosciences) was used. For analysis, FACS Verse (BD Biosciences) was used. For every washing operation and dilution of antibodies, 10% inactivated goat serum (Invitrogen)-supplemented PBS was used.

The experimental results are shown in FIG. 67. Rat anti-bovine PD-1 antibody 5D2 strongly bound to water buffalo CD4⁺ T cells (IgM-CD4⁺) and CD8⁺ T cells (IgM-CD8⁺) that had been activated by PMA/ionomycin stimulation.

### [Example 7] Binding to Bovine Fcγ Receptors of Rat-Bovine Chimeric Anti-Bovine PD-1 Antibody Having Wild-Type or Mutated Bovine IgG1

### 1. Introduction

The present inventors have established a rat-bovine chimeric anti-bovine PD-1 antibody in Example 5 with a view to establishing a novel therapy for bovine infections. In the process, mutations were added to putative binding sites for Fcγ receptors in bovine IgG1 CH2 domain in order to suppress ADCC activity mediated by the chimeric antibody (FIGS. 1 and 11). In the subject Example, in order to examine the effect of these amino acid mutations, the present inventors prepared rat-bovine chimeric anti-bovine PD-1 antibodies having mutated bovine IgG1 (“IgG1 ADCC—” described above) and wild-type bovine IgG1 (“IgG1 WT”), respectively, and confirmed their binding to known bovine Fcγ receptors.

### 2. Materials, Methods and Experimental Results

### 2.1 Preparation of Rat-Bovine Chimeric Anti-Bovine PD-1 Antibody Expressing Vector

Rat-bovine chimeric anti-bovine PD-1 antibody ch5D2 having wild-type bovine IgG1 (IgG1 WT) or mutated bovine IgG1 (IgG1 ADCC—described above) was established.

An expression plasmid encoding rat-bovine chimeric anti-bovine PD-1 antibody ch5D2 having mutated bovine IgG1 (IgG1 ADCC—) was prepared according to the procedures described in Example 5 (SEQ ID NOS: 157 and 158 (amino acid sequences), SEQ ID NOS: 162 and 163 (nucleotide sequences after codon optimization)). It should be noted that in order to suppress ADCC activity, the bovine IgG1 used in ch5D2 IgG1 ADCC—had mutations added to the putative binding sites for Fcγ receptors in CH2 domain (see FIGS. 1 and 11 for amino acid numbers and mutations: 251 E->P, 252 L->V, 253 P->A, 254 G->deletion, 348 A->S, 349 P->S; Ikebuchi R, Konnai S, Okagawa T, Yokoyama K, Nakajima C, Suzuki Y, Murata S, Ohashi K. Immunology, 142(4):551-561; August 2014). Hereinafter, the thus prepared plasmid is designated as pDN112-boPD-1ch5D2 IgG1 ADCC—.

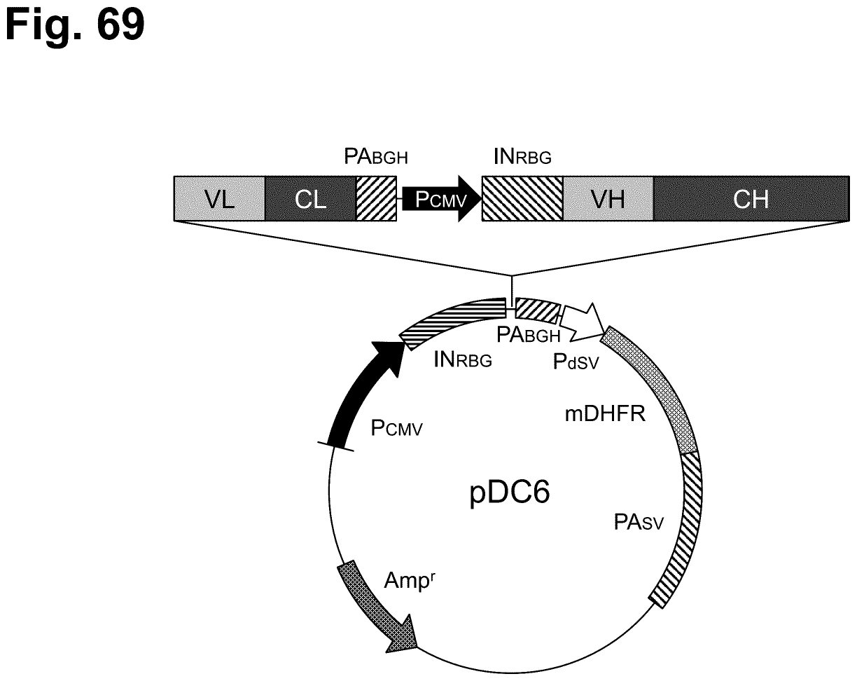

An expression plasmid encoding rat-bovine chimeric anti-bovine PD-1 antibody ch5D2 having wild-type IgG1 (IgG1 WT) was prepared according to the procedures described below. First, in order to amplify the gene encoding the constant region of wild-type bovine IgG1 (GenBank accession number X62916), PCR was performed using a synthesized bovine PBMC-derived cDNA as a template and designed primers that have NheI and XbaI recognition sites added on the 5′ side (boIgG1 CH1 F and boIgG1 CH3 R). The amplified gene strand was digested with NheI (Takara) and XbaI (Takara), purified with FastGene Gel/PCR Extraction Kit (NIPPON Genetics), and cloned into pDN112-boPD-1ch5D2 IgG1 ADCC—that had been treated with the restriction enzymes in the same manner. Further, the resultant plasmid was purified with QIAGEN Plasmid Midi kit (Qiagen) and digested with Nod (Takara) and XbaI (Takara) to thereby obtain an expression cassette for ch5D2's light chain (SEQ ID NO: 157 (amino acid sequence), SEQ ID NO: 162 (nucleotide sequence)) and heavy chain (IgG1 WT) (SEQ ID NO: 223 (amino acid sequence), SEQ ID NO: 224 (nucleotide sequence)). This gene fragment was purified with FastGene Gel/PCR Extraction Kit (NIPPON Genetics) and cloned into the cloning site (Nod and XbaI restriction enzyme recognition sequences downstream of PCMV and between INRBG and PABGH) of expression vector pDC6 (kindly provided by Prof. S. Suzuki, Hokkaido University Research Center for Zoonosis Control) (FIG. 69). The resultant expression plasmid of interest was extracted with QIAGEN Plasmid Midi kit (Qiagen) and stored at −30° C. until use in experiments. Hereinafter, the thus prepared expression plasmid is designated as pDC6-boPD-1ch5D2 IgG1 WT.

Primer (boIgG1 CH1 F):
(SEQ ID NO: 225)
CTAGCTAGCACCACAGCCCCGAAAGTCT
Primer (boIgG1 CH3 R):
(SEQ ID NO: 226)
TGCTCTAGATTATTTACCCGCAGACTTAGA

### 2.2. Expression and Purification of Rat-Bovine Chimeric Anti-Bovine PD-1 Antibody

Thirty micrograms of pDC6-boPD-1ch5D2 IgG1 WT or pDN112-boPD-1ch5D2 IgG1 ADCC—was introduced into 7.5×10⁷ Expi293F cells (Life Technologies) using Expifectamine (Life Technologies) and the transfected cells were then cultured under shaking for 5 to 7 days, followed by collection of a culture supernatant. Each chimeric antibody was purified from the culture supernatant using Ab Capcher Extra (ProteNova). An open column method was used for binding to resin; 1.5 M Glycine/3 M NaCl (pH 8.0) was used as equilibration buffer and wash buffer. As elution buffer, 0.1 M Glycine-HCl (pH 2.8) was used. As neutralization buffer, 1M Tris (pH 9.0) was used. The purified antibody was subjected to buffer exchange with PBS (pH 7.4) using PD-10 Desalting Column (GE Healthcare) and concentrated using Amicon Ultra-15 (50 kDa, Millipore). The thus purified chimeric antibody was passed through a 0.22 μm syringe filter (Pall Life Science) for sterilization and stored at 4° C. until use in experiments. The concentration of each chimeric antibody as purified was quantitatively determined with the absorbance (280 nm) measured with Nanodrop8000 Spectrophotometer (Thermo Fisher Scientific).

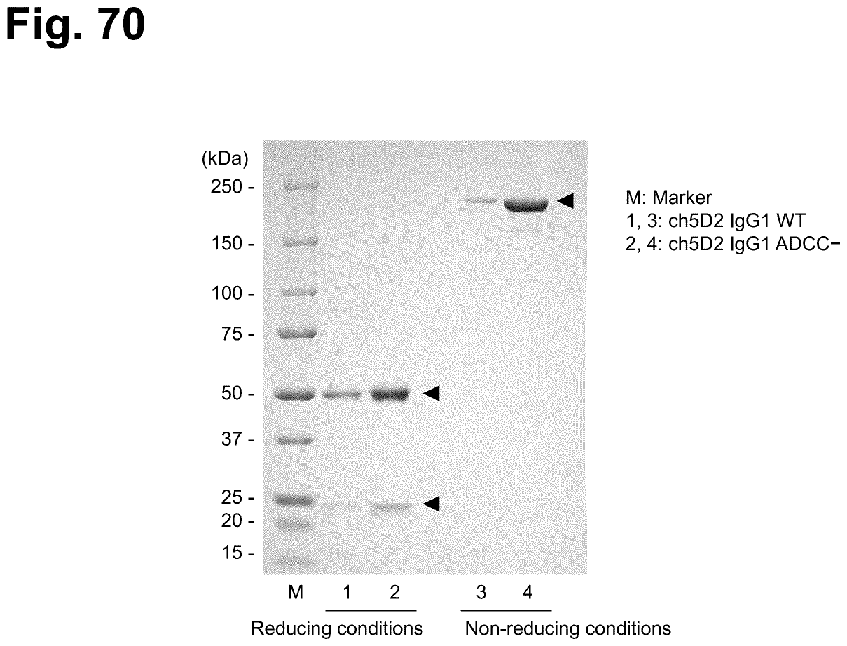

### 2.3. Confirmation of the Purity of Purified Rat-Bovine Chimeric Anti-Bovine PD-1 Antibodies (FIG. 70)

In order to confirm the purity of purified rat-bovine chimeric anti-bovine PD-1 antibodies (ch5D2 IgG1 WT and ch5D2 IgG1 ADCC—), antibody proteins were detected by SDS-PAGE and CBB staining. Each chimeric antibody purified was suspended in Laemmli Sample Buffer (Bio-Rad) and denatured at 95° C. for 5 min under reducing conditions (reduced with 2-mercaptoethaanol; Sigma-Aldrich) or under non-reducing conditions. The thus prepared samples were electrophoresed using SuperSep Ace 5%-20% gradient polyacrylamide gel (Wako). As molecular weight markers, Precision Plus Protein All Blue Standards (Bio-Rad) were used. After electrophoresis, the gel was stained with Quick-CBB kit (Wako) and decolored in distilled water.

The results are shown in FIG. 70. Bands of ch5D2IgG1 WT and ch5D2 IgG1 ADCC—were observed at predicted positions, that is, at 25 kDa (light chain) and 50 kDa (heavy chain) under reducing conditions and at 150 kDa under non-reducing conditions.

### 2.4. Construction of Soluble Bovine Fcγ Receptors (FcγRs)

Bovine FcγRI-His, FcγRII-His, FcγRIII-His and Fcγ2R-His expressing plasmids were constructed according to the procedures described below. In order to amplify the signal peptide and the extracellular region of bovine FcγRI, FcγRII, FcγRIII and Fcγ2R (GenBank accession numbers NM_174538, NM_174539, NM_001077402 and NM_001001138), primers were designed which had NotI and XhoI recognition sites added on the 5′ side (boFγyRI-His F and R; boFγyRIII-His F and R; or boFcγ2R-His F and R) or NheI and EcoRV recognition sites added on the 5′ side (boFcγRIII-His F and R). A gene sequence encoding a 6xHis tag was added to reverse primers. PCR was performed using a synthesized bovine PBMC-derived cDNA as a template. The respective PCR products were digested with Nod (Takara) and XhoI (Takara) (FcγRI-His, FcγRIII-His and Fcγ2R-His) or NheI (Takara) and EcoRV (Takara) (FcγRII-His), purified with FastGene Gel/PCR Extraction Kit (NIPPON Genetics) and cloned into pCXN2.1(+) vector (Niwa H, Yamamura K, Miyazaki J. Gene, 108(2):193-199; Dec. 15, 1991; kindly provided by Dr. T. Yokomizo, Juntendo University Graduate School of Medicine). The resultant expression plasmids were purified with FastGene Xpress Plasmid PLUS Kit (NIPPON Genetics) and stored at −30° C. until use in experiments. Hereinafter, the thus prepared expression plasmid is designated as pCXN2.1-boFcγRI-His, pCXN2.1-boFcγRII-His, pCXN2.1-boFcγRIII-His or pCXN2.1-boFcγ2R-His.

Primer (boFcγRI-His F):
(SEQ ID NO: 227)
ATAAGAATGCGGCCGCCACCATGTGGCTCATAATAGCTCT
Primer (boFcγRI-His R):
(SEQ ID NO: 228)
GCCCTCGAGTTAATGGTGATGGTGATGGTGAGGAGTTGTTGACTGGAGGC
Primer (boFcγRII-His F):
(SEQ ID NO: 229)
ATAAGAATGCTAGCCACCATGGGGATCCCCTCATTCCT
Primer (boFcγRII-His R):
(SEQ ID NO: 230)
GCCGATATCTTAATGGTGATGGTGATGGTGCGATGAGGGGCCGCTCGAGC
Primer (boFcγRIII-His F):
(SEQ ID NO: 231)
ATAAGAATGCGGCCGCCACCATGTGGCAACTGCTACCACC
Primer (boFcγRIII-His R):
(SEQ ID NO: 232)
GCCCTCGAGTTAATGGTGATGGTGATGGTGGTGCCAAGGTAGAAAGAATG
Primer (boFcγ2R-His F):
(SEQ ID NO: 233)
ATAAGAATGCGGCCGCCACCATGGCCCCCACCCTCCCTGCCTTGCTCT
Primer (boFcγ2R-His R):
(SEQ ID NO: 234)
GCCCTCGAGTTAATGGTGATGGTGATGGTGATTCTGCATCGTGTAGTCTG

Soluble bovine FcγRI-His, FcγRII-His, FcγRIII-His and Fcγ2R-His expressing cells were prepared according to the procedures described below. Briefly, 30 μg of pCXN2.1-boFcγRI-His, pCXN2.1-boFcγRII-His, pCXN2.1-boFcγRIII-His or pCXN2.1-boFcγ2R-His was introduced into 7.5×10⁷ Expi293F cells (Life Technologies) using Expifectamine (Life Technologies) and the transfected cells were then cultured under shaking for 5 to 7 days, followed by collection of a culture supernatant. Recombinant proteins were purified from the culture supernatant using TALON Metal Affinity Resin (Clontech). After purification, the buffer was exchanged with PBS (pH 7.4) using Amicon Ultra-15 Centrifugal Filter Unit (10 kDa, Millipore), and the recombinant proteins were stored at −30° C. until use in experiments (bovine PD-1-His). The concentrations of purified bovine FcγRI-His, FcγRII-His, FcγRIII-His and Fcγ2R-His were quantitatively determined in terms of the absorbance (280 nm) measured with Nanodrop8000 Spectrophotometer (Thermo Fisher Scientific).

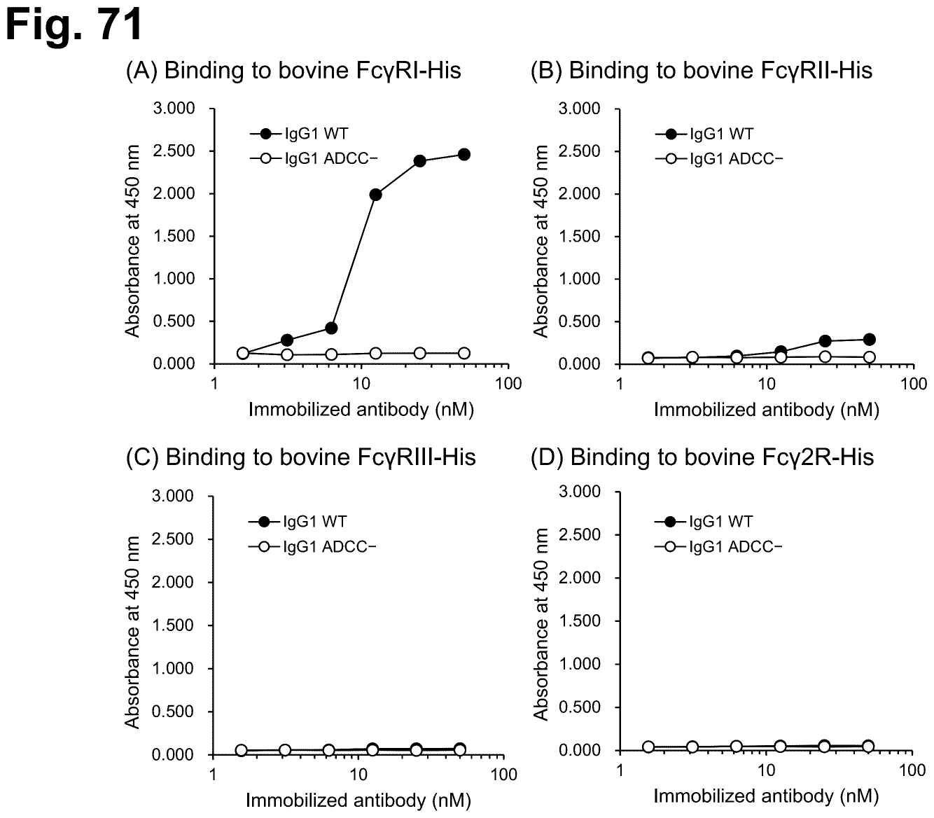

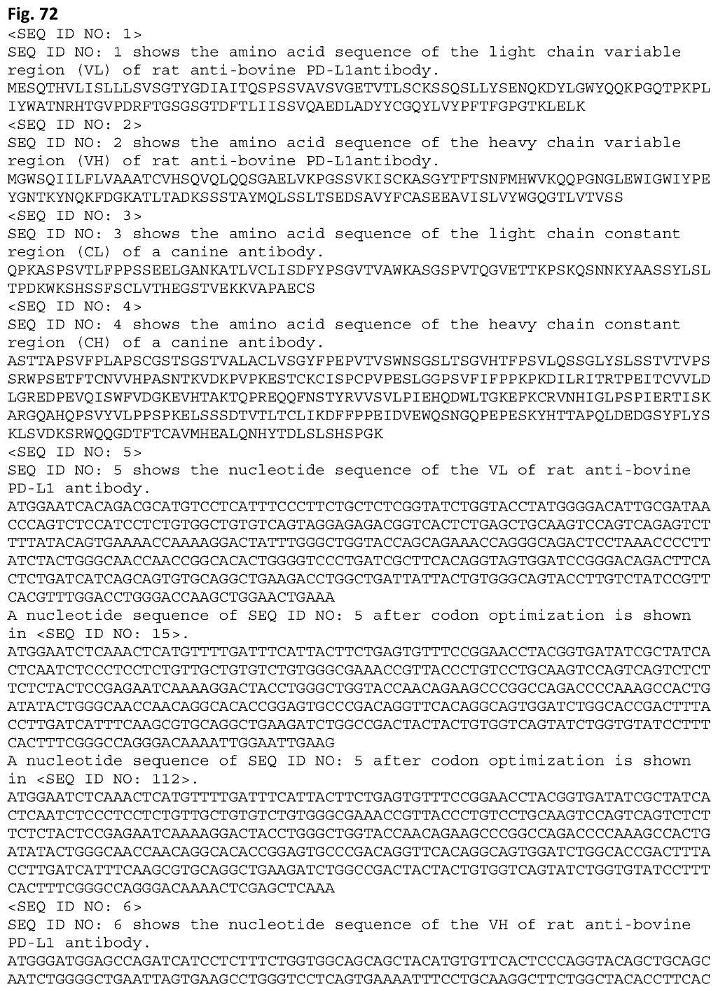

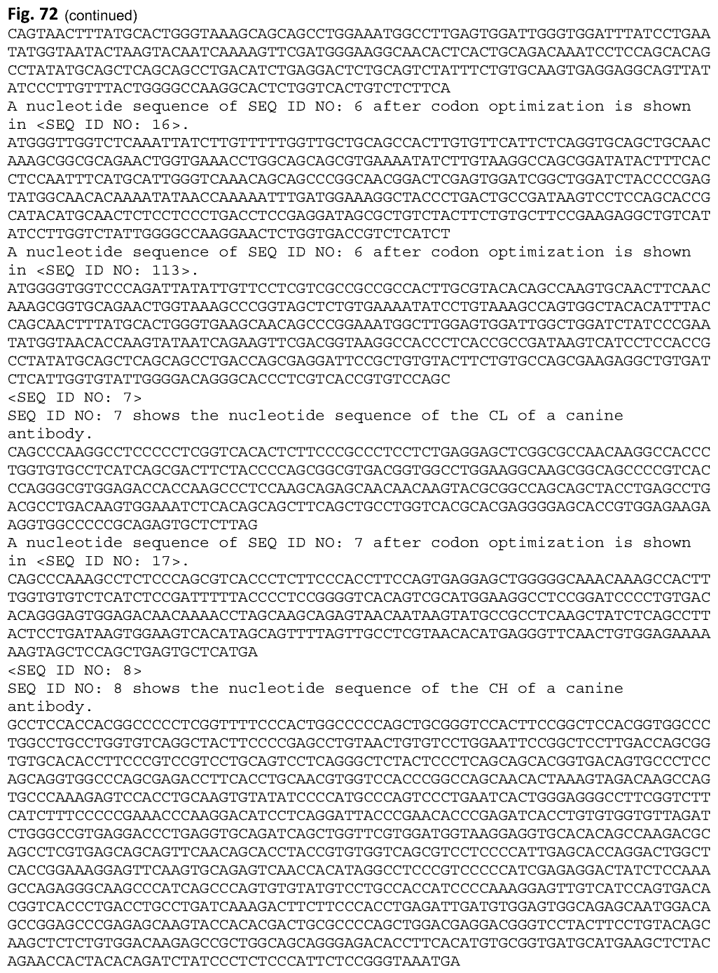

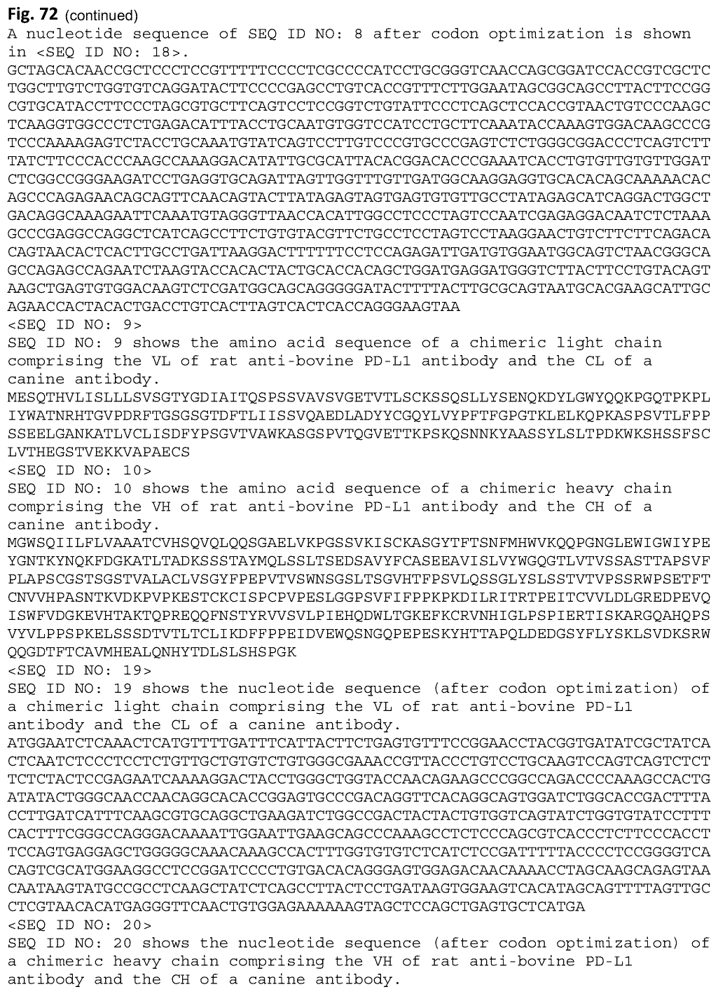

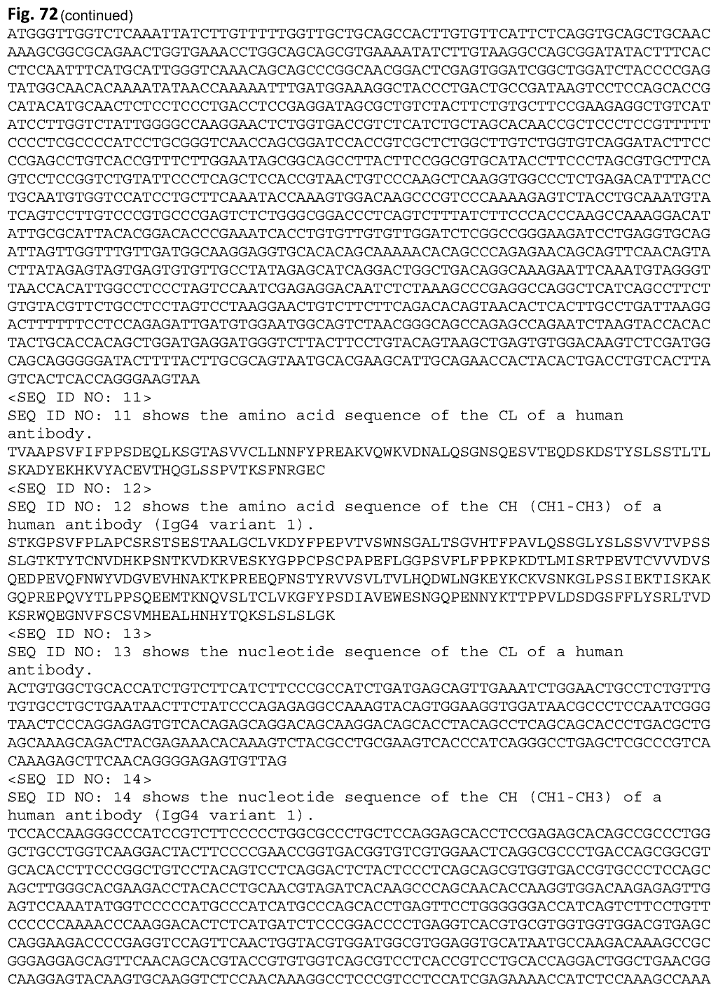

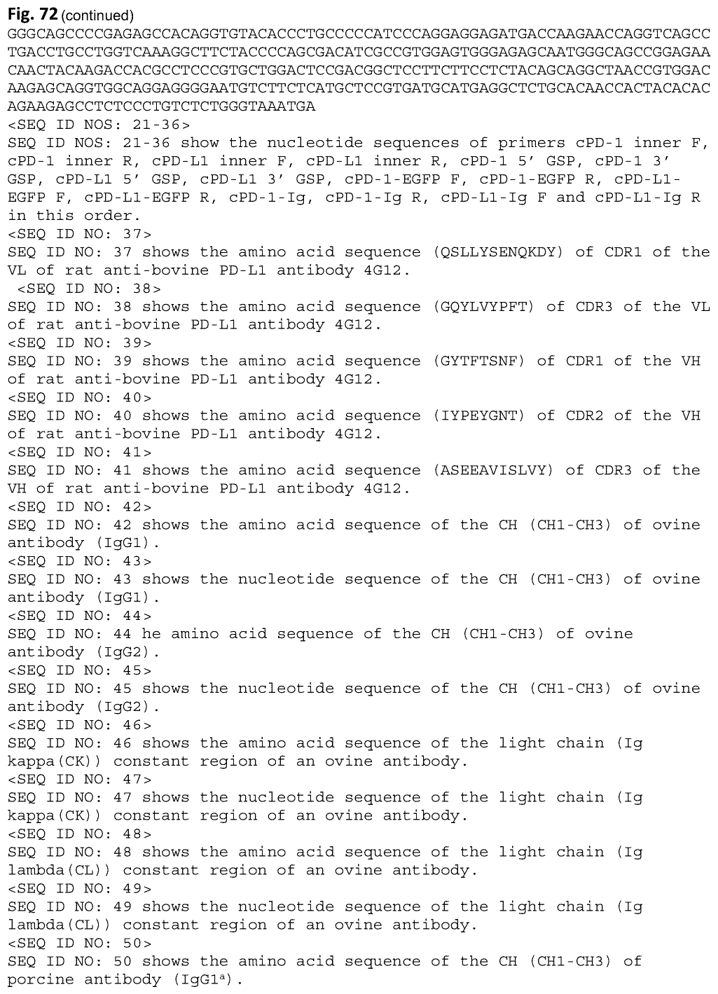

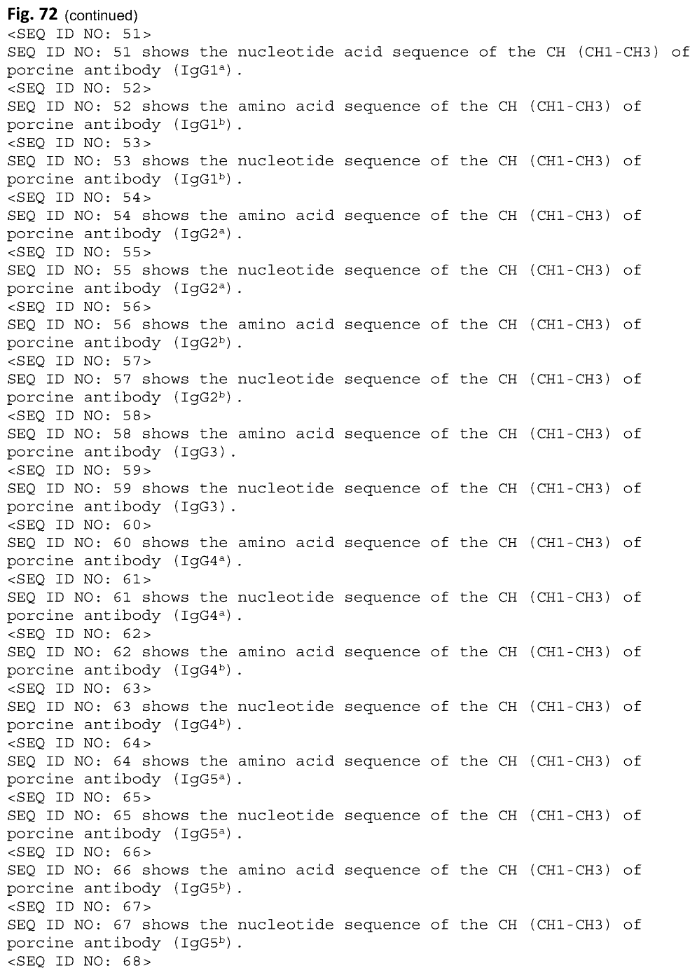

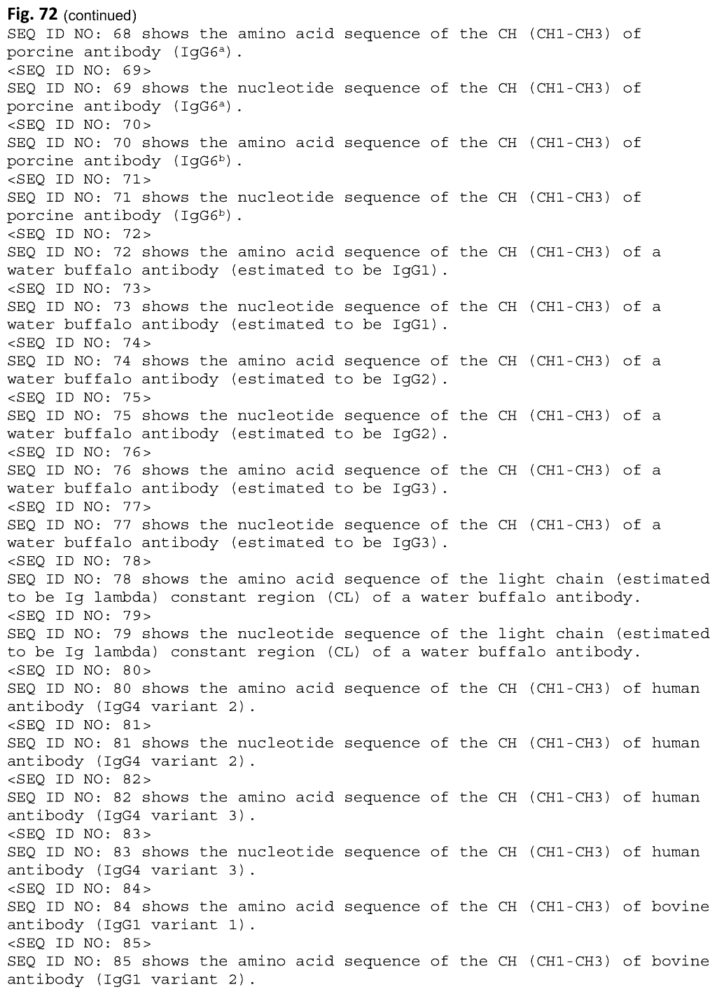

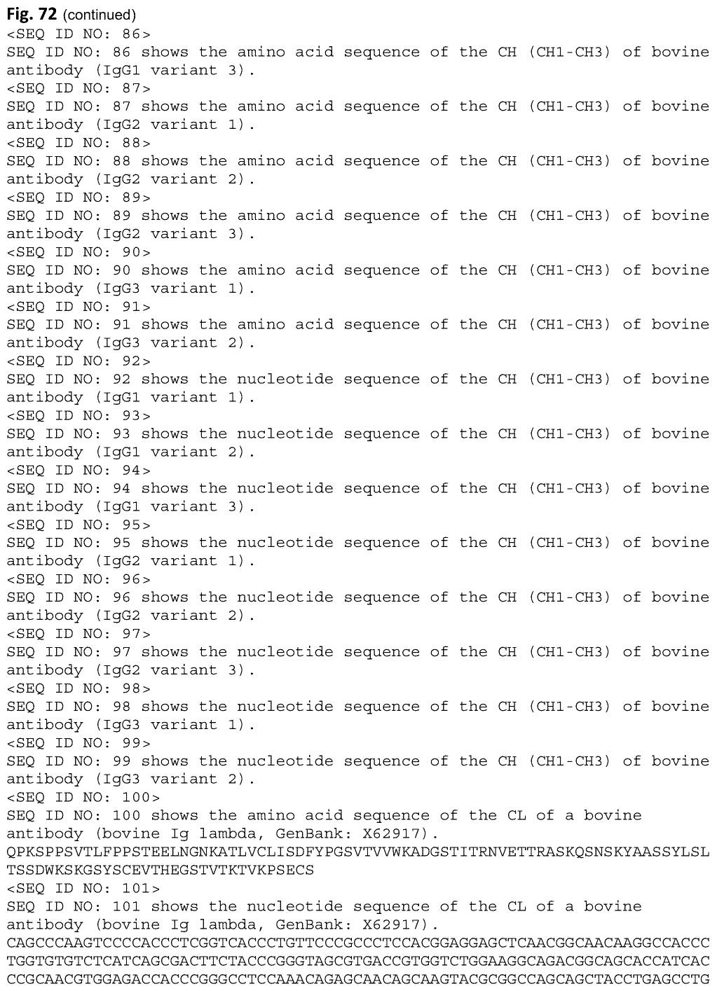

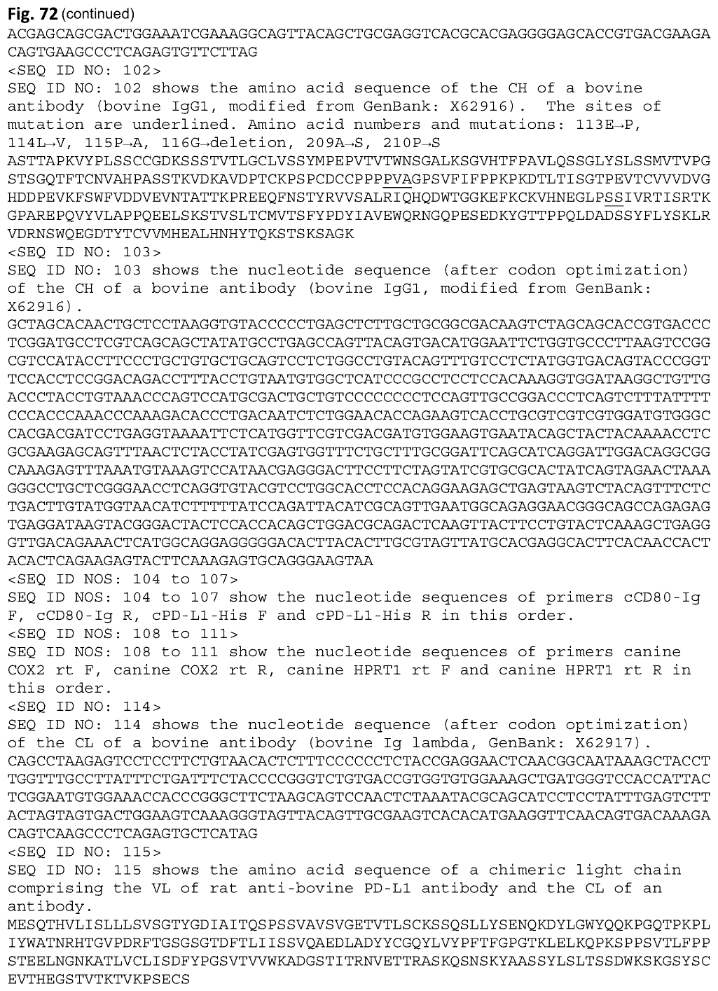

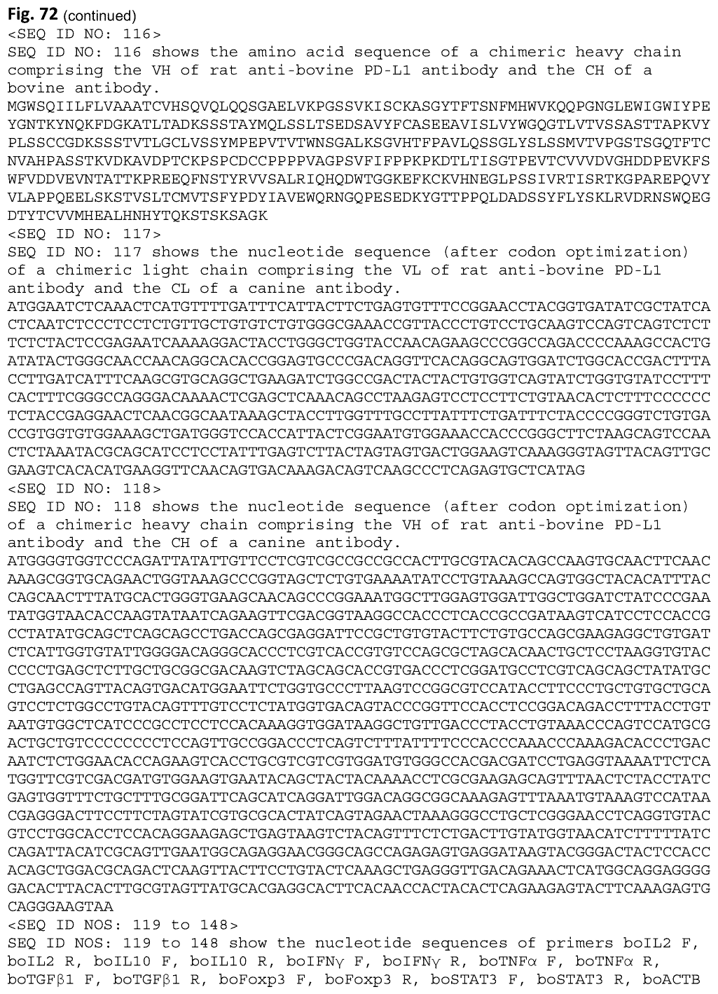

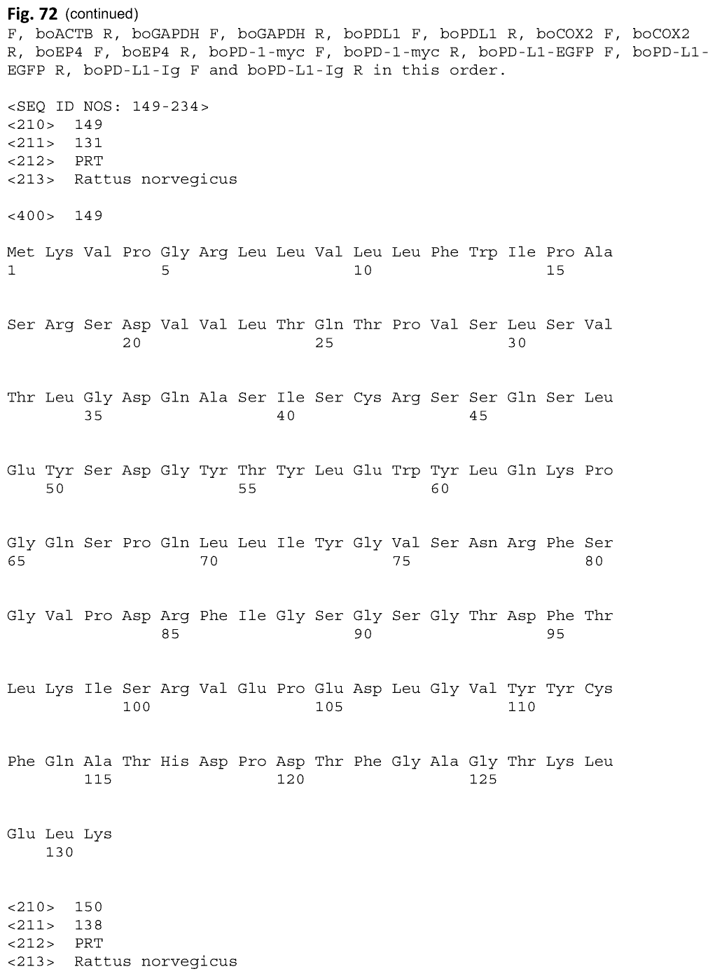

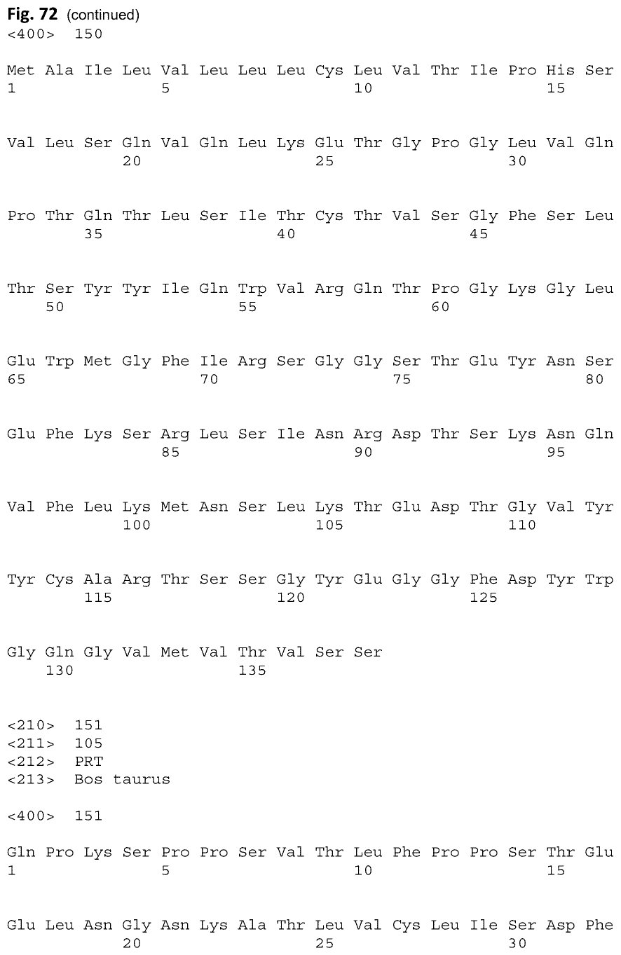

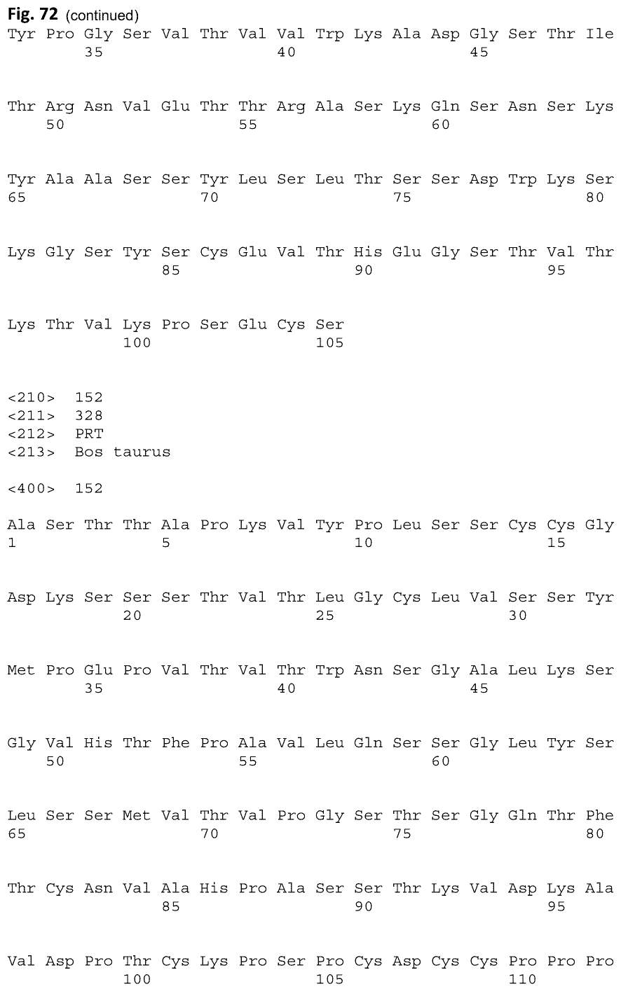

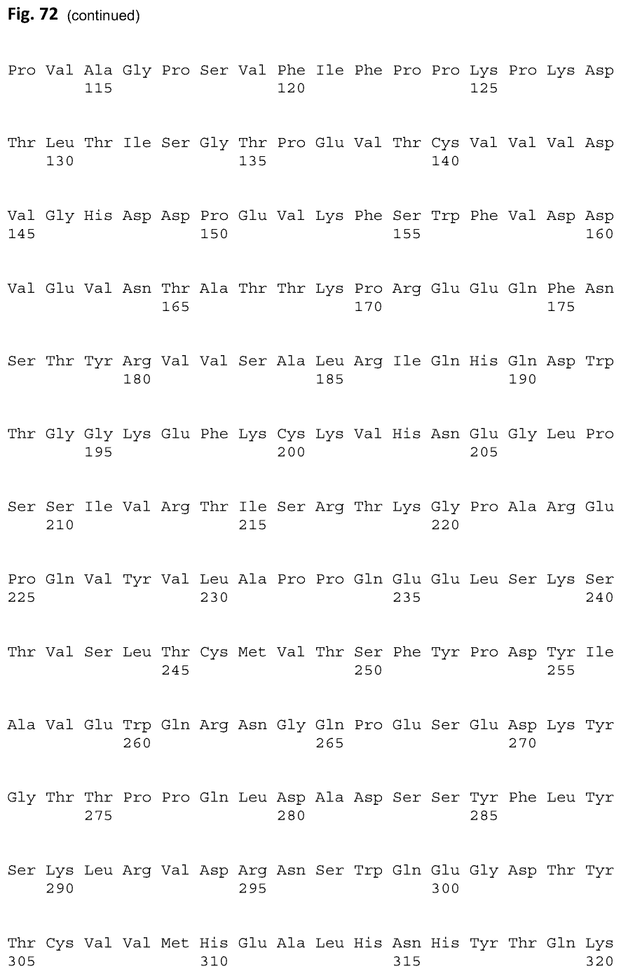

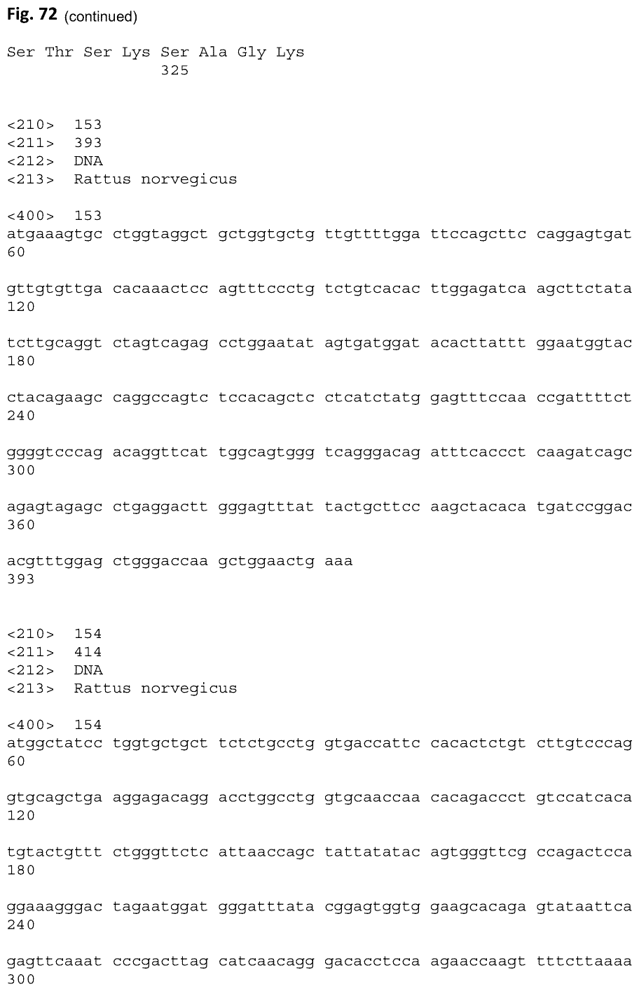

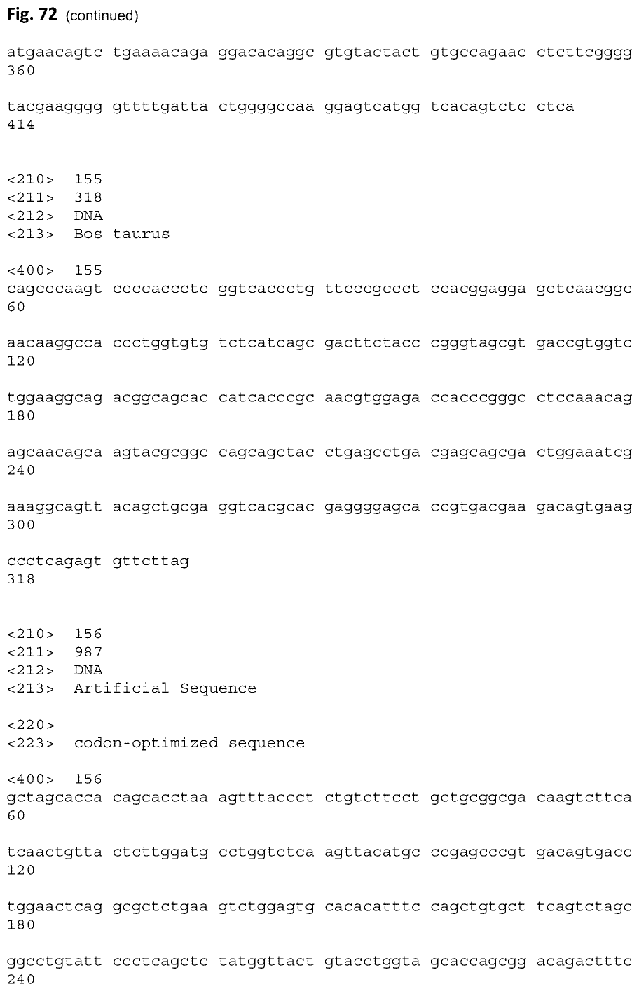

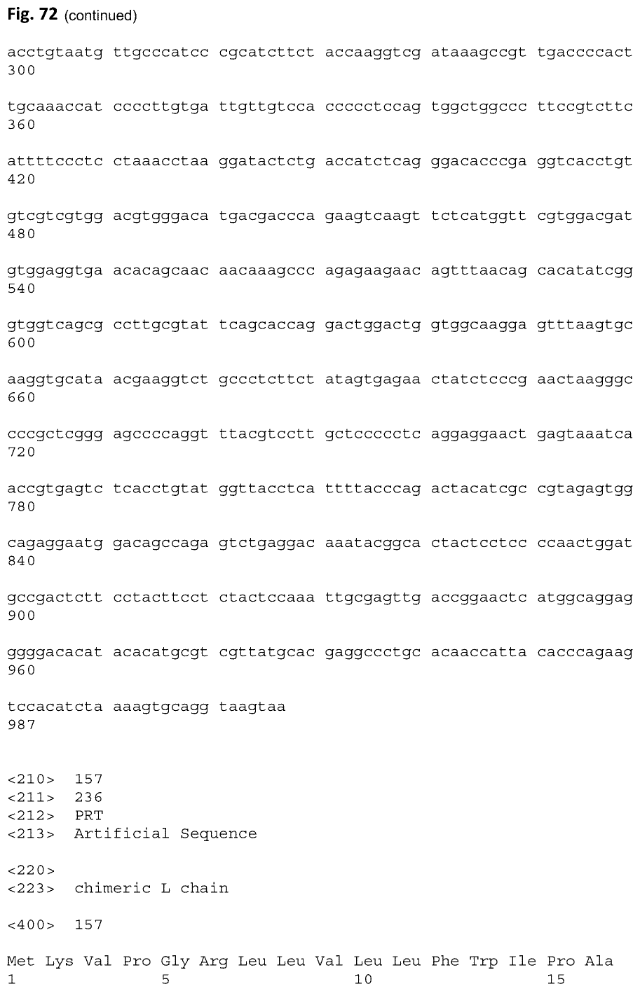

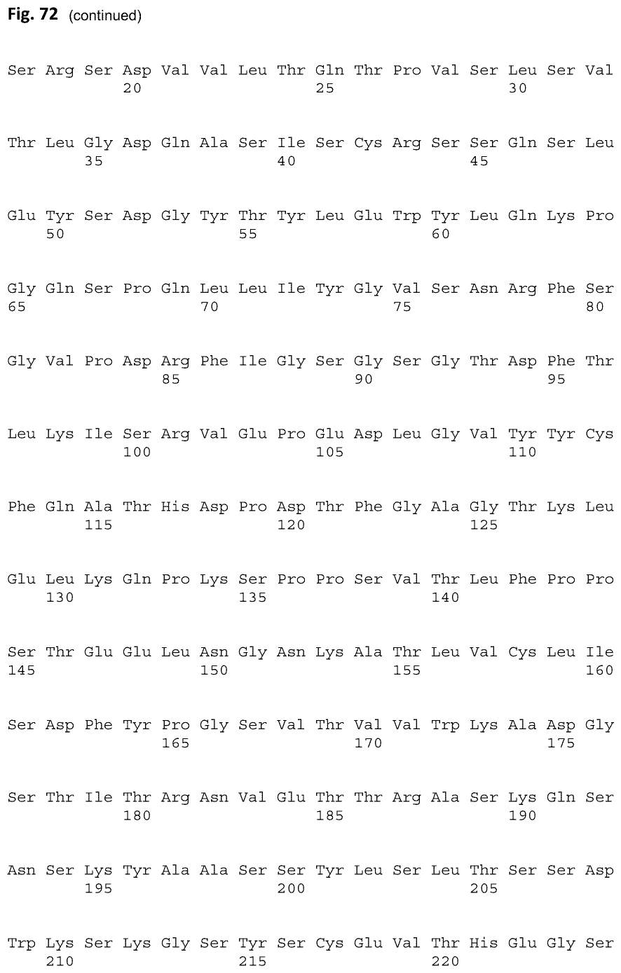

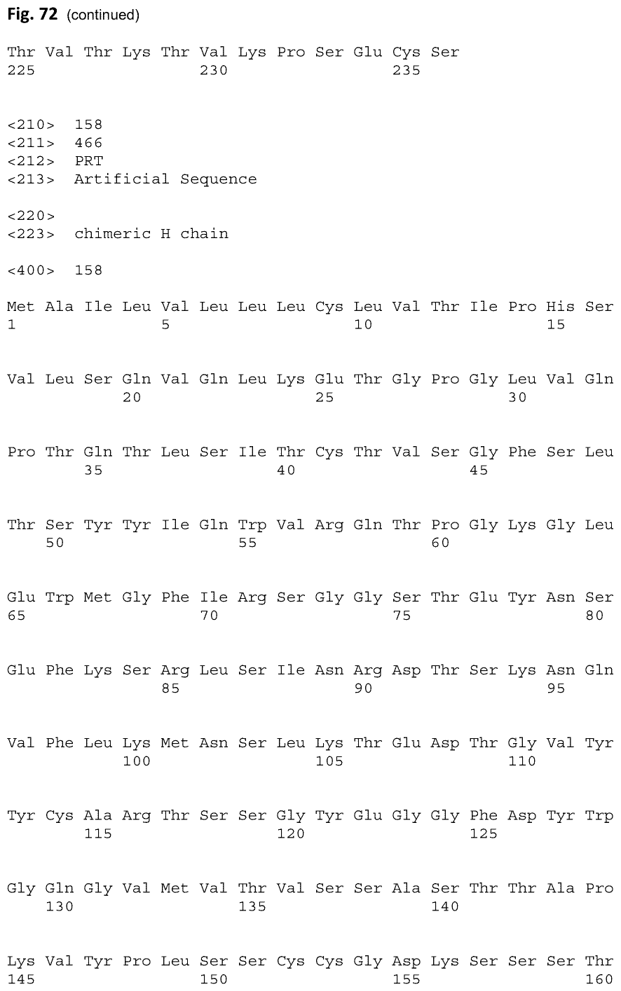

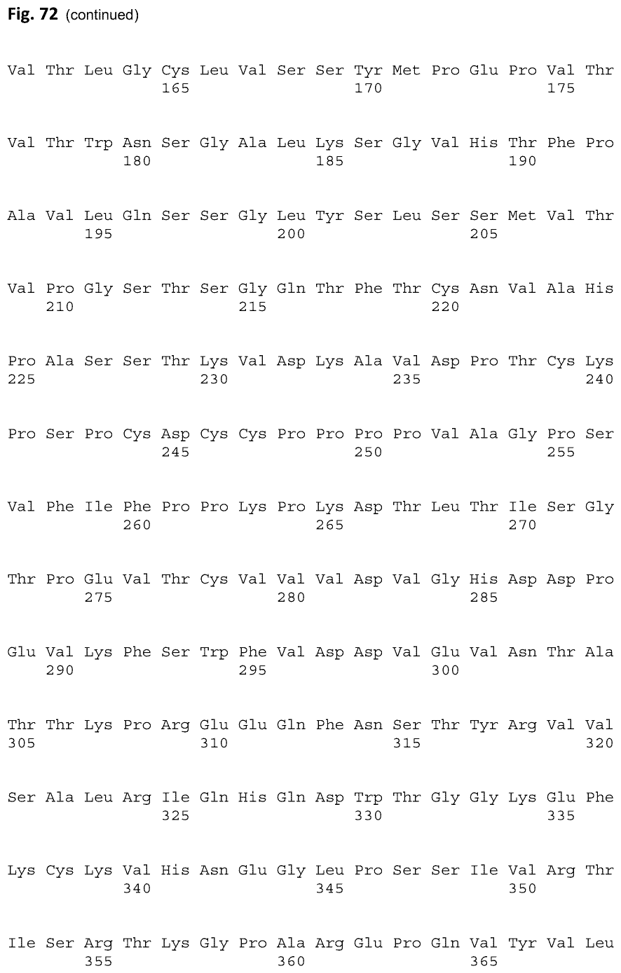

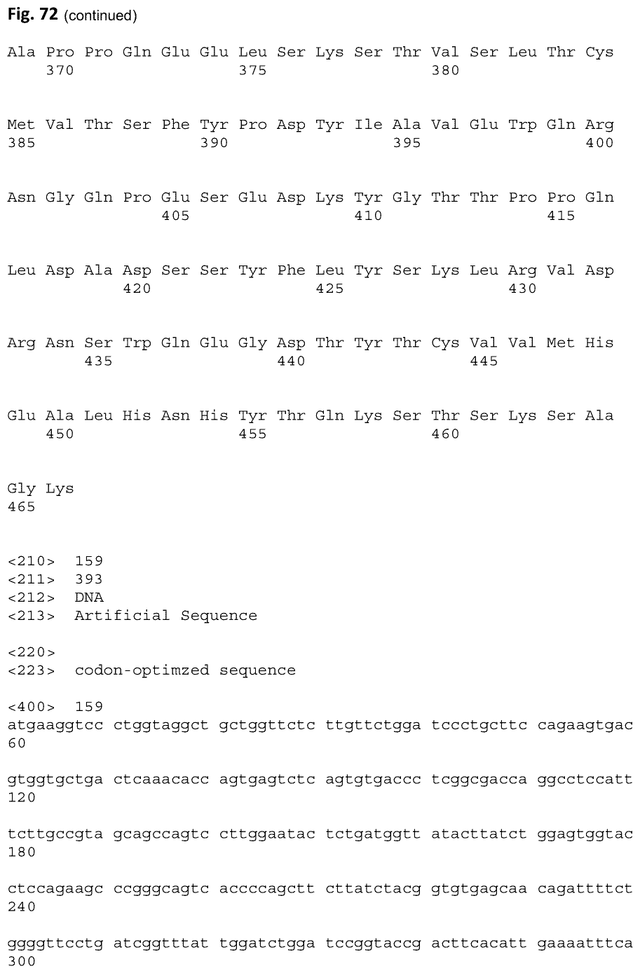

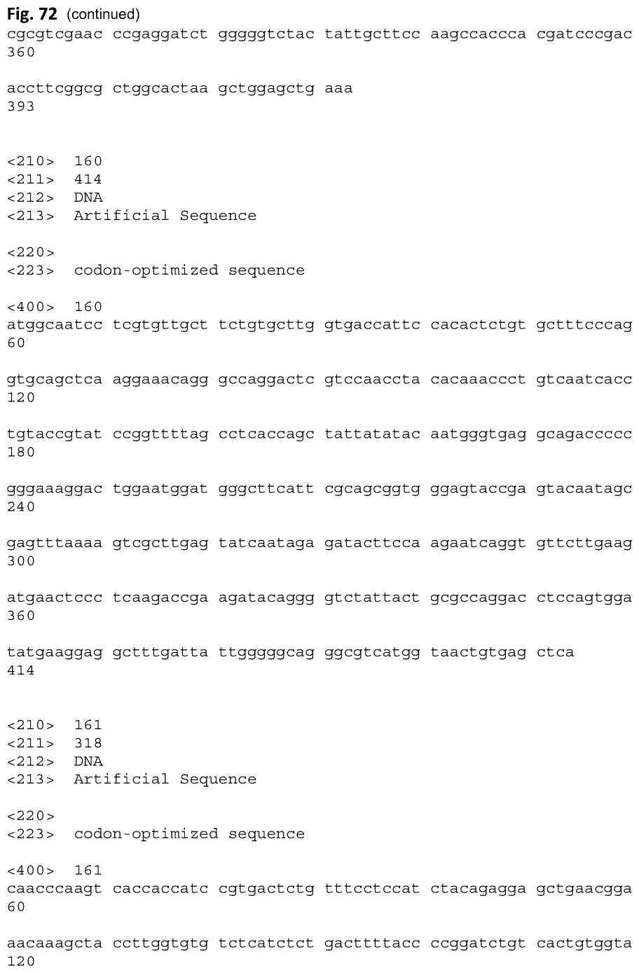

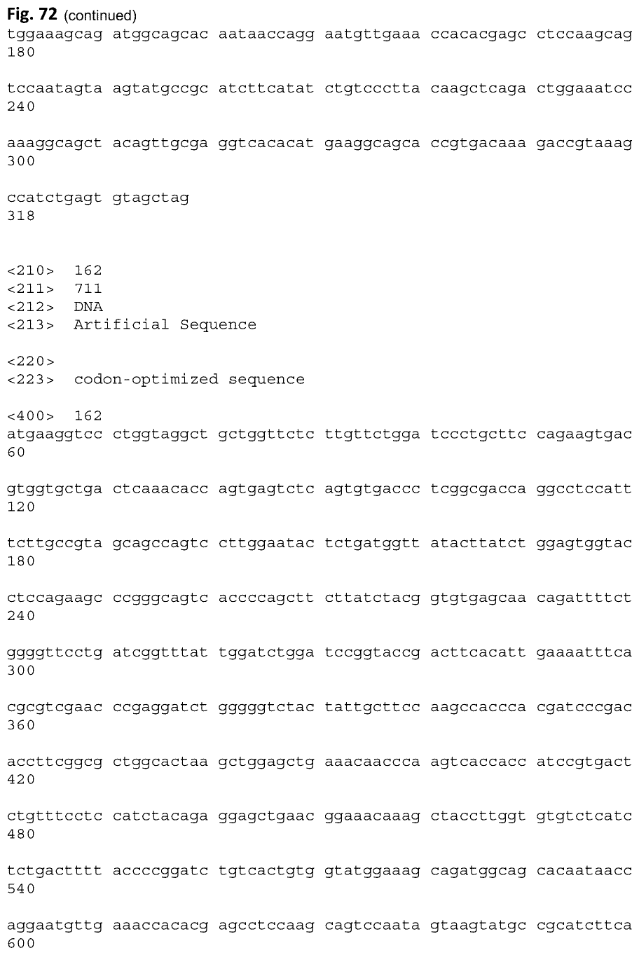

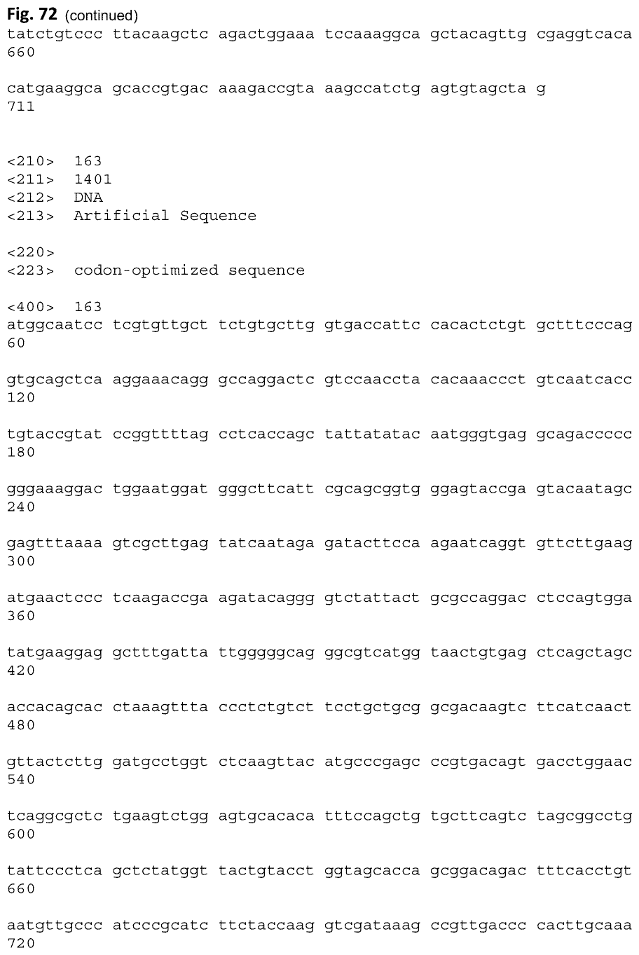

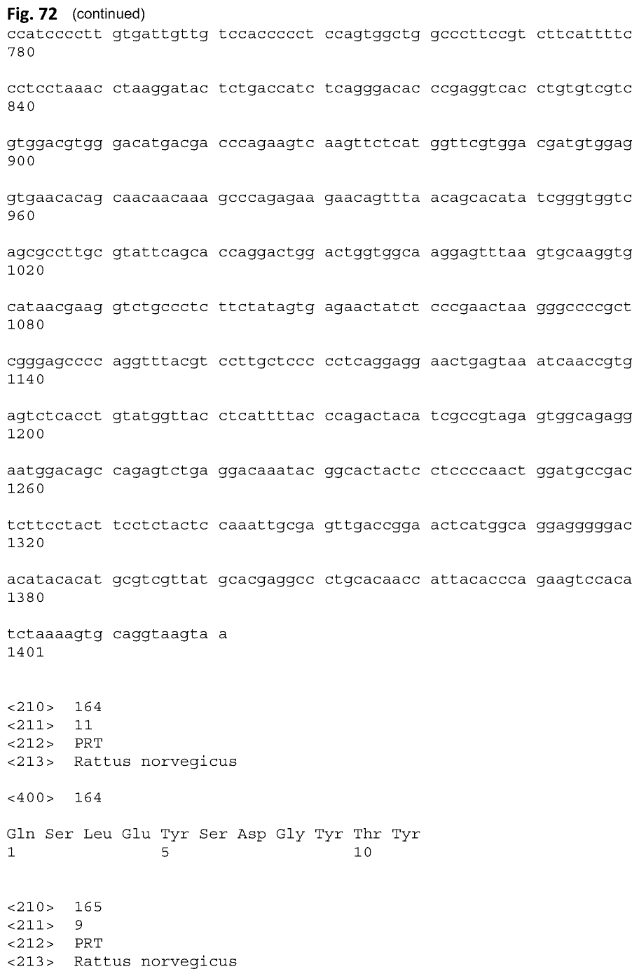

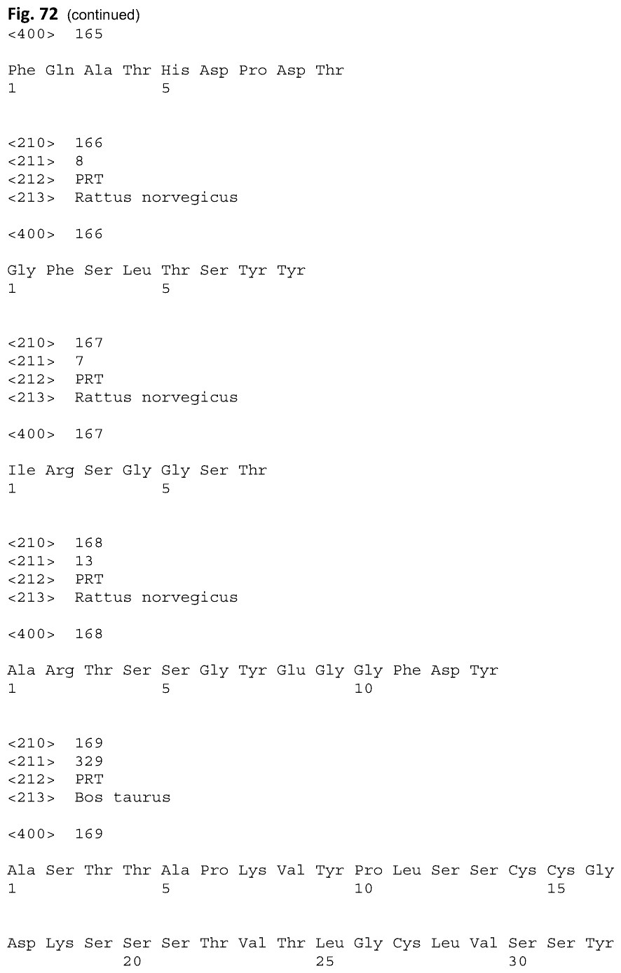

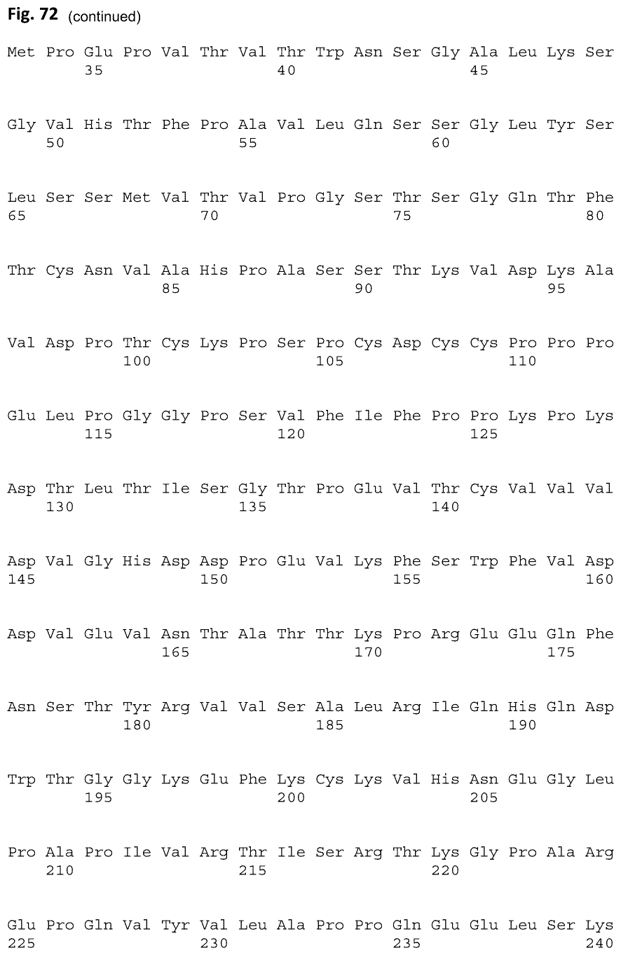

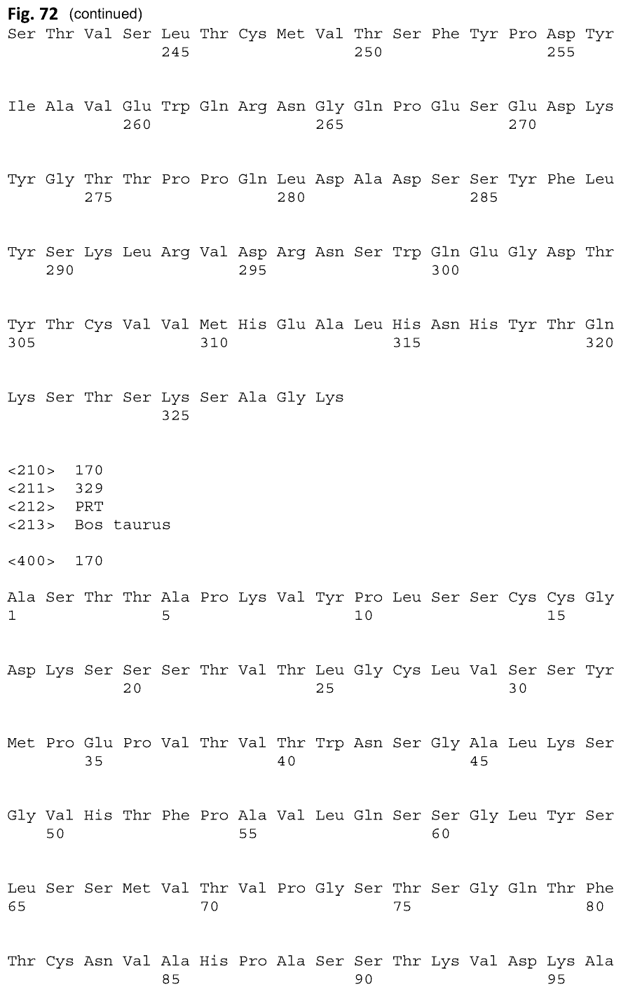

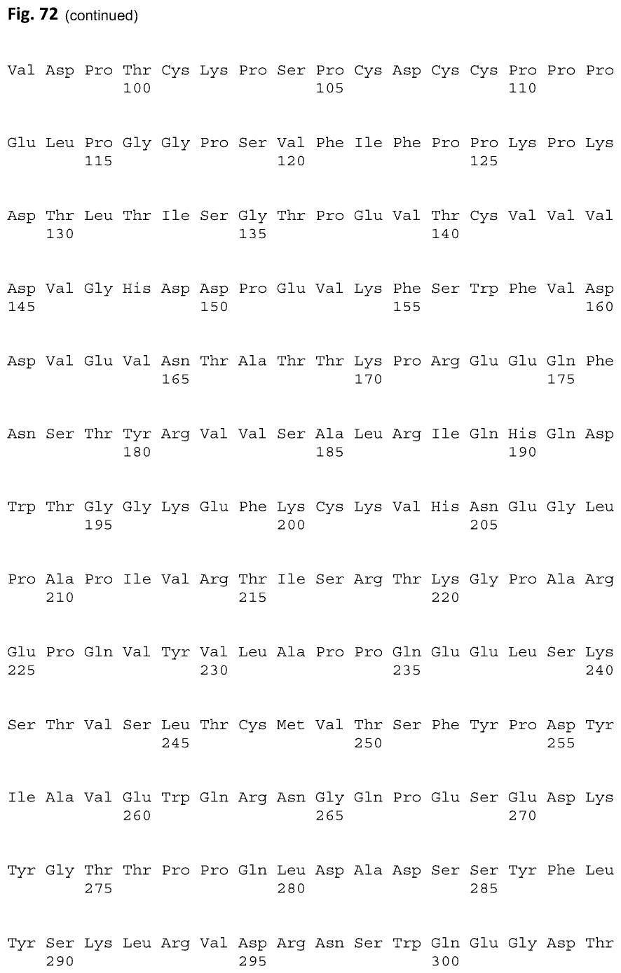

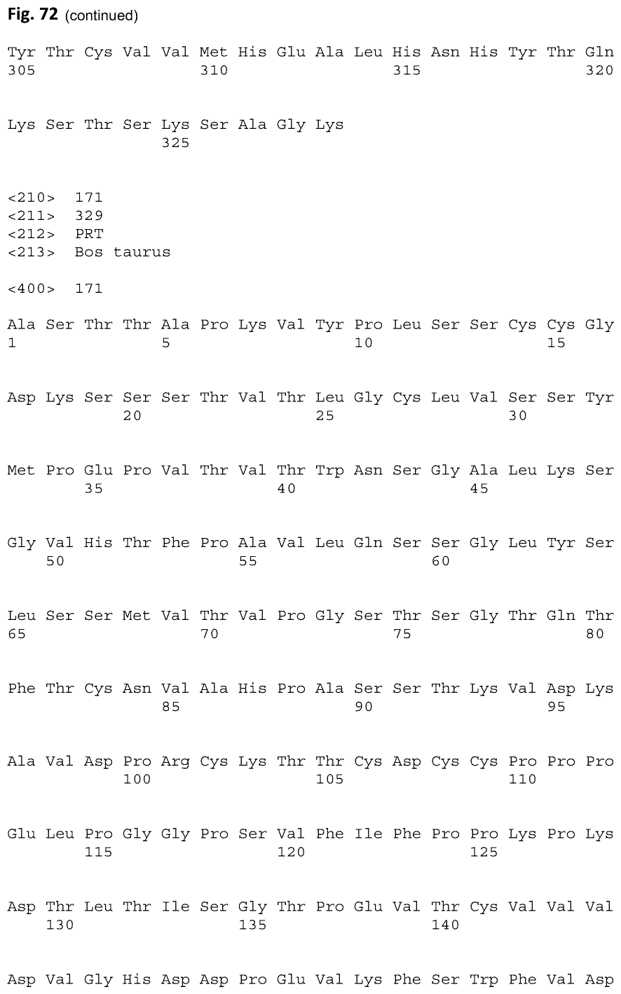

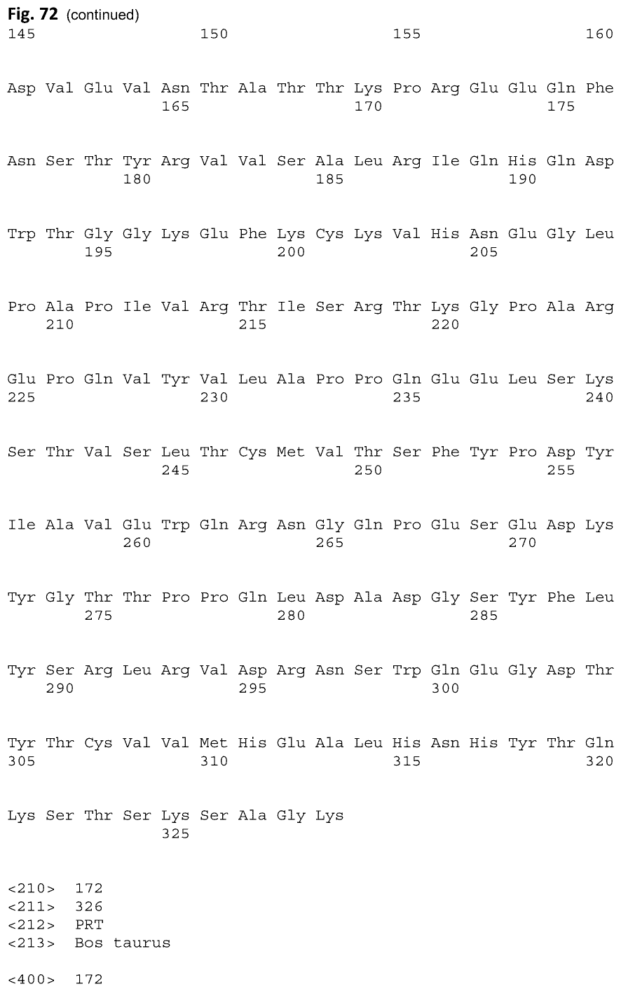

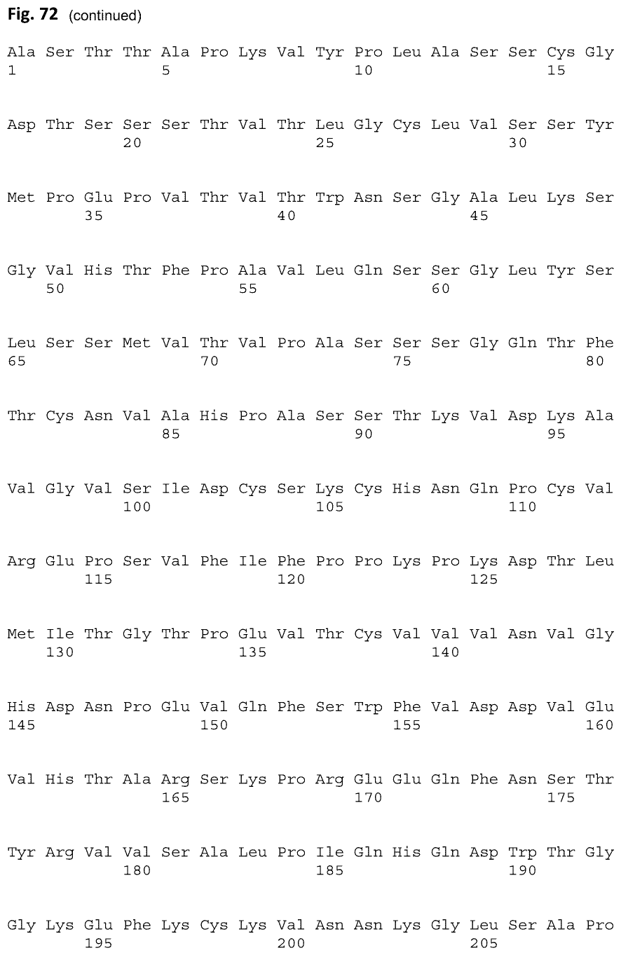

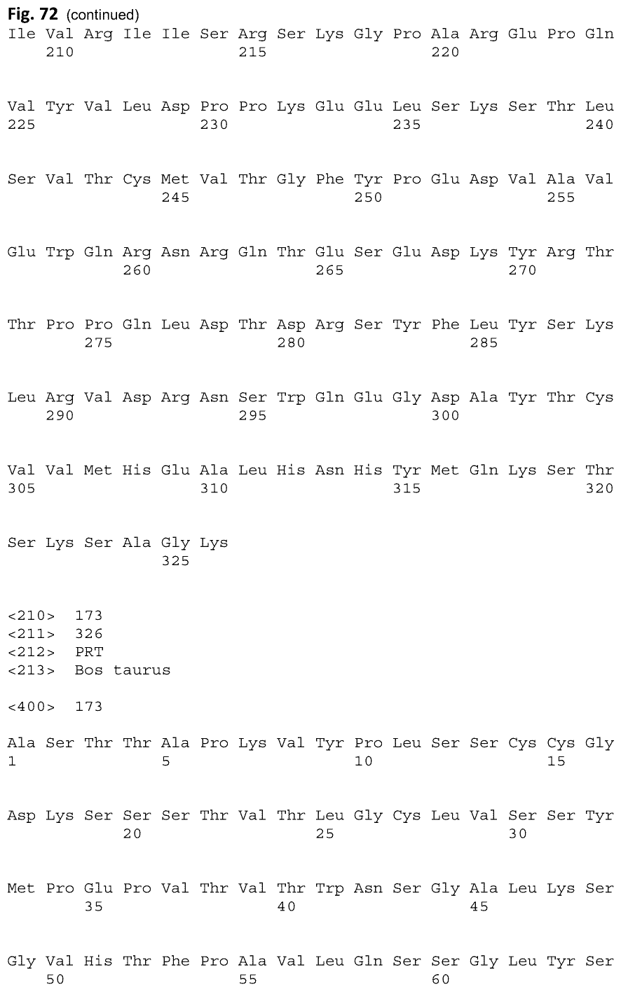

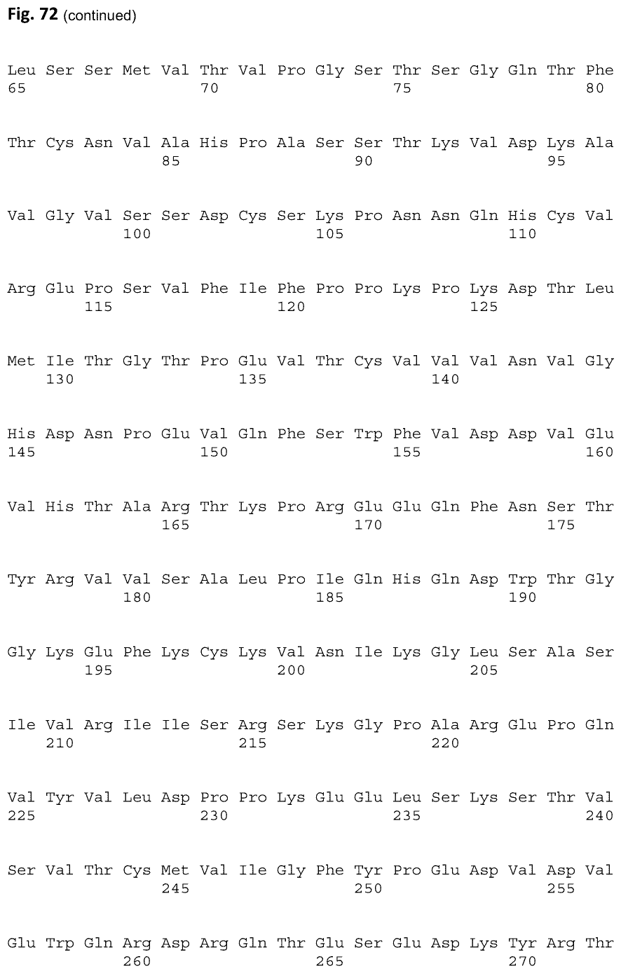

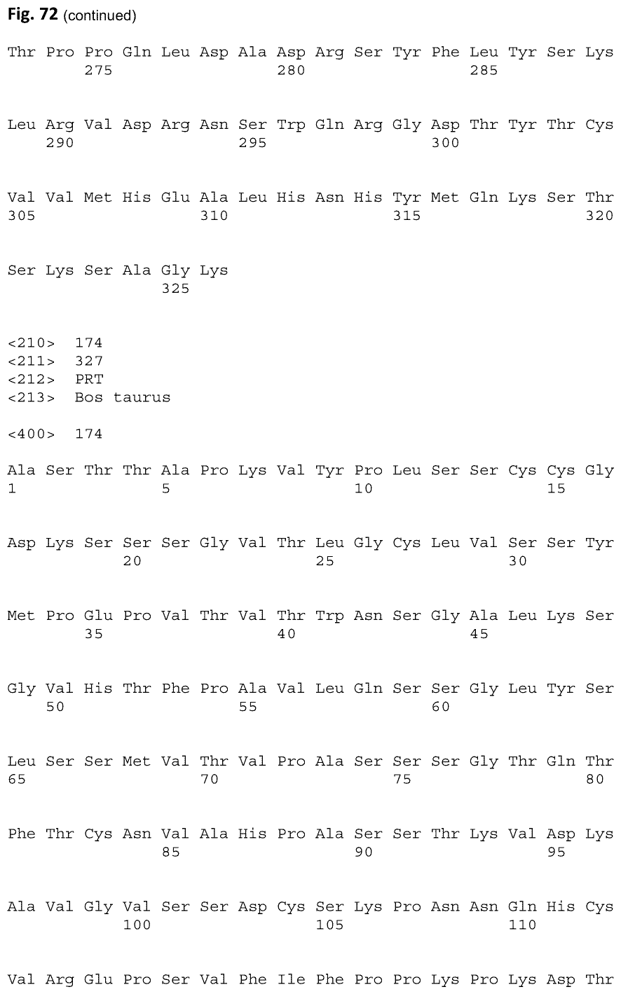

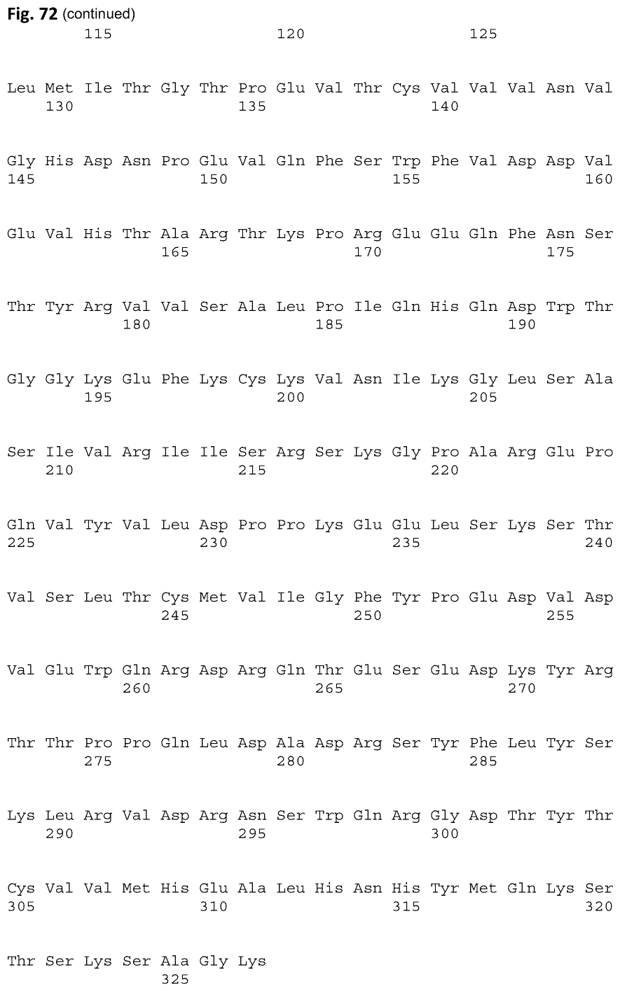

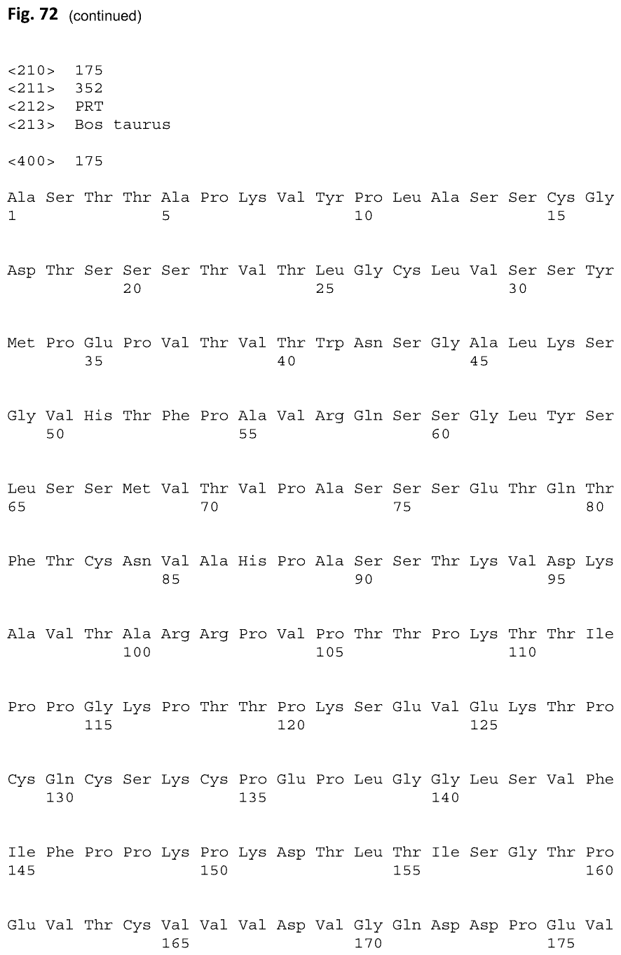

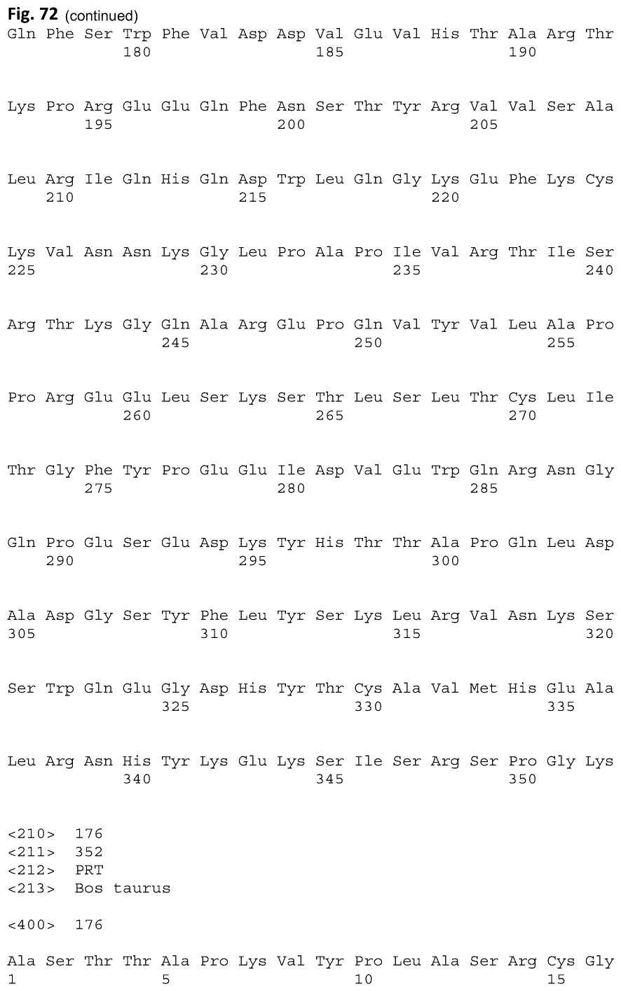

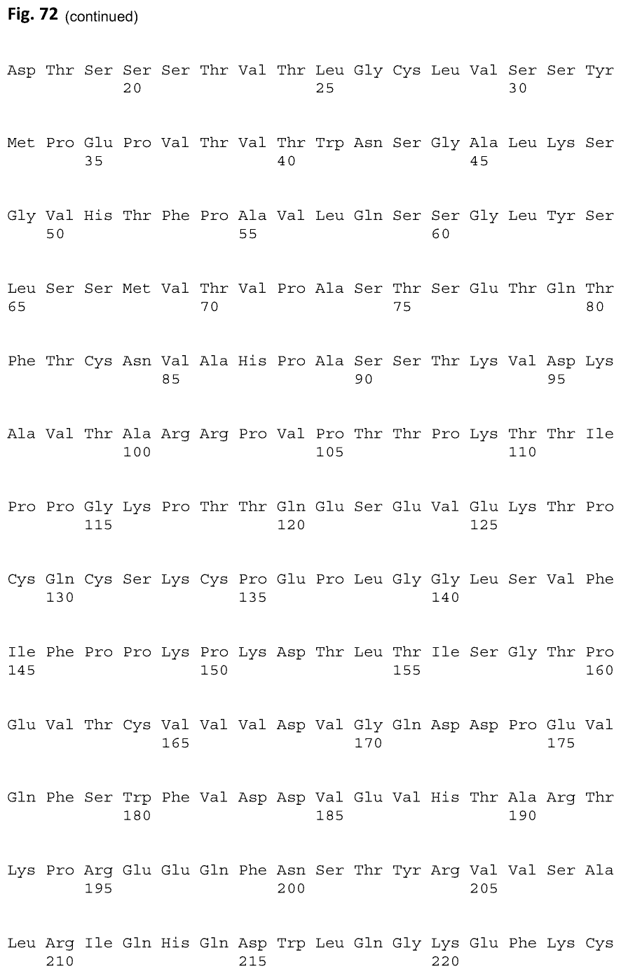

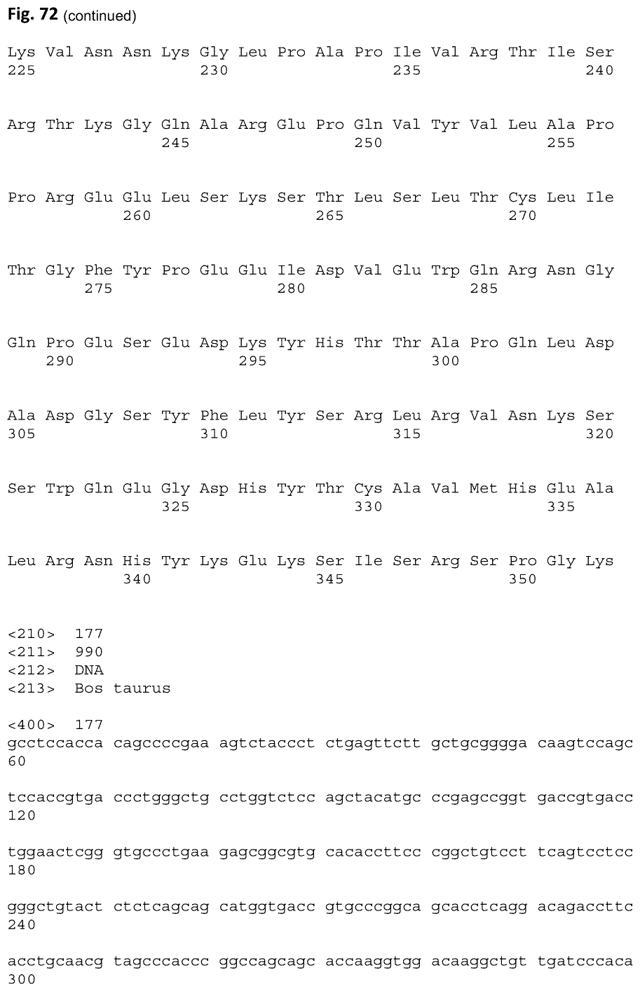

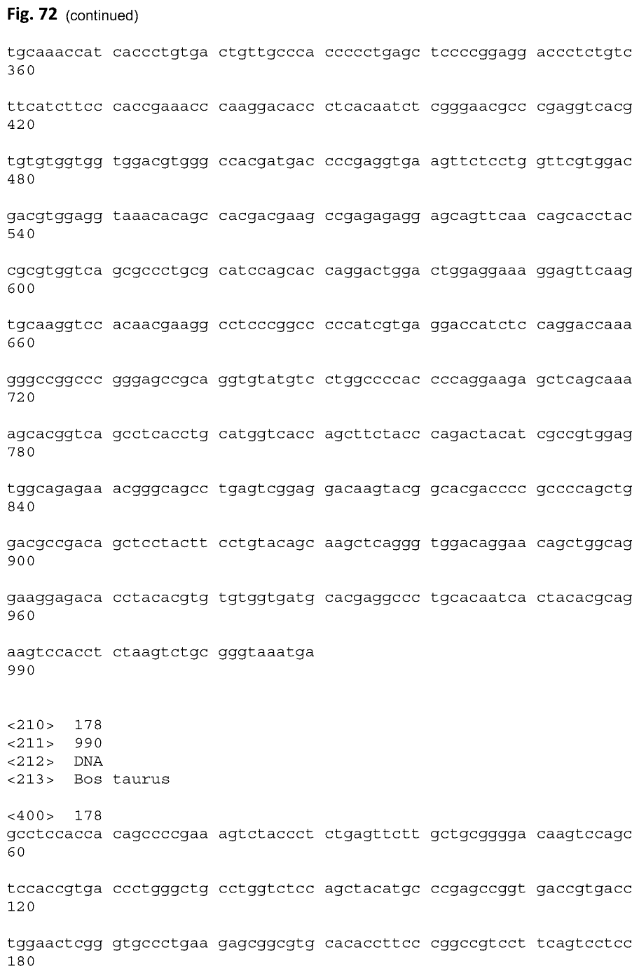

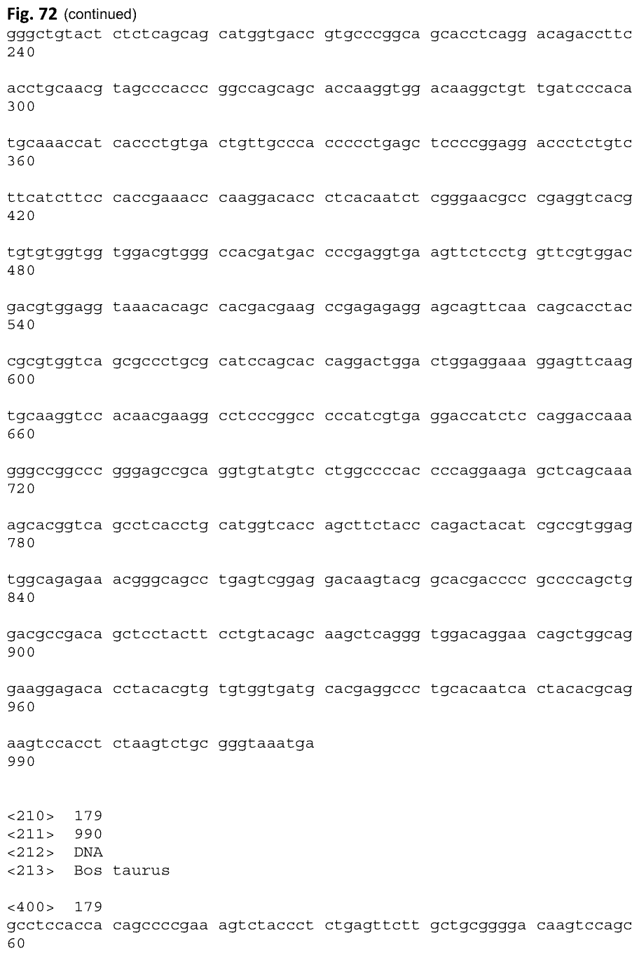

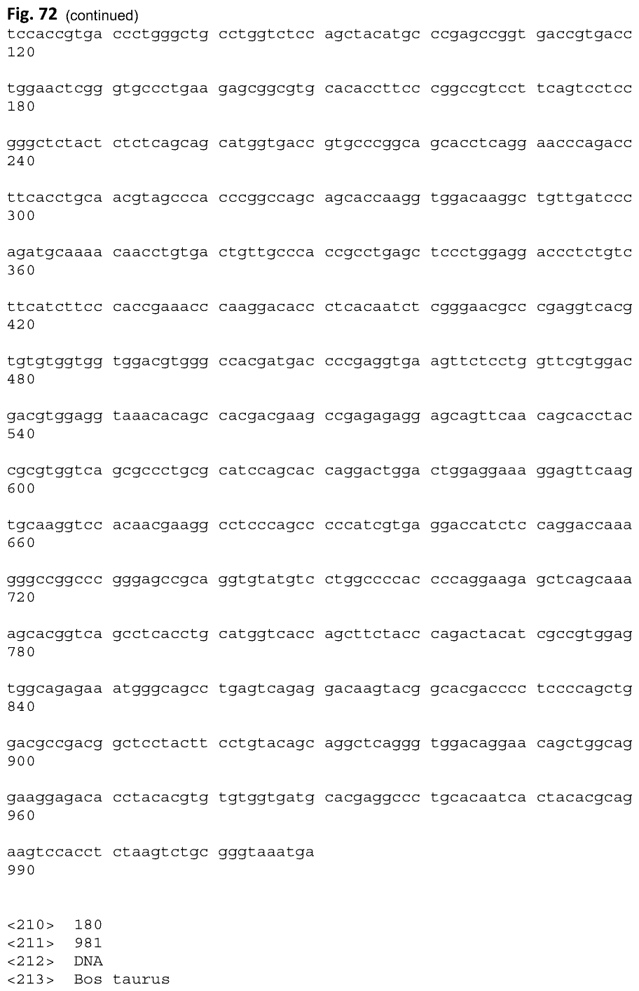

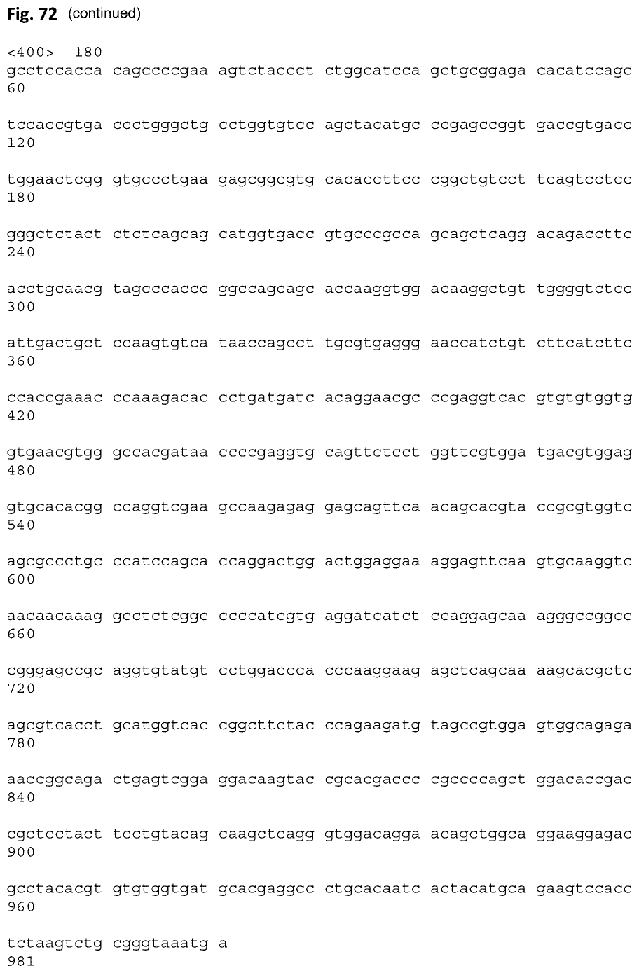

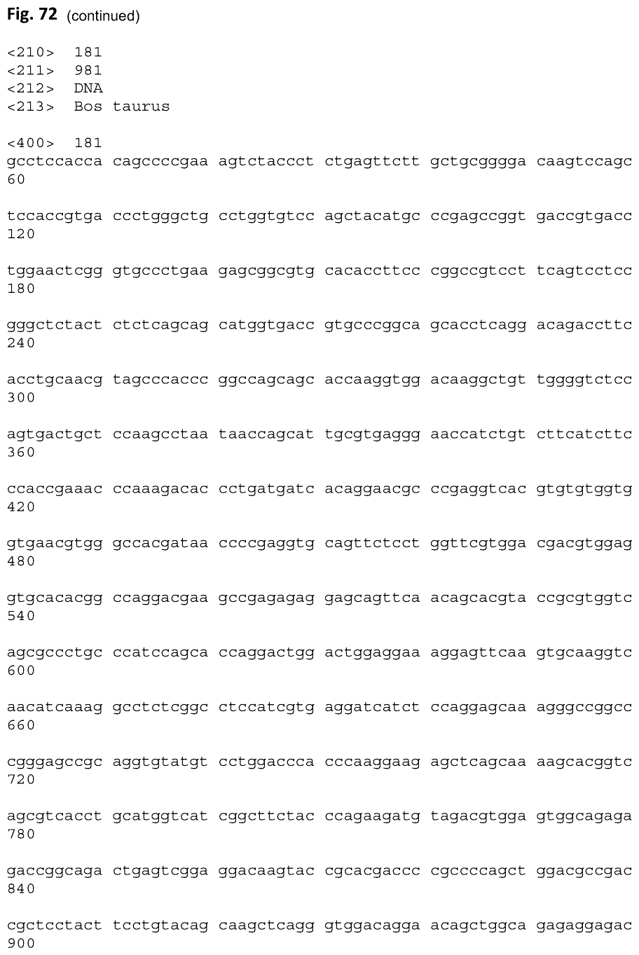

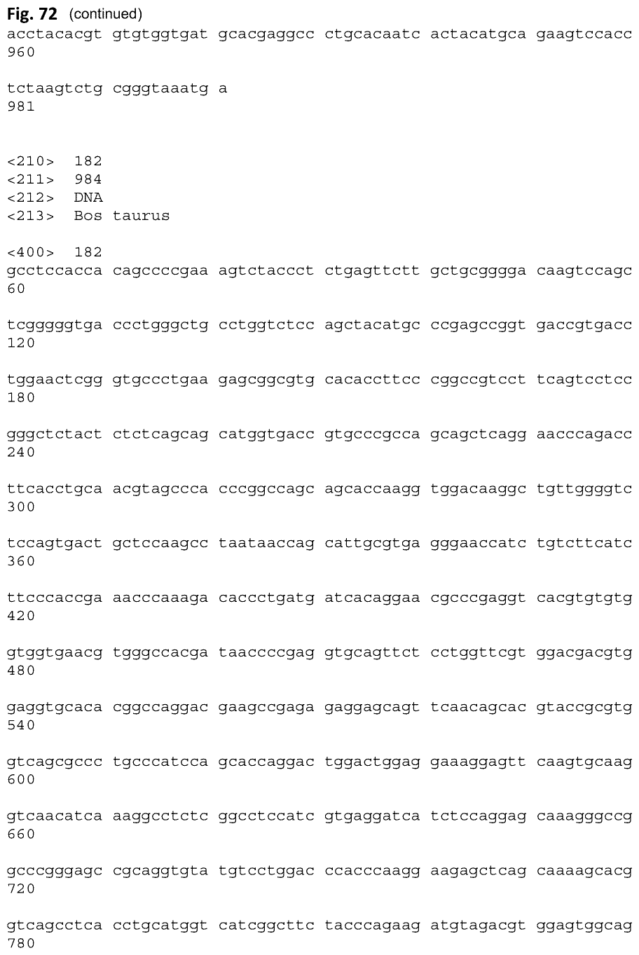

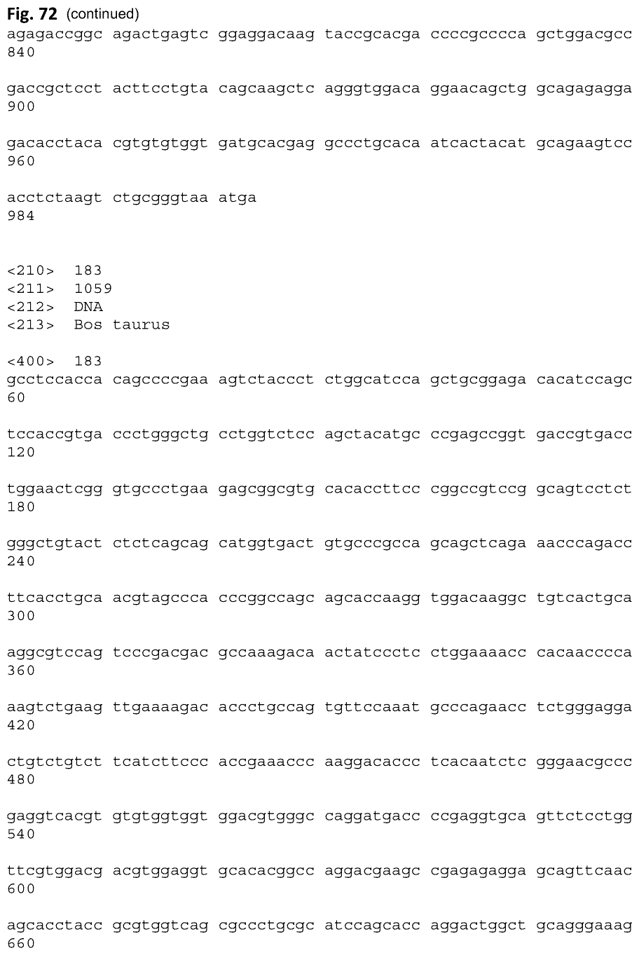

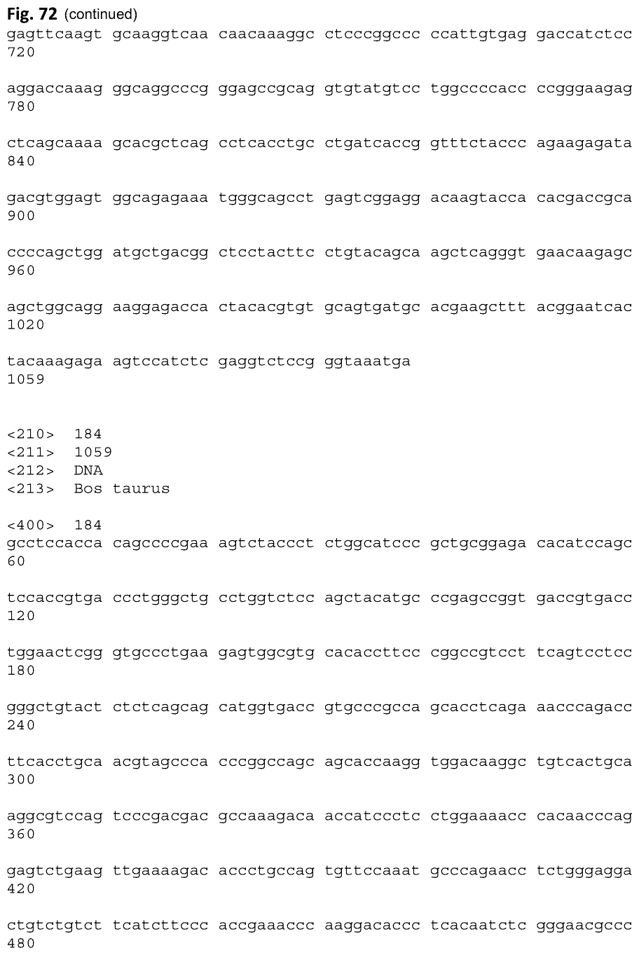

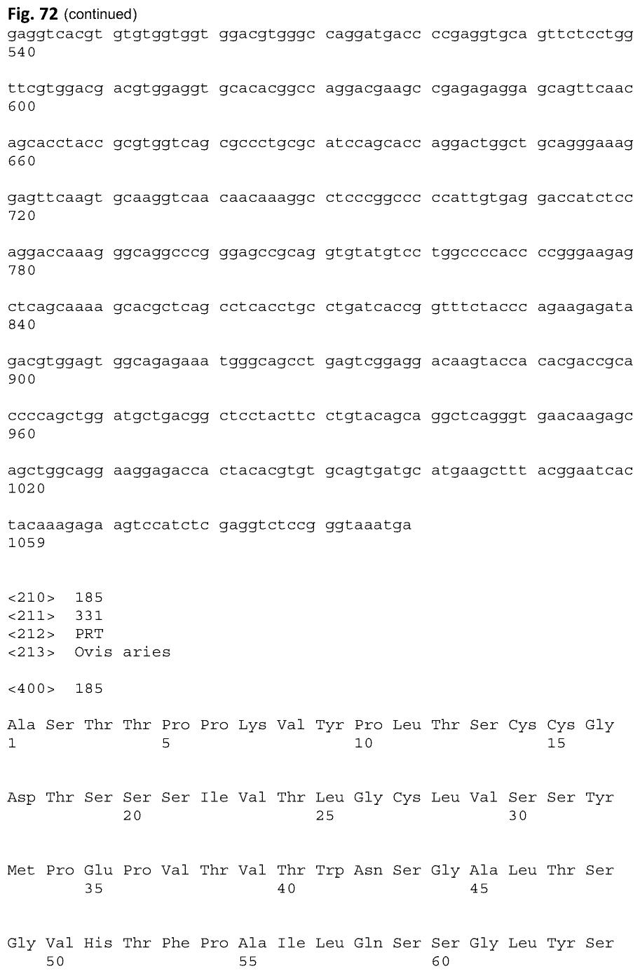

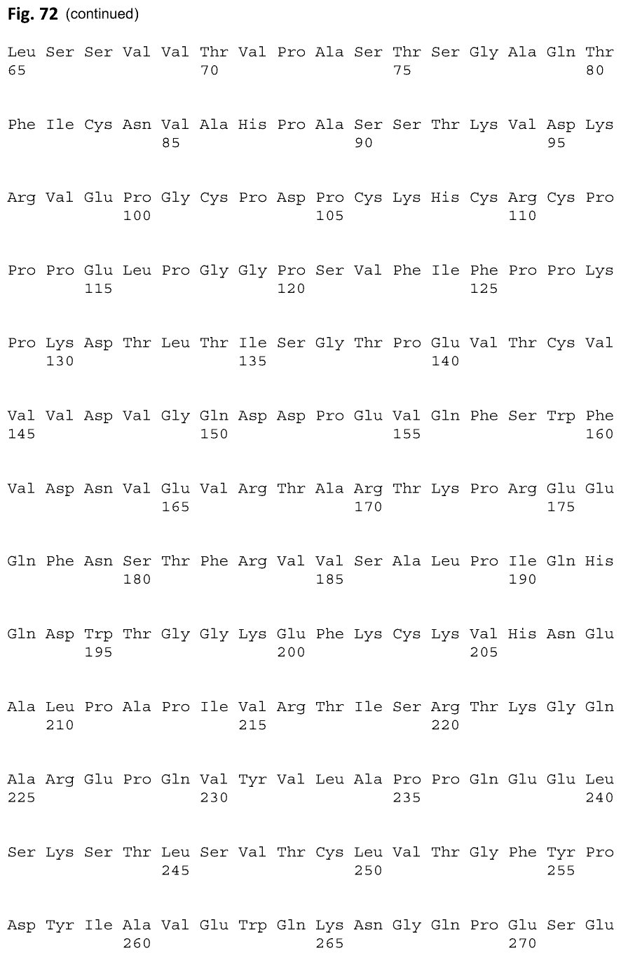

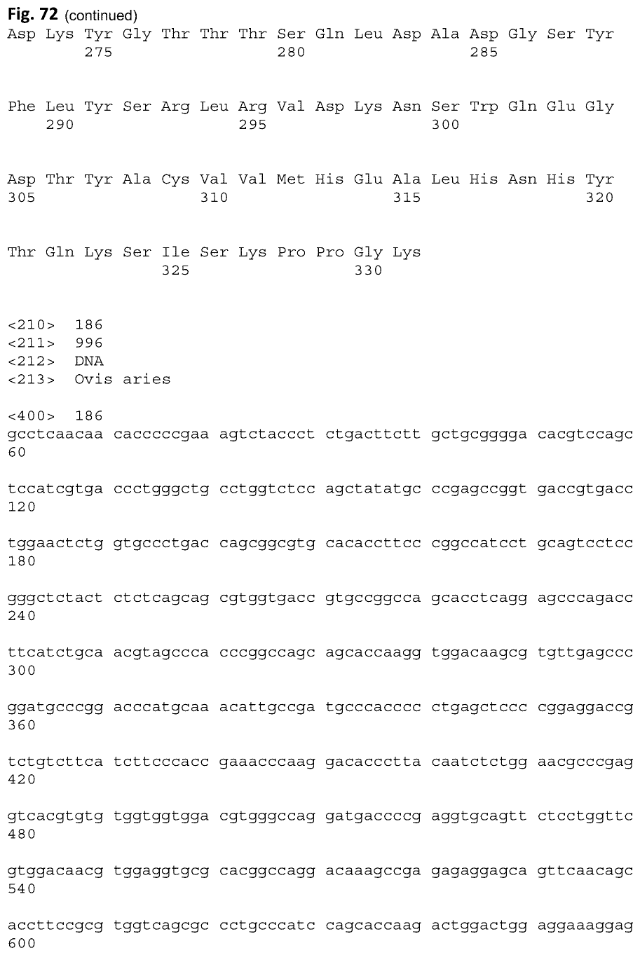

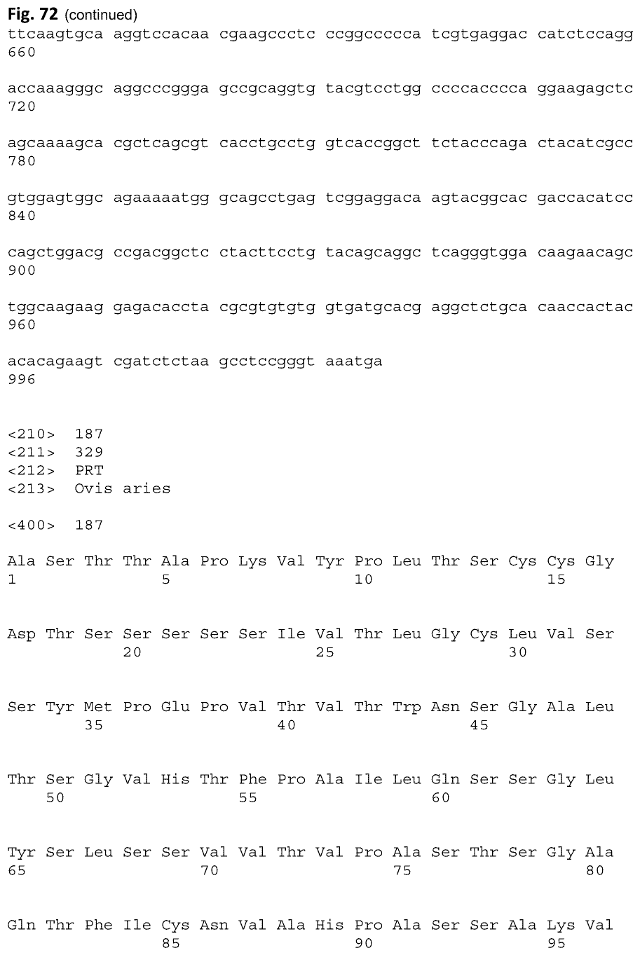

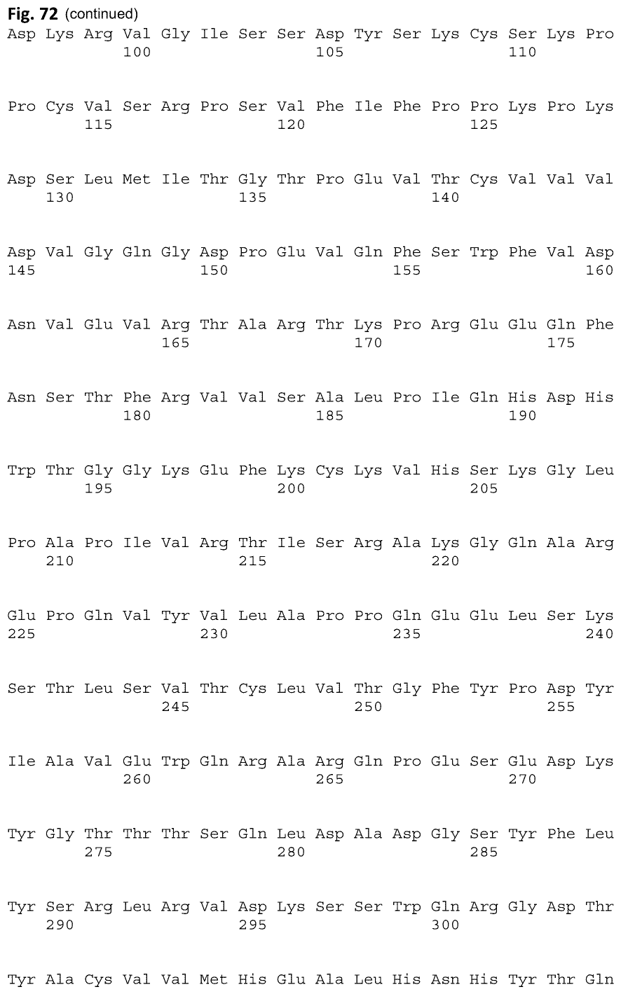

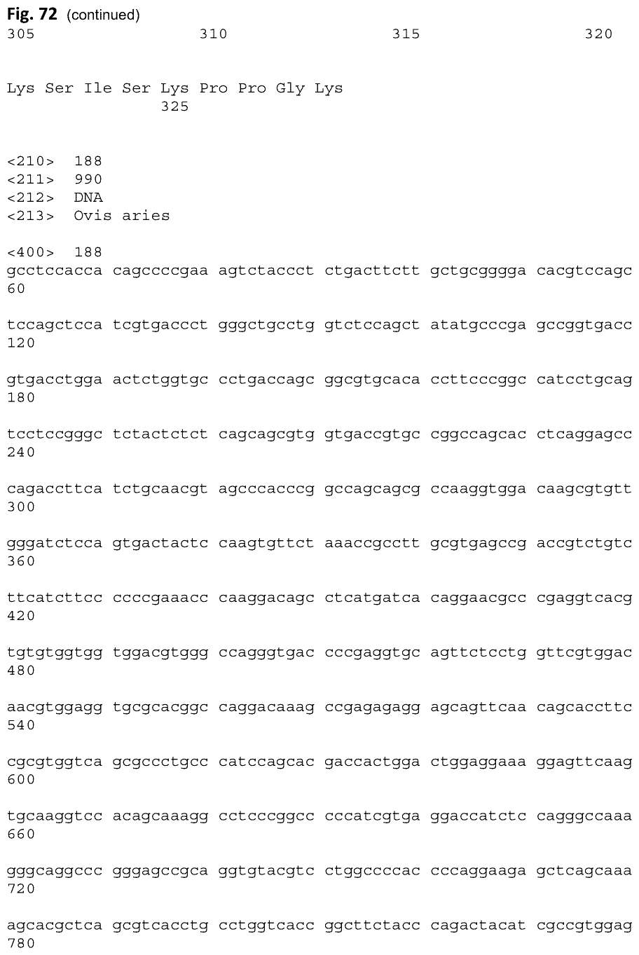

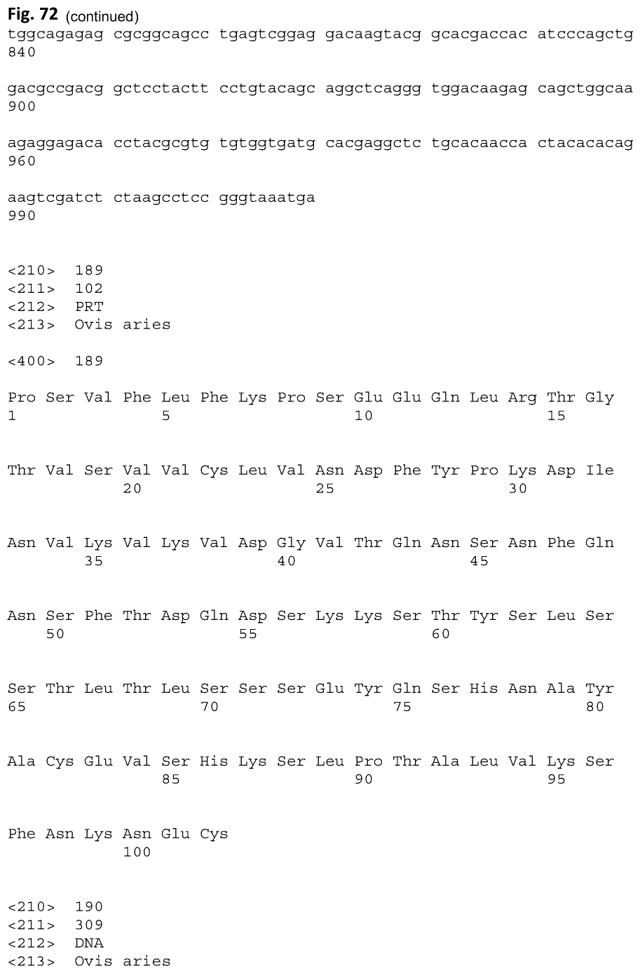

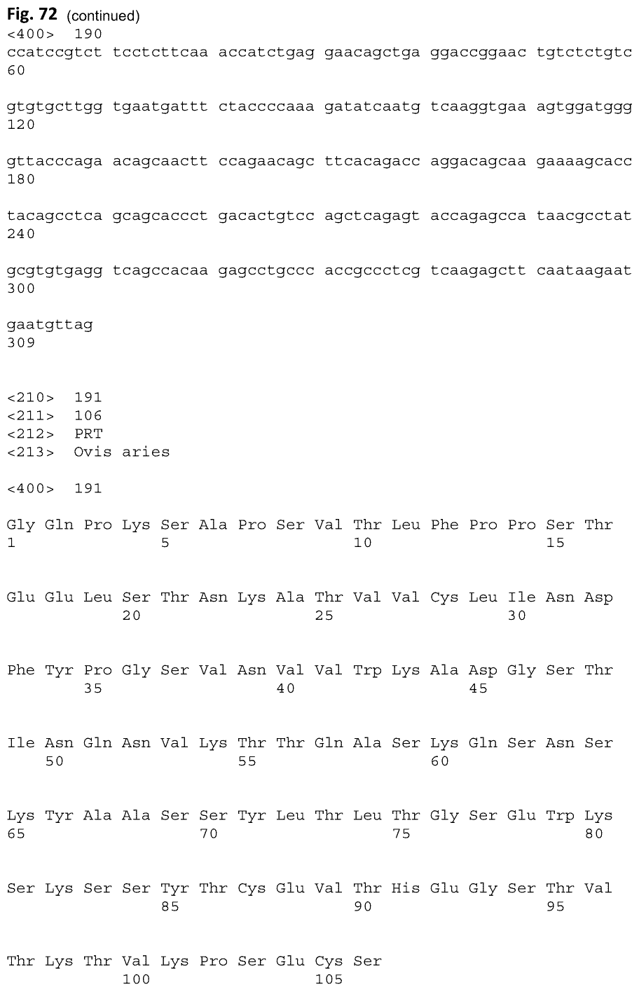

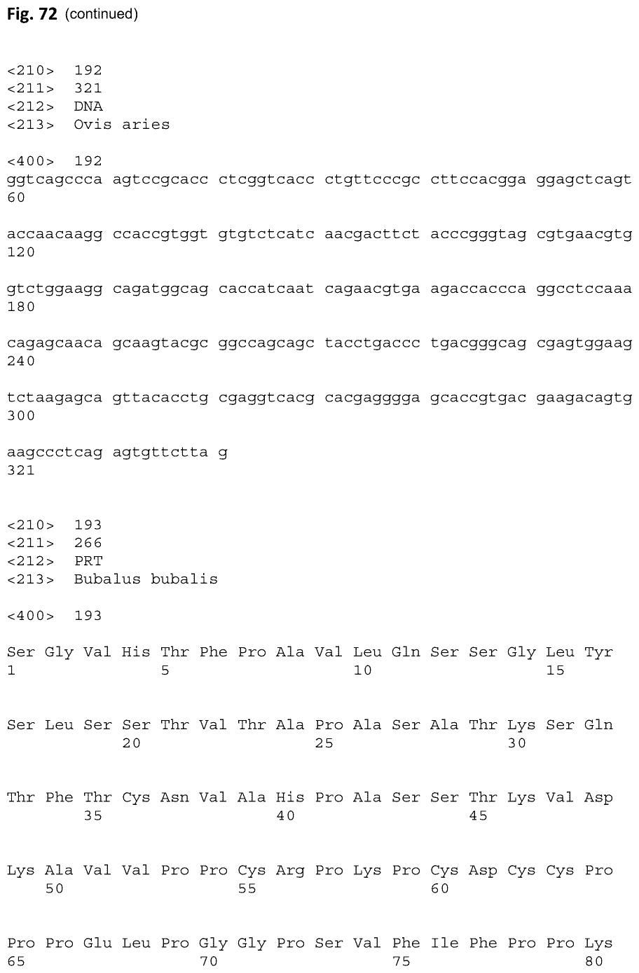

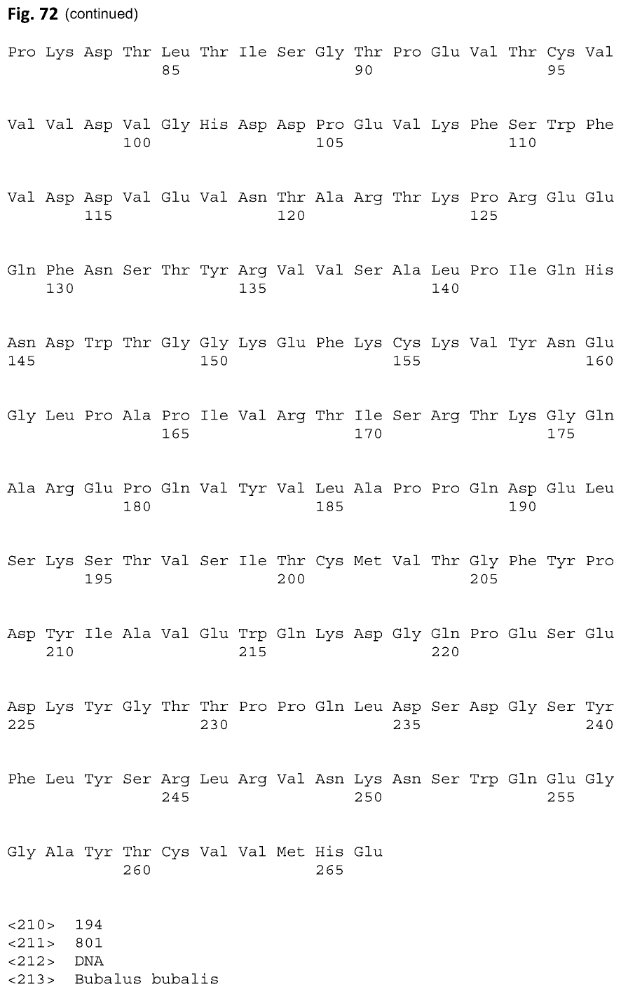

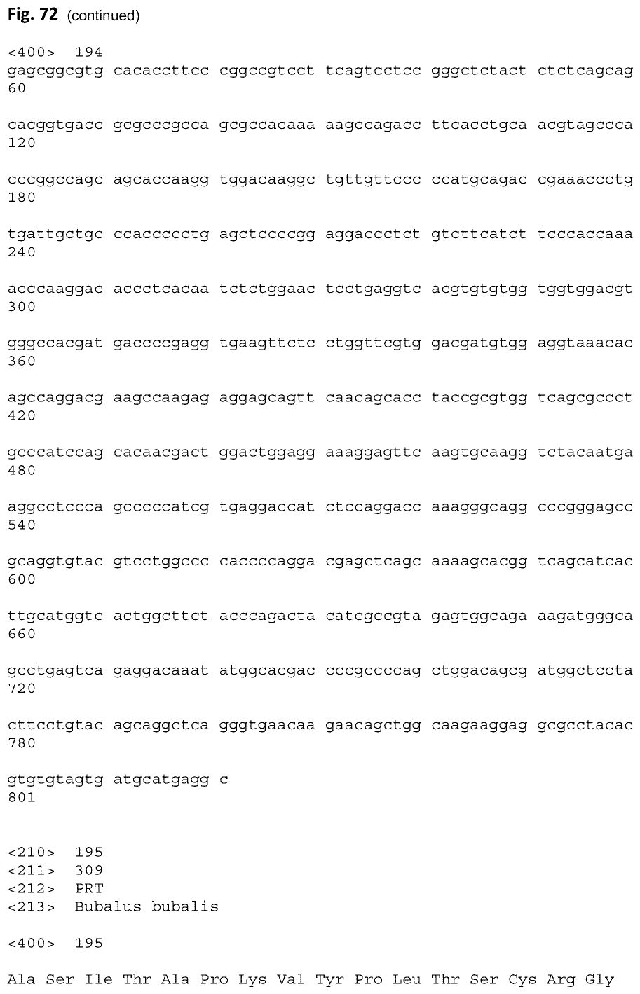

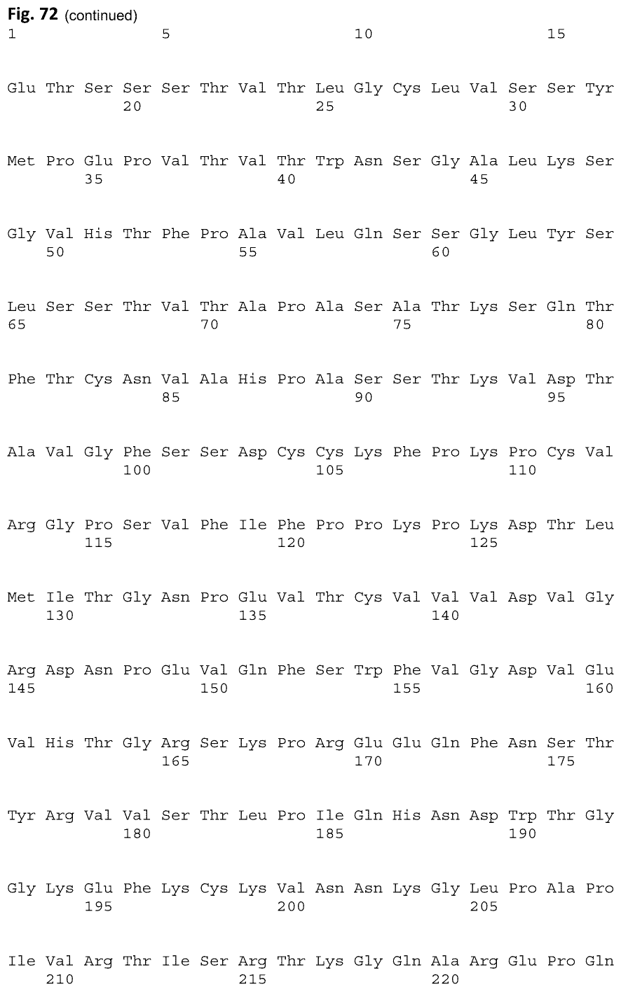

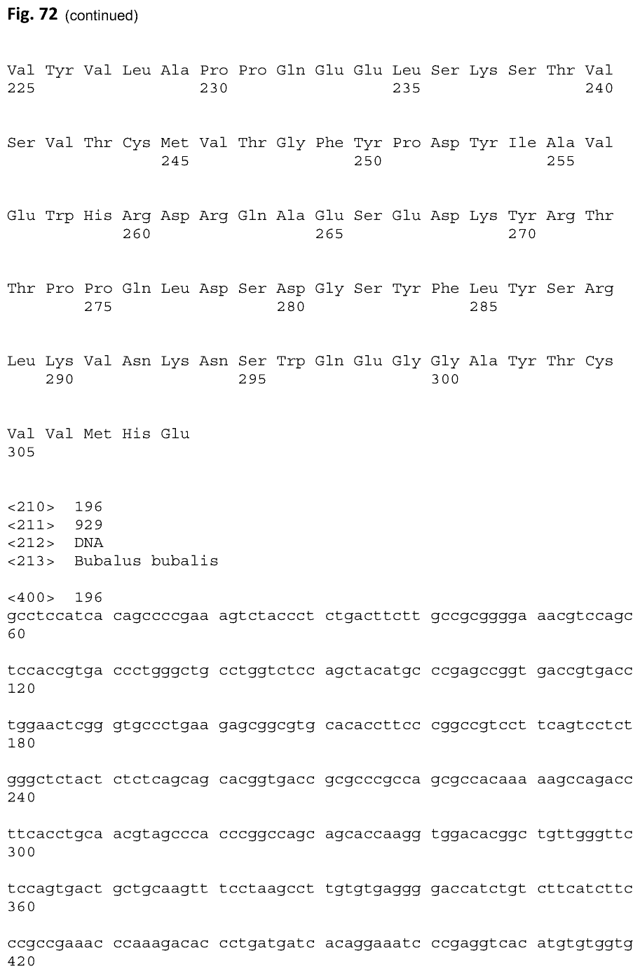

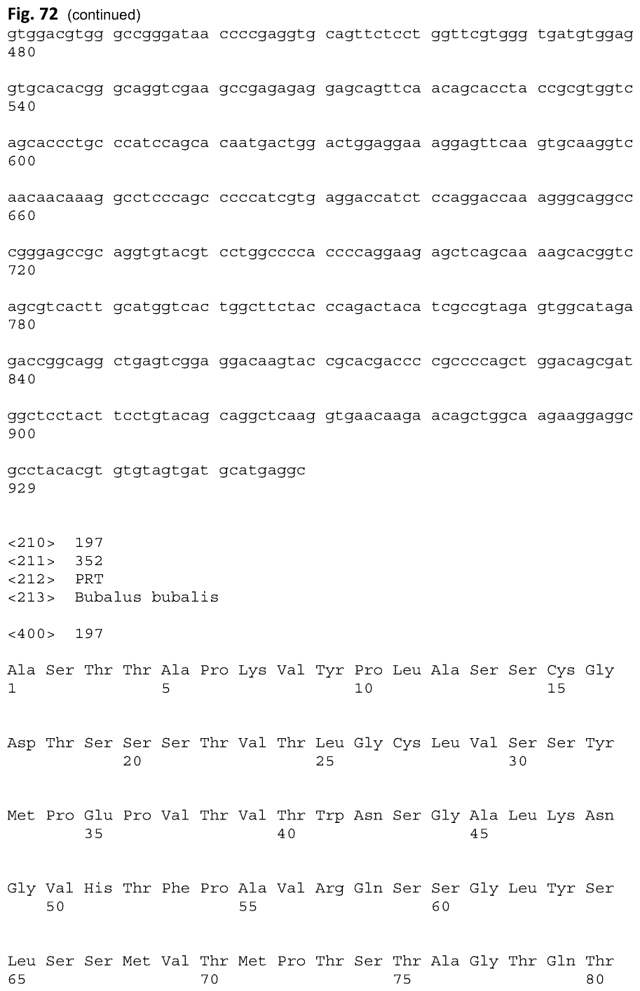

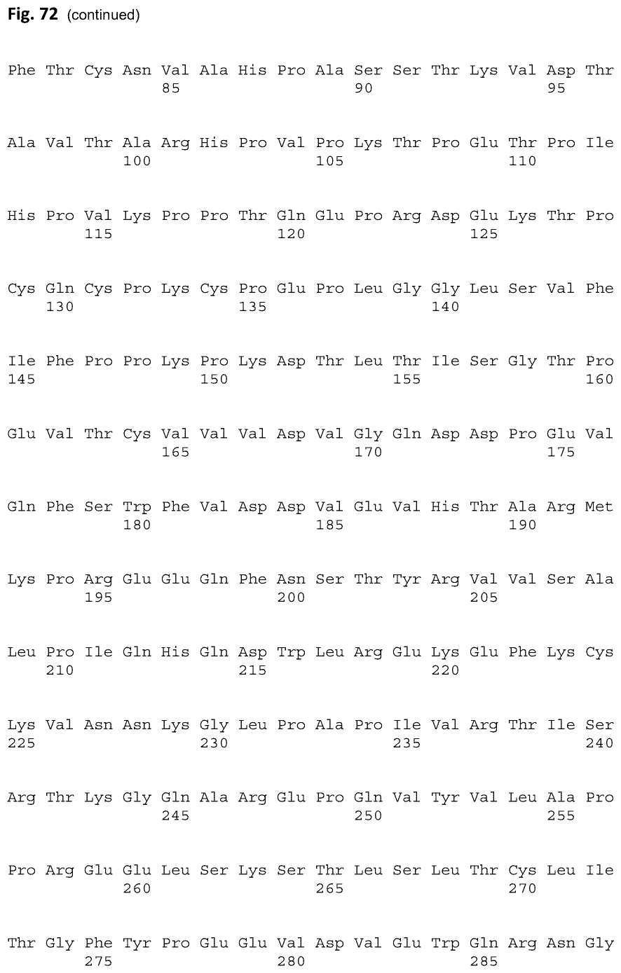

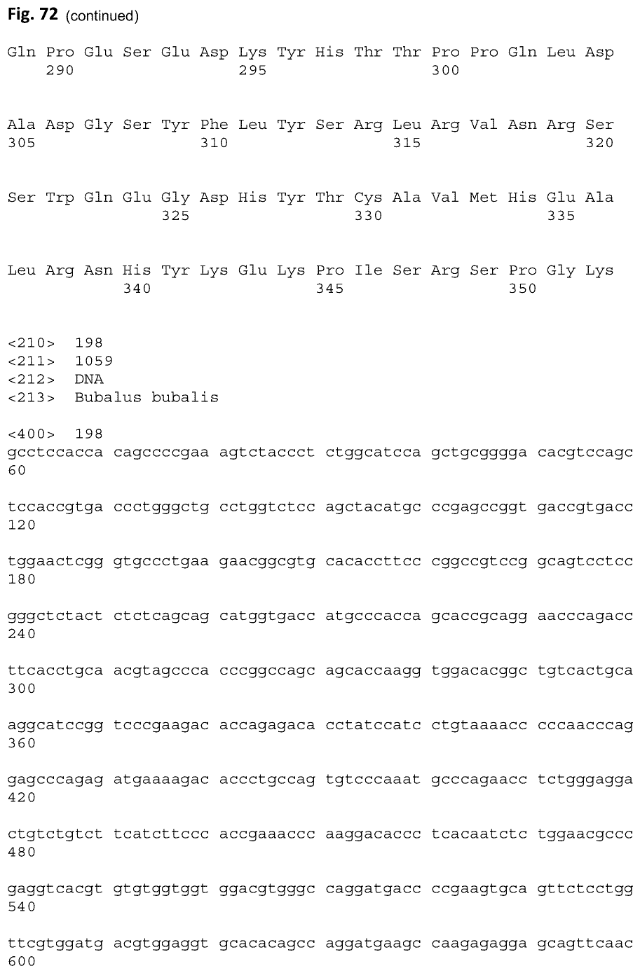

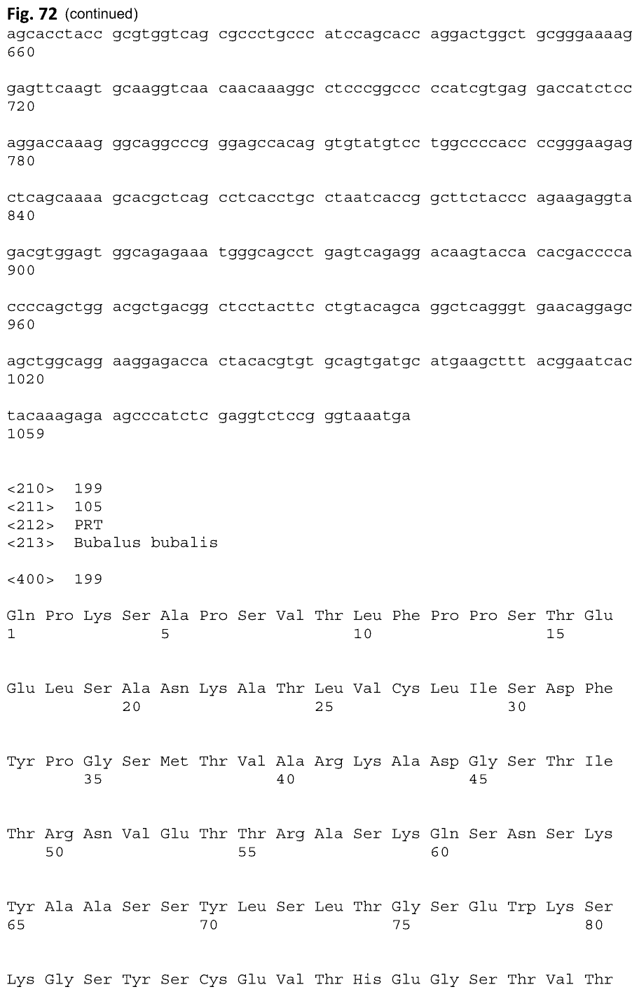

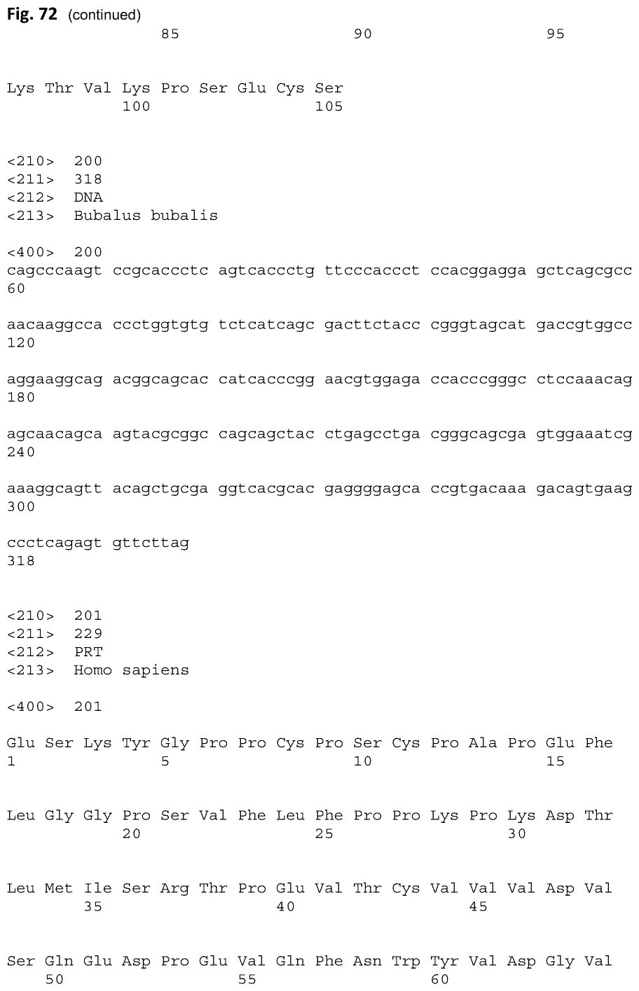

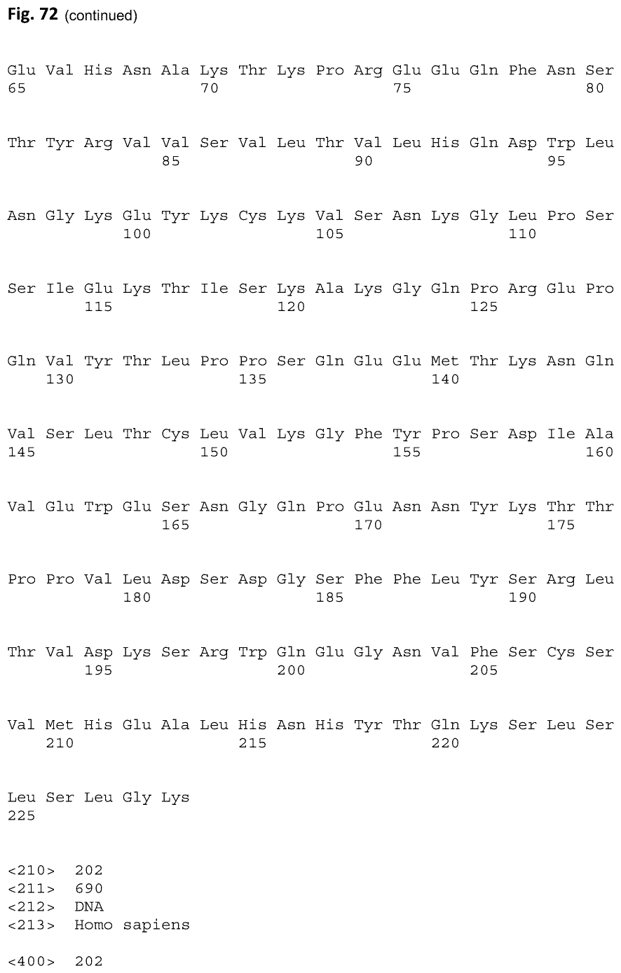

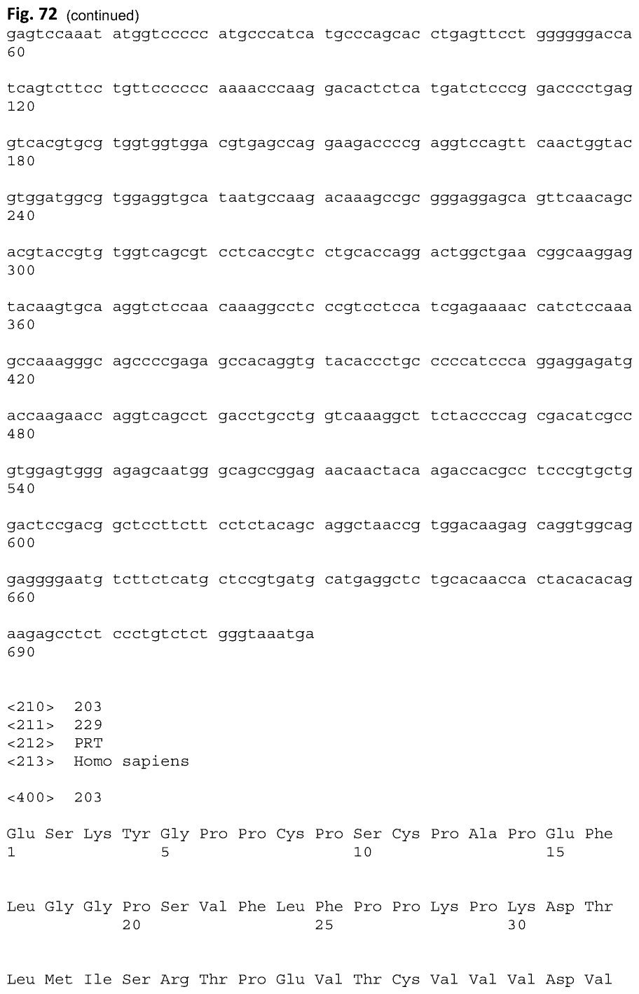

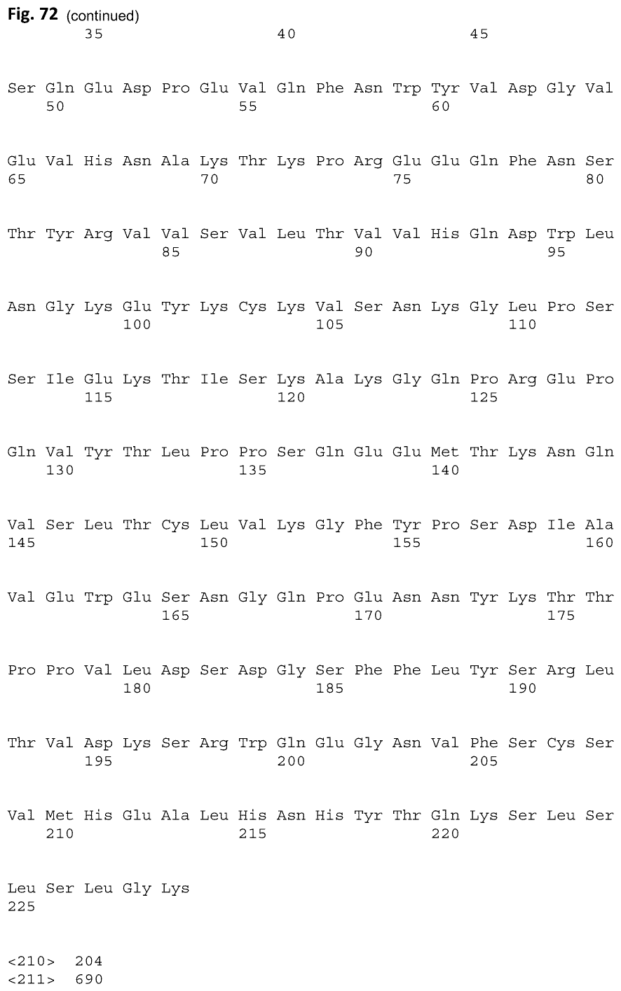

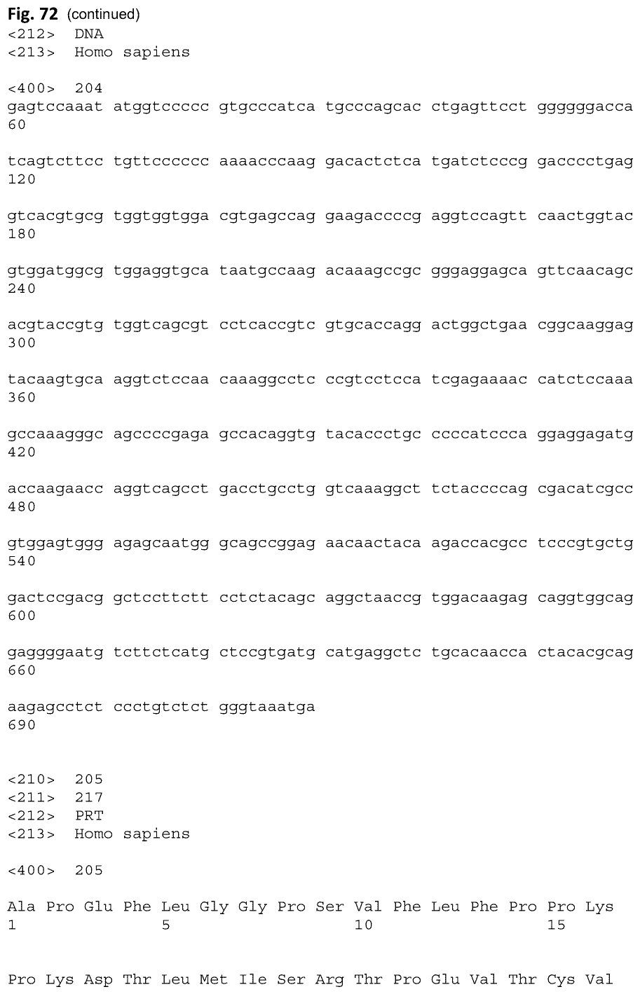

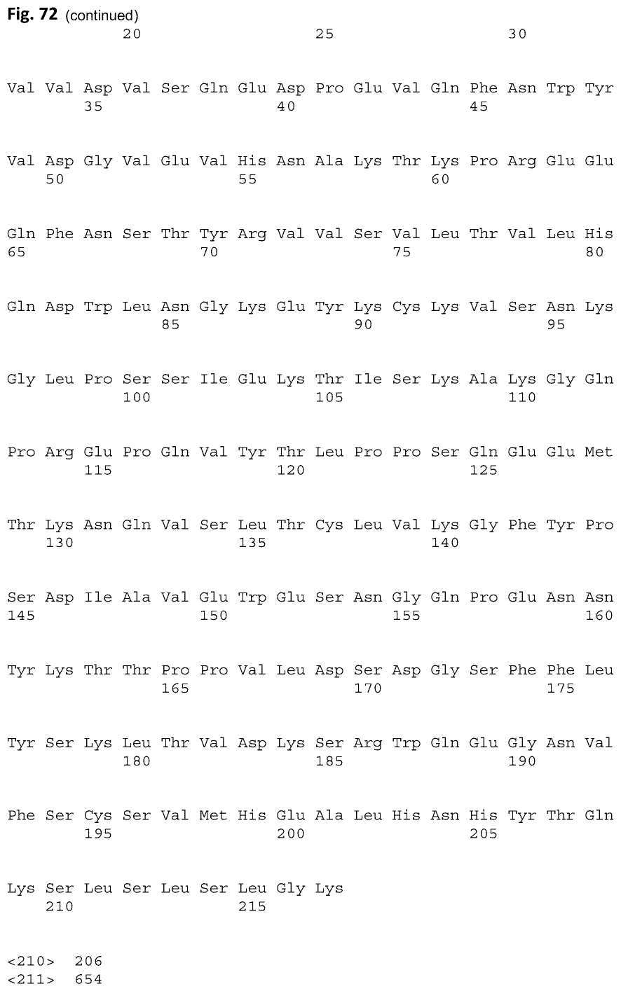

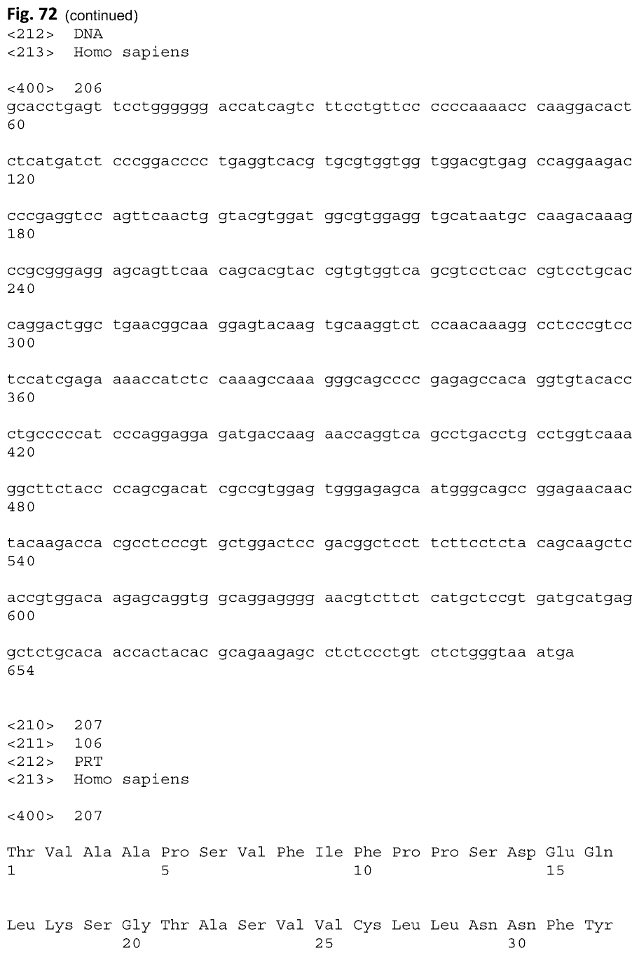

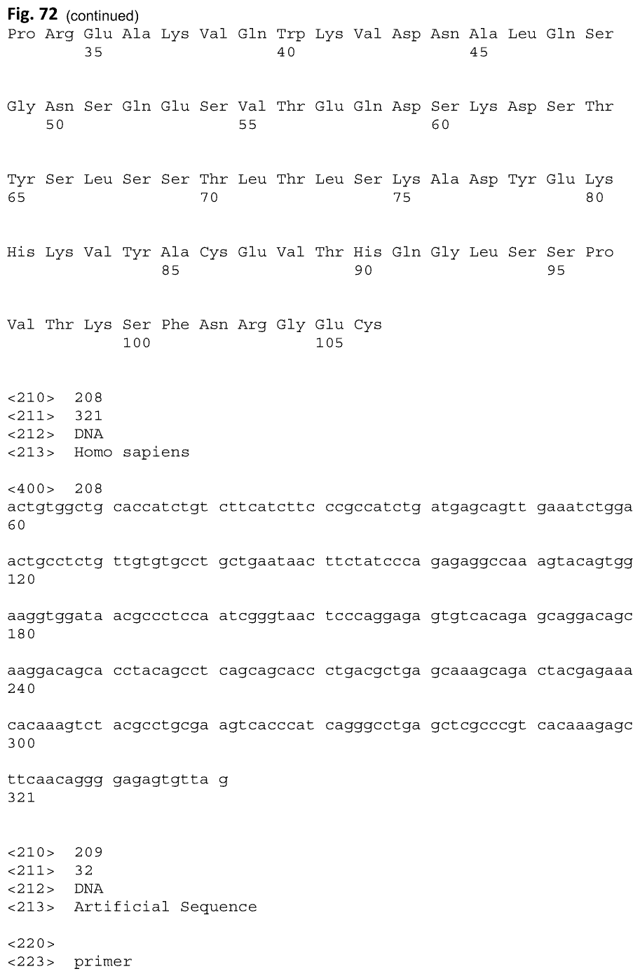

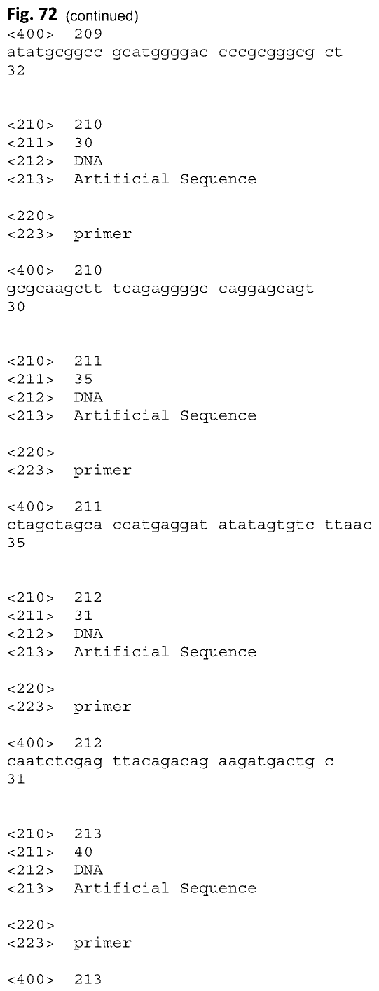

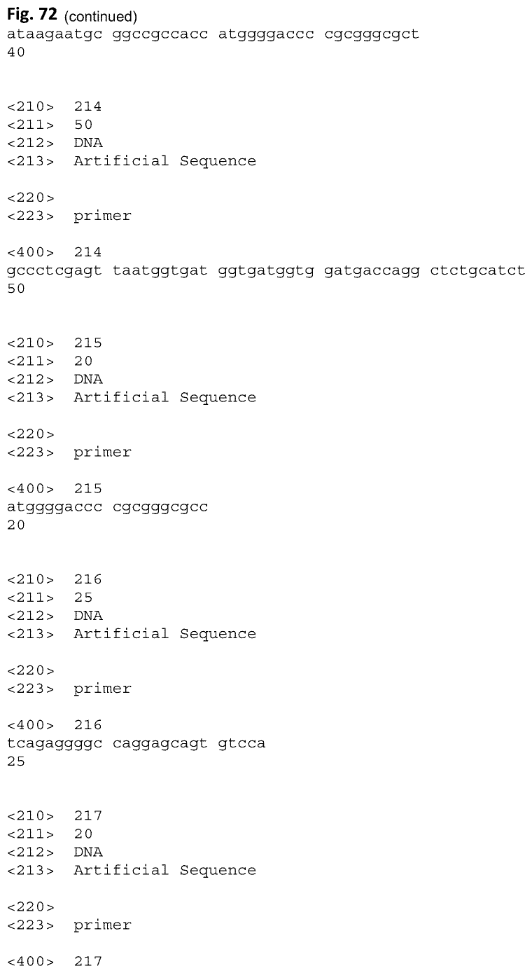

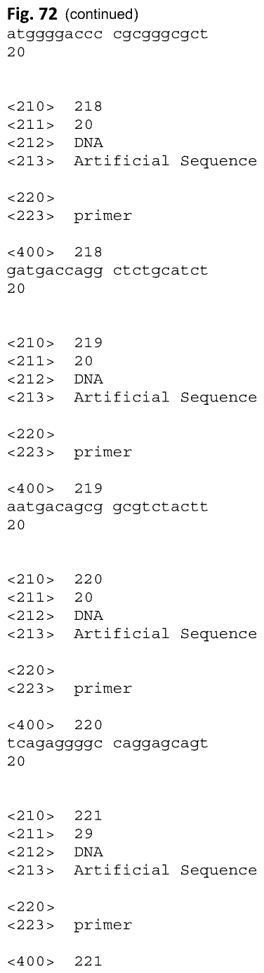

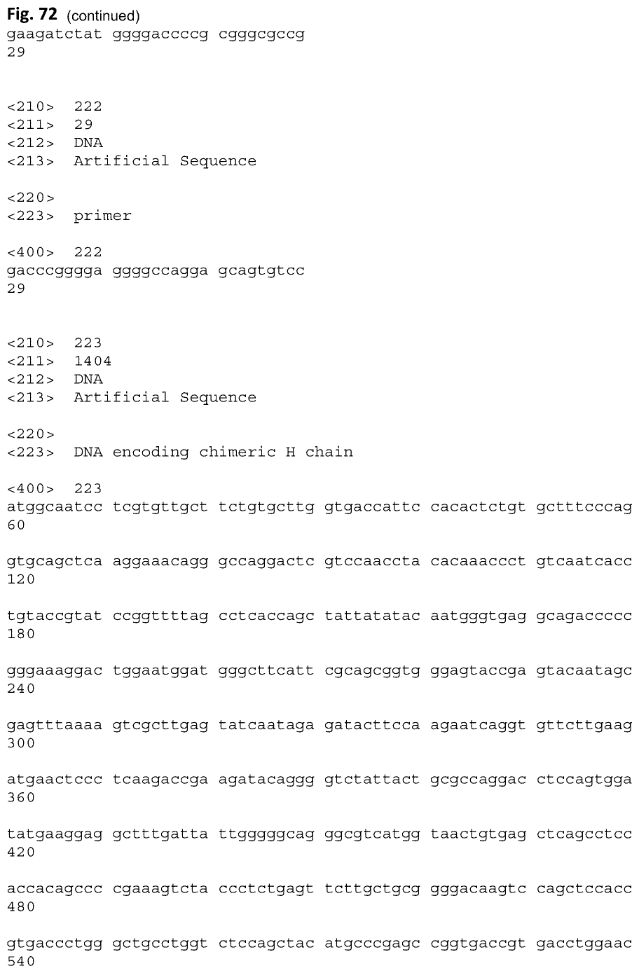

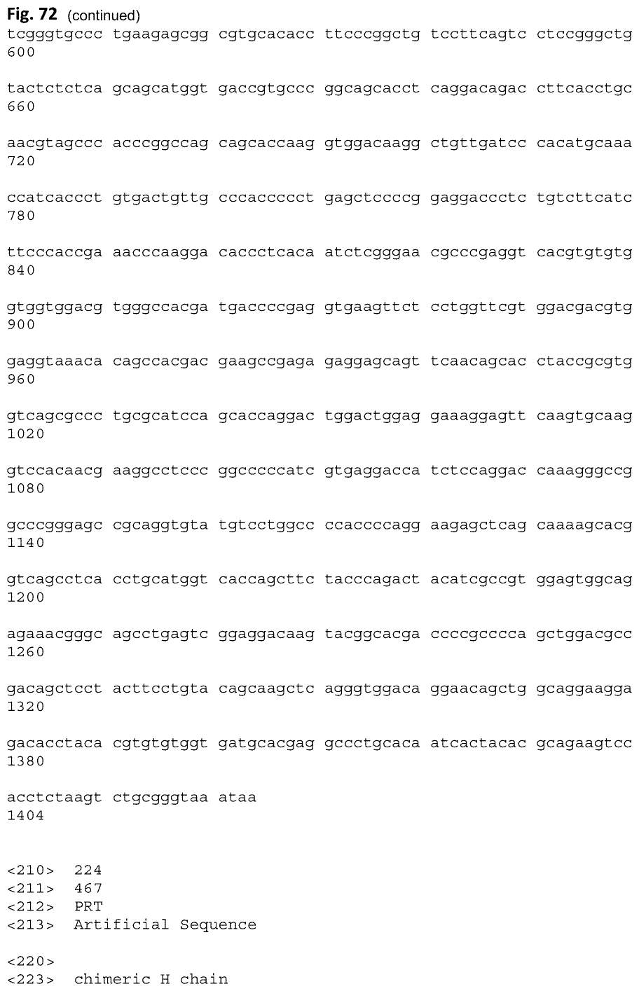

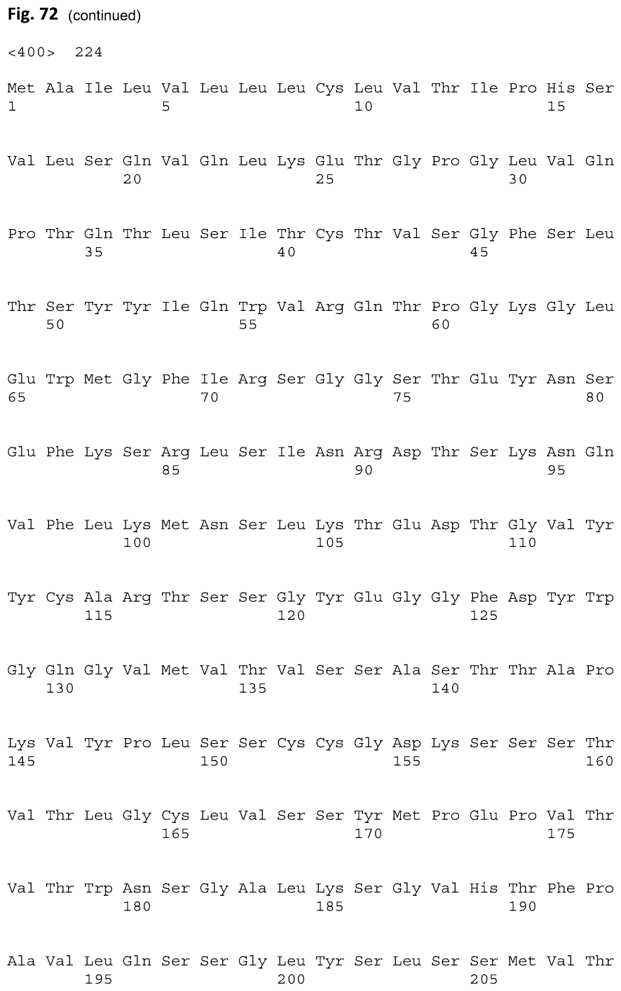

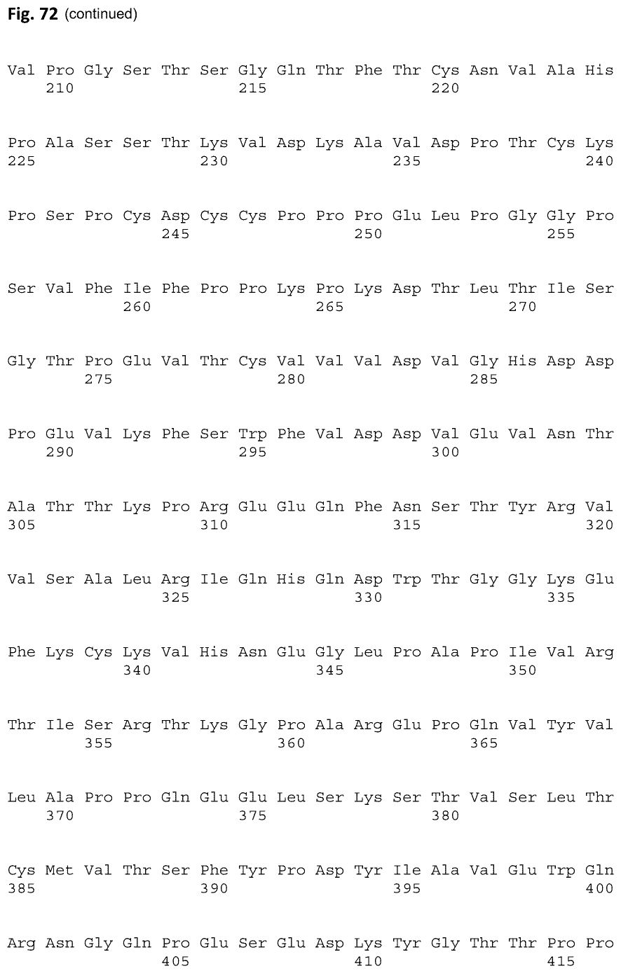

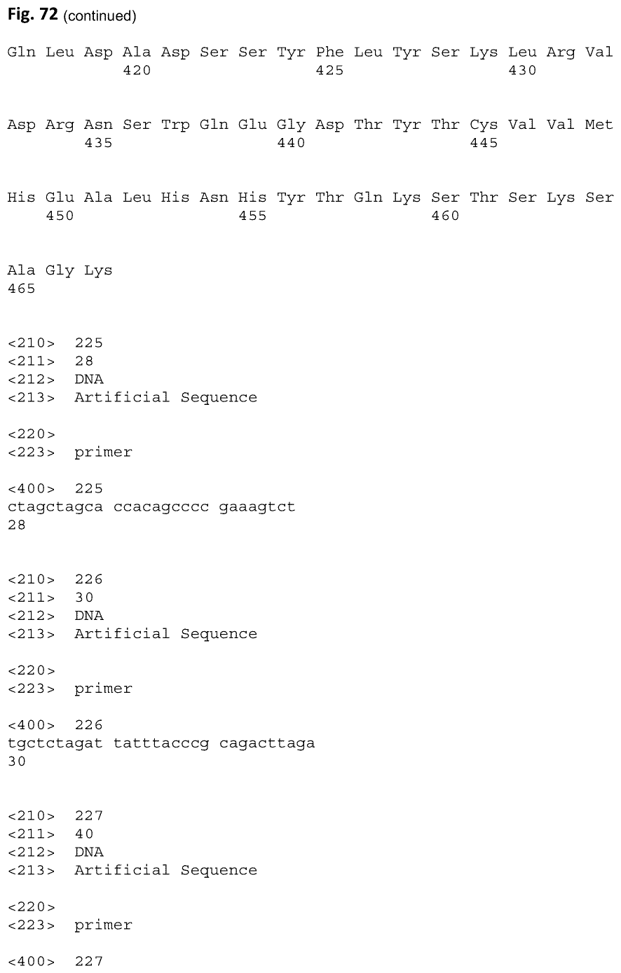

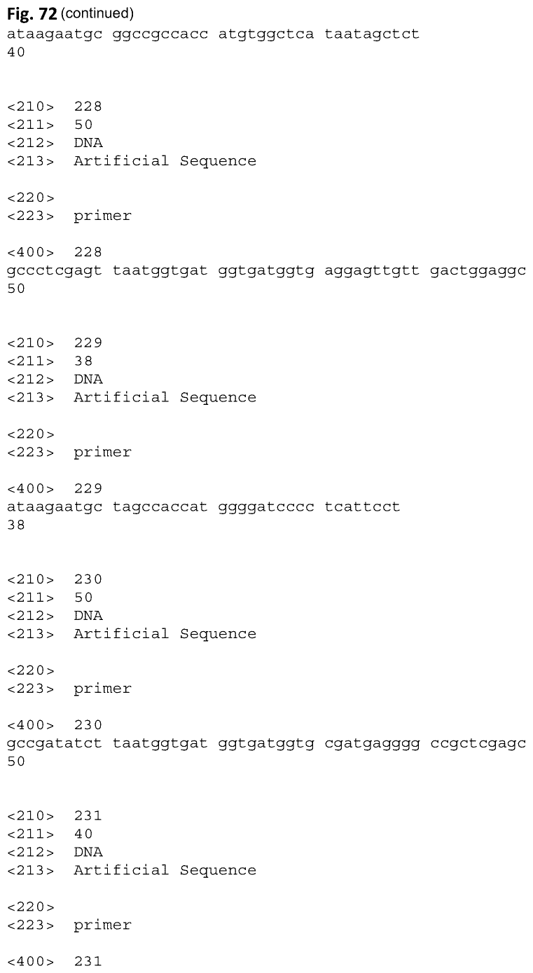

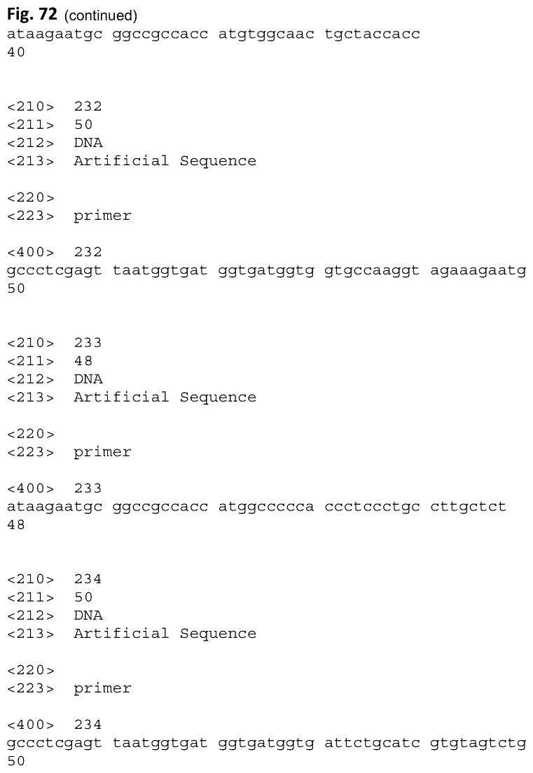

2.5. Binding to Bovine FcγRs of Rat-Bovine Chimeric Anti-Bovine PD-1 Antibody ch5D2 IgG1 WT and IgG1 ADCC—(FIG. 71)

Rat-bovine chimeric anti-bovine PD-1 antibody ch5D2 IgG1 WT or IgG1 ADCC—was immobilized on Nunc MaxiSorp ELISA plates (Nunc) at a final concentration of 50, 25, 12.5, 6.25, 3.12 or 1.5610 nM at 37° C. for 2 hr. Subsequently, each well was washed with 200 μl of 0.05% Tween 20-supplemented PBS (PBS-T) five times, followed by blocking with SuperBlock (PBS) Blocking Buffer (Thermo Fisher Scientific) at 37° C. for 30 min. Each well was washed again in the same manner. Then, bovine FcγRI-His, FcγRII-His, FcγRIII-His or Fcγ2R-His was added to each well at a final concentration of 10 μg/ml and reacted at 37° C. for 1 hr. After washing, anti-polyhistidine tag mouse monoclonal antibody (Abcam) was reacted at 37° C. for 30 min. Subsequently, each well was washed, and horseradish peroxidase-labeled anti-mouse IgG goat polyclonal antibody (MP Biomedicals) was reacted at 37° C. for 30 min. Each well was washed again, and then TMB One Component Substrate (Bethyl) was added for coloring. Thereafter, the enzyme reaction was terminated with 0.18 M dilute sulfuric acid, and absorbance (450 nm) was measured with Microplate Reader MTP-900 (Corona Electric). For every plate washing operation, Auto Plate Washer BIO WASHER 50 (DS Pharma Biomedical) was used.

The experimental results are shown in FIG. 71. IgG1 WT strongly bound to bovine FcγRI-His and weakly bound to bovine FcγRII-His. On the other hand, IgG1 ADCC—did not bind to bovine FcγRI-His or FcγRII-His. Neither IgG1 WT nor IgG1 ADCC—bound to bovine FcγRIII-H is or Fcγ2R-His.

All publications, patents and patent applications cited herein are incorporated herein by reference in their entirety.

### INDUSTRIAL APPLICABILITY

The anti-PD-L1 antibody of the present disclosure is applicable to prevention and/or treatment of cancers and infections in animals. Further, the pharmaceutical composition of the present disclosure comprising a COX-2 inhibitor and an inhibitor targeting PD-1/PD-L1 is applicable to prevention and/or treatment of cancer and/or infection.

1. A pharmaceutical composition which comprises a COX-2 inhibitor and is administered before, after or simultaneously with the administration of an inhibitor targeting PD-1/PD-L1.

2. The pharmaceutical composition of claim 1, wherein the inhibitor targeting PD-1/PD-L1 is an antibody.

3. The pharmaceutical composition of claim 1, wherein the antibody is at least one antibody selected from the group consisting of anti-PD-1 antibody and anti-PD-L1 antibody.

4. The pharmaceutical composition of claim 1, wherein the COX-2 inhibitor is at least one compound selected from the group consisting of meloxicam, piroxicam, celecoxib, firocoxib, robenacoxib, carprofen and etodolac.

5. The pharmaceutical composition of claim 1 for use in prevention and/or treatment of cancer and/or infection.

6. The pharmaceutical composition of claim 1, wherein the inhibitor targeting PD-1/PD-L1 and the COX-2 inhibitor are administered separately.

7. The pharmaceutical composition of claim 1, which is a combination drug comprising the inhibitor targeting PD-1/PD-L1 and the COX-2 inhibitor.

8. (canceled)

9. A method of preventing and/or treating cancer and/or infection, comprising administering to a human or animal subject a pharmaceutically effective amount of a COX-2 inhibitor before, after or simultaneously with the administration of an inhibitor targeting PD-1/PD-L1.

10. An anti-PD-1 antibody comprising (a) a light chain comprising a light chain variable region containing CDR1 having the amino acid sequence of QSLEYSDGYTY (SEQ ID NO: 16), CDR2 having the amino acid sequence of GVS and CDR3 having the amino acid sequence of FQATHDPDT (SEQ ID NO: 165) and the light chain constant region of an antibody of an animal other than rat; and (b) a heavy chain comprising a heavy chain variable region containing CDR1 having the amino acid sequence of GFSLTSYY (SEQ ID NO: 166), CDR2 having the amino acid sequence of IRSGGST (SEQ ID NO: 167) and CDR3 having the amino acid sequence of ARTSSGYEGGFDY (SEQ ID NO: 168) and the heavy chain constant region of an antibody of an animal other than rat.

11. The antibody of claim 10, wherein the light chain variable region and the heavy chain variable region are derived from rat.

12. The antibody of claim 11, wherein the light chain variable region is the light chain variable region of a rat anti-bovine PD-1 antibody and the heavy chain variable region is the heavy chain variable region of a rat anti-bovine PD-1 antibody.

13. The antibody of claim 12, wherein the light chain variable region has the amino acid sequence as shown in SEQ ID NO. 149 and the heavy chain variable region has the amino acid sequence as shown in SEQ ID NO: 150.

14. The antibody of claim 10, wherein the light chain constant region of an antibody of an animal other than rat has the amino acid sequence of the constant region of lambda chain or kappa chain.

15. The antibody of claim 10, wherein the heavy chain constant region of an antibody of an animal other than rat has the amino acid sequence of the constant region of an immunoglobulin equivalent to human IgG4, or has mutations introduced thereinto that reduce ADCC activity and/or CDC activity.

16. The antibody of claim 15, wherein the animal other than rat is bovine; the light chain constant region of the bovine antibody has the amino acid sequence of the constant region of lambda chain; and the heavy chain constant region of the bovine antibody has mutations introduced thereinto that reduce ADCC activity and/or CDC activity.

17. The antibody of claim 16, wherein the light chain constant region of the bovine antibody has the amino acid sequence as shown in SEQ ID NO: 151 and the heavy chain constant region of the bovine antibody has the amino acid sequence as shown in SEQ ID NO: 152.

18. The antibody of claim 10 which has a four-chain structure comprising two light chains and two heavy chains.

19. A pharmaceutical composition comprising the antibody of claim 10 as an active ingredient.

20. The composition of claim 19 for prevention and/or treatment of cancers and/or inflammations.

21-27. (canceled)
